(19) United States
(12) Patent Application Publication
(10) Pub. No.: US 2020/0017566 A1
(43) Pub. Date: Jan. 16, 2020

(54) HEPCIDIN AND MINI-HEPCIDIN ANALOGUES AND USES THEROF

(71) Applicant: PROTAGONIST THERAPEUTICS, INC., NEWARK, CA (US)

(72) Inventors: Gregory Thomas BOURNE, Moggill (AU); Mark Leslie SMYTHE, Bardon (AU); Brian Troy FREDERICK, Ben Lomond, CA (US); Simone VINK, Taringa (AU)

(21) Appl. No.: 16/439,435

(22) Filed: Jun. 12, 2019

Related U.S. Application Data
(63) Continuation of application No. 15/321,124, filed on Dec. 21, 2016, now abandoned.
(60) Provisional application No. 62/018,382, filed on Jun. 27, 2014.

(51) Int. Cl. C07K 14/575

(52) U.S. Cl. CPC C07K 14/575; A61K 38/00

(57) ABSTRACT

The present invention provides novel hepcidin analogues, and related methods of using these hepcidin analogues to treat or prevent a variety of diseases and disorders, including iron overload diseases such as hereditary hemochromatosis, iron-loading anemias, and other conditions and disorders described herein.

### CROSS REFERENCE TO RELATED APPLICATIONS

This application is a continuation of application Ser. No. 15/321,124, filed Jun. 29, 2015, which is a § 371 national phase application of International Application No. PCT/US2015/038370, filed Jun. 29, 2015, which claims priority to U.S. Provisional Application No. 62/018,382, filed on Jun. 27, 2014, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is PRTH_005_02US_ST25.txt. The text file is 244 KB, created on Aug. 29, 2019, and is being submitted electronically via EFS-Web.

### FIELD OF THE INVENTION

The present invention relates, inter alia, to certain hepcidin peptide analogues, including both peptide monomers and peptide dimers, and conjugates and derivatives thereof, as well as compositions comprising the peptide analogues, and to the use of the peptide analogues in the treatment and/or prevention of a variety of diseases, conditions or disorders, including treatment and/or prevention of iron overload diseases such as hereditary hemochromatosis, iron-loading anemias, and other conditions and disorders described herein.

### BACKGROUND

Hepcidin (also referred to as LEAP-1), a peptide hormone produced by the liver, is a regulator of iron homeostasis in humans and other mammals. Hepcidin acts by binding to its receptor, the iron export channel ferroportin, causing its internalization and degradation. Human hepcidin is a 25-amino acid peptide (Hep25). See Krause et al. (2000) FEBS Lett 480:147-150, and Park et al. (2001) J Biol Chem 276:7806-7810. The structure of the bioactive 25-amino acid form of hepcidin is a simple hairpin with 8 cysteines that form 4 disulfide bonds as described by Jordan et al. J Biol Chem 284:24155-67. The N terminal region is required for iron-regulatory function, and deletion of 5 N-terminal amino acid residues results in a loss of iron-regulatory function. See Nemeth et al. (2006) Blood 107:328-33.

Abnormal hepcidin activity is associated with iron overload diseases, including hereditary hemochromatosis (HH) and iron-loading anemias. Hereditary hemochromatosis is a genetic iron overload disease that is mainly caused by hepcidin deficiency or in some cases by hepcidin resistance. This allows excessive absorption of iron from the diet and development of iron overload. Clinical manifestations of HH may include liver disease (e.g., hepatic cirrhosis and hepatocellular carcinoma), diabetes, and heart failure. Currently, the only treatment for HH is regular phlebotomy, which is very burdensome for the patients. Iron-loading anemias are hereditary anemias with ineffective erythropoiesis such as 3-thalassemia, which are accompanied by severe iron overload. Complications from iron overload are the main cause of morbidity and mortality for these patients. Hepcidin deficiency is the main cause of iron overload in non-transfused patients, and contributes to iron overload in transfused patients. The current treatment for iron overload in these patients is iron chelation which is very burdensome, sometimes ineffective, and accompanied by frequent side effects.

Hepcidin has a number of limitations which restrict its use as a drug, including a difficult synthesis process due in part to aggregation and precipitation of the protein during folding, which in turn leads to high cost of goods. What are needed in the art are compounds having hepcidin activity and also possessing other beneficial physical properties such as improved solubility, stability, and/or potency, so that hepcidin-like biologics might be produced affordably, and used to treat hepcidin-related diseases and disorders such as, e.g., those described herein.

The present invention addresses such needs, providing novel peptide analogues, including both peptide monomer analogues and peptide dimer analogues, having hepcidin activity and also having other beneficial properties making the peptides of the present invention suitable alternatives to hepcidin.

### BRIEF SUMMARY OF THE INVENTION

The present invention generally relates to peptide analogues, including both monomer and dimers, exhibiting hepcidin activity and methods of using the same.

In some embodiments, the invention provides peptides, which may be isolated and/or purified, comprising, consisting essentially of, or consisting of, the following structural formula I:

(I)

(SEQ ID NO: 1)

R¹-X-Y-R²

or a pharmaceutically acceptable salt or solvate thereof,

wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;

R² is OH or NH₂;

X is a peptide sequence having the formula Ia:

(Ia)

(SEQ ID NO: 2)

X1-X2-X3-X4-X5-X6-X7-X8-X9-X10

wherein

X1 is Asp, Ser, Glu, Ida, pGlu, bhAsp, D-Asp or absent;
X2 is Thr, Ser, Lys, Glu, Pro, Ala or absent;

### X3 is His, Ala, or Glu;

### X4 is Phe, Ile or Dpa;

X5 is Pro, bhPro, Val, Glu, Sarc or Gly;

### X6 is Cys or (D)-Cys;

X7 is absent or any amino acid except Ile, Cys or (D)-Cys;
X8 is absent or any amino acid except Cys or (D)-Cys;
X9 is Phe, Ala, Ile, Thr, Tyr, Lys, Arg, bhPhe, D-Phe or absent; and
X10 is Lys, Phe or absent; and

Y is absent or present;

provided that if Y is present, Y is a peptide having the formula Im:

(Im)

(SEQ ID NO: 3)

Y1-Y2-Y3-Y4-Y5-Y6-Y7-Y8-Y9-Y10-Y11-Y12

wherein

Y1 is Gly, PEG3, Sarc, Lys, Glu, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Glu, Tyr or absent;
Y5 is Lys, Met, Ser, Arg, Ala or absent;
Y6 is Gly, Sarc, Glu, Lys, Arg, Ser, Lys, Ile, Ala, Pro, Val or absent;
Y7 is Trp, Lys, Gly, Ala, Ile, Val or absent;
Y8 is Val, Trp, His, Thr, Gly, Cys, Met, Tyr, Ala, Glu, Lys, Asp, Arg or absent;
Y9 is Val, Asp, Asn, Cys, Tyr or absent;
Y10 is Cys, Met, Lys, Arg, Tyr or absent;
Y11 is Arg, Met, Cys, Lys or absent; and
Y12 is Arg, Lys, Ala or absent.

In one alternative embodiment, the present invention provides a hepcidin analogue peptide of formula Ia, wherein X5 is Pro, bhPro, Val, Glu, Sarc, Gly, or any N-methylated amino acid.

In one embodiment, the invention provides peptides, which may be isolated and/or purified, comprising, consisting essentially of, or consisting of formula I, wherein X is a peptide sequence having the formula Ib:

(Ib)

SEQ ID NO: 18

X1-X2-X3-X4-X5-X6-X7-X8-X9-X10

wherein

X1 is Asp, Glu, Ida, pGlu, bhAsp, D-Asp or absent;
X2 is Thr, Ser, Lys, Glu, Pro, Ala or absent;

### X3 is His, Ala, or Glu;

### X4 is Phe, Ile or Dpa;

X5 is Pro, bhPro, Sarc or Gly;

### X6 is Cys;

X7 is absent or any amino acid except Ile, Cys or (D)-Cys;
X8 is absent or any amino acid except Cys or (D)-Cys;
X9 is Phe, Ile, Tyr, bhPhe or D-Phe or absent; and
X10 is Lys, Phe or absent; and

wherein Y is absent or present, provided that if Y is present, Y is a peptide having the formula In:

(In)

SEQ ID NO: 19

Y1-Y2-Y3-Y4-Y5-Y6-Y7-Y8-Y9-Y10-Y11-Y12

wherein

Y1 is Gly, PEG3, Sarc, Lys, Glu, Ala, Phe, Pro, Glu, Lys, D-Pro, Val, Ser or absent;
Y2 is Pro, Ala, Gly or absent;
Y3 is Arg, Lys, Pro, Gly, His, Ala, or absent;
Y4 is Ser, Arg, Glu or absent;
Y5 is Lys, Ser, Met, Arg, Ala or absent;
Y6 is Gly, Sarc, Glu, Leu, Phe, His or absent;
Y7 is Trp, N-Methyl Trp, Lys, Thr, His, Gly, Ala, Ile, Val or absent;
Y8 is Val, Trp, Ala, Asn, Glu or absent;
Y9 is Val, Ala, Asn, Asp, Cys or absent;
Y10 is Cys, (D)Cys, Glu or absent;
Y11 is Tyr, Met or absent; and
Y12 is Trp or absent.

In related embodiments, the invention provides peptides, which may be isolated and/or purified, comprising, consisting essentially of, or consisting of, the following structural formula II:

(II)

(SEQ ID NO: 4)

R¹-X-Y-R²

or a pharmaceutically acceptable salt or solvate thereof,

wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;

R² is OH or NH₂;

X is a peptide sequence having the formula IIa:

(IIa)

(SEQ ID NO: 5)

X1-X2-X3-X4-X5-X6-X7-X8-X9-X10

wherein

### X1 is Asp, Glu or Ida;

X2 is Thr, Ser or absent;

### X3 is His;

### X4 is Phe or Dpa;

X5 is Pro, bhPro, Sarc or Gly;

### X6 is Cys or D-Cys;

X7 is Arg, Glu, Phe, Gln, Leu, Val, Lys, Ile, Ala, Ser, Dapa or absent;
X8 is Ile, Arg, Lys, Arg, Ala, Gln, Phe, Glu, Asp, Tyr, Ser, Leu, Val, D-Ile, D-Lys, D-Arg, Dapa or absent;
X9 is Phe, Tyr, bhPhe, D-Phe or absent; and
X10 is Lys, Phe or absent; and

wherein Y is absent or present, provided that if Y is present, Y is a peptide having the formula IIm:

(IIm)

(SEQ ID NO: 6)

Y1-Y2-Y3-Y4-Y5-Y6-Y7-Y8-Y9-Y10-Y11-Y12

wherein

Y1 is Gly, Sarc, Lys, Glu or absent;
Y2 is Pro, Ala, Gly or absent;
Y3 is Arg, Lys, Pro, Gly, His, Ala or absent;
Y4 is Ser, Arg, Glu or absent;
Y5 is Lys, Ser, Met, Arg, Ala or absent;
Y6 is Gly, Sarc, Glu, Leu, Phe, His or absent;
Y7 is Trp, N-MethylTrp, Lys, Thr, His, Gly, Ala, Ile, Val or absent;
Y8 is Val, Trp, Ala, Asn, Glu or absent;

### Y9 is Cys;

Y10 is Met or absent;
Y11 is Tyr, Met, or absent; and
Y12 is Trp or absent.

In certain embodiments, X6 in formula IIa is Cys.

In certain alternative embodiments, X7 in formula IIa is Arg, Glu, Phe, Gln, Leu, Val, Lys, Ala, Ser, Dapa or absent.

In certain embodiments, Y10 is absent.

In certain embodiments, Y11 is absent.

In certain embodiments, Y12 is absent.

In other related embodiments, the invention provides peptide homo- or heterodimers, which may be isolated and/or purified, comprising two hepcidin analogues, each hepcidin analogue comprising, consisting essentially of, or consisting of the structure of Formula I, the structure of Formula II, the structure of Formula III, the structure of Formula IV, the structure of Formula V, the structure of Formula VI, the structure of Formula VII, the structure of Formula VIII, the Structure of Formula IX, the structure of Formula X, or a sequence or structure shown in any one of Tables 2-4, 6-10, 12, 14, or 15, provided that when the dimer comprises a hepcidin analogue having the structure of Formula III, Formula IV, Formula V, or Formula VI, the two hepcidin analogues are linked via a lysine linker.

In certain embodiments, a hepcidin analogue dimer of the present invention is dimerized by more than one means. In particular embodiments, a hepcidin analogue dimer of the present invention is dimerized by at least one intermolecular disulfide bridge and at least one linker moiety (e.g., an IDA linker, such as an IDA-Palm). In particular embodiments, a hepcidin analogue dimer of the present invention is dimerized by at least one intermolecular disulfide bridge and at least one linker moiety (e.g., an IDA linker, such as an IDA-Palm), wherein the linker moiety is attached to a lysine residue in each of the peptide monomers.

In certain embodiments, one or both hepcidin analogue has the Formula III:

(III)

(SEQ ID NO: 7)

R¹-X-Y-R²

or a pharmaceutically acceptable salt or solvate thereof, wherein

R¹ is hydrogen, a C1-C6 alkyl, a C6-C12 aryl, a C6-C12 aryl C1-C6 alkyl, or a C1-C20 alkanoyl, and including PEGylated versions thereof, alone or as spacers of any of the foregoing;

R² is —NH₂ or —OH;

X is a peptide sequence having the formula (IIIa)

(IIIa)

(SEQ ID NO: 8)

X1-X2-X3-X4-X5-X6-X7-X8-X9-X10

wherein

X1 is Asp, Glu, Ala, Gly, Thr, Ida, pGlu, bhAsp, D-Asp, Tyr, Leu or absent;
X2 is Thr, Ala, Aib, D-Thr, Arg or absent;

### X3 is His, Lys, Ala, or D-His;

X4 is Phe, Ala, Dpa or bhPhe;
X5 is Pro, Glu, Ser, Gly, Arg, Lys, Val, Ala, D-Pro, bhPro, Sarc, Abu or absent;

### X6 is Ile, Cys, Arg, Leu, Lys, His, Glu, D-Ile, D-Arg, D-Cys, Val, Ser or Ala;

### X7 is Cys, Ile, Ala, Leu, Val, Ser, Phe, Dapa, D-Ile or D-Cys;

### X8 is Ile, Lys, Arg, Ala, Gln, Phe, Glu, Asp, Tyr, Ser, Leu, Val, D-Ile, D-Lys, D-Arg, or Dapa;

X9 is Phe, Ala, Ile, Tyr, Lys, Arg, bhPhe or D-Phe; and
X10 is Lys, Phe or absent; and

Y is absent or present, and when present, Y is a peptide having the formula (IIIm)

(IIIm)

(SEQ ID NO: 9)

Y1-Y2-Y3-Y4-Y5-Y6-Y7-Y8-Y9-Y10-Y11-Y12-Y13-Y14-Y15

wherein

Y1 is Gly, Cys, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Tyr or absent;
Y5 is Lys, Met, Arg, Ala or absent;
Y6 is Gly, Ser, Lys, Ile, Arg, Ala, Pro, Val or absent;
Y7 is Trp, Lys, Gly, Ala, Ile, Val or absent;
Y8 is Val, Thr, Gly, Cys, Met, Tyr, Ala, Glu, Lys, Asp, Arg or absent;
Y9 is Cys, Tyr or absent;
Y10 is Met, Lys, Arg, Tyr or absent;
Y11 is Arg, Met, Cys, Lys or absent;
Y12 is Arg, Lys, Ala or absent;
Y13 is Arg, Cys, Lys, Val or absent;
Y14 is Arg, Lys, Pro, Cys, Thr or absent; and
Y15 is Thr, Arg or absent;

wherein if Y is absent from the peptide of formula (III), X7 is Ile; and

wherein said compound of formula (III) is optionally PEGylated on R¹, X, or Y.

In certain embodiments, one or both hepcidin analogue has the structure of Formula (IV):

R¹—X—Y—R² (IV) (SEQ ID NO:10)

or a pharmaceutically acceptable salt or solvate thereof,

wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;

R² is —NH₂ or —OH;

X is a peptide sequence having the formula (IVa)

X1-X2-X3-X4-X5-X6-X7-X8-X9-X10 (IVa) (SEQ ID NO:11)

wherein

X1 is Asp, Glu, Ala, Gly, Thr, Ida, pGlu, bhAsp, D-Asp, Tyr, Leu or absent;
X2 is Thr, Ala, Aib, D-Thr, Arg or absent;

### X3 is His, Lys, Ala, or D-His;

X4 is Phe, Ala, Dpa, bhPhe or D-Phe;
X5 is Pro, Glu, Ser, Gly, Arg, Lys, Val, Ala, D-Pro, bhPro, Sarc, Abu or absent;

### X6 is Ile, Cys, Arg, Leu, Lys, His, Glu, D-Ile, D-Arg, D-Cys, Val, Ser or Ala;

### X7 is Cys, Ile, Ala, Leu, Val, Ser, Phe, Dapa, D-Ile or D-Cys;

### X8 is Ile, Lys, Arg, Ala, Gin, Phe, Glu, Asp, Tyr, Ser, Leu, Val, D-Ile, D-Lys, D-Arg or Dapa;

X9 is Phe, Ala, Ile, Tyr, Lys, Arg, bhPhe or D-Phe; and
X10 is Lys, Phe or absent;

wherein Y is present or absent, and provided that if Y is absent, X7 is Ile; and

Y is a peptide having the formula (IVm):

(IVm)

(SEQ ID NO: 12)

Y1-Y2-Y3-Y4-Y5-Y6-Y7-Y8-Y9-Y10-Y11-Y12-Y13-Y14-Y15

wherein

Y1 is Gly, Cys, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Tyr or absent;
Y5 is Lys, Met, Arg, Ala or absent;
Y6 is Gly, Ser, Lys, Ile, Arg, Ala, Pro, Val or absent;
Y7 is Trp, Lys, Gly, Ala, Ile, Val or absent;
Y8 is Val, Thr, Gly, Cys, Met, Tyr, Ala, Glu, Lys, Asp, Arg or absent;
Y9 is Cys, Tyr or absent;
Y10 is Met, Lys, Arg, Tyr or absent;
Y11 is Arg, Met, Cys, Lys or absent;
Y12 is Arg, Lys, Ala or absent;
Y13 is Arg, Cys, Lys, Val or absent;
Y14 is Arg, Lys, Pro, Cys, Thr or absent; and
Y15 is Thr, Arg or absent;

wherein said compound of formula (IV) is optionally PEGylated on R¹, X, or Y; and

wherein when said compound of formula (IV) comprises two or more cysteine residues, at least two of said cysteine residues being linked via a disulfide bond.

In certain embodiments, one or both hepcidin analogue has the structure of Formula V:

(V)

(SEQ ID NO: 13)

R¹-X-Y-R²

or a pharmaceutically acceptable salt or solvate thereof, wherein

wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;

R² is —NH₂ or —OH;

X is a peptide sequence having the formula (Va):

(Va)

(SEQ ID NO: 14)

X1-X2-X3-X4-X5-X6-X7-X8-X9-X10

wherein

X1 is Asp, Glu, Ala, Gly, Thr, Ida, pGlu, bhAsp, D-Asp, Tyr, Leu or absent;
X2 is Thr, Ala, Aib, D-Thr, Arg or absent;

### X3 is His, Lys, Ala, D-His or Lys;

X4 is Phe, Ala, Dpa, bhPhe or D-Phe;
X5 is Pro, Glu, Ser, Gly, Arg, Lys, Val, Ala, D-Pro, bhPro, Sarc, Abu or absent;

### X6 is Ile, Cys, Arg, Leu, Lys, His, Glu, D-Ile, D-Arg, D-Cys, Val, Ser or Ala;

### X7 is Cys, Ile, Ala, Leu, Val, Ser, Phe, Dapa, D-Ile or D-Cys;

### X8 is Ile, Lys, Arg, Ala, Gln, Phe, Glu, Asp, Tyr, Ser, Leu, Val, D-Ile, D-Lys, D-Arg, or Dapa;

X9 is Phe, Ala, lie, Tyr, Lys, Arg, bhPhe or D-Phe; and
X10 is Lys, Phe or absent;

wherein Y is present or absent, and provided that if Y is absent, X7 is Ile;

wherein said compound of formula V is optionally PEGylated on R¹, X, or Y; and

wherein when said compound of formula V comprises two or more cysteine residues, at least two of said cysteine residues being linked via a disulfide bond.

In certain embodiments, one or both hepcidin analogue has the structure of formula VI:

(VI)

(SEQ ID NO: 15)

R¹-X-Y-R²

or a pharmaceutically acceptable salt or solvate thereof, wherein

wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;

R² is —NH₂ or —OH;

X is a peptide sequence having the formula (VIa):

(VIa)

(SEQ ID NO: 16)

X1-X2-X3-X4-X5-X6-X7-X8-X9-X10

wherein

X1 is Asp, Glu, Ida or absent;
X2 is Thr, Ser, Pro, Ala or absent;

### X3 is His, Ala, or Glu;

### X4 is Phe or Dpa;

X5 is Pro, bhPro, Sarc or Gly;
X6 is Cys, (D)-Cys, Arg, Glu, Phe, Gln, Leu, Val, Lys, Ala, Ser, Dapa or absent;
X7 is Cys, (D)-Cys, Arg, Glu, Phe, Gln, Leu, Val, Lys, Ala, Ser, Dapa or absent;
X8 is Ile, Arg, Lys, Ala, Gln, Phe, Glu, Asp, Tyr, Ser, Leu, Val, D-Ile, D-Lys, D-Arg, Dapa or absent;
X9 is Phe, Ala, Ile, Thr, Tyr, Lys, Arg, bhPhe, D-Phe or absent; and
X10 is Lys, Phe or absent;

Y is absent or present, provided that if Y is present, Y is a peptide having the formula (VIm)

(VIm)

(SEQ ID NO: 17)

Y1-Y2-Y3

wherein

Y1 is Ile, Arg, Lys, Ala, Gln, Phe, Glu, Asp, Tyr, Ser, Leu, Val, D-Ile, D-Lys, D-Arg, Dapa or absent;
Y2 is Phe, Ala, Ile, Thr, Tyr, Lys, Arg, bhPhe or D-Phe or absent; and
Y3 is Lys, Phe or absent.

In one embodiment, the present invention provides peptide homo- or heterodimers, which may be isolated and/or purified, comprising two hepcidin analogues, each hepcidin analogue comprising, consisting essentially of, or consisting of the structure of Formula I or the structure of Formula II, wherein the two hepcidin analogues are linked via an Ida linker (e.g., an IDA-Palm linker), wherein the Ida linker is attached to a lysine (e.g., via a lysine sidechain) in each of the two hepcidin analogues. In one such embodiment, the dimer is a homodimer, and in another embodiment, the dimer is a heterodimer.

In other embodiments, the present invention includes polynucleotide comprising a sequence encoding a hepcidin analogue described herein.

In further embodiments, the present invention includes a vector comprising a polynucleotide comprising a sequence encoding a hepcidin analogue described herein.

In additional embodiments, the present invention includes a pharmaceutical composition comprising a peptide or hepcidin analogue described herein, and a pharmaceutically acceptable carrier, excipient or vehicle.

In related embodiments, the present invention includes method of binding a ferroportin or inducing ferroportin internalization and degradation, comprising contacting the ferroportin with at least one peptide or hepcidin analogue described herein.

In further related embodiments, the present invention includes a method for treating a disease of iron metabolism in a subject comprising providing to the subject an effective amount of at least one peptide or hepcidin analogue described herein.

In another embodiment, the present invention includes a device comprising a peptide or hepcidin analogue described herein, for delivery of the hepcidin analogue, dimer or composition to a subject.

In another related embodiment, the present invention includes a kit comprising at least one peptide or hepcidin analogue described herein, packaged with a reagent, a device, or an instructional material, or a combination thereof.

### BRIEF DESCRIPTION OF THE DRAWINGS

FIG. 1 shows an in vivo dose response of illustrative hepcidin analogues at two concentrations, 300 nmol/kg and 1000 nmol/kg (subcutaneous or “s.c.”; 2 h), in C-57 (mouse) presented as serum iron levels (n=4). The sequences of the hepcidin analogue monomer peptides used in this experiment are shown in Table 14.

### DETAILED DESCRIPTION OF THE INVENTION

The present invention relates generally to hepcidin analogue peptides and methods of making and using the same. In certain embodiments, the hepcidin analogues exhibit one or more hepcidin activity. In certain embodiments, the present invention relates to hepcidin peptide analogues comprising one or more peptide subunit that forms a cyclized structures through an intramolecular bond, e.g., an intramolecular disulfide bond. In particular embodiments, the cyclized structure has increased potency and selectivity as compared to non-cyclized hepcidin peptides and analogies thereof.

### Definitions and Nomenclature

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats). The term “mammal” refers to any mammalian species such as a human, mouse, rat, dog, cat, hamster, guinea pig, rabbit, livestock, and the like.

The term “peptide,” as used herein, refers broadly to a sequence of two or more amino acids joined together by peptide bonds. It should be understood that this term does not connote a specific length of a polymer of amino acids, nor is it intended to imply or distinguish whether the polypeptide is produced using recombinant techniques, chemical or enzymatic synthesis, or is naturally occurring.

The term “peptide analogue,” as used herein, refers broadly to peptide monomers and peptide dimers comprising one or more structural features and/or functional activities in common with hepcidin, or a functional region thereof. In certain embodiments, a peptide analogue includes peptides sharing substantial amino acid sequence identity with hepcidin, e.g., peptides that comprise one or more amino acid insertions, deletions, or substitutions as compared to a wild-type hepcidin, e.g., human hepcidin, amino acid sequence. In certain embodiments, a peptide analogue comprises one or more additional modification, such as, e.g., conjugation to another compound. Encompassed by the term “peptide analogue” is any peptide monomer or peptide dimer of the present invention. In certain instances, a “peptide analog” may also or alternatively be referred to herein as a “hepcidin analogue,” “hepcidin peptide analogue,” or a “hepcidin analogue peptide.”

The recitations “sequence identity”, “percent identity”, “percent homology”, or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using an NWSgapdna.CMP matrix and a gap weight of 40, 50, 60, 70, or 80 and a length weight of 1, 2, 3, 4, 5, or 6. Another exemplary set of parameters includes a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The peptide sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (Nucleic Acids Res. 25:3389-3402, 1997). When utilizing BLAST and Gapped BLAST programs, the default parameters of the respective programs (e.g., XBLAST and NBLAST) can be used.

The term “conservative substitution” as used herein denotes that one or more amino acids are replaced by another, biologically similar residue. Examples include substitution of amino acid residues with similar characteristics, e.g., small amino acids, acidic amino acids, polar amino acids, basic amino acids, hydrophobic amino acids and aromatic amino acids. See, for example, the table below. In some embodiments of the invention, one or more Met residues are substituted with norleucine (Nle) which is a bioisostere for Met, but which, as opposed to Met, is not readily oxidized. Another example of a conservative substitution with a residue normally not found in endogenous, mammalian peptides and proteins is the conservative substitution of Arg or Lys with, for example, omithine, canavanine, aminoethylcysteine or another basic amino acid. In some embodiments, one or more cysteines of a peptide analogue of the invention may be substituted with another residue, such as a serine. For further information concerning phenotypically silent substitutions in peptides and proteins, see, for example, Bowie et. al. Science 247, 1306-1310, 1990. In the scheme below, conservative substitutions of amino acids are grouped by physicochemical properties. I: neutral, hydrophilic, II: acids and amides, III: basic, IV: hydrophobic, V: aromatic, bulky amino acids.

| I | II | III | IV | V |
|---|---|---|---|---|
| A | N | H | M | F |
| S | D | R | L | Y |
| T | E | K | I | W |
| P | Q | | V | |
| G | | | C | |

In the scheme below, conservative substitutions of amino acids are grouped by physicochemical properties. VI: neutral or hydrophobic, VII: acidic, VIII: basic, IX: polar, X: aromatic.

| VI | VII | VIII | IX | X |
|---|---|---|---|---|
| A | E | H | M | F |
| L | D | R | S | Y |
| I | | K | T | W |
| P | | | C | |
| G | | | N | |
| V | | | Q | |

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The 20 “standard,” natural amino acids are listed in the above tables. The “non-standard,” natural amino acids are pyrrolysine (found in methanogenic organisms and other eukaryotes), selenocysteine (present in many noneukaryotes as well as most eukaryotes), and N-formylmethionine (encoded by the start codon AUG in bacteria, mitochondria and chloroplasts). “Unnatural” or “non-natural” amino acids are non-proteinogenic amino acids (i.e., those not naturally encoded or found in the genetic code) that either occur naturally or are chemically synthesized. Over 140 natural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

As is clear to the skilled artisan, the peptide sequences disclosed herein are shown proceeding from left to right, with the left end of the sequence being the N-terminus of the peptide and the right end of the sequence being the C-terminus of the peptide. Among sequences disclosed herein are sequences incorporating a “Hy-” moiety at the amino terminus (N-terminus) of the sequence, and either an “—OH” moiety or an “—NH₂” moiety at the carboxy terminus (C-terminus) of the sequence. In such cases, and unless otherwise indicated, a “Hy-” moiety at the N-terminus of the sequence in question indicates a hydrogen atom, corresponding to the presence of a free primary or secondary amino group at the N-terminus, while an “—OH” or an “—NH₂” moiety at the C-terminus of the sequence indicates a hydroxy group or an amino group, corresponding to the presence of an amido (CONH₂) group at the C-terminus, respectively. In each sequence of the invention, a C-terminal “—OH” moiety may be substituted for a C-terminal “—NH₂” moiety, and vice-versa. It is further understood that the moiety at the amino terminus or carboxy terminus may be a bond, e.g., a covalent bond, particularly in situations where the amino terminus or carboxy terminus is bound to a linker or to another chemical moiety, e.g., a PEG moiety.

The term “NH₂,” as used herein, refers to the free amino group present at the amino terminus of a polypeptide. The term “OH,” as used herein, refers to the free carboxy group present at the carboxy terminus of a peptide. Further, the term “Ac,” as used herein, refers to Acetyl protection through acylation of the C- or N-terminus of a polypeptide.

The term “carboxy,” as used herein, refers to —CO₂H.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. Some abbreviations useful in describing the invention are defined below in the following Table 1.

TABLE 1
Abbreviations of Non-Natural Amino Acids and Chemical Moieties

| Abbreviation | Definition |
|---|---|
| DIG | Diglycolic acid |
| Dapa | Diaminopropionic acid |
| Daba | Diaminobutyric acid |
| Pen | Penicillamine |
| Sarc | Sarcosine |
| Cit | Citroline |
| Cav | Cavanine |
| NMe-Arg | N-Methyl-Arginine |
| NMe-Trp | N-Methyl-Tryptophan |
| NMe-Phe | N-Methyl-Phenylalanine |
| Ac— | Acetyl |
| 2-Nal | 2-Napthylalanine |
| 1-Nal | 1-Napthylalanine |
| Bip | Biphenylalanine |
| βAla | beta-Alanine |
| Aib | 2-aminoisobutyric acid |
| Azt | azetidine-2-carboxylic acid |
| Tic | (3S)-1,2,3,4-Tetrahydroisoquinoline-hydroxy- |
| | 3-carboxylic acid |
| Phe(OMe) | Tyrosine (4-Methyl) |
| N-MeLys | N-Methyl-Lysine |
| N-MeLys(Ac) | N-e-Acetyl-D-lysine |
| Dpa | β,β diphenylalanine |
| NH₂ | Free Amine |
| CONH₂ | Amide |
| COOH | Acid |
| Phe(4-F) | 4-Fluoro-Phenylalanine |
| PEG3 | NH₂CH₂CH₂(OCH₂CH₂)₃CH₂CH₂CO₂H |
| m-PEG3 | CH₃OCH₂CH₂(OCH₂CH₂)₂CH₂CH₂CO₂H |
| m-PEG4 | CH₃OCH₂CH₂(OCH₂CH₂)₃CH₂CH₂CO₂H |
| m-PEG8 | CH₃OCH₂CH₂(OCH₂CH₂)₇CH₂CH₂CO₂H |
| PEG11 | O-(2-aminoethyl)-O′-(2-carboxyethyl)-undecaethyleneglycol |
| | NH₂CH₂CH₂(OCH₂CH₂)₁₁CH₂CH₂CO₂H |
| PEG13 | Bifunctional PEG linker with 13 PolyEthylene Glycol units |
| PEG25 | Bifunctional PEG linker with 25 PolyEthylene Glycol units |
| PEG1K | Bifunctional PEG linker with PolyEthylene Glycol Mol wt of |
| | 1000 Da |
| PEG2K | Bifunctional PEG linker with PolyEthylene Glycol Mol wt of |
| | 2000 Da |
| PEG3.4K | Bifunctional PEG linker with PolyEthylene Glycol Mol wt of |
| | 3400 Da |
| PEG5K | Bifunctional PEG linker with PolyEthylene Glycol Mol wt of |
| | 5000 Da |
| IDA or Ida | Iminodiacetic acid |
| IDA-Palm | (Palmityl)-Iminodiacetic acid |
| hPhe | homoPhenylalanine |
| Ahx | Aminohexanoic acid |
| DIG-OH | Glycolic monoacid |
| Triazine | Amino propyl Triazine di-acid |
| Boc-Triazine | Boc-Triazine di-acid |
| Trifluorobutyric acid | 4,4,4-Trifluorobutyric acid |
| 2-Methylltrifluorobutyric acid | 2-methyl-4,4,4-Butyric acid |
| Trifluorpentanoic acid | 5,5,5-Trifluoropentanoic acid |
| 1,4-Phenylenediacetic acid | para-Phenylenediacetic acid |
| 1,3-Phenylenediacetic acid | meta-Phenylenediacetic acid |
| DTT | Dithiothreotol |
| Nle | Norleucine |
| βhTrp or bhTrp | β-homoTryptophane |
| βhPhe or bhPhe | β-homophenylalanine |
| Phe(4-CF₃) | 4-TrifluoromethylPhenylalanine |
| βGlu or bGlu | β-Glutamic acid |
| βhGlu or bhGlu | β-homoglutamic acid |
| 2-2-Indane | 2-Aminoindane-2-carboxylic acid |
| 1-1-Indane | 1-Aminoindane-1-carboxylic acid |
| hCha | homocyclohexylalanine |
| Cyclobutyl | Cyclobutylalanine |
| hLeu | Homoleucine |
| Gla | γ-Carboxy-glutamic acid |
| Aep | 3-(2-aminoethoxy)propanoic acid |
| Aea | (2-aminoethoxy)acetic acid |
| IsoGlu-octanoic acid | octanoyl-γ-Glu |
| K-octanoic acid | octanoyl-ε-Lys |
| Dapa(Palm) | Hexadecanoyl-β-Diaminopropionic acid |
| IsoGlu-Palm | hexadecanoyl-γ-Glu |
| C-StBu | S-tert-butylthio-cysteine |
| C-tBu | S-tert-butyl-cysteine |
| Dapa(AcBr) | NY-(bromoacetyl)-2,3-diaminopropionic acid |
| Tle | tert-Leucine |
| Phg | phenylglycine |
| Oic | octahydroindole-2-carboxylic acid |
| Chg | α-cyclohexylglycine |
| GP-(Hyp) | Gly-Pro-HydroxyPro |
| Inp | isonipecotic acid |
| Amc | 4-(aminomethyl)cyclohexane carboxylic acid |
| Betaine | (CH3)3NCH2CH2CO2H |

Throughout the present specification, unless naturally occurring amino acids are referred to by their full name (e.g. alanine, arginine, etc.), they are designated by their conventional three-letter or single-letter abbreviations (e.g. Ala or A for alanine, Arg or R for arginine, etc.). In the case of less common or non-naturally occurring amino acids, unless they are referred to by their full name (e.g. sarcosine, omithine, etc.), frequently employed three- or four-character codes are employed for residues thereof, including, Sar or Sarc (sarcosine, i.e. N-methylglycine), Aib (α-aminoisobutyric acid), Daba (2,4-diaminobutanoic acid), Dapa (2,3-diaminopropanoic acid), γ-Glu (γ-glutamic acid), pGlu (pyroglutamic acid), Gaba (γ-aminobutanoic acid), β-Pro (pyrrolidine-3-carboxylic acid), 8Ado (8-amino-3,6-dioxaoctanoic acid), Abu (4-aminobutyric acid), bhPro (β-homo-proline), bhPhe (β-homo-L-phenylalanine), bhAsp (β-homo-aspartic acid]), Dpa (β,β diphenylalanine), Ida (Iminodiacetic acid), hCys (homocysteine), bhDpa (β-homo-β,β-diphenylalanine).

Furthermore, R¹ can in all sequences be substituted with isovaleric acids or equivalent. In some embodiments, wherein a peptide of the present invention is conjugated to an acidic compound such as, e.g., isovaleric acid, isobutyric acid, valeric acid, and the like, the presence of such a conjugation is referenced in the acid form. So, for example, but not to be limited in any way, instead of indicating a conjugation of isovaleric acid to a peptide by referencing isovaleroyl, in some embodiments, the present application may reference such a conjugation as isovaleric acid.

The term “L-amino acid,” as used herein, refers to the “L” isomeric form of a peptide, and conversely the term “D-amino acid” refers to the “D” isomeric form of a peptide. In certain embodiments, the amino acid residues described herein are in the “L” isomeric form, however, residues in the “D” isomeric form can be substituted for any L-amino acid residue, as long as the desired functional is retained by the peptide.

Unless otherwise indicated, reference is made to the L-isomeric forms of the natural and unnatural amino acids in question possessing a chiral center. Where appropriate, the D-isomeric form of an amino acid is indicated in the conventional manner by the prefix “D” before the conventional three-letter code (e.g. Dasp, (D)Asp or D-Asp; Dphe, (D)Phe or D-Phe).

The term “DRP,” as used herein, refers to disulfide rich peptides.

The term “dimer,” as used herein, refers broadly to a peptide comprising two or more monomer subunits. Certain dimers comprise two DRPs. Dimers of the present invention include homodimers and heterodimers. A monomer subunit of a dimer may be linked at its C- or N-terminus, or it may be linked via internal amino acid residues. Each monomer subunit of a dimer may be linked through the same site, or each may be linked through a different site (e.g., C-terminus, N-terminus, or internal site).

As used herein, in the context of certain disclosed peptide sequences (such as those depicted in Tables 2-4, 6-15), parentheticals, e.g., (_), represent side chain conjugations and brackets, e.g., [_], represent unnatural amino acid substitutions. Generally, where a linker is shown at the N-terminus of a peptide sequence, it indicates that the peptide is dimerized with another peptide, wherein the linker is attached to the N-terminus of the two peptides. Generally, where a linker is shown at the C-terminus of a peptide sequence, it indicates that the peptide is dimerized with another peptide, wherein the linker is attached to the C-terminus of the two peptides.

The term “isostere replacement” or “isostere substitution” are used interchangeably herein to refer to any amino acid or other analog moiety having chemical and/or structural properties similar to a specified amino acid. In certain embodiments, an isostere replacement is a conservative substitution with a natural or unnatural amino acid.

The term “cyclized,” as used herein, refers to a reaction in which one part of a polypeptide molecule becomes linked to another part of the polypeptide molecule to form a closed ring, such as by forming a disulfide bridge or other similar bond.

The term “subunit,” as used herein, refers to one of a pair of polypeptide monomers that are joined to form a dimer peptide composition.

The term “linker moiety,” as used herein, refers broadly to a chemical structure that is capable of linking or joining together two peptide monomer subunits to form a dimer.

The term “solvate” in the context of the present invention refers to a complex of defined stoichiometry formed between a solute (e.g., a hepcidin analogue or pharmaceutically acceptable salt thereof according to the invention) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

As used herein, a “disease of iron metabolism” includes diseases where aberrant iron metabolism directly causes the disease, or where iron blood levels are dysregulated causing disease, or where iron dysregulation is a consequence of another disease, or where diseases can be treated by modulating iron levels, and the like. More specifically, a disease of iron metabolism according to this disclosure includes iron overload diseases, iron deficiency disorders, disorders of iron biodistribution, other disorders of iron metabolism and other disorders potentially related to iron metabolism, etc. Diseases of iron metabolism include hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, sideroblastic anemia, porphyria, porphyria cutanea tarda, African iron overload, hyperferritinemia, ceruloplasmin deficiency, atransferrinemia, congenital dyserythropoietic anemia, anemia of chronic disease, anemia of inflammation, anemia of infection, hypochromic microcytic anemia, iron-deficiency anemia, iron-refractory iron deficiency anemia, anemia of chronic kidney disease, erythropoietin resistance, iron deficiency of obesity, other anemias, benign or malignant tumors that overproduce hepcidin or induce its overproduction, conditions with hepcidin excess, Friedreich ataxia, gracile syndrome, Hallervorden-Spatz disease, Wilson's disease, pulmonary hemosiderosis, hepatocellular carcinoma, cancer, hepatitis, cirrhosis of liver, pica, chronic renal failure, insulin resistance, diabetes, atherosclerosis, neurodegenerative disorders, multiple sclerosis, Parkinson's disease, Huntington's disease, and Alzheimer's disease.

In some embodiments, the disease and disorders are related to iron overload diseases such as iron hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia.

In some embodiments, the hepcidin analogues of the invention are used to treat diseases and disorders that are not typically identified as being iron related. For example, hepcidin is highly expressed in the murine pancreas suggesting that diabetes (Type I or Type II), insulin resistance, glucose intolerance and other disorders may be ameliorated by treating underlying iron metabolism disorders. See Ilyin, G. et al. (2003) FEBS Lett. 542 22-26, which is herein incorporated by reference. As such, peptides of the invention may be used to treat these diseases and conditions. Those skilled in the art are readily able to determine whether a given disease can be treated with a peptide according to the present invention using methods known in the art, including the assays of WO 2004092405, which is herein incorporated by reference, and assays which monitor hepcidin, hemojuvelin, or iron levels and expression, which are known in the art such as those described in U.S. Pat. No. 7,534,764, which is herein incorporated by reference.

In certain embodiments of the present invention, the diseases of iron metabolism are iron overload diseases, which include hereditary hemochromatosis, iron-loading anemias, alcoholic liver diseases and chronic hepatitis C.

The term “pharmaceutically acceptable salt,” as used herein, represents salts or zwitterionic forms of the peptides or compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthalenesulfonate, nicotinate, 2-naphthalenesulfonate, oxalate, pamoate, pectinate, persulfate, 3-phenylpropionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. A pharmaceutically acceptable salt may suitably be a salt chosen, e.g., among acid addition salts and basic salts. Examples of acid addition salts include chloride salts, citrate salts and acetate salts. Examples of basic salts include salts where the cation is selected among alkali metal cations, such as sodium or potassium ions, alkaline earth metal cations, such as calcium or magnesium ions, as well as substituted ammonium ions, such as ions of the type N(R1)(R2)(R3)(R4)+, where R1, R2, R3 and R4 independently will typically designate hydrogen, optionally substituted C1-6-alkyl or optionally substituted C2-6-alkenyl. Examples of relevant C1-6-alkyl groups include methyl, ethyl, 1-propyl and 2-propyl groups. Examples of C2-6-alkenyl groups of possible relevance include ethenyl, 1-propenyl and 2-propenyl. Other examples of pharmaceutically acceptable salts are described in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see Handbook of Pharmaceutical Salts: Properties, Selection, and Use by Stahl and Wermuth (Wiley-VCH, 2002). Other suitable base salts are formed from bases which form non-toxic salts. Representative examples include the aluminum, arginine, benzathine, calcium, choline, diethylamine, diolamine, glycine, lysine, magnesium, meglumine, olamine, potassium, sodium, tromethamine, and zinc salts. Hemisalts of acids and bases may also be formed, e.g., hemisulphate and hemicalcium salts.

The term “N(alpha)Methylation”, as used herein, describes the methylation of the alpha amine of an amino acid, also generally termed as an N-methylation.

The term “sym methylation” or “Arg-Me-sym”, as used herein, describes the symmetrical methylation of the two nitrogens of the guanidine group of arginine. Further, the term “asym methylation” or “Arg-Me-asym” describes the methylation of a single nitrogen of the guanidine group of arginine.

The term “acylating organic compounds”, as used herein refers to various compounds with carboxylic acid functionality that are used to acylate the N-terminus of an amino acid subunit prior to forming a C-terminal dimer. Non-limiting examples of acylating organic compounds include cyclopropylacetic acid, 4-Fluorobenzoic acid, 4-fluorophenylacetic acid, 3-Phenylpropionic acid, Succinic acid, Glutaric acid, Cyclopentane carboxylic acid, 3,3,3-trifluoropropeonic acid, 3-Fluoromethylbutyric acid, Tetrahedro-2H-Pyran-4-carboxylic acid.

The term “alkyl” includes a straight chain or branched, noncyclic or cyclic, saturated aliphatic hydrocarbon containing from 1 to 24 carbon atoms. Representative saturated straight chain alkyls include, but are not limited to, methyl, ethyl, n-propyl, n-butyl, n-pentyl, n-hexyl, and the like, while saturated branched alkyls include, without limitation, isopropyl, sec-butyl, isobutyl, tert-butyl, isopentyl, and the like. Representative saturated cyclic alkyls include, but are not limited to, cyclopropyl, cyclobutyl, cyclopentyl, cyclohexyl, and the like, while unsaturated cyclic alkyls include, without limitation, cyclopentenyl, cyclohexenyl, and the like.

As used herein, a “therapeutically effective amount” of the peptide agonists of the invention is meant to describe a sufficient amount of the peptide agonist to treat an hepcidin-related disease, including but not limited to any of the diseases and disorders described herein (for example, a disease of iron metabolism). In particular embodiments, the therapeutically effective amount will achieve a desired benefit/risk ratio applicable to any medical treatment.

### Peptide Analogues of Hepcidin

The present invention provides peptide analogues of hepcidin, which may be monomers or dimers (collectively “hepcidin analogues”).

In some embodiments, a hepcidin analogue of the present invention binds ferroportin, e.g., human ferroportin. In certain embodiments, hepcidin analogues of the present invention specifically bind human ferroportin. As used herein, “specifically binds” refers to a specific binding agent's preferential interaction with a given ligand over other agents in a sample. For example, a specific binding agent that specifically binds a given ligand, binds the given ligand, under suitable conditions, in an amount or a degree that is observable over that of any nonspecific interaction with other components in the sample. Suitable conditions are those that allow interaction between a given specific binding agent and a given ligand. These conditions include pH, temperature, concentration, solvent, time of incubation, and the like, and may differ among given specific binding agent and ligand pairs, but may be readily determined by those skilled in the art. In some embodiments, a hepcidin analogue of the present invention binds ferroportin with greater specificity than a hepcidin reference compound (e.g., any one of the hepcidin reference compounds provided herein). In some embodiments, a hepcidin analogue of the present invention exhibits ferroportin specificity that is at least about 10%, 20%, 30%, 40%, 50%, 60%, 70%, 80%, 90%, 100%, 200%, 300%, 400%, 500%, 700%, 1000%, or 10,000% higher than a hepcidin reference compound (e.g., any one of the hepcidin reference compounds provided herein. In some embodiments, a hepcidin analogue of the present invention exhibits ferroportin specificity that is at least about 5 fold, or at least about 10, 20, 50, or 100 fold higher than a hepcidin reference compound (e.g., any one of the hepcidin reference compounds provided herein.

In certain embodiments, a hepcidin analogue of the present invention exhibits a hepcidin activity. In some embodiments, the activity is an in vitro or an in vivo activity, e.g., an in vivo or an in vitro activity described herein. In some embodiments, a hepcidin analogue of the present invention exhibits at least about 50%, 60%, 70%, 80%, 90%, 95%, 97%, 98%, 99%, or greater than 99% of the activity exhibited by a hepcidin reference compound (e.g., any one of the hepcidin reference compounds provided herein.

In some embodiments, a hepcidin analogue of the present invention exhibits at least about 50%, 60%, 70%, 80%, 90%, 95%, 97%, 98%, 99%, or greater than 99% of the ferroportin binding ability that is exhibited by a reference hepcidin. In some embodiments, a hepcidin analogue of the present invention has a lower IC₅₀ (i.e., higher binding affinity) for binding to ferroportin, (e.g., human ferroportin) compared to a reference hepcidin. In some embodiments, a hepcidin analogue the present invention has an IC₅₀ in a ferroportin competitive binding assay which is at least about 10%, 20%, 30%, 40%, 50%, 60%, 70%, 80%, 90%, 100%, 200%, 300%, 400%, 500%, 700%, or 1000% lower than a reference hepcidin.

In certain embodiments, a hepcidin analogue of the present invention exhibits increased hepcidin activity as compared to a hepcidin reference peptide. In some embodiments, the activity is an in vitro or an in vivo activity, e.g., an in vivo or an in vitro activity described herein. In certain embodiments, the hepcidin analogue of the present invention exhibits 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater hepcidin activity than a reference hepcidin. In certain embodiments, the hepcidin analogue of the present invention exhibits at least about 10%, 20%, 30%, 40%, 50%, 60%, 70%, 80%, 90%, 95%, 97%, 98%, 99% or greater than 99%, 100%, 200% 300%, 400%, 500%, 700%, or 1000% greater activity than a reference hepcidin.

In some embodiments, a peptide analogue of the present invention exhibits at least about 50%, 60%, 70%, 80%, 90%, 95%, 97%, 98%, 99%, or greater than 99%, 100%, 200% 300%, 400%, 500%, 700%, or 1000% greater in vitro activity for inducing the degradation of human ferroportin protein as that of a reference hepcidin, wherein the activity is measured according to a method described herein.

In some embodiments, a peptide or a peptide dimer of the present invention exhibits at least about 50%, 60%, 70%, 80%, 90%, 95%, 97%, 98%, 99%, or greater than 99%, 100%, 200% 300%, 400%, 500%, 700%, or 1000% greater in vivo activity for inducing the reduction of free plasma iron in an individual as does a reference hepcidin, wherein the activity is measured according to a method described herein.

In some embodiments, the activity is an in vitro or an in vivo activity, e.g., an in vivo or an in vitro activity described herein. In certain embodiments, a hepcidin analogue of the present invention exhibits 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, 500%, 700%, or 1000% greater activity than a reference hepcidin, wherein the activity is an in vitro activity for inducing the degradation of ferroportin, e.g., as measured according to the Examples herein; or wherein the activity is an in vivo activity for reducing free plasma iron, e.g., as measured according to the Examples herein.

In some embodiments, the hepcidin analogues of the present invention mimic the hepcidin activity of Hep25, the bioactive human 25-amino acid form, are herein referred to as “mini-hepcidins”. As used herein, in certain embodiments, a compound (e.g., a hepcidin analogue) having a “hepcidin activity” means that the compound has the ability to lower plasma iron concentrations in subjects (e.g. mice or humans), when administered thereto (e.g. parenterally injected or orally administered), in a dose-dependent and time-dependent manner. See e.g. as demonstrated in Rivera et al. (2005), Blood 106:2196-9. In some embodiments, the peptides of the present invention lower the plasma iron concentration in a subject by at least about 1.2, 1.5, 2, 3, 4, 5, 6, 7, 8, 9, or 10-fold, or at least about 5%, 10%, 20%, 25%, 30%, 40%, 50%, 60%, 70%, 80%, 90%, or about 99%.

In some embodiments, the hepcidin analogues of the present invention have in vitro activity as assayed by the ability to cause the internalization and degradation of ferroportin in a ferroportin-expressing cell line as taught in Nemeth et al. (2006) Blood 107:328-33. In some embodiments, in vitro activity is measured by the dose-dependent loss of fluorescence of cells engineered to display ferroportin fused to green fluorescent protein as in Nemeth et al. (2006) Blood 107:328-33. Aliquots of cells are incubated for 24 hours with graded concentrations of a reference preparation of Hep25 or a mini-hepcidin. As provided herein, the EC₅₀ values are provided as the concentration of a given compound (e.g. a hepcidin analogue peptide or peptide dimer of the present invention) that elicits 50% of the maximal loss of fluorescence generated by a reference compound. The EC₅₀ of the Hep25 preparations in this assay range from 5 to 15 nM and in certain embodiments, preferred hepcidin analogues of the present invention have EC₅₀ values in in vitro activity assays of about 1,000 nM or less. In certain embodiments, a hepcidin analogue of the present invention has an EC₅₀ in an in vitro activity assay (e.g., as described in Nemeth et al. (2006) Blood 107:328-33 or the Example herein) of less than about any one of 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, a hepcidin analogue or biotherapeutic composition (e.g., any one of the pharmaceutical compositions described herein) has an EC₅₀ value of about 1 nM or less.

Other methods known in the art for calculating the hepcidin activity and in vitro activity of the hepcidin analogues according to the present invention may be used. For example, in certain embodiments, the in vitro activity of the hepcidin analogues or the reference peptides is measured by their ability to internalize cellular ferroportin, which is determined by immunohistochemistry or flow cytometry using antibodies which recognizes extracellular epitopes of ferroportin. Alternatively, in certain embodiments, the in vitro activity of the hepcidin analogues or the reference peptides is measured by their dose-dependent ability to inhibit the efflux of iron from ferroportin-expressing cells that are preloaded with radioisotopes or stable isotopes of iron, as in Nemeth et al. (2006) Blood 107:328-33.

In some embodiments, the hepcidin analogues of the present invention exhibit increased stability (e.g., as measured by half-life, rate of protein degradation) as compared to a reference hepcidin. In certain embodiments, the stability of a hepcidin analogue of the present invention is increased at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, or 500% greater than a reference hepcidin. In some embodiments, the stability is a stability that is described herein. In some embodiments, the stability is a plasma stability, e.g., as optionally measured according to the method described herein.

In particular embodiments, a hepcidin analogue of the present invention exhibits a longer half-life than a reference hepcidin. In particular embodiments, a hepcidin analogue of the present invention has a half-life under a given set of conditions (e.g., temperature, pH) of at least about 5 minutes, at least about 10 minutes, at least about 20 minutes, at least about 30 minutes, at least about 45 minutes, at least about 1 hour, at least about 2 hour, at least about 3 hours, at least about 4 hours, at least about 5 hours, at least about 6 hours, at least about 12 hours, at least about 18 hours, at least about 1 day, at least about 2 days, at least about 4 days, at least about 7 days, at least about 10 days, at least about two weeks, at least about three weeks, at least about 1 month, at least about 2 months, at least about 3 months, or more, or any intervening half-life or range in between, about 5 minutes, about 10 minutes, about 20 minutes, about 30 minutes, about 45 minutes, about 1 hour, about 2 hour, about 3 hours, about 4 hours, about 5 hours, about 6 hours, about 12 hours, about 18 hours, about 1 day, about 2 days, about 4 days, about 7 days, about 10 days, about two weeks, about three weeks, about 1 month, about 2 months, about 3 months, or more, or any intervening half-life or range in between. In some embodiments, the half-life of a hepcidin analogue of the present invention is extended due to its conjugation to one or more lipophilic substituent, e.g., any of the lipophilic substituents disclosed herein. In some embodiments, the half-life of a hepcidin analogue of the present invention is extended due to its conjugation to one or more polymeric moieties, e.g., any of the polymeric moieties disclosed herein. In certain embodiments, a hepcidin analogue of the present invention has a half-life as describe above under the given set of conditions wherein the temperature is about 25° C., about 4° C., or about 37° C., and the pH is a physiological pH, or a pH about 7.4.

In some embodiments, the half-life is measured in vitro using any suitable method known in the art, e.g., in some embodiments, the stability of a hepcidin analogue of the present invention is determined by incubating the hepcidin analogue with pre-warmed human serum (Sigma) at 37° C. Samples are taken at various time points, typically up to 24 hours, and the stability of the sample is analyzed by separating the hepcidin analogue from the serum proteins and then analyzing for the presence of the hepcidin analogue of interest using LC-MS.

In some embodiments, the stability of the hepcidin analogue is measured in vivo using any suitable method known in the art, e.g., in some embodiments, the stability of a hepcidin analogue is determined in vivo by administering the peptide or peptide dimer to a subject such as a human or any mammal (e.g., mouse) and then samples are taken from the subject via blood draw at various time points, typically up to 24 hours. Samples are then analyzed as described above in regard to the in vitro method of measuring half-life. In some embodiments, in vivo stability of a hepcidin analogue of the present invention is determined via the method disclosed in the Examples herein.

In some embodiments, the present invention provides a hepcidin analogue as described herein, wherein the hepcidin analogue exhibits improved solubility or improved aggregation characteristics as compared to a reference hepcidin. Solubility may be determined via any suitable method known in the art. In some embodiments, suitable methods known in the art for determining solubility include incubating peptides (e.g., a hepcidin analogue of the present invention) in various buffers (Acetate pH4.0, Acetate pH5.0, Phos/Citrate pH5.0, Phos Citrate pH6.0, Phos pH 6.0, Phos pH 7.0, Phos pH7.5, Strong PBS pH 7.5, Tris pH7.5, Tris pH 8.0, Glycine pH 9.0, Water, Acetic acid (pH 5.0 and other known in the art) and testing for aggregation or solubility using standard techniques. These include, but are not limited to, visual precipitation, dynamic light scattering, Circular Dichroism and fluorescent dyes to measure surface hydrophobicity, and detect aggregation or fibrillation, for example. In some embodiments, improved solubility means the peptide (e.g., the hepcidin analogue of the present invention) is more soluble in a given liquid than is a reference hepcidin.

In certain embodiments, the present invention provides a hepcidin analogue as described herein, wherein the hepcidin analogue exhibits a solubility that is increased at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, or 500% greater than a reference hepcidin in a particular solution or buffer, e.g., in water or in a buffer known in the art or disclosed herein.

In certain embodiments, the present invention provides a hepcidin analogue as described herein, wherein the hepcidin analogue exhibits decreased aggregation, wherein the aggregation of the peptide in a solution is at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold less or at least about 10%, 20%, 30%, 40%, 50%, 60%, 70%, 80%, 90%, 100%, 200%, 300%, 400%, or 500% less than a reference hepcidin in a particular solution or buffer, e.g., in water or in a buffer known in the art or disclosed herein.

In some embodiments, the present invention provides a hepcidin analogue, as described herein, wherein the hepcidin analogue exhibits less degradation (i.e., more degradation stability), e.g., greater than or about 10% less, greater than or about 20% less, greater than or about 30% less, greater than or about 40 less, or greater than or about 50% less than a reference hepcidin. In some embodiments, degradation stability is determined via any suitable method known in the art. In some embodiments, suitable methods known in the art for determining degradation stability include the method described in Hawe et al J Pharm Sci, VOL. 101, NO. 3, 2012, p 895-913, incorporated herein in its entirety. Such methods are in some embodiments used to select potent sequences with enhanced shelf lives.

In some embodiments, the hepcidin analogue of the present invention is synthetically manufactured. In other embodiments, the hepcidin analogue of the present invention is recombinantly manufactured.

The various hepcidin analogue monomer and dimer peptides of the invention may be constructed solely of natural amino acids. Alternatively, these hepcidin analogues may include unnatural or non-natural amino acids including, but not limited to, modified amino acids. In certain embodiments, modified amino acids include natural amino acids that have been chemically modified to include a group, groups, or chemical moiety not naturally present on the amino acid. The hepcidin analogues of the invention may additionally include D-amino acids. Still further, the hepcidin analogue peptide monomers and dimers of the invention may include amino acid analogs. In particular embodiments, a peptide analogue of the present invention comprises any of those described herein, wherein one or more natural amino acid residues of the peptide analogue is substituted with an unnatural or non-natural amino acid, or a D-amino acid.

In certain embodiments, the hepcidin analogues of the present invention include one or more modified or unnatural amino acids. For example, in certain embodiments, a hepcidin analogue includes one or more of Daba, Dapa, Pen, Sar, Cit, Cav, HLeu, 2-Nal, 1-Nal, d-1-Nal, d-2-Nal, Bip, Phe(4-OMe), Tyr(4-OMe), βhTrp, βhPhe, Phe(4-CF₃), 2-2-Indane, 1-1-Indane, Cyclobutyl, βhPhe, hLeu, Gla, Phe(4-NH₂), hPhe, 1-Nal, Nle, 3-3-diPhe, cyclobutyl-Ala, Cha, Bip, β-Glu, Phe(4-Guan), homo amino acids, D-amino acids, and various N-methylated amino acids. One having skill in the art will appreciate that other modified or unnatural amino acids, and various other substitutions of natural amino acids with modified or unnatural amino acids, may be made to achieve similar desired results, and that such substitutions are within the teaching and spirit of the present invention.

The present invention includes any of the hepcidin analogues described herein, e.g., in a free or a salt form.

The hepcidin analogues of the present invention include any of the peptide monomers or dimers described herein linked to a linker moiety, including any of the specific linker moieties described herein.

The hepcidin analogues of the present invention include peptides, e.g., monomers or dimers, comprising a peptide monomer subunit having at least 85%, at least 90%, at least 95%, at least 98%, or at least 99% amino acid sequence identity to a hepcidin analogue peptide sequence described herein (e.g., any one of the peptides disclosed in Tables 1-4 or 6-15).

In certain embodiments, a peptide analogue of the present invention, or a monomer subunit of a dimer peptide analogue of the present invention, comprises or consists of 7 to 35 amino acid residues, 8 to 35 amino acid residues, 9 to 35 amino acid residues, 10 to 35 amino acid residues, 7 to 25 amino acid residues, 8 to 25 amino acid residues, 9 to 25 amino acid residues, 10 to 25 amino acid residues, 7 to 18 amino acid residues, 8 to 18 amino acid residues, 9 to 18 amino acid residues, or 10 to 18 amino acid residues, and, optionally, one or more additional non-amino acid moieties, such as a conjugated chemical moiety, e.g., a PEG or linker moiety. In particular embodiments, a monomer subunit of a hepcidin analogue comprises or consists of 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acid residues. In particular embodiments, a monomer subunit of a hepcidin analogue of the present invention comprises or consists of 10 to 18 amino acid residues and, optionally, one or more additional non-amino acid moieties, such as a conjugated chemical moiety, e.g., a PEG or linker moiety. In various embodiments, the monomer subunit comprises or consists of 7 to 35 amino acid residues, 9 to 18 amino acid residues, or 10 to 18 amino acid residues. In particular embodiments of any of the various Formulas described herein, X comprises or consists of 7 to 35 amino acid residues, 8 to 35 amino acid residues, 9 to 35 amino acid residues, 10 to 35 amino acid residues, 7 to 25 amino acid residues, 8 to 25 amino acid residues, 9 to 25 amino acid residues, 10 to 25 amino acid residues, 7 to 18 amino acid residues, 8 to 18 amino acid residues, 9 to 18 amino acid residues, or 10 to 18 amino acid residues.

### Peptide Monomer Hepcidin Analogues

In certain embodiments, hepcidin analogues of the present invention comprise a single peptide subunit. In certain embodiments, these hepcidin analogues form cyclized structures through intramolecular disulfide or other bonds. In one embodiment, the present invention provides a cyclized form of any one of the hepcidin analogues listed in Tables 2-4, or 12-15, provided that the analogue has two or more Cys residues.

In certain embodiments, the present invention includes a peptide analogue, wherein the peptide analogue has the structure of Formula I:

(I)

(SEQ ID NO: 1)

R¹-X-Y-R²

or a pharmaceutically acceptable salt or solvate thereof,

wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;

R² is OH or NH₂; and

X is a peptide sequence having the formula Ia:

(Ia)

(SEQ ID NO: 2)

X1-X2-X3-X4-X5-X6-X7-X8-X9-X10

wherein

X1 is Asp, Ser, Glu, Ida, pGlu, bhAsp, D-Asp or absent;
X2 is Thr, Ser, Lys, Glu, Pro, Ala or absent;

### X3 is His, Ala, or Glu;

### X4 is Phe, Ile or Dpa;

X5 is Pro, bhPro, Val, Glu, Sarc or Gly;

### X6 is Cys or (D)-Cys;

X7 is absent or any amino acid except Ile, Cys or (D)-Cys;
X8 is absent or any amino acid except Cys or (D)-Cys;
X9 is Phe, Ala, Ile, Thr, Tyr, Lys, Arg, bhPhe, D-Phe or absent; and
X10 is Lys, Phe or absent;
Y is absent or present; and

provided that if Y is present, Y is a peptide having the formula Im:

(Im)

(SEQ ID NO: 3)

Y1-Y2-Y3-Y4-Y5-Y6-Y7-Y8-Y9-Y10-Y11-Y12

wherein

Y1 is Gly, PEG3, Sarc, Lys, Glu, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Glu, Tyr or absent;
Y5 is Lys, Met, Ser, Arg, Ala or absent;
Y6 is Gly, Sarc, Glu, Lys, Arg, Ser, Lys, Ile, Ala, Pro, Val or absent;
Y7 is Trp, Lys, Gly, Ala, Ile, Val or absent;
Y8 is Val, Trp, His, Thr, Gly, Cys, Met, Tyr, Ala, Glu, Lys, Asp, Arg or absent;
Y9 is Val, Asp, Asn, Cys, Tyr or absent;
Y10 is Cys, Met, Lys, Arg, Tyr or absent;
Y11 is Arg, Met, Cys, Lys or absent; and
Y12 is Arg, Lys, Ala or absent.

In certain alternative embodiments, X7 is absent or any amino acid except Cys, or (D)-Cys.

In certain embodiments, X7 is Arg, Glu, Phe, Gln, Leu, Val, Lys, Ala, Ser, Dapa or absent.

In certain embodiments, X8 is Ile, Arg, Lys, Ala, Gln, Phe, Glu, Asp, Tyr, Ser, Leu, Val, D-Ile, D-Lys, D-Arg, Dapa or absent.

In certain embodiments of any of the peptide analogues having any of the various Formulae set forth herein, R¹ is selected from methyl, acetyl, formyl, benzoyl, trifluoroacetyl, isovaleryl, isobutyryl, octanyl, and conjugated amides of lauric acid, hexadecanoic acid, and γ-Glu-hexadecanoic acid.

In certain embodiments of any of the Formulae set forth herein, wherein the amino acid residue immediately carboxy to X6 is not Ile. In particular embodiments, wherein X6 is Cys or (D)-Cys, the amino acid residue immediately carboxy to X6 is not Ile. For example, in certain embodiments, wherein X7 is absent and X8 is present, X8 is not Ile, or wherein X7 and X8 are absent, X9 is not Ile.

In certain embodiments of any of the Formulae set forth herein, X either or both does not comprise or does not consist of an amino acid sequence set forth in U.S. Pat. No. 8,435,941.

In certain embodiments of the peptide analogue of Formula I,

X is a peptide sequence having the formula Ib:

(Ib)

(SEQ ID NO: 18)

X1-X2-X3-X4-X5-X6-X7-X8-X9-X10

wherein

X1 is Asp, Glu, Ida, pGlu, bhAsp, D-Asp or absent;
X2 is Thr, Ser, Lys, Glu, Pro, Ala or absent;

### X3 is His, Ala, Glu or Ala;

### X4 is Phe, Ile or Dpa;

X5 is Pro, bhPro, Sarc or Gly;

### X6 is Cys;

X7 is absent or any amino acid except Ile, Cys or (D)-Cys;
X8 is absent or any amino acid except Cys or (D)-Cys;
X9 is Phe, Ile, Tyr, bhPhe or D-Phe or absent; and
X10 is Lys, Phe or absent;

wherein Y is absent or present, provided that if Y is present, Y is a peptide having the formula In:

(In)

(SEQ ID NO: 19)

Y1-Y2-Y3-Y4-Y5-Y6-Y7-Y8-Y9-Y10-Y11-Y12

wherein

Y1 is Gly, PEG3, Sarc, Lys, Glu, Ala, Phe, Pro, Glu, Lys, D-Pro, Val, Ser or absent;
Y2 is Pro, Ala, Gly or absent;
Y3 is Arg, Lys, Pro, Gly, His, Ala, or absent;
Y4 is Ser, Arg, Glu or absent;
Y5 is Lys, Ser, Met, Arg, Ala or absent;
Y6 is Gly, Sarc, Glu, Leu, Phe, His or absent;
Y7 is Trp, NMe-Trp, Lys, Thr, His, Gly, Ala, Ile, Val or absent;
Y8 is Val, Trp, Ala, Asn, Glu or absent;
Y9 is Val, Ala, Asn, Asp, Cys or absent;
Y10 is Cys, (D)Cys, Glu or absent;
Y11 is Tyr, Met or absent; and
Y12 is Trp or absent.

In certain embodiments, X7 is Arg, Glu, Phe, Gln, Leu, Val, Lys, Ala, Ser, Dapa or absent.

In certain embodiments, X7 is Arg, Glu, Phe, Gln, Leu, Ile, Val, Lys, Ala, Ser, Dapa or absent.

In certain alternative embodiments, X7 is absent or any amino acid except Cys, or (D)-Cys.

In certain embodiments, X8 is Ile, Arg, Lys, Ala, Gln, Phe, Glu, Asp, Tyr, Ser, Leu, Val, D-Ile, D-Lys, D-Arg, Dapa or absent.

In some embodiments, the peptides of formula (I) comprise at least three, at least four, at least five, at least six, at least seven, at least eight, at least nine, at least ten, at least eleven, or at least 12 amino acid residues in Y.

In some embodiments, Y1 to Y3 are present and Y4 to Y12 are absent.

In some embodiments, Y1 to Y11 are present and Y12 is absent.

In some embodiments, Y1 to Y10 are present and Y11 to Y12 are absent.

Illustrative embodiments of peptide analogues of Formula I are provide in Table 2. In particular embodiments, a peptide analogue of the present invention comprises or consists of an amino acid sequence set forth in Table 2, or has a structure shown in Table 2. Table 2 also provides the EC₅₀ values of illustrative peptide analogues as determined via the ferroportin internalization/degradation assay described in the accompanying Examples.

TABLE 2
Illustrative Peptide Monomer Hepcidin Analogues

| SEQ | | |
|---|---|---|
| ID | | EC₅₀ |
| No. | Sequence | (nM) |
| 440 | Hy-DTHFPCAIF-NH₂ | >1000 |
| 441 | Hy-DTHFPCRRF-NH₂ | >10 μM |
| 442 | [IDA]-TH-[Dpa]-[bhPro]CRR-[bhPhe]-NH₂ | 206 |
| 443 | Hy-DTHFPCEIF-NH₂ | >1000 |
| 444 | Hy-DTHFPCFIF-NH₂ | 1191.8 |
| 445 | Hy-DTHFPCQIF-NH₂ | >1000 |
| 446 | Hy-DTHFPCRIF-NH₂ | >1000 |
| 447 | Hy-[pGlu]-THFPCRKF-NH₂ | >1000 |
| 448 | Hy-DTHFPCLIF-NH₂ | >10 μM |
| 449 | Hy-DTHFPCVIF-NH₂ | 81% at |
| | | 10 uM |
| 450 | Hy-DTHFPCEIF-NH₂ | 19% at |
| | | 10 uM |
| 451 | Hy-DTHFPCRIF-NH₂ | 31% at |
| | | 10 uM |
| 452 | Hy-DTHFPCKIF-NH₂ | 9% at 10 |
| | | uM |
| 453 | Hy-DTHFPCLF-NH₂ | 39% at 1 |
| | | uM |
| 454 | Hy-DTHFPCEF-NH₂ | 17% at |
| | | 10 uM |
| 455 | Hy-DTHFPCRF-NH₂ | 31% at |
| | | 10 uM |
| 456 | Hy-DTHFPRRFGPRSKGWVC-NH₂ | >1000 |
| 457 | [IDA]-THF-[bhPro]-CRR-[bhPhe]GPRSKGWVC- | >1000 |
| | NH₂ | |
| 458 | Hy-DTHFPCIFGPRSKGWVC-NH₂ | >1000 |
| 459 | Hy-DTHFPCRIFGPRSRGWVCK-NH₂ | >1000 |
| 460 | Isovaleric acid-DTHFPCLIFGPRSKGWVCK-NH₂ | 19.2 |
| 461 | Isovaleric acid-DTHFPCVIFGPRSKGWVCK-NH₂ | 41 |
| 462 | Isovaleric acid-DTHFPCSIFGPRSKGWVCK-NH₂ | 78 |
| 463 | Isovaleric acid-DTHFPCQIFGPRSKGWVCK-NH₂ | 157 |
| 464 | Isovaleric acid-DTHFPCKIFGPRSKGWVCK-NH₂ | 86 |
| 465 | Isovaleric acid-DTHFPC-[Dapa]- | 65 |
| | IFGPRSKGWDCK-NH₂ | |
| 466 | Isovaleric acid-DTHFPC-[Dapa]- | 151 |
| | IFGPRSKGWECK-NH₂ | |
| 467 | Isovaleric acid-DTHFPCKIFGPRSKGWECK-NH₂ | 163 |
| 468 | Isovaleric acid-DTHFPCRRFGPRSKGWVCK-NH₂ | >1000 |
| 469 | Isovaleric acid-DTHFPCTIFGPRSKGWVCK-NH₂ | Not |
| | | Tested |

In certain embodiments, the present invention includes a peptide analogue, wherein the peptide analogue has the structure of Formula II:

R¹—X—Y—R² (II) (SEQ ID NO:4)

or a pharmaceutically acceptable salt or solvate thereof,

wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;

R² is OH or NH₂; and

X is a peptide sequence having the formula IIa:

(IIa)

(SEQ ID NO: 5)

X1-X2-X3-X4-X5-X6-X7-X8-X9-X10

wherein

### X1 is Asp, Glu or Ida;

X2 is Thr, Ser or absent;

### X3 is His;

### X4 is Phe or Dpa;

X5 is Pro, bhPro, Sarc or Gly;

### X6 is Cys or (D)-Cys;

X7 is Arg, Glu, Phe, Gln, Leu, Val, Lys, Ile, Ala, Ser, Dapa or absent;
X8 is Ile, Arg, Lys, Arg, Ala, Gln, Phe, Glu, Asp, Tyr, Ser, Leu, Val, D-Ile, D-Lys, D-Arg, Dapa or absent;
X9 is Phe, Tyr, bhPhe, D-Phe or absent; and
X10 is Lys, Phe or absent; and

wherein Y is absent or present, provided that if Y is present, Y is a peptide having the formula IIm:

(IIm)

(SEQ ID NO: 6)

Y1-Y2-Y3-Y4-Y5-Y6-Y7-Y8-Y9-Y10-Y11-Y12

wherein
Y1 is Gly, Sarc, Lys, Glu or absent;
Y2 is Pro, Ala, Gly or absent;
Y3 is Arg, Lys, Pro, Gly, His, Ala or absent;
Y4 is Ser, Arg, Glu or absent;
Y5 is Lys, Ser, Met, Arg, Ala or absent;
Y6 is Gly, Sarc, Glu, Leu, Phe, His or absent;
Y7 is Trp, NMe-Trp, Lys, Thr, His, Gly, Ala, Ile, Val or absent;
Y8 is Val, Trp, Ala, Asn, Glu or absent;

### Y9 is Cys;

Y10 is Met or absent;
Y11 is Tyr, Met or absent; and
Y12 is Trp or absent.

In certain embodiments, X6 is Cys.

In some embodiments, X7 is Arg, Glu, Phe, Gln, Leu, Val, Lys, Ala, Ser, Dapa or absent.

In certain embodiments, Y10 is absent.

In certain embodiments, Y11 is Tyr.

In certain embodiments, Y11 is absent.

In certain embodiments, Y12 is absent.

In certain embodiments, Y11 and Y12 or Y10, Y11 and Y12 are absent.

In certain embodiments of any of the peptide analogues having any of the Formulae set forth herein, R¹ is selected from methyl, acetyl, formyl, benzoyl, trifluoroacetyl, isovaleryl, isobutyryl, octanyl, and the conjugated amides of lauric acid, hexadecanoic acid, and γ-Glu-hexadecanoic acid.

In certain embodiments of any of the Formulae set forth herein, X either or both does not comprise or does not consist of an amino acid sequence set forth in U.S. Pat. No. 8,435,941.

In some embodiments, the peptides of formula (II) comprise at least three, at least four, at least five, at least six, at least seven, at least eight, at least nine, at least ten, at least eleven, or at least 12 amino acid residues in Y.

In some embodiments, Y1 to Y3 are present and Y4 to Y12 are absent.

In some embodiments, Y1 to Y11 are present and Y12 is absent.

In some embodiments, Y1 to Y10 are present and Y11 to Y12 are absent.

Illustrative embodiments of peptide analogues of Formula II are provide in Table 3. In particular embodiments, a peptide analogue of the present invention comprises or consists of an amino acid sequence set forth in Table 3, or has a structure shown in Table 3. Table 3 also provides the EC₅₀ values of illustrative peptide analogues as determined via the ferroportin internalization/degradation assay described herein.

TABLE 3
Illustrative Peptide Monomer Hepcidin Analogues

| SEQ | | |
|---|---|---|
| ID | | Ferroportin internalization |
| No. | Sequence | assay EC₅₀ (nM) |
| 470 | Hy-DTHFPIAICI-NH₂ | Not Active |
| 471 | Hy-DTHFPIICI-NH₂ | Not Active |
| 472 | Hy-DTHICIAIF-NH₂ | Not Active |
| 473 | Hy-DTHCPIA1F-NH₂ | Not Active |
| 474 | Hy-ATHFPCIIF-NH₂ | >1000 |
| 475 | Hy-ADHFPCIIF-NH₂ | >1000 |
| 476 | Hy-DTHFPCIIFKC-NH₂ | 6398.0 |
| 477 | Hy-DTHFPCIIFAC-NH₂ | >1000 |
| 478 | Hy-DTHFPCIIFAA-NH₂ | 59% at 1 uM |
| 479 | Hy-DEHFPCIIF-NH₂ | 34% at 10 uM |
| 480 | Hy-DPHFPCIIF-NH₂ | 64% at 10 uM |
| 481 | Hy-DTHKPCIIF-NH₂ | 45% at 10 uM |
| 482 | Hy-DTHVPCIIF-NH₂ | 34% at 10 uM |
| 483 | Hy-DTHFVCIIF-NH₂ | 50% at 10 uM |
| 484 | Hy-DTHFPCIIY-NH₂ | 75% at 10 uM |
| 485 | Hy-DTHFPCIIT-NH₂ | 23% at 1 uM |
| 486 | Hy-DTHFPCILY-NH₂ | 85% at 1 uM |
| 487 | Hy-DTHFPCIEY-NH₂ | 8% at 1 uM |
| 488 | Isovaleric acid-DTHFPCIIFGPRSKG-[N-MeTrp]-VC-NH₂ | 32 |
| 489 | Isovaleric acid-DTHFPCIIF-[Sarc]-PRSKG-[N-MeTrp]-VC-NH₂ | 10 |
| 490 | Isovaleric acid-DTHFPCIIF-[Sarc]-PHSKG-[N-MeTrp]-VC-NH₂ | 9 |
| 491 | Isovaleric acid-DTHFPCIIFEPRSKHWVCK-NH₂ | 15 |
| 492 | Isovaleric acid-DTHFPCIIFEPRSKEWVCK-NH₂ | 19 |
| 493 | Isovaleric acid-DTHFPCIIFEPRSKLWVCK-NH₂ | 7 |
| 494 | Isovaleric acid-DTHFPCIIFEPRSKFWVCK-NH₂ | 10 |
| 495 | Isovaleric acid-DTHFPCIKFEPHSK-[Sarc]-CK-NH₂ | 28 |
| 496 | Isovaleric acid-DTHFPCIKFKPHSKEWVCE-NH₂ | 46 |
| 497 | Isovaleric acid-DTHFPCIKFEPRSKEWVCK-NH₂ | 20 |
| 498 | Isovaleric acid-DTHFPCIKFEPRSKLWVCK-NH₂ | 9 |
| 499 | Isovaleric acid-DTHFPCIKFEPRSKEWVCK-OH | 46 |
| 500 | Isovaleric acid-DTHFPCIKFEPRS-K(isoGlu-octanoic acid)-ECK- | 48 |
| | NH₂ | |
| 501 | Hy-DTHFPCIIFGPRSKGWAVCYW-NH₂ | 197 |
| 502 | Hy-DTHFPICIFGPHRSKGWVCM-NH₂ | 149 |
| 503 | Hy-DTHFPCIIFGPRSKGWVAC-NH₂ | 281 |
| 504 | Hy-DTHFP-[(D)Cys]-IIFGPRSKGWVA-[(D)Cys]-NH₂ | Not active |
| 505 | Hy-DTHFPCIIFGPRSKGWVACY-NH₂ | Not active |
| 506 | Hy-DTHFPCIIFGPRSRGHVCK-NH₂ | >1000 |
| 507 | Hy-DTHFPCIIFGPRSKGWNCK-NH₂ | >1000 |
| 508 | Hy-DTHFPCINFGPRSKGWVCK-NH₂ | >1000 |
| 509 | Hy-DTHFPCIDFGPRSKGWVCK-NH₂ | >1000 |
| 510 | Isovaleric acid-DTHFECIIFGPRSKGWVCK-NH₂ | >1000 |
| 511 | Hy-DTHFPCIIFGGPRSRGWVCK-NH₂ | 520 |
| 512 | Hy-DTHFPCIIFGGPRSKGWNCK-NH₂ | 404 |
| 513 | Hy-DTHFPCIIFGGPRSKGWDCK-NH₂ | 679 |
| 514 | Isovaleric acid-DTHFPCIFEPRSKGTCK-NH₂ | 57 |
| 515 | Isovaleric acid-DTHFPCIIF-[PEG3]-C-NH₂ | 157 |
| 516 | Isovaleric acid-DTHAPCIKF-[Sarc]-PRSKGWECK-NH₂ | Not active |
| 517 | Isovaleric acid-DTHAPCIKFEPRSK-[Sarc]-WECK-NH₂ | Not active |
| 518 | Isovaleric acid-DTHAPCIKFEPRSKEWECK-NH₂ | Not active |
| 519 | Isovaleric acid-STHAPCIKFEPRSKGWECK-NH₂ | Not active |
| 520 | Isovaleric acid-SKHAPCIKFEPRSKGWECK-NH₂ | Not active |
| 521 | Isovaleric acid-DTHFPCIKFEPHSKEWVCK-NH₂ | 80 |
| 522 | Isovaleric acid-DTAFPCIKFEPRSKEC-NH₂ | Not active |
| 523 | Isovaleric acid-DTHFGCIKFEPRSKEWVCK-NH₂ | >1000 |
| 524 | Isovaleric acid-DTEFPCIKFEPRSKEWVCK-NH₂ | >1000 |
| 525 | Isovaleric acid-DTHFPCIKFEPRS-K(octanoic acid)-EWVCK- | 62 |
| | NH₂ | |
| 526 | Isovaleric acid-ETHFPCIKFEPRSKEWVCK-NH₂ | 181 |

### Peptide Dimer Hepcidin Analogues

In certain embodiments, the present invention includes dimers of the monomer hepcidin analogues described herein, including dimers comprising any of the monomer peptides sequences or structures set forth in Tables 2-4, and certain dimers of sequences or structures set forth in Tables 6-10, 12, 14, and 15. In particular embodiments, the invention includes dimers of any of the monomer peptide sequences or structure set forth in Table 11 or 13. These dimers fall within the scope of the general term “hepcidin analogues” as used herein. The term “dimers,” as in peptide dimers, refers to compounds in which two peptide monomer subunits are linked. A peptide dimer of the present invention may comprise two identical monomer subunits, resulting in a homodimer, or two non-identical monomer subunits, resulting in a heterodimer. A cysteine dimer comprises two peptide monomer subunits linked through a disulfide bond between a cysteine residue in one monomer subunit and a cysteine residue in the other monomer subunit.

In particular embodiments, a peptide dimer hepcidin analogue comprises one or more, e.g., two, peptide monomer subunits shown in Table 4 or described in U.S. Pat. No. 8,435,941, which is herein incorporated by reference in its entirety.

TABLE 4
Illustrative peptide monomer subunits

| SEQ ID NO | Sequence |
|---|---|
| 376 | DTHFPICIFC |
| 377 | FPIC |
| 378 | HFPIC |
| 379 | HFPICI |
| 380 | HFPICIF |
| 381 | DTHFPIC |
| 381 | DTHFPICI |
| 382 | DTHFPICIF |
| 383 | DTHFPIAIFC |
| 384 | DTHAPICIF |
| 385 | DTHAPI-[C-StBu]-IF |
| 386 | DTHAPI-[C-tBu]-IF |
| 387 | DTHFPIAIF |
| 388 | DTHFPISIF |
| 389 | DTHFPI-([D)-Cys]-IF |
| 390 | DTHFPI-[homoCys]-IF |
| 391 | DTHFPI-[Pen]-IF |
| 392 | DTHFPI-[(D)-Pen]-IF |
| 393 | DTHFPI-[Dapa(AcBr)]-IF |
| 394 | CDTHFPICIF |
| 395 | DTHFPICIF-NHCH₂CH₂S |
| 396 | CHFPICIF |
| 397 | HFPICIF-NHCH₂CH₂S |
| 398 | D-[Tle]-H-[Phg]-[Oic]-[Chg]-C-[Chg]-F |
| 399 | D-[Tle]-HP-[Oic]-[Chg]-C-[Chg]-F |
| 400 | [(D)Phe]-[(D)Ile]-[(D)Cys]-[(D)Ile]-[(D)pro]-[(D)Phe]-[(D)His]-[(D)Thr]- |
| | [(D)Asp] |
| 401 | [(D)Phe]-[(D)Ile]-[(D)Cys]-[(D)Ile]-[(D)Pro]-[(D)Phe]-[(D)His] |
| 402 | Chenodeoxycholate-(PEG11)-[(D)Phe]-[(D)Ile]-[(D)Cys]-[(D)Ile]-[(D)Pro]- |
| | [(D)Phe]-[(D)His]-[(D)Thr]-[(D)Asp] |
| 403 | Ursodeoxycholate-(PEG11)-[(D)Phe]-[(D)Ile]-[(D)Cys]-[(D)Ile]-[(D)Pro]- |
| | [(D)Phe]-[(D)His]-[(D)Thr]-[(D)Asp] |
| 404 | F-[(D)Ile]-[(D)Cys]-[(D)Ile]-[(D)Pro]-[(D)Phe]-[(D)His]-[(D)Thr]-[(D)Asp]- |
| | (Peg11)-GYIPEAPRDGQAYVRKDGEWVLLSTFL |
| 405 | F-[(D)Ile]-[(D)Cys]-[(D)Ile]-[(D)pro]-[(D)Phe]-[(D)His]-[(D)Thr]-[(D)Asp]- |
| | [GP-(Hyp)]₁₀ |
| 406 | Palmitoyl-(PEG11)-[(D)Phe]-[(D)Ile]-[(D)Cys]-[(D)Ile]-[(D)Pro]-[(D)Phe]- |
| | [(D)His]-[(D)Thr]-[(D)Asp] |
| 407 | 2(Palmitoyl)-[Dapa]-(Peg11)-[(D)Phe]-[(D)Ile]-[(D)Cys]-[(D)Ile]-[(D)Pro]- |
| | [(D)Phe]-[(D)His]-[(D)Thr]-[(D)Asp] |
| 408 | DTH-[bhPhe]-PIICIF |
| 409 | DTH-[Dpa]-PICI. |
| 410 | DTH-[Bip]-PICIF |
| 411 | DTH[1-Nal]-PICIF |
| 412 | DTH-[bhDpa]-PICIF |
| 413 | DTHFP-ICI-bhPhe |
| 414 | DTHFPICI-[Dpa] |
| 415 | DTHFPICI-[Bip] |
| 416 | DTHFPICI-[1-Nal] |
| 417 | DTHFPICI-[bhDpa] |
| 418 | DTH-[Dpa]-PICI-[Dpa] |
| 419 | D-[Dpa]-PICIF |
| 420 | D-[Dpa]-PICI-[Dpa] |
| 421 | DTH-[Dpa]-P-[(D)Arg]-CR-[Dpa] |
| 422 | DTH-[Dpa]-P-[(D)Arg]-C-[(D)Arg]-[Dpa] |
| 423 | DTH-[Dpa]-[Oic]-ICIF |
| 424 | DTH-[Dpa]-[Oic]-ICI-[Dpa] |
| 425 | DTH-[Dpa]-PCCC-[Dpa] |
| 426 | DTHFPICIF-[(D)Pro]-PK |
| 427 | DTHFPICIF-[(D)Pro]-PR |
| 428 | DTHFPICIF-[bhPro]-PK |
| 429 | DTHFPICIF-[bhPro]-PR |
| 430 | DTHFPICIF-[(D)Pro]-[bhPro]-K |
| 431 | DTHFPICIF-[(D)-Pro]-[bhPro]-R |
| 432 | DTHFPICI-[bhPhe]-[(D)Pro]-PK |
| 433 | DTHFPICI-[bhPhe]-[(D)Pro]-PR |
| 434 | DTHFPICI-[bhPhe]-[(D)Pro]-[bhPro]-K |
| 435 | DTHFPICI-[bhPhe]-[(D)Pro]-[bhPro]-R |
| 436 | C-[Inp]-[(D)Dpa]-[Amc]-R-[Amc]-[Inp]-[Dpa]-Cysteamide |
| 437 | CP-[(D)Dpa]-[Amc]-R-[Amc]-[Inp]-[Dpa]-Cysteamide |
| 438 | C-[(D)Pro]-[(D)Dpa]-[Amc]-R-[Amc]-[Inp]-[Dpa]-Cysteamide |
| 439 | CG-[(D)Dpa]-[Amc]-R-[Amc]-[Inp]-[Dpa]-Cysteamide |

In some embodiments, the hepcidin analogues of the present invention are active in a dimer conformation, in particular when free cysteine residues are present in the peptide. In certain embodiments, this occurs either as a synthesized dimer or, in particular, when a free cysteine monomer peptide is present and under oxidizing conditions, dimerizes. In some embodiments, the dimer is a homodimer. In other embodiments, the dimer is a heterodimer.

In certain embodiments, a hepcidin analogue dimer of the present invention is a peptide dimer comprising two hepcidin analogue peptide monomers of the invention.

In various embodiments, the amino acid sequences listed in Tables 2-4 and Tables 6-15 are shown using one letter codes for amino acids. Wherein only the hepcidin analogue monomer peptide sequence is shown, it is understood that, in certain embodiments, these hepcidin analogue monomer peptides, i.e., monomer subunits, are dimerized to form peptide dimer hepcidin analogues, in accordance with the present teachings. Thus, in one embodiment, the present invention provides a dimer of a peptide monomer shown in any one of Tables 2-4, 6-10, 12, 14, or 15.

The monomer subunits may be dimerized by a disulfide bridge between two cysteine residues, one in each peptide monomer subunit, or they may be dimerized by another suitable linker moiety, as defined herein. Some of the monomer subunits are shown having C- and N-termini that both comprise free amine. Thus, to produce a peptide dimer inhibitor, the monomer subunit may be modified to eliminate either the C- or N-terminal free amine, thereby permitting dimerization at the remaining free amine. Further, in some instances, a terminal end of one or more monomer subunits is acylated with an acylating organic compound selected from the group consisting of 2-me-Trifluorobutyl, Trifluoropentyl, Acetyl, Octonyl, Butyl, Pentyl, Hexyl, Palmityl, Trifluoromethyl butyric, cyclopentane carboxylic, cyclopropylacetic, 4-fluorobenzoic, 4-fluorophenyl acetic, 3-Phenylpropionic, tetrahedro-2H-pyran-4carboxylic, succinic acid, and glutaric acid. In some instances, monomer subunits comprise both a free carboxy terminal and a free amino terminal, whereby a user may selectively modify the subunit to achieve dimerization at a desired terminus. One having skill in the art will, therefore, appreciate that the monomer subunits of the instant invention may be selectively modified to achieve a single, specific amine for a desired dimerization.

It is further understood that the C-terminal residues of the monomer subunits disclosed herein are amides, unless otherwise indicated. Further, it is understood that, in certain embodiments, dimerization at the C-terminus is facilitated by using a suitable amino acid with a side chain having amine functionality, as is generally understood in the art. Regarding the N-terminal residues, it is generally understood that dimerization may be achieved through the free amine of the terminal residue, or may be achieved by using a suitable amino acid side chain having a free amine, as is generally understood in the art.

Moreover, it is understood that the side chains of one or more internal residue comprised in the hepcidin analogue peptide monomers of the present invention can be utilized for the purpose of dimerization. In such embodiments, the side chain is in some embodiments a suitable natural amino acid (e.g., Lys), or alternatively it is an unnatural amino acid comprising a side chain suitable for conjugation, e.g., to a suitable linker moiety, as defined herein.

The linker moieties connecting monomer subunits may include any structure, length, and/or size that is compatible with the teachings herein. In at least one embodiment, a linker moiety is selected from the non-limiting group consisting of: cysteine, lysine, DIG, PEG4, PEG4-biotin, PEG13, PEG25, PEG1K, PEG2K, PEG3.4K, PEG4K, PEG5K, IDA, IDA-Palm, ADA, Boc-IDA, Glutaric acid, Isophthalic acid, 1,3-phenylenediacetic acid, 1,4-phenylenediacetic acid, 1,2-phenylenediacetic acid, Triazine, Boc-Triazine, IDA-biotin, PEG4-Biotin, AADA, suitable aliphatics, aromatics, heteroaromatics, and polyethylene glycol based linkers having a molecular weight from approximately 400 Da to approximately 40,000 Da. Non-limiting examples of suitable linker moieties are provided in Table 5.

TABLE 5
Illustrative Linker Moieties

| Abbreviation | Description | Structure |
|---|---|---|
| DIG | DIGlycolic acid | 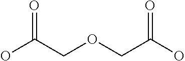 |
| PEG4 | Bifunctional PEG linker with 4 PolyEthylene Glycol units | 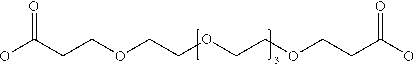 |
| PEG13 | Bifunctional PEG linker with 13 PolyEthylene Glycol units | 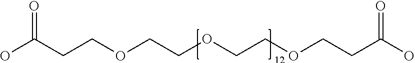 |
| PEG25 | Bifunctional PEG linker with 25 PolyEthylene Glycol units | 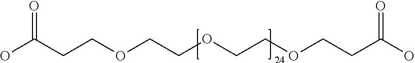 |
| PEG1K | Bifunctional PEG linker with | |
| | PolyEthylene Glycol Mol wt of | |
| | 1000Da | |
| PEG2K | Bifunctional PEG linker with | |
| | PolyEthylene Glycol Mol wt of | |
| | 2000Da | |
| PEG3.4K | Bifunctional PEG linker with | |
| | PolyEthylene Glycol Mol wt of | |
| | 3400Da | |
| PEG5K | Bifunctional PEG linker with | |
| | PolyEthylene Glycol Mol wt of | |
| | 5000Da | |
| DIG | Diglycolic acid | 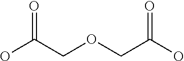 |
| β-Ala-IDA | β-Ala-Iminodiacetic acid | 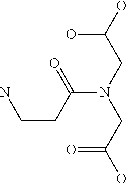 [SMILES: NCCC(=O)N(CC(=O)O)CC(O)O] |
| Boc-β-Ala- IDA | Boc-β-Ala-Iminodiacetic acid | 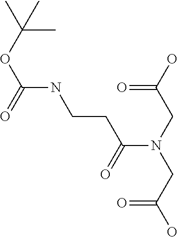 [SMILES: CC(C)(C)OC(=O)NCCC(=O)N(CC(=O)O)CC(=O)O] |
| Ac-β-Ala- IDA | Ac-β-Ala-Iminodiacetic acid | 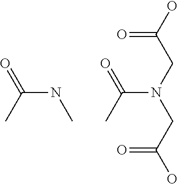 |
| Palm-β-Ala- IDA- | Palmityl-β-Ala-Iminodiacetic acid | 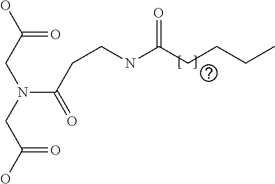 |
| GTA | Glutaric acid | 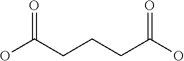 |
| PMA | Pemilic acid | 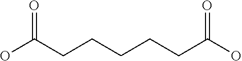 [SMILES: O=C(O)CCCCCC(=O)O] |
| AZA | Azelaic acid | 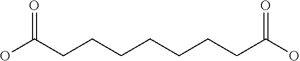 [SMILES: O=C(O)CCCCCCCC(=O)O] |
| DDA | Dodecanedioic acid | 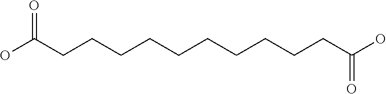 [SMILES: O=C(O)CCCCCCCCCCC(=O)O] |
| IPA | Isopthalic acid | 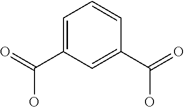 [SMILES: O=C(O)c1cccc(C(=O)O)c1] |
| 1,3-PDA | 1,3-Phenylenediacetic acid | 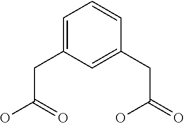 [SMILES: O=C(O)Cc1cccc(CC(=O)O)c1] |
| 1,4-PDA | 1,4-Phenylenediacetic acid | 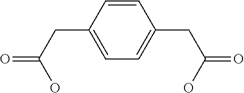 [SMILES: O=C(O)Cc1ccc(CC(=O)O)cc1] |
| 1,2-PDA | 1,2-Phenylenediacetic acid | 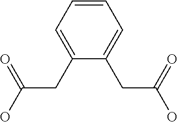 [SMILES: O=C(O)Cc1ccccc1CC(=O)O] |
| Triazine | Amino propyl Triazine di-acid | 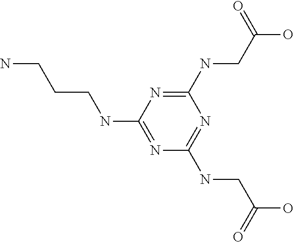 [SMILES: NCCCNc1nc(NCC(=O)O)nc(NCC(=O)O)n1] |
| Boc-Triazine | Boc-Triazine di-acid | 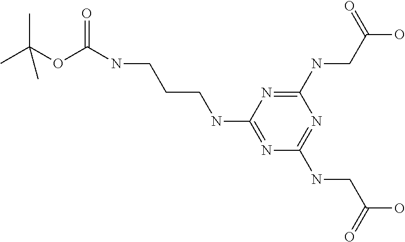 [SMILES: CC(C)(C)OC(=O)NCCCNc1nc(NCC(=O)O)nc(NCC(=O)O)n1] |
| IDA | Iminodiacetic acid | 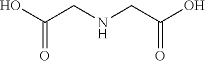 |
| AIDA | n-Acetyl imino acetic acid | 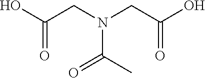 |
| Biotin-β-ala- IDA- | N-Biotin-β-Ala-Iminodiacetic acid | 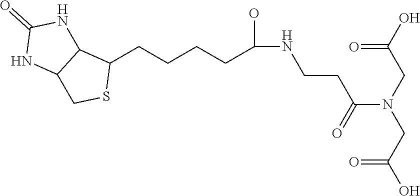 [SMILES: O=C(O)CN(CC(=O)O)C(=O)CCNC(O)CCCCC1SCC2NC(=O)NC21] |
| Lys | Lysine | 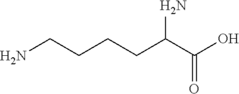 |

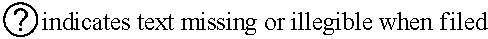

One having skill in the art will appreciate that the C- and N-terminal and internal linker moieties disclosed herein are non-limiting examples of suitable linker moieties, and that the present invention may include any suitable linker moiety. Thus, some embodiments of the present invention comprise a homo- or heterodimer hepcidin analogue comprised of two monomer subunits selected from the peptides shown herein, e.g., in Tables 2-4 and 11-15 or comprising or consisting of a sequence presented herein, e.g., in Tables 2-4 and 11-15, wherein the C- or N-termini of the respective monomer subunits are linked by any suitable linker moiety to provide a hepcidin analogue dimer peptide having hepcidin activity. In some embodiments the present invention comprises a homo- or heterodimer hepcidin analogue comprised of two monomer subunits described herein, e.g., selected from the peptides shown in Tables 2-4 and 11-15 or comprising or consisting of a sequence presented in Tables 2-4 or 10-15, wherein the respective monomer subunits are linked internally by any suitable linker moiety conjugated to the side chain of one or more internal amino acids to provide a hepcidin analogue dimer peptide having hepcidin activity.

In particular embodiments, a hepcidin analogue of the present invention comprises two or more polypeptide sequences of the monomer hepcidin analogues described herein.

In one embodiment, a peptide dimer hepcidin analogue of the present invention comprises two peptide monomer subunits connected via one or more linker moieties or intermolecular linkages (e.g., a cysteine disulfide bridge), wherein each peptide monomer subunit is a compound of Formula I, wherein X is hepcidin analogue of the present invention comprises two peptide monomer subunits connected via one or more linker moieties or intermolecular linkages (e.g., a cysteine disulfide bridge), or wherein each peptide monomer subunit is a compound of Formula II, e.g., wherein X is IIa and Y is IIm. In certain embodiments, a peptide dimer hepcidin analogue of the present invention comprises two peptide monomer subunits connected via one or more linker moieties or intermolecular linkages (e.g., a cysteine disulfide bridge), wherein each peptide monomer subunit is a compound of Formula I, wherein X is Ia and Y is Im, or wherein X is Ib and Y is In, or a compound of Formula II, wherein X is IIa and Y is IIm. In certain embodiments, the peptide dimer is a homodimer, and in other embodiments, the peptide dimer is a heterodimer.

In certain embodiments, a peptide dimer inhibitor has the structure of Formula VII:

(VII)

SEQ ID NO: 20

(R¹-X-Y-R²)₂-L

or a pharmaceutically acceptable salt or solvate thereof,

wherein each R¹ is independently selected from a bond (e.g., a covalent bond), hydrogen, a C1-C6 alkyl, a C6-C12 aryl, a C6-C12 aryl C1-C6 alkyl, a C1-C20 alkanoyl, and including PEGylated versions alone or as spacers of any of the foregoing;

each R² is independently absent, a bond (e.g., a covalent bond), or selected from OH or NH₂;

L is a linker moiety; and

wherein each X and Y combination is independently selected from those present in any of the Formulae described herein, such as Formulas I, II, III, IV, V, or VI. In certain embodiments, each X and Y combination is independently selected from the group consisting of:

Ia and Im;

Ib and In;

IIa and IIm;

IIIa-IIId and IIIm-IIIs;

IVa-IVd and IVm-Ivs;

Va-Vd and Vm-Vn; and

VIa and VIm.

In one embodiment of the peptide dimer of Formula VII, each X is an independently selected peptide sequence having the formula VIIa:

(VIIa)

SEQ ID NO: 21

X1-X2-X3-X4-X5-X6-X7-X8-X9-X10

wherein
X1 is Asp, Glu, Ida, Lys or absent;
X2 is Thr, Ser, Lys or absent;

### X3 is His, Ala or Lys;

### X4 is Phe, Dpa or Lys,

X5 is Pro, bhPro, Gly or Lys;

### X6 is Cys,

X7 is Arg, Glu, Phe, Gln, Leu, Val, Lys, Ala, Ser, Dapa, Thr or absent;
X8 is Ile, Arg, Lys, Glu, Asn, Asp, Ala, Gln, Phe, Glu, Tyr, Ser, Leu, Val, D-Ile, D-Lys, D-Arg, or Dapa or absent;
X9 is Phe, Tyr, bhPhe, Lys or absent; and
X10 is Lys, Phe or absent; and

each Y is absent.

In certain alternative embodiments of Formula VII, X7 is Arg, Glu, Phe, Gln, Leu, Val, Ile, Lys, Ala, Ser, Dapa, Thr or absent.

In certain embodiments of Formula VII, the linker is Lys or Phe. In particular embodiments, the linker is Lys.

In certain embodiments of Formula VII, the two X peptides are linked via a disulfide bond.

In some embodiments, the invention provides peptides, which may be isolated and/or purified, comprising, consisting essentially of, or consisting of the following structural formula VIII:

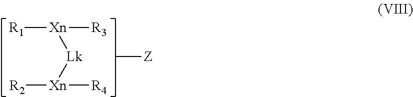

or a pharmaceutically acceptable salt or solvate thereof,

wherein

R₁ and R₂ are each independently selected from a bond, a hydrogen, a C1-C6 alkyl, a C6-C12 aryl, a C6-C12 aryl C1-C6 alkyl, and a C1-C20 alkanoyl, and including PEGylated versions (e.g. PEG3 to PEG11), alone or as spacers of any of the foregoing;

R₃ and R₄ are each independently selected from a bond, —NH₂ and —OH;

Xn and Yn are each independently selected peptide sequences having the formula Villa

(VIIIa)

SEQ ID NO: 22

X1-X2-X3-X4-X5-X6-X7-X8-X9-X10

wherein
X1 is Asp, Glu, Ida, Lys or absent;
X2 is Thr, Ser, Lys or absent;

### X3 is His, Ala, Lys;

### X4 is Phe, Dpa or Lys;

X5 is Pro, bhPro, Gly or Lys;

### X6 is Cys;

X7 is Arg, Glu, Phe, Gln, Leu, Val, Lys, Ala, Ser, Dapa, Thr or absent;
X8 is Ile, Arg, Lys, Glu, Asn, Asp, Ala, Gln, Phe, Glu, Tyr, Ser, Leu, Val, D-Ile, D-Lys, D-Arg, or Dapa or absent;
X9 is Phe, Tyr, bhPhe, Lys or absent; and
X10 is Lys, Phe or absent;

Lk is a linker or absent;

Xn and Yn are optionally linked by a disulfide bond; and

wherein Z is absent or it is a conjugate as described herein, (e.g., a conjugate to enhance drug like characteristics of the hepcidin analogue, such as extending in vivo half-life solubility, etc.), wherein if Z is present, it is optionally linked to the Xn peptide (e.g., at its N-terminus, C-terminus, or internally via a side chain, e.g., a lysine side chain), the Yn peptide (e.g., at its N-terminus, C-terminus, or internally via a side chain, e.g., a lysine side chain), or to an Lk linker.

In certain embodiments, Z is a palmyltyl moiety, a PEG moiety, or a lipidic moiety.

In certain embodiments, Lk links the two monomer subunits via an amino acid residue in Xn and/or an amino acid residue in Yn.

In certain alternative embodiments, R1, R2, R3, and R4 are selected from a bond, —NH₂ and —OH, hydrogen, a C1-C6 alkyl, a C6-C12 aryl, a C6-C12 aryl C1-C6 alkyl, and a C1-C20 alkanoyl, and including PEGylated versions (e.g. PEG3 to PEG11), alone or as spacers of any of the foregoing.

In certain embodiments, Lk links the two monomer subunits via R₃ and/or R₄.

In certain embodiments, Lk links the monomer subunits via R₁ and/or R₂.

In certain embodiments, Lk links the monomer subunits via any one of R₁, Xn or R₃ and any one of R₂, Yn and R₄.

In certain embodiments of Formula VIII, the linker is Lys or Phe. In particular embodiments, the linker is Lys.

In certain embodiments of Formula VIII, the two X peptides are linked via a disulfide bond.

In some embodiments, the present invention provides a hepcidin analogue monomer, or a homodimer or heterodimer thereof, comprising a peptide that comprises, consists of, or consists essentially of a sequence DTX₁FPC, wherein X₁ is any amino acid. In one embodiment, the present invention provides a peptide that comprises, consists of, or consists essentially of a sequence DTX₁FPCX₂X₃F, wherein X₁ is any amino acid, X₂ is any amino acid, and X₃ is any amino acid or it is absent. In one such embodiment, X₂ is any amino acid except for Cys. In one embodiment, X₁, X₂, and/or X₃ is an unnatural amino acid. In some embodiments, a dimer comprising such a hepcidin analogue monomer comprises a linker (e.g., a lysine linker). In some embodiments, such a dimer comprises a first hepcidin analogue monomer and a second monomer (which monomers are optionally identical in sequence), and the dimer further comprises at least one intermolecular disulfide bridge linking a Cys in the first monomer (e.g., the Cys shown in either one of the above formulae) to a Cys in the second monomer.

In some embodiments, the present invention provides a hepcidin analogue monomer, or a homodimer or heterodimer thereof, comprising a peptide that comprises, consists of, or consists essentially of a sequence X1X2X3FX₄CY₁X₅F, wherein any of X₁, X₂, and X₃ are absent or any amino acid, X₄ and X₅ are any amino acid, and Y₁ is any amino acid except for D-Cys, D-Ser, D-Ala, Cys(S-tBut), homoC, Pen, (D)Pen, Dap(AcBr), Inp, or D-His. In one such embodiment, Y₁ is any lipidic amino acid. In particular embodiments, Y₁ is selected from Val, Ile, and Leu. In one embodiment, Y₁ is Ile. In one embodiment, X5 is Lys. In one embodiment, any of X₁, X2, X3, X4, X₅, and/or Y₁ is an unnatural amino acid. In some embodiments, a dimer comprising such a hepcidin analogue monomer comprises a linker (e.g., a lysine linker). In some embodiments, such a dimer comprises a first hepcidin analogue monomer and a second monomer (which monomers are optionally identical in sequence), and the dimer further comprises at least one intermolecular disulfide bridge linking a Cys in the first monomer (e.g., the Cys shown in either one of the above formulae) to a Cys in the second monomer.

In some embodiments, the present invention provides a hepcidin analogue monomer, or a homodimer or heterodimer thereof, comprising a peptide that comprises, consists of, or consists essentially of a sequence DTX₁FX₂CY₁X₃F, wherein X₁ is any amino acid, Y₁ is any amino acid except for D-Cys, D-Ser, D-Ala, Cys(S-tBut), homoC, Pen, (D)Pen, Dap(AcBr), Inp, or D-His, and X₂ is any amino acid or it is absent. In one such embodiment, Y₁ is any amino acid except for Cys. In one such embodiment, Y₁ is any lipidic amino acid. In particular embodiments, Y₁ is selected from Val, Ile, and Leu. In one embodiment, Y₁ is Ile. In one embodiment, X₁, X2 (if not absent), and/or Y₁ is an unnatural amino acid. In some embodiments, a dimer comprising such a hepcidin analogue monomer comprises a linker (e.g., a lysine linker). In some embodiments, such a dimer comprises a first hepcidin analogue monomer and a second monomer (which monomers are optionally identical in sequence), and the dimer further comprises at least one intermolecular disulfide bridge linking a Cys in the first monomer (e.g., the Cys shown in either one of the above formulae) to a Cys in the second monomer.

In some embodiments, the present invention provides a hepcidin analogue homodimer or heterodimer comprising a hepcidin analogue monomer peptide that comprises, consists of, or consists essentially of a sequence DTX₁FPX₂C, wherein X₁ is any amino acid. In one embodiment, the present invention provides a hepcidin analogue homodimer or heterodimer comprising a hepcidin analogue monomer peptide that comprises, consists of, or consists essentially of a sequence DTX₁FPX₂CX₃F, wherein X₁ is any amino acid, X₂ is any amino acid, and X₃ is any amino acid or it is absent. In one such embodiment, X₂ is any amino acid except for Cys. In one embodiment, X₁, X₂, and/or X₃ is an unnatural amino acid. In some embodiments, a dimer comprising such a hepcidin analogue monomer comprises a linker (e.g., a lysine linker). In some embodiments, such a dimer comprises a first hepcidin analogue monomer and a second monomer (which monomers are optionally identical in sequence), and the dimer further comprises at least one intermolecular disulfide bridge linking a Cys in the first monomer (e.g., the Cys shown in either one of the above formulae) to a Cys in the second monomer.

In some embodiments, the present invention provides a hepcidin analogue monomer, or a homodimer or heterodimer thereof, comprising a peptide that comprises, consists of, or consists essentially of a sequence X₁X₁X₁FX₂X₂CY₁F wherein X₁ is absent or it is any amino acid, X₂ is any amino acid, and Y₁ is any amino acid. In one such embodiment, Y is any natural amino acid. In particular embodiments, Y₁ is selected from Arg, Val, Ile, and Leu. In one embodiment, Y₁ is Ile. In one embodiment, X₁, X₂, and/or Y₁ is an unnatural amino acid. In some embodiments, a dimer comprising such a hepcidin analogue monomer comprises a linker (e.g., a lysine linker). In some embodiments, such a dimer comprises a first hepcidin analogue monomer and a second monomer (which monomers are optionally identical in sequence), and the dimer further comprises at least one intermolecular disulfide bridge linking a Cys in the first monomer (e.g., the Cys shown in either one of the above formulae) to a Cys in the second monomer.

In some embodiments, the present invention provides a hepcidin analogue monomer, or a homodimer or heterodimer thereof, comprising a peptide that comprises, consists of, or consists essentially of a sequence DTX₁FX₂X₃CY₁F, wherein X₁ is any amino acid, X₂ is any amino acid or it is absent, X₃ is any amino acid, and Y₁ is any amino acid. In one such embodiment, Y₁ is any lipidic amino acid. In particular embodiments, Y₁ is selected from Val, Ile, and Leu. In one embodiment, Y₁ is Ile. In one embodiment, X₁, X₂ (if not absent), and/or Y₁ is an unnatural amino acid. In some embodiments, a dimer comprising such a hepcidin analogue monomer comprises a linker (e.g., a lysine linker). In some embodiments, such a dimer comprises a first hepcidin analogue monomer and a second monomer (which monomers are optionally identical in sequence), and the dimer further comprises at least one intermolecular disulfide bridge linking a Cys in the first monomer (e.g., the Cys shown in either one of the above formulae) to a Cys in the second monomer.

In some embodiments, the present invention provides a homodimer or heterodimer of one or more hepcidin analogue monomer that comprises, consists of, or consists essentially of a sequence X₁X₁X₁FX₂X₂CX₃F wherein X₁ is absent or it is any amino acid, X₂ is any amino acid, and X₃ is any amino acid. In one such embodiment, X₃ is any natural amino acid. In particular embodiments, X₃ is selected from Arg, Val, Ile, and Leu. In one embodiment, X₃ is Ile. In one embodiment, X₁, X₂, and/or X₃ is an unnatural amino acid. In some embodiments, a dimer comprising such a hepcidin analogue monomer comprises a linker (e.g., a lysine linker). In some embodiments, such a dimer comprises a first hepcidin analogue monomer and a second monomer (which monomers are optionally identical in sequence), and the dimer further comprises at least one intermolecular disulfide bridge linking a Cys in the first monomer (e.g., the Cys shown in either one of the above formulae) to a Cys in the second monomer.

In some embodiments, the present invention provides a homodimer or heterodimer of one or more hepcidin analogue monomer that comprises, consists of, or consists essentially of a sequence DTX₁FX₂X₃CX₄F, wherein X₁ is any amino acid, X₂ is any amino acid or it is absent, X₃ is any amino acid, and X₄ is any amino acid. In one such embodiment, X₄ is any amino acid except for Cys. In one such embodiment, X₄ is any lipidic amino acid. In particular embodiments, X₄ is selected from Val, Ile, and Leu. In one embodiment, X₄ is Ile. In one embodiment, X₁, X₂ (if not absent), and/or X₄ is an unnatural amino acid. In one embodiment, Cys is linked through a disulphide forming a dimer. In some embodiments, a dimer comprising such a hepcidin analogue monomer comprises a linker (e.g., a lysine linker). In some embodiments, such a dimer comprises a first hepcidin analogue monomer and a second monomer (which monomers are optionally identical in sequence), and the dimer further comprises at least one intermolecular disulfide bridge linking a Cys in the first monomer (e.g., the Cys shown in either one of the above formulae) to a Cys in the second monomer.

In certain embodiments, a peptide dimer (e.g., a hepcidin analogue or inhibitor) of the present invention comprises two peptide monomer subunits connected via one or more linker moieties or intermolecular linkages (e.g., a cysteine disulfide bridge), wherein each peptide monomer subunit comprises a sequence shown in any of Tables 2-4 or Tables 11-15. In certain embodiments, the peptide dimer is a homodimer, and in other embodiments, the peptide dimer is a heterodimer. In some embodiments, a linker moiety or intermolecular linkage that dimerizes two monomers is bound to any of the N-terminus, the C-terminus, or an internal amino acid (e.g., a lysine sidechain) of one or more of the monomer peptides.

In certain embodiments, a peptide dimer (e.g., a hepcidin analogue or inhibitor) of the present invention comprises two peptide monomer subunits connected via one or more linker moieties or intermolecular linkages (e.g., a cysteine disulfide bridge), wherein each peptide monomer subunit is: a compound of Formula I, wherein X is Ia and Y is Im, or wherein X is Ib and Y is In; a compound of Formula II, wherein X is IIa and Y is IIm; or a compound having a sequence shown in any of Tables 2-4, 10, 12, 14, and 15. In certain embodiments, the peptide dimer is a homodimer, and in other embodiments, the peptide dimer is a heterodimer. In particular embodiments, the peptide dimer is a peptide dimer as shown in any one of Tables 6-10, and 15.

In certain embodiments, at least two cysteine residues of the hepcidin analogue peptide dimers are linked by a disulfide bridge.

In particular embodiments of the hepcidin analogue peptide dimer of the present invention, the linker moiety (L) is any of the linkers shown in Table 5. In certain embodiments, the linker is a lysine linker, a diethylene glycol linker, an iminodiacetic acid (IDA) linker, a β-Ala-iminodiaceticacid (β-Ala-IDA) linker, or a PEG linker.

In certain embodiments of any of the hepcidin analogue peptide dimers, the N-terminus of each peptide monomer subunit is connected by a linker moiety.

In certain embodiments of any of the hepcidin analogue peptide dimers, the C-terminus of each peptide monomer subunit is connected by a linker moiety.

In certain embodiments, the side chains of one or more internal amino acid residues (e.g., Lys residues) comprised in each peptide monomer subunit of a hepcidin analogue peptide dimer are connected by a linker moiety.

In certain embodiments of any of the hepcidin analogue peptide dimers, the C-terminus, the N terminus, or an internal amino acid (e.g., a lysine sidechain) of each peptide monomer subunit is connected by a linker moiety and at least two cysteine residues of the hepcidin analogue peptide dimers are linked by a disulfide bridge. In some embodiments, a peptide dimer has a general structure shown below. Non-limiting schematic examples of such hepcidin analogues are shown in the following illustration:

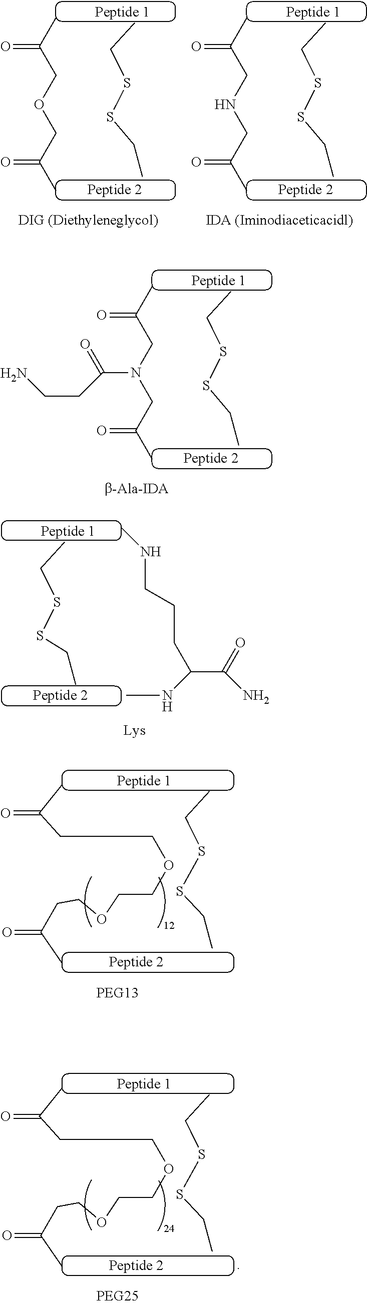

Illustrative examples of peptide dimer hepcidin analogues of the present invention are provided in Tables 6-8 with in vitro activity data in the ferroportin internalization/degradation assay described in the accompanying Examples.

TABLE 6
Illustrative Peptide Dimer Hepcidin Analogues

| | | Potency |
|---|---|---|
| SEQ ID | | EC₅₀ |
| NO | Sequence | (nM) |
| 527 | ([pGlu]-THFPCRKF-NH₂)₂ | 31% at 10 |
| | | uM |
| 528 | (Hy-DTHFPCLF-NH₂)₂ | 297 |

TABLE 7
Illustrative Peptide Dimer Hepcidin Analogues

| SEQ | | Potency |
|---|---|---|
| ID | | EC₅₀ |
| NO | Sequence | (nM) |
| 529 | (isovaleric acid-DTHFPICIFK(Palm)-NH₂)₂ | 580 |
| 530 | (isovaleric acid-DTHFPCIK(Palm)-F-NH₂)₂ | 294 |
| 531 | (isovaleric acid-DTHFPCIKFAA-NH₂)₂ | 47 |

TABLE 8
Illustrative Peptide Dimer Hepcidin Analogues

| SEQ | | Potency |
|---|---|---|
| ID | | EC₅₀ |
| NO | Sequence | (nM) |
| 532 | ([(D)Phe]-[(D)Ile]-[(D)Cys]- | Not active |
| | [(D)Ile]-[(D)Pro]-[(D)Phe]- | at 10 uM |
| | [(D)His]-[(D)Thr]-[(D)Asp])₂ | |
| 533 | (Hy-DTHFPICIF-NH₂)₂ | 146 |
| 534 | (Ida-TH-Dpa-bhPro-RCR-bhPhe-PEG3- | 31 |
| | Palm)₂ | |

In one embodiment, a peptide monomers of the present invention has the following structure:

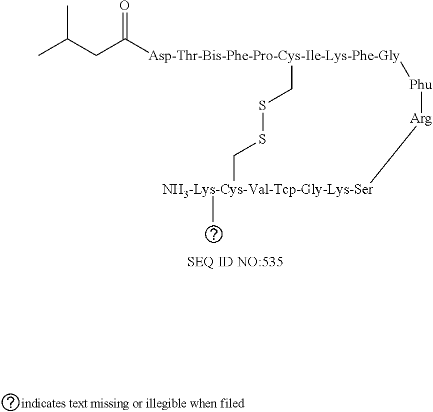

In one embodiment, a peptide monomers of the present invention has the following structure:

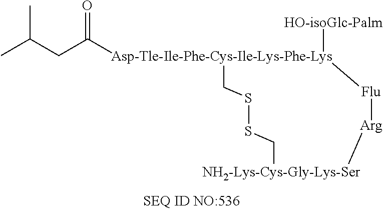

In one embodiment, a peptide dimer of the present invention has the following structure:

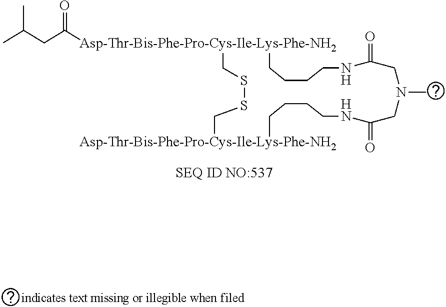

In one embodiment, the peptide dimer of the present invention has the following structure:

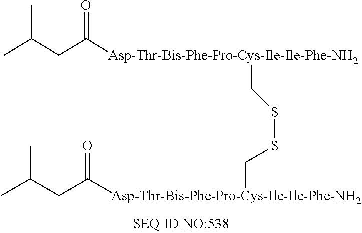

In certain embodiments, a peptide dimer inhibitor has the structure of Formula X:

(X)

SEQ ID NO: 23

(R¹-X-R²)₂-L

or a pharmaceutically acceptable salt or solvate thereof,

wherein each R¹ is independently absent, a bond (e.g., a covalent bond), or selected from hydrogen, a C1-C6 alkyl, a C6-C12 aryl, a C6-C12 aryl C1-C6 alkyl, a C1-C20 alkanoyl, and including PEGylated versions alone or as spacers of any of the foregoing;

each R² is independently absent, a bond (e.g., a covalent bond), or selected from OH or NH₂;

L is a linker moiety; and

each X is an independently selected peptide monomer subunit comprising or consisting of 7 to 35 amino acid residues, 8 to 35 amino acid residues, 9 to 35 amino acid residues, 10 to 35 amino acid residues, 7 to 25 amino acid residues, 8 to 25 amino acid residues, 9 to 25 amino acid residues, 10 to 25 amino acid residues, 7 to 18 amino acid residues, 8 to 18 amino acid residues, 9 to 18 amino acid residues, or 10 to 18 amino acid residues amino acids in length, each comprising or consisting of the sequence of Formula I or Formula II, or set forth in Tables 2-4, Tables 12-14, or a monomer sequence set forth in Table 15.

### Lysine Dimer Hepcidin Analogues

In certain embodiments, a peptide dimer hepcidin analogue of the present invention comprises two peptide monomer subunits linked via a lysine linker.

In some embodiments, a peptide dimer hepcidin analogue of the present invention has a structure of Formula IX:

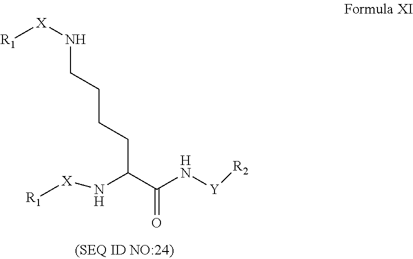

or a pharmaceutically acceptable salt of solvate thereof,

wherein each X is an independently selected peptide sequence having the formula IXa:

(IXa)

SEQ ID NO: 25

X1-X2-X3-X4-X5-X6-X7-X8-X9-X10

wherein

X1 is Asp, Glu, Ida or absent;
X2 is Thr, Ser, Pro, Ala or absent;

### X3 is His, Ala, Glu or Ala;

### X4 is Phe or Dpa;

X5 is Pro, bhPro, Sarc or Gly;
X6 is Cys, (D)-Cys, Arg, Glu, Phe, Gln, Leu, Val, Lys, Ala, Ser, Dapa or absent;
X7 is Cys, (D)-Cys, Arg, Glu, Phe, Gln, Leu, Val, Lys, Ala, Ser, Dapa or absent;
X8 is Ile, Arg, Lys, Ala, Gln, Phe, Glu, Asp, Tyr, Ser, Leu, Val, D-Ile, D-Lys, D-Arg, Dapa or absent;
X9 is Phe, Ala, Ile, Thr, Tyr, Lys, Arg, bhPhe, D-Phe or absent; and
X10 is Lys, Phe or absent;

wherein each R¹ is independently absent, a bond (e.g., a covalent bond), or selected from hydrogen, a C1-C6 alkyl, a C6-C12 aryl, a C6-C12 aryl C1-C6 alkyl, a C1-C20 alkanoyl, and including PEGylated versions alone or as spacers of any of the foregoing;

each R² is independently absent, a bond (e.g., a covalent bond), or selected from OH or NH₂;

Y is absent or present, and provided that if Y is present, Y is a peptide having the formula IXm:

(IXm)

SEQ ID NO: 26

Y1-Y2-Y3

wherein

Y1 is Ile, Arg, Lys, Ala, Gln, Phe, Glu, Asp, Tyr, Ser, Leu, Val, D-Ile, D-Lys, D-Arg, Dapa or absent;
Y2 is Phe, Ala, Ile, Thr, Tyr, Lys, Arg, bhPhe, D-Phe or absent; and
Y3 is Lys, Phe or absent.

In certain embodiments, one or more of Y1, Y2 and Y3 is present.

In certain embodiments, Y is conjugated to one or more chemical substituents, including but not limited to any of those described herein.

In some embodiments, one or both X is cyclized via a disulfide bond.

In some embodiments, the two X peptides are linked via a disulfide bond.

In certain embodiments, a lysine linked peptide dimer hepcidin analogue of the present has a structure set forth in Table 9.

TABLE 9
Illustrative Lysine-linked Dimer Hepcidin
Analogues

| SEQ | | EC₅₀ |
|---|---|---|
| ID | | (nM) |
| NO | Sequence | (n > 3) |
| 539 | (isobutyric acid- | 24 |
| | DTHFPCIKF)₂[Lys]K(iso-Glu-Palm)-NH₂ | |
| 540 | (isovaleric acid- | 14 |
| | DTHFPCIKF)₂[Lys]K(iso-Glu-Palm)-NH₂ | |
| 541 | (cyclohexanoic acid- | 17 |
| | DTHFPCIKF)₂[Lys]K(iso-Glu-Palm)-NH₂ | |
| 542 | (Isovaleric acid-DTHFPCIRF)₂[Lys]- | 4 |
| | K(iso-Glu-Palm)-NH₂ | |
| 543 | (Isovaleric acid-DTHFPCIKF)₂[Lys]-NH₂ | 30 |
| 544 | (Isovaleric acid-DTHFPCIKF)₂[Lys]- | 17 |
| | Lys(Palm)-NH₂ | |

In certain embodiments, each of the peptide monomer subunits of a lysine-linked peptide dimer hepcidin analogue of the present invention comprises or consists of a structure of Formula III:

(III)

SEQ ID NO: 7

R¹-X-Y-R²

or a pharmaceutically acceptable salt or solvate thereof, wherein

R¹ is hydrogen, a C1-C6 alkyl, a C6-C12 aryl, a C6-C12 aryl C1-C6 alkyl, or a C1-C20 alkanoyl, and including PEGylated versions thereof, alone or as spacers of any of the foregoing;

R² is —NH₂ or —OH;

X is a peptide sequence having the formula (IIIa)

(IIIa)

SEQ ID NO: 8

X1-X2-X3-X4-X5-X6-X7-X8-X9-X10

wherein

X1 is Asp, Glu, Ala, Gly, Thr, Ida, pGlu, bhAsp, D-Asp, Tyr, Leu or absent;
X2 is Thr, Ala, Aib, D-Thr, Arg or absent;

### X3 is His, Lys, Ala, or D-His;

X4 is Phe, Ala, Dpa or bhPhe;
X5 is Pro, Glu, Ser, Gly, Arg, Lys, Val, Ala, D-Pro, bhPro, Sarc, Abu or absent;

### X6 is Ile, Cys, Arg, Leu, Lys, His, Glu, D-Ile, D-Arg, D-Cys, Val, Ser or Ala;

### X7 is Cys, Ile, Ala, Leu, Val, Ser, Phe, Dapa, D-Ile or D-Cys;

### X8 is Ile, Lys, Arg, Ala, Gln, Phe, Glu, Asp, Tyr, Ser, Leu, Val, D-Ile, D-Lys, D-Arg, or Dapa;

X9 is Phe, Ala, lie, Tyr, Lys, Arg, bhPhe or D-Phe; and
X10 is Lys, Phe or absent;
Y is absent or present, and when present, Y is a peptide having the formula (IIIm)

(IIIm)

SEQ ID NO: 9

Y1-Y2-Y3-Y4-Y5-Y6-Y7-Y8-Y9-Y10-Y11-Y12-Y13-Y14-Y15

wherein

Y1 is Gly, Cys, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Tyr or absent;
Y5 is Lys, Met, Arg, Ala or absent;
Y6 is Gly, Ser, Lys, Ile, Arg, Ala, Pro, Val or absent;
Y7 is Trp, Lys, Gly, Ala, Ile, Val or absent;
Y8 is Val, Thr, Gly, Cys, Met, Tyr, Ala, Glu, Lys, Asp, Arg or absent;
Y9 is Cys, Tyr or absent;
Y10 is Met, Lys, Arg, Tyr or absent;
Y11 is Arg, Met, Cys, Lys or absent;
Y12 is Arg, Lys, Ala or absent;
Y13 is Arg, Cys, Lys, Val or absent;
Y14 is Arg, Lys, Pro, Cys, Thr or absent; and
Y15 is Thr, Arg or absent;

wherein if Y is absent from the peptide of formula (III), X7 is Ile; and

wherein said compound of formula (III) is optionally PEGylated on R¹, X, or Y.

In certain embodiments, R¹ is selected from methyl, acetyl, formyl, benzoyl, trifluoroacetyl, isovaleryl, isobutyryl, octanyl, and the conjugated amides of lauric acid, hexadecanoic acid, and γ-Glu-hexadecanoic acid.

In certain embodiments, X does not comprise and/or does not consist of an amino acid sequence set forth in U.S. Pat. No. 8,435,941.

In some embodiments, the compound or peptide of formula (III) comprises two or more cysteine residues, wherein at least two of said cysteine residues are linked via a disulfide bond.

In some embodiments, X is a peptide sequence according to formula (IIIa), described herein, wherein

X1 is Asp, Ala, Ida, pGlu, bhAsp, Leu, D-Asp or absent;

### X2 is Thr, Ala, or D-Thr;

### X3 is His, Lys, or D-His;

### X4 is Phe, Ala, or Dpa;

X5 is Pro, Gly, Arg, Lys, Ala, D-Pro or bhPro;

### X6 is Ile, Cys, Arg, Lys, D-Ile or D-Cys;

### X7 is Cys, Ile, Leu, Val, Phe, D-Ile or D-Cys;

### X8 is Ile, Arg, Phe, Gln, Lys, Glu, Val, Leu or D-Ile;

X9 is Phe or bhPhe; and
X10 is Lys, Phe or absent.

In some embodiments, X is a peptide sequence having the formula (IIIb)

(IIIb)

SEQ ID NO: 27

X1-Thr-His-X4-X5-X6-X7-X8-Phe-X10

wherein

X1 is Asp, Ida, pGlu, bhAsp or absent;

### X4 is Phe or Dpa;

X5 is Pro or bhPro;

### X6 is Ile, Cys or Arg;

### X7 is Cys, Ile, Leu or Val;

### X8 is Ile, Lys, Glu, Phe, Gln or Arg; and

X10 is Lys, Phe or absent;

In some embodiments, X is a peptide sequence according to formula (IIIb), as described herein, wherein

X1 is Asp, Glu, Ida, pGlu, bhAsp or absent;

### X4 is Phe or Dpa;

X5 is Pro or bhPro;

### X6 is Ile, Cys or Arg;

### X7 is Cys, Ile, Leu or Val;

### X8 is Ile, Lys, Glu, Phe, Gln or Arg; and

X10 is Lys or absent.

In some embodiments, X is a peptide sequence having the formula (IIIc)

(IIIc)

SEQ ID NO: 571

X1-Thr-His-X4-X5-Cys-Ile-X8-Phe-X10

wherein

X1 is Asp, Glu, Ida, pGlu, bhAsp or absent;

### X4 is: Phe or Dpa;

X5 is Pro or bhPro;

### X8 is Ile Lys, Glu, Phe, Gln or Arg; and

X10 is Lys or absent.

In some embodiments, X is a peptide sequence having the formula (IIId)

(IIId)

SEQ ID NO: 572

X1-Thr-His-Phe-X5-Cys-Ile-X8-Phe-X10

wherein

### X1 is Asp, Glu, or Ida;

### X4 is: Phe;

X5 is Pro or bhPro;

### X8 is Ile, Lys or Phe; and

X10 is absent.

In some embodiments, Y is a peptide sequence having the formula IIIn

(IIIn)

SEQ ID NO: 573

Y1-Y2-Y3-Y4-Y5-Y6-Y7-Y8-Cys-Y10

wherein

### Y1 is Gly, Ala, Lys, Pro or D-Pro;

### Y2 is Pro, Ala or Gly;

### Y3 is Arg, Ala, Lys or Trp;

### Y4 is Ser, Gly or Ala;

### Y5 is Lys, Met, Arg or Ala;

### Y6 is Gly, Arg or Ala;

Y7 is Trp, Ala or absent;
Y8 is Val, Thr, Lys, Ala, Glu or absent; and
Y10 is Met, Lys or absent.

In some embodiments, Y is a peptide sequence according to formula (IIIn), as described herein,

wherein

### Y1 is Gly, Ala, Lys, Pro or D-Pro;

### Y2 is Pro, Ala or Gly;

### Y3 is Arg, Ala, Lys or Trp;

### Y4 is Ser, Gly or Ala;

### Y5 is Lys, Met, Arg or Ala;

### Y6 is Gly, Arg or Ala;

### Y7 is Trp or Ala;

### Y8 is Val, Thr, Ala, or Glu; and

Y10 is Met, Lys or absent.

In some embodiments, Y is a peptide sequence having the formula (IIIo)

(IIIo)

SEQ ID NO: 574

Y1-Y2-Y3-Ser-Lys-Gly-Trp-Y8-Cys-Y10

wherein

### Y1 is Gly, Pro or D-Pro;

### Y2 is Pro or Gly;

### Y3 is Arg or Lys;

### Y8 is Val or Thr; and

Y10 is Met, Lys or absent.

In some embodiments, Y is a peptide sequence having the formula (IIIp)

(IIIp)

SEQ ID NO: 575

Y1-Cys-Y3-Y4-Arg-Y6-Y7-Y8-Cys-Y10-Y11-Y12-Y13-Y14-
Y15

wherein

Y1 is Val, Ala or absent;
Y3 is Gly, Pro or absent;

### Y4 is His, Trp or Tyr;

### Y6 is Ser, Gly or Pro;

### Y7 is Ile, Gly or Lys;

Y8 is Gly, Met or absent;

### Y10 is Tyr or Cys;

### Y11 is Arg, Lys, Met or Ala;

### Y12 is Arg or Ala;

Y13 is Cys or Val or absent;
Y14 is Cys, Lys, Pro, Arg, Thr or absent; and
Y15 is Arg, Thr or absent.

In some embodiments, Y is a peptide sequence having the formula (IIIq)

(IIIq)

SEQ ID NO

Val-Cys-Y3-His-Arg-Y6-Y7-Y8-Cys-Tyr-Arg-Y12-Y13-
Y14-Y15

wherein

Y3 is Gly or absent;

### Y6 is Ser or Pro;

### Y7 is Ile or Lys;

Y8 is Gly or absent;

### Y12 is Arg or Ala;

Y13 is Cys, Val or absent;
Y14 is Cys, Arg, Thr or absent; and
Y15 is Arg or absent.

In some embodiments, Y is a peptide sequence having the formula (IIIr)

(IIIr)

SEQ ID NO: 576

Y1-Pro-Y3-Ser-Y5-Y6-Y7-Y8-Cys-Y10

wherein

### Y1 is Gly, Glu, Val, or Lys;

### Y3 is Arg or Lys;

### Y5 is Arg or Lys;

### Y6 is Gly, Ser, Lys, Ile or Arg;

Y7 is Trp or absent;
Y8 is Val, Thr, Asp, Glu or absent; and
Y10 is Lys or absent.

In some embodiments, Y is a peptide sequence having the formula (IIIs)

(IIIs)

SEQ ID NO: 577

Y1-Pro-Y3-Ser-Y5-Y6-Y7-Y8-Cys-Y10

wherein

### Y1 is Glu or Lys;

### Y3 is Arg or Lys;

### Y5 is Arg or Lys;

### Y6 is Gly, Ser, Lys, Ile or Arg;

Y7 is Trp or absent;
Y8 is Val or absent; and
Y10 is Lys or absent.

In some embodiments, the peptide of formula (III) comprises at least three, at least four, at least five, at least six, at least seven, at least eight, at least nine, at least ten, at least eleven, at least twelve, at least thirteen, at least fourteen or at least fifteen Y residues in Y.

In some embodiments, Y1 to Y3 are present and Y4 to Y15 are absent.

In some embodiments, Y1 to Y11 are present and Y12 to Y15 are absent.

In some embodiments, Y1 to Y10 are present and Y11 to Y15 are absent.

In some embodiments, Y8 and Y15 are absent.

In some embodiments, Y3 and Y15 are absent.

In some embodiments, Y3, Y14 and Y15 are absent.

In some embodiment Y5 is absent.

In some embodiments Y1, Y5, Y7, Y12, Y13, Y14 and Y15 are absent.

In some embodiments Y1, Y5, and Y7 are absent. In some embodiments, Y8 is absent. In some embodiments, Y3 is absent. In some embodiments Y1, Y5, Y7, and Y11-Y15 are absent. In some embodiments, Y8 and Y11-Y15 are absent. In some embodiments, Y3 and Y11-Y15 are absent.

In certain embodiments, a peptide dimer hepcidin analogue of the present invention comprises two peptide monomer subunits linked via a lysine linker, comprising, consisting essentially of, or consisting of, the following structural formula:

(IV)

SEQ ID NO: 10

R¹-X-Y-R²

or a pharmaceutically acceptable salt or solvate thereof, wherein

wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;

R² is —NH₂ or —OH;

X is a peptide sequence having the formula (IVa)

(IVa)

SEQ ID NO: 11

X1-X2-X3-X4-X5-X6-X7-X8-X9-X10

wherein

X1 is Asp, Glu, Ala, Gly, Thr, Ida, pGlu, bhAsp, D-Asp, Tyr, Leu or absent;
X2 is Thr, Ala, Aib, D-Thr, Arg or absent;

### X3 is His, Lys, Ala, or D-His;

X4 is Phe, Ala, Dpa, bhPhe or D-Phe;
X5 is Pro, Glu, Ser, Gly, Arg, Lys, Val, Ala, D-Pro, bhPro, Sarc, Abu or absent;

### X6 is Ile, Cys, Arg, Leu, Lys, His, Glu, D-Ile, D-Arg, D-Cys, Val, Ser or Ala;

### X7 is Cys, Ile, Ala, Leu, Val, Ser, Phe, Dapa, D-Ile or D-Cys;

### X8 is Ile, Lys, Arg, Ala, Gln, Phe, Glu, Asp, Tyr, Ser, Leu, Val, D-Ile, D-Lys, D-Arg or Dapa;

X9 is Phe, Ala, Ile, Tyr, Lys, Arg, bhPhe or D-Phe; and
X10 is Lys, Phe or absent;

and provided that if Y′ is absent, X7 is Ile; and

Y is absent or is a peptide having the formula (IVm):

(IVm)

SEQ ID NO: 12

Y1-Y2-Y3-Y4-Y5-Y6-Y7-Y8-Y9-Y10-Y11-Y12-Y13-Y14-Y15

wherein

Y1 is Gly, Cys, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Tyr or absent;
Y5 is Lys, Met, Arg, Ala or absent;
Y6 is Gly, Ser, Lys, Ile, Arg, Ala, Pro, Val or absent;
Y7 is Trp, Lys, Gly, Ala, Ile, Val or absent;
Y8 is Val, Thr, Gly, Cys, Met, Tyr, Ala, Glu, Lys, Asp, Arg or absent;
Y9 is Cys, Tyr or absent;
Y10 is Met, Lys, Arg, Tyr or absent;
Y11 is Arg, Met, Cys, Lys or absent;
Y12 is Arg, Lys, Ala or absent;
Y13 is Arg, Cys, Lys, Val or absent;
Y14 is Arg, Lys, Pro, Cys, Thr or absent; and
Y15 is Thr, Arg or absent;

wherein said compound of formula (IV) is optionally PEGylated on R¹, X, or Y; and

wherein when said compound of formula (IV) comprises two or more cysteine residues, at least two of said cysteine residues being linked via a disulfide bond.

In certain embodiments, R¹ is selected from methyl, acetyl, formyl, benzoyl, trifluoroacetyl, isovaleryl, isobutyryl, octanyl, and the conjugated amides of lauric acid, hexadecanoic acid, and γ-Glu-hexadecanoic acid.

In some embodiments, R¹′ is hydrogen, isovaleric acid, isobutyric acid or acetyl.

In certain embodiments, X either or both does not comprise or does not consist of an amino acid sequence set forth in U.S. Pat. No. 8,435,941.

In some embodiments of the peptide compound of formula (IV), X is a peptide sequence according to formula (IVa), wherein

X1 is Asp, Ala, Ida, pGlu, bhAsp, Leu, D-Asp or absent;

### X2 is Thr, Ala, or D-Thr;

### X3 is His, Lys, D-His or Lys;

### X4 is Phe, Ala, Dpa or D-Phe;

X5 is Pro, Gly, Arg, Lys, Ala, D-Pro or bhPro;

### X6 is Ile, Cys, Arg, Lys, D-Ile or D-Cys;

### X7 is Cys, Ile, Leu, Val, Phe, D-Ile or D-Cys;

### X8 is Ile, Arg, Phe, Gln, Lys, Glu, Val, Leu or D-Ile;

X9 is Phe or bhPhe; and
X10 is Lys, Phe or absent.

In some embodiments of the peptide compound of formula IV, X is a peptide sequence having the formula (IVb)

(IVb)

SEQ ID NO: 578

X1-Thr-His-X4-X5-X6-X7-X8-Phe-X10

wherein

X1 is Asp, Ida, pGlu, bhAsp or absent;

### X4 is Phe or Dpa;

X5 is Pro or bhPro;

### X6 is Ile, Cys or Arg;

### X7 is Cys, Ile, Leu or Val;

### X8 is Ile Lys, Glu, Phe, Gin or Arg; and

X10 is Lys or absent.

In some embodiments of the peptide compound of formula IV, X is a peptide sequence having the formula (IVc)

(IVc)

SEQ ID NO: 579

X1-Thr-His-X4-X5-Cys-Ile-X8-Phe-X10

wherein

X1 is Asp, Ida, pGlu, bhAsp or absent;

### X4 is: Phe or Dpa;

X5 is Pro or bhPro;

### X8 is Ile Lys, Glu, Phe, Gin or Arg; and

X10 is Lys or absent;

In some embodiments of the peptide compound of formula IV, X is a peptide sequence having the formula (IVd)

(IVd)

SEQ ID NO: 580

X1-Thr-His-Phe-X5-Cys-Ile-X8-Phe-X10

wherein

### X1 is Asp, Glu, or Ida;

### X4 is: Phe;

X5 is Pro or bhPro;

### X8 is Ile, Lys, or Phe; and

X10 is absent;

In some embodiments of the peptide compound of formula IV, Y is a peptide sequence having the formula (IVn)

(IVn)

SEQ ID NO: 581

Y1-Y2-Y3-Y4-Y5-Y6-Y7-Y8-Cys-Y10

wherein

### Y1 is Gly, Ala, Lys, Pro or D-Pro;

### Y2 is Pro, Ala or Gly;

### Y3 is Arg, Ala, Lys or Trp;

### Y4 is Ser, Gly or Ala;

### Y5 is Lys, Met, Arg or Ala;

### Y6 is Gly, Arg or Ala;

### Y7 is Trp or Ala;

### Y8 is Val, Thr, Ala or Glu; and

Y10 is Met, Lys or absent.

In some embodiments of the peptide compound of formula IV, Y is a peptide sequence having the formula (IVo)

(IVo)

SEQ ID NO: 582

Y1-Y2-Y3-Ser-Lys-Gly-Trp-Y8-Cys-Y10

wherein

### Y1 is Gly, Pro or D-Pro;

### Y2 is Pro or Gly;

### Y3 is Arg or Lys;

### Y8 is Val or Thr; and

Y10 is Met, Lys or absent.

In some embodiments of the peptide compound of formula IV, Y is a peptide sequence having the formula (IVp)

(IVp)

SEQ ID NO: 583

Y1-Cys-Y3-Y4-Arg-Y6-Y7-Y8-Cys-Y10-Y11-Y12-Y13-Y14-
Y15

wherein

Y1 is Val or Ala or absent;
Y3 is Gly, Pro or absent;

### Y4 is His, Trp or Tyr;

### Y6 is Ser, Gly or Pro;

### Y7 is Ile, Gly or Lys;

Y8 is Gly, Met or absent;

### Y10 is Tyr or Cys;

### Y11 is Arg, Lys, Met or Ala;

### Y12 is Arg or Ala;

Y13 is Cys or Val or absent;
Y14 is Cys, Lys, Pro, Arg, Thr or absent; and
Y15 is Arg, Thr or absent.

In some embodiments of the peptide compound of formula IV, Y is a peptide sequence having the formula (IVq)

(IVq)

SEQ ID NO

Val-Cys-Y3-His-Arg-Y6-Y7-Y8-Cys-Tyr-Arg-Y12-Y13-
Y14-Y15

wherein

Y3 is Gly or absent;

### Y6 is Ser or Pro;

### Y7 is Ile or Lys;

Y8 is Gly or absent;

### Y12 is Arg or Ala;

Y13 is Cys, Val or absent;
Y14 is Cys, Arg, Thr or absent; and
Y15 is Arg or absent.

In some embodiments of the peptide compound of formula IV, Y is a peptide sequence having the formula (IVr)

(IVr)

SEQ ID NO

Y1-Pro-Y3-Ser-Y5-Y6-Y7-Y8-Cys-Y10

wherein

### Y1 is Gly, Glu, Val, or Lys;

### Y3 is Arg or Lys;

### Y5 is Arg or Lys;

### Y6 is Gly, Ser, Lys, Ile or Arg;

Y7 is Trp or absent;

Y8 is Val, Thr, Asp, Glu or absent; and

Y10 is Lys or absent.

In some embodiments of the peptide compound of formula IV, Y is a peptide sequence having the formula (IVs)

(IVs)

SEQ ID NO

Y1-Pro-Y3-Ser-Y5-Y6-Y7-Y8-Cys-Y10

wherein

### Y1 is Glu or Lys;

### Y3 is Arg or Lys;

### Y5 is Arg or Lys;

### Y6 is Gly, Ser, Lys, Ile or Arg;

Y7 is Trp or absent;
Y8 is Val or absent; and
Y10 is Lys or absent.

In some embodiments, the peptide of formula IV comprises at least three, at least four, at least five, at least six, at least seven, at least eight, at least nine, at least ten, at least eleven, at least twelve, at least thirteen, at least fourteen or at least fifteen Y residues in Y.

In some embodiments, Y1 to Y3 are present and Y4 to Y15 are absent.

In some embodiments, Y1 to Y11 are present and Y12 to Y15 are absent.

In some embodiments, Y1 to Y10 are present and Y11 to Y15 are absent.

In some embodiments, Y8 and Y15 are absent.

In some embodiments, Y3 and Y15 are absent

In some embodiments, Y3, Y14 and Y15 are absent.

In some embodiment Y5 is absent.

In some embodiments Y1, Y5, Y7, Y12, Y13, Y14 and Y15 are absent.

In certain embodiments, a peptide dimer hepcidin analogue of the present invention comprises two peptide monomer subunits linked via a lysine linker, comprising, consisting essentially of, or consisting of, the following structural formula:

(V)

SEQ ID NO: 13

R¹-X-Y-R²

or a pharmaceutically acceptable salt or solvate thereof, wherein

R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;

R² is —NH₂ or —OH;

X is a peptide sequence having the formula (Va)

(Va)

SEQ ID NO: 14

X1-X2-X3-X4-X5-X6-X7-X8-X9-X10

wherein

X1 is Asp, Glu, Ala, Gly, Thr, Ida, pGlu, bhAsp, D-Asp, Tyr, Leu or absent;
X2 is Thr, Ala, Aib, D-Thr, Arg or absent;

### X3 is His, Lys, Ala, D-His or Lys;

X4 is Phe, Ala, Dpa, bhPhe or D-Phe;
X5 is Pro, Glu, Ser, Gly, Arg, Lys, Val, Ala, D-Pro, bhPro, Sarc, Abu or absent;

### X6 is Ile, Cys, Arg, Leu, Lys, His, Glu, D-Ile, D-Arg, D-Cys, Val, Ser or Ala;

### X7 is Cys, Ile, Ala, Leu, Val, Ser, Phe, Dapa, D-Ile or D-Cys;

### X8 is Ile, Lys, Arg, Ala, Gln, Phe, Glu, Asp, Tyr, Ser, Leu, Val, D-Ile, D-Lys, D-Arg, or Dapa;

X9 is Phe, Ala, Ile, Tyr, Lys, Arg, bhPhe or D-Phe; and
X10 is Lys, Phe or absent;
wherein Y is present or absent, and provided that if Y is absent, X7 is Ile;

wherein said compound of formula V is optionally PEGylated on R¹, X, or Y; and

wherein when said compound of formula V comprises two or more cysteine residues, at least two of said cysteine residues being linked via a disulfide bond.

In certain embodiments, R¹ is selected from methyl, acetyl, formyl, benzoyl, trifluoroacetyl, isovaleryl, isobutyryl, octanyl, and the conjugated amides of lauric acid, hexadecanoic acid, and γ-Glu-hexadecanoic acid.

In some embodiments, R¹′ is hydrogen, isovaleric acid, isobutyric acid or acetyl.

In certain embodiments, X either or both does not comprise or does not consist of an amino acid sequence set forth in U.S. Pat. No. 8,435,941.

In some embodiments of the compound of formula (V), X is a peptide sequence according to formula (Va), wherein

X1 is Asp, Ala, Ida, pGlu, bhAsp, Leu, D-Asp or absent;

### X2 is Thr, Ala, or D-Thr;

### X3 is His, Lys, or D-His;

### X4 is Phe, Ala, or Dpa;

X5 is Pro, Gly, Arg, Lys, Ala, D-Pro or bhPro;

### X6 is Ile, Cys, Arg, Lys, D-Ile or D-Cys;

### X7 is Cys, Ile, Leu, Val, Phe, D-Ile or D-Cys;

### X8 is Ile, Arg, Phe, Gln, Lys, Glu, Val, Leu or D-Ile;

X9 is Phe or bhPhe; and
X10 is Lys or absent.

In some embodiments of the compound of formula (V), X is a peptide sequence having the formula (Vb)

(Vb)

SEQ ID NO: 584

X1-Thr-His-X4-X5-X6-X7-X8-Phe-X10

wherein

X1 is Asp, Ida, pGlu, bhAsp or absent;

### X4 is Phe or Dpa;

X5 is Pro or bhPro;

### X6 is Ile, Cys or Arg;

### X7 is Cys, Ile, Leu or Val;

### X8 is Ile, Lys, Glu, Phe, Gin or Arg; and

X10 is Lys, Phe or absent.

In some embodiments of the compound of formula (V), X is a peptide sequence having the formula (Ic″)

(Vc)

SEQ ID NO: 585

X1-Thr-His-X4-X5-Cys-Ile-X8-Phe-X10

wherein

X1 is Asp, Ida, pGlu, bhAsp or absent;

### X4 is Phe or Dpa;

X5 is Pro or bhPro;

### X8 is Ile, Lys, Glu, Phe, Gin or Arg; and

X10 is Lys or absent.

In some embodiments of the compound of formula (V), X is a peptide sequence having the formula (Vd)

(Vd)

SEQ ID NO: 586

X1-Thr-His-Phe-X5-Cys-Ile-X8-Phe-X10

wherein

### X1 is Asp, Glu or Ida;

### X4 is Phe;

X5 is Pro or bhPro;

### X8 is Ile, Lys, or Phe; and

X10 is absent.

In embodiments of the compound of formula (V) where Y is present, Y is a peptide having the formula (Vm)

(Vm)

SEQ ID NO: 587

Y1-Y2-Y3-Y4-Y5-Y6-Y7-Y8-Cys-Y10

wherein

### Y1 is Gly, Ala, Lys, Pro or D-Pro;

### Y2 is Pro, Ala or Gly;

### Y3 is Arg, Ala, Lys or Trp;

### Y4 is Ser, Gly or Ala;

### Y5 is Lys, Met, Arg or Ala;

### Y6 is Gly, Arg or Ala;

Y7 is Trp, Ala or absent;
Y8 is Val, Thr, Lys, Ala, Glu or absent; and
Y10 is Met, Lys or absent.

In some embodiments of the compound of formula (V), Y is a peptide sequence according to formula (Vm), wherein

### Y1 is Gly, Glu, Val, or Lys

### Y2 is Pro

### Y3 is Arg or Lys;

### Y4 is Ser

### Y5 is Arg or Lys;

### Y6 is Gly, Ser, Lys, Ile or Arg

Y7 is Trp or absent
Y8 is Val, Thr, Asp, Glu or absent; and
Y10 is Lys or absent.

In some embodiments of the compound of formula (V), Y is a peptide sequence according to formula (Vm), wherein

### Y1 is Glu or Lys

### Y2 is Pro

### Y3 is Arg or Lys;

### Y4 is Ser

### Y5 is Arg or Lys;

### Y6 is Gly, Ser, Lys, Ile or Arg;

Y7 is Trp or absent;
Y8 is Val or absent; and
Y10 is Lys or absent

In some embodiments of the compound of formula (V), Y is a peptide sequence according to formula (Vm), wherein

### Y1 is Gly, Pro or D-Pro;

### Y2 is Pro or Gly;

### Y3 is Arg or Lys;

### Y4 is Ser;

### Y5 is Lys;

### Y6 is Gly;

### Y7 is Trp;

### Y8 is Val or Thr; and

Y10 is Met, Lys or absent.

In some embodiments of the compound of formula (V), Y is a peptide sequence having the formula (Vn):

(Vn)

SEQ ID NO: 588

Y1-Y2-Y3-Ser-Lys-Gly-Trp-Y8-Cys-Y10

wherein

### Y1 is Gly, Pro or D-Pro;

### Y2 is Pro or Gly;

### Y3 is Arg or Lys;

### Y8 is Val or Thr; and

Y10 is Met, Lys or absent.

In some embodiments the peptide of formula (V) comprises at least three, at least four, at least five, at least six, at least seven, at least eight, at least nine, or at least ten amino acid residues of Y. In some embodiments, Y1 to Y3 are present and Y4 to Y10 are absent.

In some embodiments, Y5 is absent. In some embodiments Y1, Y5, and Y7 are absent. In some embodiments, Y8 is absent. In some embodiments, Y3 is absent.

In certain embodiments, a peptide dimer hepcidin analogue of the present invention comprises two peptide monomer subunits linked via a lysine linker, comprising, consisting essentially of, or consisting of, the following structural formula VI:

(VI)

SEQ ID NO: 15

R¹-X-Y-R²

or a pharmaceutically acceptable salt or solvate thereof, wherein

wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;

R² is —NH₂ or —OH;

X is a peptide sequence having the formula (VIa):

(VIa)

SEQ ID NO: 16

X1-X2-X3-X4-X5-X6-X7-X8-X9-X10

wherein

X1 is Asp, Glu, Ida or absent;
X2 is Thr, Ser, Pro, Ala or absent;

### X3 is His, Ala, or Glu;

### X4 is Phe or Dpa;

X5 is Pro, bhPro, Sarc or Gly;
X6 is Cys, (D)-Cys, Arg, Glu, Phe, Gln, Leu, Val, Lys, Ala, Ser, Dapa or absent;
X7 is Cys, (D)-Cys, Arg, Glu, Phe, Gln, Leu, Val, Lys, Ala, Ser, Dapa or absent;
X8 is Ile, Arg, Lys, Ala, Gln, Phe, Glu, Asp, Tyr, Ser, Leu, Val, D-Ile, D-Lys, D-Arg, Dapa or absent;
X9 is Phe, Ala, Ile, Thr, Tyr, Lys, Arg, bhPhe, D-Phe or absent; and
X10 is Lys, Phe or absent;

Y is absent or present, provided that if Y is present, Y is a peptide having the formula (VIm)

(VIm)

SEQ ID NO: 17

Y1-Y2-Y3

wherein

Y1 is Ile, Arg, Lys, Ala, Gln, Phe, Glu, Asp, Tyr, Ser, Leu, Val, D-Ile, D-Lys, D-Arg, Dapa or absent;
Y2 is Phe, Ala, Ile, Thr, Tyr, Lys, Arg, bhPhe or D-Phe or absent; and
Y3 is Lys, Phe or absent;

and wherein said compound of formula VI is optionally PEGylated on R¹, X, or Y.

As used herein, the term “having” means “comprising,” “consisting of” or “consisting essentially of” and encompasses each of these various embodiments in each instance.

In certain embodiments, a peptide analogue of formula VI comprises two or more cysteine residues, at least two of said cysteine residues being linked via a disulfide bond.

In certain embodiments, R¹ is selected from methyl, acetyl, formyl, benzoyl, trifluoroacetyl, isovaleryl, isobutyryl, octanyl, and the conjugated amides of lauric acid, hexadecanoic acid, and γ-Glu-hexadecanoic acid.

In some embodiments, R¹′ is hydrogen, isovaleric acid, isobutyric acid or acetyl.

In certain embodiments, X either or both does not comprise or does not consist of an amino acid sequence set forth in U.S. Pat. No. 8,435,941.

In certain embodiments, a dimer hepcidin analogue of the present invention, e.g., a lysine dimer hepcidin analogue of the present invention, comprises one or two peptide monomers having an amino acid sequence shown as any one of compound numbers 1-361 in Table 12 with ferroportin internalization/degradation assay EC₅₀ values.

For certain compounds comprising an N-terminal PEG11 moiety, the following was used in their synthesis:

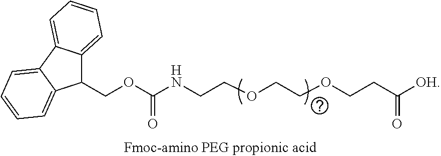

In certain embodiments, a lysine dimer peptide analogue of the present invention has a structure or comprises a peptide sequence shown in Table 10 with ferroportin internalization/degradation assay EC50 values.

TABLE 10
Illustrative Lysine dimer peptide analogues

| SEQ | | EC₅₀ |
|---|---|---|
| ID | | (nM) |
| NO | Sequence | (n > 3) |
| 539 | (isobutyric acid-DTHFPCIKF)₂[Lys]K(iso- | 24 |
| | Glu-Palm)-NH₂ | |
| 540 | (isovaleric acid-DTHFPCIKF)₂[Lys]K(iso- | 14 |
| | Glu-Palm)-NH₂ | |
| 541 | (cyclohexanoic acid-DTHFPCIKF)₂ | 17 |
| | [Lys]K(iso-Glu-Palm)-NH₂ | |
| 542 | (Isovaleric acid-DTHFPCIRF)₂[Lys]- | 4 |
| | K(iso-Glu-Palm)-NH₂ | |
| 543 | (Isovaleric acid-DTHFPCIKF)₂[Lys]-NH₂ | 30 |
| 544 | (Isovaleric acid-DTHFPCIKF)₂[Lys]- | 16 |
| | Lys(Palm)-NH₂ | |
| 570 | (Isovaleric acid-DTHFPCIKF)₂[Lys]- | 17 |
| | Lys[(isoGlu(octanoic acid)]-NH2 | |

### Peptide Analogue Conjugates and Analogues

In certain embodiments, hepcidin analogues of the present invention, including both monomers and dimers, comprise one or more conjugated chemical substituents, such as lipophilic substituents and polymeric moieties. Without wishing to be bound by any particular theory, it is believed that the lipophilic substituent binds to albumin in the bloodstream, thereby shielding the hepcidin analogue from enzymatic degradation, and thus enhancing its half-life. In addition, it is believed that polymeric moieties enhance half-life and reduce clearance in the bloodstream, and in some cases enhance permeability through the epithelium and retention in the lamina propria. Moreover, it is also surmised that these substituents in some cases may enhance permeability through the epithelium and retention in the lamina propria. The skilled person will be well aware of suitable techniques for preparing the compounds employed in the context of the invention. For examples of non-limiting suitable chemistry, see, e.g., WO98/08871, WO00/55184, WO00/55119, Madsen et al (J. Med. Chem. 2007, 50, 6126-32), and Knudsen et al. 2000 (J. Med Chem. 43, 1664-1669).

In one embodiment, the side chains of one or more amino acid residues (e.g., Lys residues) in a hepcidin analogue of the invention is further conjugated (e.g., covalently attached) to a lipophilic substituent. The lipophilic substituent may be covalently bonded to an atom in the amino acid side chain, or alternatively may be conjugated to the amino acid side chain via one or more spacers. The spacer, when present, may provide spacing between the hepcidin analogue and the lipophilic substituent.

In certain embodiments, the lipophilic substituent comprises a hydrocarbon chain having from 4 to 30 C atoms, for example at least 8 or 12 C atoms, and preferably 24 C atoms or fewer, or 20 C atoms or fewer. The hydrocarbon chain may be linear or branched and may be saturated or unsaturated. In certain embodiments, the hydrocarbon chain is substituted with a moiety which forms part of the attachment to the amino acid side chain or the spacer, for example an acyl group, a sulfonyl group, an N atom, an O atom or an S atom. In some embodiments, the hydrocarbon chain is substituted with an acyl group, and accordingly the hydrocarbon chain may form part of an alkanoyl group, for example palmitoyl, caproyl, lauroyl, myristoyl or stearoyl.

A lipophilic substituent may be conjugated to any amino acid side chain in a hepcidin analogue of the invention. In certain embodiment, the amino acid side chain includes a carboxy, hydroxyl, thiol, amide or amine group, for forming an ester, a sulphonyl ester, a thioester, an amide or a sulphonamide with the spacer or lipophilic substituent. For example, the lipophilic substituent may be conjugated to Asn, Asp, Glu, Gln, His, Lys, Arg, Ser, Thr, Tyr, Trp, Cys or Dbu, Dpr or Orn. In certain embodiments, the lipophilic substituent is conjugated to Lys. An amino acid shown as Lys in any of the formula provided herein may be replaced by, e.g., Dbu, Dpr or Orn where a lipophilic substituent is added.

In further embodiments of the present invention, alternatively or additionally, the side-chains of one or more amino acid residues in a hepcidin analogue of the invention may be conjugated to a polymeric moiety, for example, in order to increase solubility and/or half-life in vivo (e.g. in plasma) and/or bioavailability. Such modifications are also known to reduce clearance (e.g. renal clearance) of therapeutic proteins and peptides.

As used herein, “Polyethylene glycol” or “PEG” is a polyether compound of general formula H—(O—CH2-CH2)n-OH. PEGs are also known as polyethylene oxides (PEOs) or polyoxyethylenes (POEs), depending on their molecular weight PEO, PEE, or POG, as used herein, refers to an oligomer or polymer of ethylene oxide. The three names are chemically synonymous, but PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. Throughout this disclosure, the 3 names are used indistinguishably. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g. viscosity) due to chain length effects, their chemical properties are nearly identical. The polymeric moiety is preferably water-soluble (amphiphilic or hydrophilic), non-toxic, and pharmaceutically inert. Suitable polymeric moieties include polyethylene glycols (PEG), homo- or co-polymers of PEG, a monomethyl-substituted polymer of PEG (mPEG), or polyoxyethylene glycerol (POG). See, for example, Int. J. Hematology 68:1 (1998); Bioconjugate Chem. 6:150 (1995); and Crit. Rev. Therap. Drug Carrier Sys. 9:249 (1992). Also encompassed are PEGs that are prepared for purpose of half-life extension, for example, mono-activated, alkoxy-terminated polyalkylene oxides (POA's) such as mono-methoxy-terminated polyethyelene glycols (mPEG's); bis activated polyethylene oxides (glycols) or other PEG derivatives are also contemplated. Suitable polymers will vary substantially by weights ranging from about 200 to about 40,000 are usually selected for the purposes of the present invention. In certain embodiments, PEGs having molecular weights from 200 to 2,000 or from 200 to 500 are used. Different forms of PEG may also be used, depending on the initiator used for the polymerization process, e.g., a common initiator is a monofunctional methyl ether PEG, or methoxypoly(ethylene glycol), abbreviated mPEG. Other suitable initiators are known in the art and are suitable for use in the present invention.

Lower-molecular-weight PEGs are also available as pure oligomers, referred to as monodisperse, uniform, or discrete. These are used in certain embodiments of the present invention.

PEGs are also available with different geometries: branched PEGs have three to ten PEG chains emanating from a central core group; star PEGs have 10 to 100 PEG chains emanating from a central core group; and comb PEGs have multiple PEG chains normally grafted onto a polymer backbone. PEGs can also be linear. The numbers that are often included in the names of PEGs indicate their average molecular weights (e.g. a PEG with n=9 would have an average molecular weight of approximately 400 daltons, and would be labeled PEG 400.

As used herein, “PEGylation” is the act of coupling (e.g., covalently) a PEG structure to the hepcidin analogue of the invention, which is in certain embodiments referred to as a “PEGylated hepcidin analogue”. In certain embodiments, the PEG of the PEGylated side chain is a PEG with a molecular weight from about 200 to about 40,000. In some embodiments, a spacer of a peptide of formula I, formula I′, or formula I″ is PEGylated. In certain embodiments, the PEG of a PEGylated spacer is PEG3, PEG4, PEGS, PEG6, PEG7, PEG8, PEG9, PEG10, or PEG11. In certain embodiments, the PEG of a PEGylated spacer is PEG3 or PEG8.

In some embodiments, the present invention includes a hepcidin analogue peptide (or a dimer thereof) conjugated with a PEG that is attached covalently, e.g., through and amide, a thiol, via click chemistry, or via any other suitable means known in the art. In particular embodiments PEG is attached through an amide bond and, as such, certain PEG derivatives used will be appropriately functionalized. For example, in certain embodiments, PEG11, which is O-(2-aminoethyl)-O′-(2-carboxyethyl)-undecaethyleneglycol, has both an amine and carboxylic acid for attachment to a peptide of the present invention. In certain embodiments, PEG25 contains a diacid and 25 glycol moieties.

Other suitable polymeric moieties include poly-amino acids such as poly-lysine, poly-aspartic acid and poly-glutamic acid (see for example Gombotz, et al. (1995), Bioconjugate Chem., vol. 6: 332-351; Hudecz, et al. (1992), Bioconjugate Chem., vol. 3, 49-57 and Tsukada, et al. (1984), J. Natl. Cancer Inst., vol. 73: 721-729. The polymeric moiety may be straight-chain or branched. In some embodiments, it has a molecular weight of 500-40,000 Da, for example 500-10,000 Da, 1000-5000 Da, 10,000-20,000 Da, or 20,000-40,000 Da.

In some embodiments, a hepcidin analogue of the invention may comprise two or more such polymeric moieties, in which case the total molecular weight of all such moieties will generally fall within the ranges provided above.

In some embodiments, the polymeric moiety may be coupled (by covalent linkage) to an amino, carboxyl or thiol group of an amino acid side chain. Certain examples are the thiol group of Cys residues and the epsilon amino group of Lys residues, and the carboxyl groups of Asp and Glu residues may also be involved.

The skilled worker will be well aware of suitable techniques which can be used to perform the coupling reaction. For example, a PEG moiety bearing a methoxy group can be coupled to a Cys thiol group by a maleimido linkage using reagents commercially available from Nektar Therapeutics AL. See also WO 2008/101017, and the references cited above, for details of suitable chemistry. A maleimide-functionalised PEG may also be conjugated to the side-chain sulfhydryl group of a Cys residue.

As used herein, disulfide bond oxidation can occur within a single step or is a two-step process. As used herein, for a single oxidation step, the trityl protecting group is often employed during assembly, allowing deprotection during cleavage, followed by solution oxidation. When a second disulfide bond is required, one has the option of native or selective oxidation. For selective oxidation requiring orthogonal protecting groups, Acm and Trityl is used as the protecting groups for cysteine. Cleavage results in the removal of one protecting pair of cysteine allowing oxidation of this pair. The second oxidative deprotection step of the cysteine protected Acm group is then performed. For native oxidation, the trityl protecting group is used for all cysteines, allowing for natural folding of the peptide.

A skilled worker will be well aware of suitable techniques which can be used to perform the oxidation step.

### Illustrative Hepcidin Analogue Peptide Monomers and Hepcidin Analogue Peptide Dimers

Illustrative hepcidin analogues and hepcidin analogue peptide dimers of the present invention are shown in Tables 2-4, 6-10, 12, 14, and 15. These tables provides the amino acid sequence of selected monomer hepcidin analogues and hepcidin analogue peptide dimers, and in some cases indicate the linker moiety present in the hepcidin analogue peptide dimers. According to the protocols discussed herein, a number of the hepcidin analogues monomer peptides and hepcidin analogue peptide dimers shown were synthesized. The IC50 values for selected monomer hepcidin analogues and hepcidin analogue peptide dimers for inducing the internalization/degradation of human ferroportin protein in vitro are provided.

The present invention thus provides various hepcidin analogues which bind or associate with ferroportin (e.g., human ferroportin), inducing internalization of the transporter.

In some embodiments, the present invention provides a dimer of any one of the peptide monomers disclosed herein. In one embodiment, the present invention provides a hepcidin analogue dimer that is a homodimer of any one of the monomer peptide sequences disclosed herein. In one embodiment, the present invention provides a hepcidin analogue dimer that is a heterodimer of any two different monomer peptide sequences disclosed herein.

In one embodiment, the present invention provides a hepcidin analogue dimer that is a heterodimer of any one monomer peptide sequence disclosed herein and any other peptide sequence known in the art to have hepcidin activity including a wildtype hepcidin peptide or a hepcidin analogue. In various embodiments, the present invention provides hepcidin homodimers and heterodimers that are dimerized by a disulfide linkage. In various embodiments, the present invention provides hepcidin homodimers and heterodimers that are dimerized via a linker, e.g., any one or more of the linkers disclosed herein or known in the art. In still further embodiments, the present invention provides hepcidin homodimers and heterodimers that are dimerized by one or more disulfide linkages and one or more linker, e.g., any one or more of the linkers disclosed herein or known in the art.

The hepcidin analogues of the present invention may be synthesized by many techniques that are known to those skilled in the art. In certain embodiments, monomer subunits are synthesized, purified, and dimerized using the techniques described in the accompanying Examples.

In related embodiments, the present invention includes polynucleotides that encode a polypeptide having a sequence set forth in any one of Formula I-IX, or as shown in any of Tables 2-4, 6-10, 12, 14, or 15.

In addition, the present invention includes vectors, e.g., expression vectors, comprising a polynucleotide of the present invention.

In certain embodiments, the present invention provides a hepcidin analogue monomer, or a homodimer or heterodimer comprising such a monomer, according to any one of the formulae disclosed herein, wherein the monomer comprises a Cys in position 6 or 7 and wherein the amino acid directly C-terminal to such a Cys is any natural or unnatural amino acid except for Ile.

### Methods of Treatment

In some embodiments, the present invention provides methods for treating a subject afflicted with a disease or disorder associated with dysregulated hepcidin signaling, wherein the method comprises administering to the subject a hepcidin analogue of the present invention. In some embodiments, the hepcidin analogue that is administered to the subject is present in a composition (e.g., a pharmaceutical composition). In one embodiment, a method is provided for treating a subject afflicted with a disease or disorder characterized by increased activity or expression of ferroportin, wherein the method comprises administering to the individual a hepcidin analogue or composition of the present invention in an amount sufficient to (partially or fully) bind to and agonize ferroportin in the subject. In one embodiment, a method is provided for treating a subject afflicted with a disease or disorder characterized by dysregulated iron metabolism, wherein the method comprises administering to the subject a hepcidin analogue or composition of the present invention.

In some embodiments, methods of the present invention comprise providing a hepcidin analogue or a composition of the present invention to a subject in need thereof. In particular embodiments, the subject in need thereof has been diagnosed with or has been determined to be at risk of developing a disease or disorder characterized by dysregulated iron levels (e.g., diseases or disorders of iron metabolism; diseases or disorders related to iron overload; and diseases or disorders related to abnormal hepcidin activity or expression). In particular embodiments, the subject is a mammal (e.g., a human).

In certain embodiments, the disease or disorder is a disease of iron metabolism, such as, e.g., an iron overload disease, iron deficiency disorder, disorder of iron biodistribution, or another disorder of iron metabolism and other disorder potentially related to iron metabolism, etc. In particular embodiments, the disease of iron metabolism is hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, beta thalassemia, sideroblastic anemia, porphyria, porphyria cutanea tarda, African iron overload, hyperferritinemia, ceruloplasmin deficiency, atransferrinemia, congenital dyserythropoietic anemia, anemia of chronic disease, anemia of inflammation, anemia of infection, hypochromic microcytic anemia, iron-deficiency anemia, iron-refractory iron deficiency anemia, anemia of chronic kidney disease, transfusion-dependent anemia, hemolytic anemia, erythropoietin resistance, iron deficiency of obesity, other anemias, benign or malignant tumors that overproduce hepcidin or induce its overproduction, conditions with hepcidin excess, Friedreich ataxia, gracile syndrome, Hallervorden-Spatz disease, Wilson's disease, pulmonary hemosiderosis, hepatocellular carcinoma, cancer (e.g., liver cancer), hepatitis, cirrhosis of liver, pica, chronic renal failure, insulin resistance, diabetes, atherosclerosis, neurodegenerative disorders, dementia, multiple sclerosis, Parkinson's disease, Huntington's disease, or Alzheimer's disease.

In certain embodiments, the disease or disorder is related to iron overload diseases such as iron hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia.

In certain embodiments, the disease or disorder is one that is not typically identified as being iron related. For example, hepcidin is highly expressed in the murine pancreas suggesting that diabetes (Type I or Type II), insulin resistance, glucose intolerance and other disorders may be ameliorated by treating underlying iron metabolism disorders. See Ilyin, G. et al. (2003) FEBS Lett. 542 22-26, which is herein incorporated by reference. As such, peptides of the invention may be used to treat these diseases and conditions. Those skilled in the art are readily able to determine whether a given disease can be treated with a peptide according to the present invention using methods known in the art, including the assays of WO 2004092405, which is herein incorporated by reference, and assays which monitor hepcidin, hemojuvelin, or iron levels and expression, which are known in the art such as those described in U.S. Pat. No. 7,534,764, which is herein incorporated by reference.

In certain embodiments, the disease or disorder is postmenopausal osteoporosis.

In certain embodiments of the present invention, the diseases of iron metabolism are iron overload diseases, which include hereditary hemochromatosis, iron-loading anemias, alcoholic liver diseases, heart disease and/or failure, cardiomyopathy, and chronic hepatitis C.

In particular embodiments, any of these diseases, disorders, or indications are caused by or associated with a deficiency of hepcidin or iron overload.

In some embodiments, methods of the present invention comprise providing a hepcidin analogue of the present invention (i.e., a first therapeutic agent) to a subject in need thereof in combination with a second therapeutic agent. In certain embodiments, the second therapeutic agent is provided to the subject before and/or simultaneously with and/or after the pharmaceutical composition is administered to the subject. In particular embodiments, the second therapeutic agent is iron chelator. In certain embodiments, the second therapeutic agent is selected from the iron chelators Deferoxamine and Deferasirox (Exjade™). In another embodiment, the method comprises administering to the subject a third therapeutic agent.

The present invention provides compositions (for example pharmaceutical compositions) comprising one or more hepcidin analogues of the present invention.

In certain embodiments, the compositions comprise two or more hepcidin analogues disclosed herein. In certain embodiments, the combination is selected from one of the following: (i) any two or more of the hepcidin analogue peptide monomers, such as, e.g., any one of those disclosed in Tables 2-4, 6-10, 12, 14, or 15, or dimers of any monomers shown therein; (ii) any two or more of the hepcidin analogue peptide dimers disclosed in Tables 2-4 or 6-10, 12, 14, or 15; (iii) any one or more of the hepcidin analogue peptide monomers disclosed herein, and any one or more of the hepcidin analogue peptide dimers disclosed herein.

In certain embodiments, the present invention includes pharmaceutical compositions comprising one or more hepcidin analogues of the present invention and a pharmaceutically acceptable carrier, diluent or excipient. A pharmaceutically acceptable carrier, diluent or excipient refers to a non-toxic solid, semi-solid or liquid filler, diluent, encapsulating material or formulation auxiliary of any type. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like.

The term “pharmaceutically acceptable carrier” includes any of the standard pharmaceutical carriers. Pharmaceutically acceptable carriers for therapeutic use are well known in the pharmaceutical art and are described, for example, in “Remington's Pharmaceutical Sciences”, 17th edition, Alfonso R. Gennaro (Ed.), Mark Publishing Company, Easton, Pa., USA, 1985. For example, sterile saline and phosphate-buffered saline at slightly acidic or physiological pH may be used. Suitable pH-buffering agents may, e.g., be phosphate, citrate, acetate, tris(hydroxymethyl)aminomethane (TRIS), N-tris(hydroxymethyl)methyl-3-aminopropanesulfonic acid (TAPS), ammonium bicarbonate, diethanolamine, histidine, arginine, lysine or acetate (e.g. as sodium acetate), or mixtures thereof. The term further encompasses any carrier agents listed in the US Pharmacopeia for use in animals, including humans.

It is to be understood that the inclusion of a hepcidin analogue of the invention (i.e., one or more hepcidin analogue peptide monomers of the invention or one or more hepcidin analogue peptide dimers of the present invention) in a pharmaceutical composition also encompasses inclusion of a pharmaceutically acceptable salt or solvate of a hepcidin analogue of the invention. In particular embodiments, the pharmaceutical compositions further comprise one or more pharmaceutically acceptable carrier, excipient, or vehicle.

In certain embodiments, the invention provides a pharmaceutical composition comprising a hepcidin analogue, or a pharmaceutically acceptable salt or solvate thereof, for treating a variety of conditions, diseases, or disorders as disclosed herein or elsewhere (see, e.g., Methods of Treatment, herein). In particular embodiments, the invention provides a pharmaceutical composition comprising a hepcidin analogue peptide monomer, or a pharmaceutically acceptable salt or solvate thereof, for treating a variety of conditions, diseases, or disorders as disclosed herein elsewhere (see, e.g., Methods of Treatment, herein).

In particular embodiments, the invention provides a pharmaceutical composition comprising a hepcidin analogue peptide dimer, or a pharmaceutically acceptable salt or solvate thereof, for treating a variety of conditions, diseases, or disorders as disclosed herein elsewhere (see, e.g., Methods of Treatment, herein).

The hepcidin analogues of the present invention may be formulated as pharmaceutical compositions which are suited for administration with or without storage, and which typically comprise a therapeutically effective amount of at least one hepcidin analogue of the invention, together with a pharmaceutically acceptable carrier, excipient or vehicle.

In some embodiments, the hepcidin analogue pharmaceutical compositions of the invention are in unit dosage form. In such forms, the composition is divided into unit doses containing appropriate quantities of the active component or components. The unit dosage form may be presented as a packaged preparation, the package containing discrete quantities of the preparation, for example, packaged tablets, capsules or powders in vials or ampoules. The unit dosage form may also be, e.g., a capsule, cachet or tablet in itself, or it may be an appropriate number of any of these packaged forms. A unit dosage form may also be provided in single-dose injectable form, for example in the form of a pen device containing a liquid-phase (typically aqueous) composition. Compositions may be formulated for any suitable route and means of administration, e.g., any one of the routes and means of administration disclosed herein.

In particular embodiments, the hepcidin analogue, or the pharmaceutical composition comprising a hepcidin analogue, is suspended in a sustained-release matrix. A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. One embodiment of a biodegradable matrix is a matrix of one of either polylactide, polyglycolide, or polylactide co-glycolide (co-polymers of lactic acid and glycolic acid).

In certain embodiments, the compositions are administered enterally or parenterally. In particular embodiments, the compositions are administered orally, intracistemally, intravaginally, intraperitoneally, intrarectally, topically (as by powders, ointments, drops, suppository, or transdermal patch, including delivery intravitreally, intranasally, and via inhalation) or buccally. The term “parenteral” as used herein refers to modes of administration which include intravenous, intramuscular, intraperitoneal, intrastemal, subcutaneous, intradermal and intraarticular injection and infusion. Accordingly, in certain embodiments, the compositions are formulated for delivery by any of these routes of administration.

In certain embodiments, pharmaceutical compositions for parenteral injection comprise pharmaceutically acceptable sterile aqueous or nonaqueous solutions, dispersions, suspensions or emulsions, or sterile powders, for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, beta-cyclodextrin, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants. These compositions may also contain adjuvants such as preservative, wetting agents, emulsifying agents, and dispersing agents. Prolonged absorption of an injectable pharmaceutical form may be brought about by the inclusion of agents which delay absorption, such as aluminum monostearate and gelatin.

Injectable depot forms include those made by forming microencapsule matrices of the hepcidin analogue in one or more biodegradable polymers such as polylactide-polyglycolide, poly(orthoesters), poly(anhydrides), and (poly)glycols, such as PEG. Depending upon the ratio of peptide to polymer and the nature of the particular polymer employed, the rate of release of the hepcidin analogue can be controlled. Depot injectable formulations are also prepared by entrapping the hepcidin analogue in liposomes or microemulsions compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Topical administration includes administration to the skin or mucosa, including surfaces of the lung and eye. Compositions for topical lung administration, including those for inhalation and intranasal, may involve solutions and suspensions in aqueous and nonaqueous formulations and can be prepared as a dry powder which may be pressurized or non-pressurized. In non-pressurized powder compositions, the active ingredient may be finely divided form may be used in admixture with a larger-sized pharmaceutically acceptable inert carrier comprising particles having a size, for example, of up to 100 micrometers in diameter. Suitable inert carriers include sugars such as lactose.

Alternatively, the composition may be pressurized and contain a compressed gas, such as nitrogen or a liquefied gas propellant. The liquefied propellant medium and indeed the total composition may be such that the active ingredient does not dissolve therein to any substantial extent. The pressurized composition may also contain a surface active agent, such as a liquid or solid non-ionic surface active agent or may be a solid anionic surface active agent. It is preferred to use the solid anionic surface active agent in the form of a sodium salt.

A further form of topical administration is to the eye. A hepcidin analogue of the invention may be delivered in a pharmaceutically acceptable ophthalmic vehicle, such that the hepcidin analogue is maintained in contact with the ocular surface for a sufficient time period to allow the hepcidin analogue to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically acceptable ophthalmic vehicle may, for example, be an ointment, vegetable oil or an encapsulating material. Alternatively, the hepcidin analogues of the invention may be injected directly into the vitreous and aqueous humour.

Compositions for rectal or vaginal administration include suppositories which may be prepared by mixing the hepcidin analogues of this invention with suitable non-irritating excipients or carriers such as cocoa butter, polyethylene glycol or a suppository wax, which are solid at room temperature but liquid at body temperature and, therefore, melt in the rectum or vaginal cavity and release the active compound.

Hepcidin analogues of the present invention may also be administered in liposomes or other lipid-based carriers. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a hepcidin analogue of the present invention, stabilizers, preservatives, excipients, and the like. In certain embodiments, the lipids comprise phospholipids, including the phosphatidyl cholines (lecithins) and serines, both natural and synthetic. Methods to form liposomes are known in the art.

Pharmaceutical compositions to be used in the invention suitable for parenteral administration may comprise sterile aqueous solutions and/or suspensions of the peptide inhibitors made isotonic with the blood of the recipient, generally using sodium chloride, glycerin, glucose, mannitol, sorbitol, and the like.

In some aspects, the invention provides a pharmaceutical composition for oral delivery. Compositions and hepcidin analogues of the instant invention may be prepared for oral administration according to any of the methods, techniques, and/or delivery vehicles described herein. Further, one having skill in the art will appreciate that the hepcidin analogues of the instant invention may be modified or integrated into a system or delivery vehicle that is not disclosed herein, yet is well known in the art and compatible for use in oral delivery of peptides.

In certain embodiments, formulations for oral administration may comprise adjuvants (e.g. resorcinols and/or nonionic surfactants such as polyoxyethylene oleyl ether and n-hexadecylpolyethylene ether) to artificially increase the permeability of the intestinal walls, and/or enzymatic inhibitors (e.g. pancreatic trypsin inhibitors, diisopropylfluorophosphate (DFF) or trasylol) to inhibit enzymatic degradation. In certain embodiments, the hepcidin analogue of a solid-type dosage form for oral administration can be mixed with at least one additive, such as sucrose, lactose, cellulose, mannitol, trehalose, raffinose, maltitol, dextran, starches, agar, alginates, chitins, chitosans, pectins, gum tragacanth, gum arabic, gelatin, collagen, casein, albumin, synthetic or semisynthetic polymer, or glyceride. These dosage forms can also contain other type(s) of additives, e.g., inactive diluting agent, lubricant such as magnesium stearate, paraben, preserving agent such as sorbic acid, ascorbic acid, alpha-tocopherol, antioxidants such as cysteine, disintegrators, binders, thickeners, buffering agents, pH adjusting agents, sweetening agents, flavoring agents or perfuming agents.

In particular embodiments, oral dosage forms or unit doses compatible for use with the hepcidin analogues of the present invention may include a mixture of hepcidin analogue and nondrug components or excipients, as well as other non-reusable materials that may be considered either as an ingredient or packaging. Oral compositions may include at least one of a liquid, a solid, and a semi-solid dosage forms. In some embodiments, an oral dosage form is provided comprising an effective amount of hepcidin analogue, wherein the dosage form comprises at least one of a pill, a tablet, a capsule, a gel, a paste, a drink, a syrup, ointment, and suppository. In some instances, an oral dosage form is provided that is designed and configured to achieve delayed release of the hepcidin analogue in the subject's small intestine and/or colon.

In one embodiment, an oral pharmaceutical composition comprising a hepcidin analogue of the present invention comprises an enteric coating that is designed to delay release of the hepcidin analogue in the small intestine. In at least some embodiments, a pharmaceutical composition is provided which comprises a hepcidin analogue of the present invention and a protease inhibitor, such as aprotinin, in a delayed release pharmaceutical formulation. In some instances, pharmaceutical compositions of the instant invention comprise an enteric coat that is soluble in gastric juice at a pH of about 5.0 or higher. In at least one embodiment, a pharmaceutical composition is provided comprising an enteric coating comprising a polymer having dissociable carboxylic groups, such as derivatives of cellulose, including hydroxypropylmethyl cellulose phthalate, cellulose acetate phthalate and cellulose acetate trimellitate and similar derivatives of cellulose and other carbohydrate polymers.

In one embodiment, a pharmaceutical composition comprising a hepcidin analogue of the present invention is provided in an enteric coating, the enteric coating being designed to protect and release the pharmaceutical composition in a controlled manner within the subject's lower gastrointestinal system, and to avoid systemic side effects. In addition to enteric coatings, the hepcidin analogues of the instant invention may be encapsulated, coated, engaged or otherwise associated within any compatible oral drug delivery system or component. For example, in some embodiments a hepcidin analogue of the present invention is provided in a lipid carrier system comprising at least one of polymeric hydrogels, nanoparticles, microspheres, micelles, and other lipid systems.

To overcome peptide degradation in the small intestine, some embodiments of the present invention comprise a hydrogel polymer carrier system in which a hepcidin analogue of the present invention is contained, whereby the hydrogel polymer protects the hepcidin analogue from proteolysis in the small intestine and/or colon. The hepcidin analogues of the present invention may further be formulated for compatible use with a carrier system that is designed to increase the dissolution kinetics and enhance intestinal absorption of the peptide. These methods include the use of liposomes, micelles and nanoparticles to increase GI tract permeation of peptides.

Various bioresponsive systems may also be combined with one or more hepcidin analogue of the present invention to provide a pharmaceutical agent for oral delivery. In some embodiments, a hepcidin analogue of the instant invention is used in combination with a bioresponsive system, such as hydrogels and mucoadhesive polymers with hydrogen bonding groups (e.g., PEG, poly(methacrylic) acid [PMAA], cellulose, Eudragit®, chitosan and alginate) to provide a therapeutic agent for oral administration. Other embodiments include a method for optimizing or prolonging drug residence time for a hepcidin analogue disclosed herein, wherein the surface of the hepcidin analogue surface is modified to comprise mucoadhesive properties through hydrogen bonds, polymers with linked mucins or/and hydrophobic interactions. These modified peptide molecules may demonstrate increase drug residence time within the subject, in accordance with a desired feature of the invention. Moreover, targeted mucoadhesive systems may specifically bind to receptors at the enterocytes and M-cell surfaces, thereby further increasing the uptake of particles containing the hepcidin analogue.

Other embodiments comprise a method for oral delivery of a hepcidin analogue of the present invention, wherein the hepcidin analogue is provided to a subject in combination with permeation enhancers that promote the transport of the peptides across the intestinal mucosa by increasing paracellular or transcellular permeation. For example, in one embodiment, a permeation enhancer is combined with a hepcidin analogue, wherein the permeation enhancer comprises at least one of a long-chain fatty acid, a bile salt, an amphiphilic surfactant, and a chelating agent. In one embodiment, a permeation enhancer comprising sodium N-[hydroxybenzoyl)amino] caprylate is used to form a weak noncovalent association with the hepcidin analogue of the instant invention, wherein the permeation enhancer favors membrane transport and further dissociation once reaching the blood circulation. In another embodiment, a hepcidin analogue of the present invention is conjugated to oligoarginine, thereby increasing cellular penetration of the peptide into various cell types. Further, in at least one embodiment a noncovalent bond is provided between a peptide inhibitor of the present invention and a permeation enhancer selected from the group consisting of a cyclodextrin (CD) and a dendrimers, wherein the permeation enhancer reduces peptide aggregation and increasing stability and solubility for the hepcidin analogue molecule.

Other embodiments of the invention provide a method for treating a subject with a hepcidin analogue of the present invention having an increased half-life. In one aspect, the present invention provides a hepcidin analogue having a half-life of at least several hours to one day in vitro or in vivo (e.g., when administered to a human subject) sufficient for daily (q.d.) or twice daily (b.i.d.) dosing of a therapeutically effective amount. In another embodiment, the hepcidin analogue has a half-life of three days or longer sufficient for weekly (q.w.) dosing of a therapeutically effective amount. Further, in another embodiment, the hepcidin analogue has a half-life of eight days or longer sufficient for bi-weekly (b.i.w.) or monthly dosing of a therapeutically effective amount. In another embodiment, the hepcidin analogue is derivatized or modified such that is has a longer half-life as compared to the underivatized or unmodified hepcidin analogue. In another embodiment, the hepcidin analogue contains one or more chemical modifications to increase serum half-life.

When used in at least one of the treatments or delivery systems described herein, a hepcidin analogue of the present invention may be employed in pure form or, where such forms exist, in pharmaceutically acceptable salt form.

### Dosages

The total daily usage of the hepcidin analogues and compositions of the present invention can be decided by the attending physician within the scope of sound medical judgment. The specific therapeutically effective dose level for any particular subject will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific hepcidin analogue employed; e) the duration of the treatment; f) drugs used in combination or coincidental with the specific hepcidin analogue employed, and like factors well known in the medical arts.

In particular embodiments, the total daily dose of the hepcidin analogues of the invention to be administered to a human or other mammal host in single or divided doses may be in amounts, for example, from 0.0001 to 300 mg/kg body weight daily or 1 to 300 mg/kg body weight daily. In certain embodiments, a dosage of a hepcidin analogue of the present invention is in the range from about 0.0001 to about 100 mg/kg body weight per day, such as from about 0.0005 to about 50 mg/kg body weight per day, such as from about 0.001 to about 10 mg/kg body weight per day, e.g. from about 0.01 to about 1 mg/kg body weight per day, administered in one or more doses, such as from one to three doses.

In various embodiments, a hepcidin analogue of the invention may be administered continuously (e.g. by intravenous administration or another continuous drug administration method), or may be administered to a subject at intervals, typically at regular time intervals, depending on the desired dosage and the pharmaceutical composition selected by the skilled practitioner for the particular subject. Regular administration dosing intervals include, e.g., once daily, twice daily, once every two, three, four, five or six days, once or twice weekly, once or twice monthly, and the like.

Such regular hepcidin analogue administration regimens of the invention may, in certain circumstances such as, e.g., during chronic long-term administration, be advantageously interrupted for a period of time so that the medicated subject reduces the level of or stops taking the medication, often referred to as taking a “drug holiday.” Drug holidays are useful for, e.g., maintaining or regaining sensitivity to a drug especially during long-term chronic treatment, or to reduce unwanted side-effects of long-term chronic treatment of the subject with the drug. The timing of a drug holiday depends on the timing of the regular dosing regimen and the purpose for taking the drug holiday (e.g., to regain drug sensitivity and/or to reduce unwanted side effects of continuous, long-term administration). In some embodiments, the drug holiday may be a reduction in the dosage of the drug (e.g. to below the therapeutically effective amount for a certain interval of time). In other embodiments, administration of the drug is stopped for a certain interval of time before administration is started again using the same or a different dosing regimen (e.g. at a lower or higher dose and/or frequency of administration). A drug holiday of the invention may thus be selected from a wide range of time-periods and dosage regimens. An exemplary drug holiday is two or more days, one or more weeks, or one or more months, up to about 24 months of drug holiday. So, for example, a regular daily dosing regimen with a peptide, a peptide analogue, or a dimer of the invention may, for example, be interrupted by a drug holiday of a week, or two weeks, or four weeks, after which time the preceding, regular dosage regimen (e.g. a daily or a weekly dosing regimen) is resumed. A variety of other drug holiday regimens are envisioned to be useful for administering the hepcidin analogues of the invention.

Thus, the hepcidin analogues may be delivered via an administration regime which comprises two or more administration phases separated by respective drug holiday phases.

During each administration phase, the hepcidin analogue is administered to the recipient subject in a therapeutically effective amount according to a pre-determined administration pattern. The administration pattern may comprise continuous administration of the drug to the recipient subject over the duration of the administration phase. Alternatively, the administration pattern may comprise administration of a plurality of doses of the hepcidin analogue to the recipient subject, wherein said doses are spaced by dosing intervals.

A dosing pattern may comprise at least two doses per administration phase, at least five doses per administration phase, at least 10 doses per administration phase, at least 20 doses per administration phase, at least 30 doses per administration phase, or more.

Said dosing intervals may be regular dosing intervals, which may be as set out above, including once daily, twice daily, once every two, three, four, five or six days, once or twice weekly, once or twice monthly, or a regular and even less frequent dosing interval, depending on the particular dosage formulation, bioavailability, and pharmacokinetic profile of the hepcidin analogue of the present invention.

An administration phase may have a duration of at least two days, at least a week, at least 2 weeks, at least 4 weeks, at least a month, at least 2 months, at least 3 months, at least 6 months, or more.

Where an administration pattern comprises a plurality of doses, the duration of the following drug holiday phase is longer than the dosing interval used in that administration pattern. Where the dosing interval is irregular, the duration of the drug holiday phase may be greater than the mean interval between doses over the course of the administration phase. Alternatively the duration of the drug holiday may be longer than the longest interval between consecutive doses during the administration phase.

The duration of the drug holiday phase may be at least twice that of the relevant dosing interval (or mean thereof), at least 3 times, at least 4 times, at least 5 times, at least 10 times, or at least 20 times that of the relevant dosing interval or mean thereof.

Within these constraints, a drug holiday phase may have a duration of at least two days, at least a week, at least 2 weeks, at least 4 weeks, at least a month, at least 2 months, at least 3 months, at least 6 months, or more, depending on the administration pattern during the previous administration phase.

An administration regime comprises at least 2 administration phases. Consecutive administration phases are separated by respective drug holiday phases. Thus the administration regime may comprise at least 3, at least 4, at least 5, at least 10, at least 15, at least 20, at least 25, or at least 30 administration phases, or more, each separated by respective drug holiday phases.

Consecutive administration phases may utilise the same administration pattern, although this may not always be desirable or necessary. However, if other drugs or active agents are administered in combination with a hepcidin analogue of the invention, then typically the same combination of drugs or active agents is given in consecutive administration phases. In certain embodiments, the recipient subject is human.

In some embodiments, the present invention provides compositions and medicaments comprising at least one hepcidin analogue as disclosed herein. In some embodiments, the present invention provides a method of manufacturing medicaments comprising at least one hepcidin analogue as disclosed herein for the treatment of diseases of iron metabolism, such as iron overload diseases. In some embodiments, the present invention provides a method of manufacturing medicaments comprising at least one hepcidin analogue as disclosed herein for the treatment of diabetes (Type I or Type II), insulin resistance, or glucose intolerance. Also provided are methods of treating a disease of iron metabolism in a subject, such as a mammalian subject, and preferably a human subject, comprising administering at least one hepcidin analogue, or composition as disclosed herein to the subject. In some embodiments, the hepcidin analogue or the composition is administered in a therapeutically effective amount. Also provided are methods of treating diabetes (Type I or Type II), insulin resistance, or glucose intolerance in a subject, such as a mammalian subject, and preferably a human subject, comprising administering at least one hepcidin analogue or composition as disclosed herein to the subject. In some embodiments, the hepcidin analogue or composition is administered in a therapeutically effective amount.

In some embodiments, the invention provides a process for manufacturing a hepcidin analogue or a hepcidin analogue composition (e.g., a pharmaceutical composition), as disclosed herein.

In some embodiments, the invention provides a device comprising at least one hepcidin analogue of the present invention, or pharmaceutically acceptable salt or solvate thereof for delivery of the hepcidin analogue to a subject.

In some embodiments, the present invention provides methods of binding a ferroportin or inducing ferroportin internalization and degradation which comprises contacting the ferroportin with at least one hepcidin analogue, or hepcidin analogue composition as disclosed herein.

In some embodiments, the present invention provides kits comprising at least one hepcidin analogue, or hepcidin analogue composition (e.g., pharmaceutical composition) as disclosed herein packaged together with a reagent, a device, instructional material, or a combination thereof.

In some embodiments, the present invention provides a method of administering a hepcidin analogue or hepcidin analogue composition (e.g., pharmaceutical composition) of the present invention to a subject via implant or osmotic pump, by cartridge or micro pump, or by other means appreciated by the skilled artisan, as well-known in the art.

In some embodiments, the present invention provides complexes which comprise at least one hepcidin analogue as disclosed herein bound to a ferroportin, preferably a human ferroportin, or an antibody, such as an antibody which specifically binds a hepcidin analogue as disclosed herein, Hep25, or a combination thereof.

In some embodiments, the hepcidin analogue of the present invention has a measurement (e.g., an EC50) of less than 500 nM within the Fpn internalization assay. As a skilled person will realize, the function of the hepcidin analogue is dependent on the tertiary structure of the hepcidin analogue and the binding surface presented. It is therefore possible to make minor changes to the sequence encoding the hepcidin analogue that do not affect the fold or are not on the binding surface and maintain function. In other embodiments, the present invention provides a hepcidin analogue having 85% or higher (e.g., 85%, 90%, 91%, 92%, 93%, 94%, 95%, 96%, 97%, 98%, 99%, or 99.5%) identity or homology to an amino acid sequence of any hepcidin analogue described herein that exhibits an activity (e.g., hepcidin activity), or lessens a symptom of a disease or indication for which hepcidin is involved.

In other embodiments, the present invention provides a hepcidin analogue having 85% or higher (e.g., 85%, 90%, 91%, 92%, 93%, 94%, 95%, 96%, 97%, 98%, 99%, or 99.5%) identity or homology to an amino acid sequence of any hepcidin analogue presented herein, e.g., in any one of Tables 2-4 or Tables 6-10, 12, 14, or 15, or a peptide according to any one of the formulae described herein, e.g., formulae I, II, III, IV, V, and VI.

In some embodiments, a hepcidin analogue of the present invention may comprise functional fragments or variants thereof that have at most 10, 9, 8, 7, 6, 5, 4, 3, 2, or 1 amino acid substitutions compared to one or more of the specific peptide analogue sequences recited herein.

In addition to the methods described in the Examples herein, the hepcidin analogue peptides and the peptide dimers of the present invention may be produced using methods known in the art including chemical synthesis, biosynthesis or in vitro synthesis using recombinant DNA methods, and solid phase synthesis. See e.g. Kelly & Winkler (1990) Genetic Engineering Principles and Methods, vol. 12, J. K. Setlow ed., Plenum Press, NY, pp. 1-19; Merrifield (1964) J Amer Chem Soc 85:2149; Houghten (1985) PNAS USA 82:5131-5135; and Stewart & Young (1984) Solid Phase Peptide Synthesis, 2ed. Pierce, Rockford, Ill., which are herein incorporated by reference. The hepcidin analogues of the present invention may be purified using protein purification techniques known in the art such as reverse phase high-performance liquid chromatography (HPLC), ion-exchange or immunoaffinity chromatography, filtration or size exclusion, or electrophoresis. See Olsnes, S. and A. Pihl (1973) Biochem. 12(16):3121-3126; and Scopes (1982) Protein Purification, Springer-Verlag, NY, which are herein incorporated by reference. Alternatively, the hepcidin analogues of the present invention may be made by recombinant DNA techniques known in the art. Thus, polynucleotides that encode the polypeptides of the present invention are contemplated herein. In certain preferred embodiments, the polynucleotides are isolated. As used herein “isolated polynucleotides” refers to polynucleotides that are in an environment different from that in which the polynucleotide naturally occurs.

### EXAMPLES

The following examples demonstrate certain specific embodiments of the present invention. The following examples were carried out using standard techniques that are well known and routine to those of skill in the art, except where otherwise described in detail. It is to be understood that these examples are for illustrative purposes only and do not purport to be wholly definitive as to conditions or scope of the invention. As such, they should not be construed in any way as limiting the scope of the present invention.

### ABBREVIATIONS

DCM: dichloromethane
DMF: N,N-dimethylformamide
NMP: N-methylpyrolidone
HBTU: O-(Benzotriazol-1-yl)-N,N,N′,N′-tetramethyluronium hexafluorophosphate
HATU: 2-(7-aza-1H-benzotriazole-1-yl)-1,1,3,3-tetramethyluronium hexafluorophosphate
DCC: Dicyclohexylcarbodiimide
NHS: N-hydroxysuccinimide
DIPEA: diisopropylethylamine
EtOH: ethanol
Et2O: diethyl ether
Hy: hydrogen
TFA: trifluoroacetic acid
TIS: triisopropylsilane
ACN: acetonitrile
HPLC: high performance liquid chromatography
ESI-MS: electron spray ionization mass spectrometry
PBS: phosphate-buffered saline
Boc: t-butoxycarbonyl
Fmoc: Fluorenylmethyloxycarbonyl
Acm: acetamidomethyl
IVA: Isovaleric acid (or Isovaleryl)
K( ): In the peptide sequences provided herein, wherein a compound or chemical group is presented in parentheses directly after a Lysine residue, it is to be understood that the compound or chemical group in the parentheses is a side chain conjugated to the Lysine residue. So, e.g., but not to be limited in any way, K-[(PEG8)]-indicates that a PEG8 moiety is conjugated to a side chain of this Lysine. For a few non-limiting examples of such a conjugated Lysines, please see, e.g., compounds 54 and 90.
Palm: Indicates conjugation of a palmitic acid (palmitoyl).

As used herein “C( )” refers to a cysteine residue involved in a particular disulfide bridge. For example, in Hepcidin, there are four disulfide bridges: the first between the two C(1) residues; the second between the two C(2) residues; the third between the two C(3) residues; and the fourth between the two C(4) residues. Accordingly, in some embodiments, the sequence for Hepcidin is written as follows:

(SEQ ID NO: 335)

Hy-DTHFPIC(1)IFC(2)C(3)GC(2)C(4)HRSKC(3)GMC(4)C(1)
KT-OH;

and the sequence for other peptides may also optionally be written in the same manner.

### Example 1

### Synthesis of Peptide Analogues

Unless otherwise specified, reagents and solvents employed in the following were available commercially in standard laboratory reagent or analytical grade, and were used without further purification.

### Procedure for Solid-Phase Synthesis of Peptides

Peptide analogues of the invention were chemically synthesized using optimized 9-fluorenylmethoxy carbonyl (Fmoc) solid phase peptide synthesis protocols. For C-terminal amides, rink-amide resin was used, although wang and trityl resins were also used to produce C-terminal acids. The side chain protecting groups were as follows: Glu, Thr and Tyr: O-tButyl; Trp and Lys: t-Boc (t-butyloxycarbonyl); Arg: N-gamma-2,2,4,6,7-pentamethyldihydrobenzofuran-5-sulfonyl; His, Gln, Asn, Cys: Trityl. For selective disulfide bridge formation, Acm (acetamidomethyl) was also used as a Cys protecting group. For coupling, a four to ten-fold excess of a solution containing Fmoc amino acid, HBTU and DIPEA (1:1:1.1) in DMF was added to swelled resin [HBTU: O-(Benzotriazol-1-yl)-N,N,N′,N′-tetramethyluronium hexafluorophosphate; DIPEA: diisopropylethylamine; DMF: dimethylformamide]. HATU (O-(7-azabenzotriazol-1-yl)-1,1,3,3,-tetramethyluronium hexafluorophosphate) was used instead of HBTU to improve coupling efficiency in difficult regions. Fmoc protecting group removal was achieved by treatment with a DMF, piperidine (2:1) solution.

### Procedure for Cleavage of Peptides Off Resin

Side chain deprotection and cleavage of the peptide analogues of the invention (e.g., Compound No. 2) was achieved by stirring dry resin in a solution containing trifluoroacetic acid, water, ethanedithiol and tri-isopropylsilane (90:5:2.5:2.5) for 2 to 4 hours. Following TFA removal, peptide was precipitated using ice-cold diethyl ether. The solution was centrifuged and the ether was decanted, followed by a second diethyl ether wash. The peptide was dissolved in an acetonitrile, water solution (1:1) containing 0.1% TFA (trifluoroacetic acid) and the resulting solution was filtered. The linear peptide quality was assessed using electrospray ionisation mass spectrometry (ESI-MS).

### Procedure for Purification of Peptides

Purification of the peptides of the invention (e.g., Compound No. 2) was achieved using reverse-phase high performance liquid chromatography (RP-HPLC). Analysis was performed using a C18 column (3 μm, 50×2 mm) with a flow rate of 1 mL/min. Purification of the linear peptides was achieved using preparative RP-HPLC with a C18 column (5 μm, 250×21.2 mm) with a flow rate of 20 mL/min. Separation was achieved using linear gradients of buffer B in A (Buffer A: Aqueous 0.05% TFA; Buffer B: 0.043% TFA, 90% acetonitrile in water).

### Procedure for Oxidation of Peptides

Method a (Single Disulfide Oxidation).

Oxidation of the unprotected peptides of the invention was achieved by adding drop-wise iodine in MeOH (1 mg per 1 mL) to the peptide in a solution (ACN:H₂O, 7:3, 0.5% TFA). After stirring for 2 min, ascorbic acid portion wise was added until the solution was clear and the sample was immediately loaded onto the HPLC for purification.

Method B (Selective Oxidation of Two Disulfides).

When more than one disulfide was present, selective oxidation was often performed. Oxidation of the free cysteines was achieved at pH 7.6 NH₄CO₃ solution at 1 mg/10 mL of peptide. After 24 h stirring and prior to purification the solution was acidified to pH 3 with TFA followed by lyophilization. The resulting single oxidized peptides (with ACM protected cysteines) were then oxidized/selective deprotection using iodine solution. The peptide (1 mg per 2 mL) was dissolved in MeOH/H₂O, 80:20 iodine dissolved in the reaction solvent was added to the reaction (final concentration: 5 mg/mL) at room temperature. The solution was stirred for 7 minutes before ascorbic acid was added portion wise until the solution is clear. The solution was then loaded directly onto the HPLC.

Method C (Native Oxidation).

When more than one disulfide was present and when not performing selective oxidations, native oxidation was performed. Native oxidation was achieved with 100 mM NH₄CO₃ (pH7.4) solution in the presence of oxidized and reduced glutathione (peptide/GSH/GSSG, 1:100:10 molar ratio) of (peptide: GSSG: GSH, 1:10, 100). After 24 h stirring and prior to RP-HPLC purification the solution was acidified to pH 3 with TFA followed by lyophilization.

Procedure of Cysteine Oxidation to Produce Dimers.

Oxidation of the unprotected peptides of the invention was achieved by adding drop-wise iodine in MeOH (1 mg per 1 mL) to the peptide in a solution (ACN:H₂O, 7:3, 0.5% TFA). After stirring for 2 min, ascorbic acid portion wise was added until the solution was clear and the sample was immediately loaded onto the HPLC for purification.

### Procedure for Dimerization.

Glyoxylic acid (DIG), IDA, or Fmoc-β-Ala-IDA was pre-activated as the N-hydroxysuccinimide ester by treating 1 equivalent (abbreviated “eq”) of the acid with 2.2 eq of both N-hydroxysuccinimide (NHS) and dicyclohexyl carbodiimide (DCC) in NMP (N-methyl pyrolidone) at a 0.1 M final concentration. For the PEG13 and PEG25 linkers, these chemical entities were purchased pre-formed as the activated succinimide ester. The activated ester ˜0.4 eq was added slowly to the peptide in NMP (1 mg/mL) portionwise. The solution was left stirring for 10 min before 2-3 additional aliquots of the linker ˜0.05 eq were slowly added. The solution was left stirring for a further 3 h before the solvent was removed under vacuo and the residue was purified by reverse phase HPLC. An additional step of stirring the peptide in 20% piperidine in DMF (2×10 min) before an additional reverse phase HPLC purification was performed.

One of skill in the art will appreciate that standard methods of peptide synthesis may be used to generate the compounds of the invention.

### Linker Activation and Dimerization

Peptide monomer subunits were linked to form hepcidin analogue peptide dimers as described below.

Small Scale DIG Linker Activation Procedure:

5 mL of NMP was added to a glass vial containing IDA diacid (304.2 mg, 1 mmol), N-hydroxysuccinimide (NHS, 253.2 mg, 2.2 eq. 2.2 mmol) and a stirring bar. The mixture was stirred at room temperature to completely dissolve the solid starting materials. N, N′-Dicyclohexylcarbodiimide (DCC, 453.9 mg, 2.2 eq., 2.2 mmol) was then added to the mixture. Precipitation appeared within 10 min and the reaction mixture was further stirred at room temperature overnight. The reaction mixture was then filtered to remove the precipitated dicyclohexylurea (DCU). The activated linker was kept in a closed vial prior to use for dimerization. The nominal concentration of the activated linker was approximately 0.20 M.

For dimerization using PEG linkers, there was no pre-activation step involved. Commercially available pre-activated bi-functional PEG linkers were used.

Dimerization Procedure:

2 mL of anhydrous DMF was added to a vial containing peptide monomer (0.1 mmol). The pH of the peptide was the adjusted to 8-9 with DIEA. Activated linker (IDA or PEG13, PEG 25) (0.48 eq relative to monomer, 0.048 mmol) was then added to the monomer solution. The reaction mixture was stirred at room temperature for one hour. Completion of the dimerization reaction was monitored using analytical HPLC. The time for completion of dimerization reaction varied depending upon the linker. After completion of reaction, the peptide was precipitated in cold ether and centrifuged. The supernatant ether layer was discarded. The precipitation step was repeated twice. The crude dimer was then purified using reverse phase HPLC (Luna C18 support, 10u, 100A, Mobile phase A: water containing 0.1% TFA, mobile phase B: Acetonitrile (ACN) containing 0.1% TFA, gradient of 15% B and change to 45% B over 60 min, flow rate 15 ml/min). Fractions containing pure product were then freeze-dried on a lyophilizer.

### Example 2

### Activity of Peptide Analogues

Peptide analogues were tested in vitro for induction of internalization of the human ferroportin protein. Following internalization, the peptides are degraded. The assay measures a decrease in fluorescence of the receptor.

The cDNA encoding the human ferroportin (SLC40A1) was cloned from a cDNA clone from Origene (NM_014585). The DNA encoding the ferroportin was amplified by PCR using primers also encoding terminal restriction sites for subcloning, but without the termination codon. The ferroportin receptor was subcloned into a mammalian GFP expression vector containing a neomycin (G418) resistance marker in such that the reading frame of the ferroportin was fused in frame with the GFP protein. The fidelity of the DNA encoding the protein was confirmed by DNA sequencing. HEK293 cells were used for transfection of the ferroportin-GFP receptor expression plasmid. The cells were grown according to standard protocol in growth medium and transfected with the plasmids using Lipofectamine (manufacturer's protocol, Invitrogen). The cells stably expressing ferroportin-GFP were selected using G418 in the growth medium (in that only cells that have taken up and incorporated the cDNA expression plasmid survive) and sorted several times on a Cytomation MoFlo™ cell sorter to obtain the GFP-positive cells (488 nm/530 nm). The cells were propagated and frozen in aliquots.

To determine activity of the hepcidin analogues (compounds) on the human ferroportin, the cells were incubated in 96 well plates in standard media, without phenol red. Compound was added to desired final concentration for at least 18 hours in the incubator. Following incubation, the remaining GFP-fluorescence was determined either by whole cell GFP fluorescence (Envision plate reader, 485/535 filter pair), or by Beckman Coulter Quanta™ flow cytometer (express as Geometric mean of fluorescence intensity at 485 nm/525 nm). Compound was added to desired final concentration for at least 18 hours but no more than 24 hours in the incubator.

Reference compounds included native Hepcidin, Mini-Hepcidin, and R1-Mini-Hepcidin, which is an analog of mini-hepcidin. The “RI” in RI-Mini-Hepcidin refers to Retro Inverse. A retro inverse peptide is a peptide with a reversed sequence in all D amino acids. An example is that Hy-Glu-Thr-His-NH₂ becomes Hy-DHis-DThr-DGlu-NH₂. The EC₅₀ of these reference compounds for ferroportin degradation was determined according to the activity assay described above. These peptides served as control standards for many of the subsequence studies.

TABLE 11
Reference compounds

| | | Potency |
|---|---|---|
| | | EC50 |
| Name | Sequence | (nM) |
| Hepcidin | Hy-DTHFPIC(1)IFC(2)C(3)GC(2)C(4)HRSKC(3)GMC(4)C(1)KT-OH | 34 |
| | (SEQ ID NO: 335) | |
| Mini- | Hy-DTHFPICIF-NH₂ | 712 |
| Hepcidin | (SEQ ID NO: 545) | |
| 1-9 | | |
| RI-Mini | Hy-DPhe-DIle-DCys-DIle-DPro-DPhe-DHis-DThr-DAsp-NH₂ | >10 μM |
| Hepcidin | (SEQ ID NO: 546) | |

The EC₅₀ values determined for various peptide analogues of the present invention are provided below and in other tables herein.

TABLE 12
Activity of Illustrative Peptide Analogues

| | SEQ | | Potency |
|---|---|---|---|
| CMPD | ID | | EC₅₀ |
| No. | No. | Sequence | (nM) |
| 1 | 28 | Hy-DTHFPCIIF-NH₂ | 133 |
| 2 | 29 | Isovaleric acid-DTHFPICIFGPRSKGWVC-NH₂ | 5 |
| 3 | 30 | Isovaleric acid-DTHFPCIIFGPRSRGWVCK-NH₂ | 15 |
| 4 | 31 | Isovaleric acid-DTHFPCIIFGPRSKGWVC-NH₂ | 19 |
| 5 | 32 | [Ida]-TH-[Dpa]-[bhPro]-ICIFGPRSKGWVCM-NH₂ | 17 |
| 6 | 33 | Isovaleric acid-DTHFPCIFFGPRSKGWVCK-NH₂ | 23 |
| 7 | 34 | Isovaleric acid-DTHFPCIIFGPRSKGWTCK-NH₂ | 24 |
| 8 | 35 | [Ida]-TH-[Dpa]-[bhPro]-CIIFGPRSRGWVCK-NH₂ | 29 |
| 9 | 36 | Isovaleric acid-DTHFPCIKFGPRSKGWVCK-NH₂ | 32 |
| 10 | 37 | Isovaleric acid-DTHFPCIQFGPRSKGWVCK-NH₂ | 35 |
| 11 | 38 | Isovaleric acid-DTHFPCIIFGPRSKGWVCK-NH₂ | 9 |
| 12 | 39 | Hy-DTHFPIC₁IFVC₂GHRSIC₂YRRC₁R-NH₂ | 77 |
| 13 | 40 | Isobutyric acid-DTHFPIC₁IFVC₂HRSKGC₂YRRC₁R-NH₂ | 63 |
| 14 | 41 | Hy-DTHFPIC₁IFVC₂HRSKGC₂YRAC₁-NH₂ | 69 |
| 15 | 42 | Isovaleric acid-DTHFPCIEFGPRSKGWVCK-NH₂ | 79 |
| 16 | 43 | Hy-DTHFPICIFGPRAKGWVCM-NH₂ | 88 |
| 17 | 44 | Isobutyric acid-DTHFPIC₁IFVC₂HRSKGC₂YRRC₁R-NH₂ | 93 |
| 18 | 45 | Hy-DTHFPICIFGPRSKGWVCM-NH₂ | 125 |
| 19 | 46 | Hy-DTHFPIC₁IFVC₂HRSKGC₂YRRC₁R-NH₂ | 140 |
| 20 | 47 | Hy-DTHFPICIFGPRSRGWVCK-NH₂ | 101 |
| 21 | 48 | Hy-DTHFPCIIFGPRSKGWVCM-NH₂ | 46 |
| 22 | 49 | Hy-DTHFPICIFAPRSKGWVCM-NH₂ | 9430 |
| 23 | 50 | Hy-DTHFPICIFGPRSKGWVCM-OH | 131 |
| 24 | 51 | Hy-DTHFPCIQF-NH₂ | 138 |
| 25 | 52 | Hy-DTHFPIC₁IFVC₂GHRSKGC₂YRRC₁R-NH₂ | 144 |
| 26 | 53 | Hy-DTHFAICIFGPRSKGWVCM-NH₂ | 147 |
| 27 | 54 | Hy-DTHFPICIFGPHRSKGWVCM-NH₂ | 149 |
| 28 | 55 | Hy-DTHFPICIFGPRAKGWVCM-NH₂ | 88 |
| 29 | 56 | Hy-DTHFPACIFGPRSKGWVCM-NH₂ | 157 |
| 30 | 57 | Hy-DTHFPC₁IIFVC₂HRPKGC₂YRRVC₁R-NH₂ | 173 |
| 31 | 58 | Hy-DTHFPICIFGPRSKAWVCM-NH₂ | 175 |
| 32 | 59 | Hy-DTHFPIC₁IFVC₂GHRGKGC₂YRRC₁R-NH₂ | 182 |
| 33 | 60 | Hy-ATHFPICIFGPRSKGWVCM-NH₂ | 184 |
| 34 | 61 | Hy-DTHFPICIFGPASKGWVCM-NH₂ | 206 |
| 35 | 62 | Hy-DTHFPIC₁IFVC₂HRSKGC₂YARC₁-NH₂ | 214 |
| 36 | 63 | Ac-DTHFPICIFGPRSKGWVCM-NH₂ | 239 |
| 37 | 64 | Hy-DTHFPICIFGPRSAGWVCM-NH₂ | 239 |
| 38 | 65 | Hy-DTHAPICIFGPRSKGWVCM-NH₂ | 254 |
| 39 | 66 | Hy-DTHFPIC₁IFVC₂HRSKGC₂YRRC₁-NH₂ | 256 |
| 40 | 67 | pGlu-THFPIC₁IFVC₂HRSKGC₂YRRC₁R-NH₂ | 260 |
| 41 | 68 | Ac-DTHFPICIFKPRSKGWVCM-NH₂ | 262 |
| 42 | 69 | Hy-DTHFPIC₁IFVC₂GHRSKGC₂YMRC₁KT-NH₂ | 265 |
| 43 | 70 | Hy-DAHFPICIFGPRSKGWVCM-NH₂ | 265 |
| 44 | 71 | Hy-DTHFPIC₁IFVC₂YRGIC₂YRRC₁R-NH₂ | 269 |
| 45 | 72 | Ac-DTHFPICIFGPRSKGWVCM-NH₂ | 272 |
| 46 | 73 | Hy-[bhAsp]-THFPICIFGPRSKGWVC-NH₂ | 274 |
| 47 | 74 | Hy-DTHFPICIFGPRSKGWACM-NH₂ | 313 |
| 48 | 75 | [Ida]-TH-[Dpa]-[bhPro]-RCR-[bhPhe]-GPRSKGWVCM- | 331 |
| | | NH₂ | |
| 49 | 76 | Hy-DTHFPCIRF-NH₂ | 334 |
| 50 | 77 | Isovaleric acid-THFPCIIFGPRSKGWVCM-NH₂ | 345 |
| 51 | 78 | Hy-DTHFPCIAF-NH₂ | 382 |
| 52 | 79 | Hy-DAHFPCIIF-NH₂ | 388 |
| 53 | 80 | Hy-DTHFPIC₁IFVC₂HRPKGC₂YRRC₁P-NH₂ | 393 |
| 54 | 81 | Ac-DTHFPICIFKPRS-K(m-PEG8)-GWVCM-NH₂ | 479 |
| 55 | 82 | Hy-DTHFPCIIFK-NH₂ | 419 |
| 56 | 83 | Hy-DTHFPCIFF-NH₂ | 441 |
| 57 | 84 | Hy-DTHFPICIFGPRSK-K(m-PEG8)-WVCM-NH₂ | 462 |
| 58 | 85 | Ac-DTHFPICIFGPRSKKWVCM-NH₂ | 472 |
| 59 | 86 | Hy-DTHFPIC₁IFC₂PWGMC₂C₁K-NH₂ | 495 |
| 60 | 87 | Hy-DTAFPICIFGPRSKGWVCM-NH₂ | 498 |
| 65 | 88 | Hy-DTHFPIC₁IFVC₂YRGIC₁YMRC₂KT-NH₂ | 763 |
| 66 | 89 | Hy-DTHFPICIFGPRSKGAVCM-NH₂ | 520 |
| 67 | 90 | Hy-DTHFPICIAGPRSKGWVCM-NH₂ | 2466 |
| 68 | 91 | Hy-DTHFPICAFGPRSKGWVCM-NH₂ | >10 μM |
| 69 | 92 | Hy-DTHFPIAIFGPRSKGWVAM-NH₂ | >10 μM |
| 70 | 93 | Hy-DTHFPCRRFGPRSKGWVC-NH₂ | >10 μM |
| 71 | 94 | [Ida]-THF-[bhPro]-CRR-[bhPhe]-GPRSKGWVC-NH₂ | N/A |
| 73 | 96 | Hy-DTHFPC₁IIFVC₂HRSKGC₂YWAVC₁-NH₂ | 2640 |
| 74 | 97 | Hy-DTHFP-(D)Cys₁-IIFVC₂HRSKGC₂YWAV-(D)Cys₁-F- | 356 |
| | | NH₂ | |
| 75 | 98 | Hy-DTHFPC₁IIFVC₂HRSKGC₂YWAVC₁FW-NH₂ | >10 μM |
| 76 | 99 | Ac-DTHFPICIF-K-[(m-PEG8)]--PRSKGWVCM-NH₂ | 610 |
| 78 | 101 | Hy-DTH-[Dpa]-PCIIFGPRSRGWVCK-NH₂ | >1 μM |
| 79 | 102 | Hy-DTHF-[bhPro]-CIIFGPRSRGWVCK-NH₂ | >1 μM |
| 80 | 103 | Hy-DTHFPCIIFGPRSRGWRCK-NH₂ | >1 μM |
| 81 | 104 | Hy-DTHFPCIRFGPRSRGWVCK-NH₂ | >1 μM |
| 82 | 105 | Hy-DTHFPCIRFGPRSRGWRCK-NH₂ | >1 μM |
| 83 | 106 | Hy-DTHFPCIIFGPRSRGWVCK-NH₂ | >1 μM |
| 84 | 107 | Hy-DTHFPCIIFGPRSRGVCK-NH₂ | >1 μM |
| 85 | 108 | Hy-DTHFPCIYFGPRSKGWVCK-NH₂ | 705 |
| 86 | 109 | Hy-DTHFPCIIFGPRSKGWVCK-NH₂ | >1 μM |
| 87 | 110 | Hy-DTHFPCIIFGPRARGWVCK-NH₂ | >1 μM |
| 88 | 111 | Octanoic acid-DTHFPCIIFGPRSRGWVCK-NH₂ | >1 μM |
| 89 | 112 | Palm-PEG11-DTHFPCIIFGPRSRGWVCK-NH₂ | >1 μM |
| 90 | 113 | Ac-DTHFPICIF-K(2K PEG)-PRSKGWVCK-NH₂ | 107 |
| 91 | 114 | Hy-DTHFPCIIFGPRSKGWKCK-NH₂ | Not |
| | | | Tested |
| 92 | 115 | Hy-DTHFPCIKFGPRSKGWKCK-NH₂ | Not |
| | | | Tested |
| 93 | 116 | Isovaleric acid-DTHFPCLIFGPRSKGWVCK-NH₂ | 19 |
| 94 | 117 | Isovaleric acid-DTHFPCVIFGPRSKGWVCK-NH₂ | 41 |
| 95 | 118 | Isovaleric acid-DTHFPCSIFGPRSKGWVCK-NH₂ | 78 |
| 96 | 119 | Isovaleric acid-DTHFPCQIFGPRSKGWVCK-NH₂ | 157 |
| 97 | 120 | Hy-THFPCIIFGPRSKGWVCK-NH₂ | >10 μM |
| 98 | 121 | Isovaleric acid-THFPCIIFGPRSKGWVCK-NH₂ | >10 μM |
| 99 | 122 | Hy-HFPCIIFGPRSKGWVCK-NH₂ | >10 μM |
| 100 | 123 | Isovaleric acid-HFPCIIFGPRSKGWVCK-NH₂ | >10 μM |
| 101 | 124 | Hy-DTHFPCISFGPRSKGWVCK-NH₂ | >1 μM |
| 102 | 125 | Hy-DTHFPCIKFGPRSKGWVCK-NH₂ | >1 μM |
| 103 | 126 | Hy-EDTHFPCIIFGPRSKGWVCK-NH₂ | >1 μM |
| 105 | 128 | Isovaleric acid-DTHFPCIIFEPRSKGWVCK-NH₂ | 10 |
| 106 | 129 | Isovaleric acid-DTHFPCIIFSPRSKGWVCK-NH₂ | 44 |
| 107 | 130 | Isovaleric acid-DTHFSCIIFGPRSKGWVCK-NH₂ | 50 |
| 108 | 131 | Octanoic acid-PEG11-DTHFPCIIFGPRSRGWVCK-NH₂ | >1 μM |
| 109 | 132 | Isobutyric acid-PEG11-DTHFPCIIFGPRSRGWVCK-NH₂ | >1 μM |
| 110 | 133 | [Ida]-THFPCIIFGPRSRGWVCK-NH₂ | >300 Nm |
| 111 | 134 | Isovaleric acid-DTHFPCIIFGPKSKGWVCK-NH₂ | 12 |
| 112 | 135 | Isovaleric acid-DTHFPCIKFGPKSKGWVCK-NH₂ | 15 |
| 113 | 136 | Isovaleric acid-DTHFPCIIFGPRSKGWCK-NH₂ | 15 |
| 114 | 137 | Isovaleric acid-DTHFPCIIFGPRSKGVC-NH₂ | 18 |
| 115 | 138 | Isovaleric acid-DTHFPCIIFGPRSKGCK-NH₂ | 21 |
| 117 | 140 | Isovaleric acid-DTHFPC-[Dapa]-IFGPRSKGWDCK-NH₂ | 65 |
| 118 | 141 | Isovaleric acid-DTHFPCI-[Dapa]-FGPRSKGWDCK-NH₂ | 17 |
| 119 | 142 | Isovaleric acid-DTHFPC-[Dapa]-IFGPRSKGWECK-NH₂ | 151 |
| 120 | 143 | Isovaleric acid-DTHFPCI-[Dapa]-FGPRSKGWECK-NH₂ | 15 |
| 121 | 144 | Isovaleric acid-DTHFPCIKFGPRSKGWECK-NH₂ | 14 |
| 122 | 145 | Isovaleric acid-DTHFGCIIFGPRSKGWVCK-NH₂ | 57 |
| 123 | 146 | Hy-DTHFGCIIFGPRSKGWVCK-NH₂ | >10 μM |
| 124 | 147 | Isovaleric acid-DTHFRCIIFGPRSKGWVCK-NH₂ | 106 |
| 125 | 148 | Hy-DTHFRCIIFGPRSKGWVCK-NH₂ | >10 μM |
| 126 | 149 | Isovaleric acid-DTHF-[Sarc]-CIIFGPRSKGWVCK-NH₂ | 31 |
| 127 | 150 | Hy-DTHF-[Sarc]-CIIFGPRSKGWVCK-NH₂ | >10 μM |
| 128 | 151 | Isovaleric acid-DTHF-[β-Ala]-CIIFGPRSKGWVCK-NH₂ | 264 |
| 129 | 152 | Hy-DTHF-[β-Ala]-CIIFGPRSKGWVCK-NH₂ | >10 μM |
| 130 | 153 | Isovaleric acid-DTHFKCIIFGPRSKGWVCK-NH₂ | 150 |
| 131 | 154 | Hy-DTHFKCIIFGPRSKGWVCK-NH₂ | >10 μM |
| 132 | 155 | Hy-THFPCIIFGPRSKGWVCM-NH₂ | >1 μM |
| 133 | 156 | Hy-HFPCIIFGPRSKGWVCM-NH₂ | >1 μM |
| 134 | 157 | Isovaleric acid-HFPCIIFGPRSKGWVCM-NH₂ | >1 μM |
| 135 | 158 | Hy-DTHFPCISFGPRSKGWVCM-NH₂ | 545 |
| 136 | 159 | Hy-DTHFPCIKFGPRSKGWVCM-NH₂ | 669 |
| 137 | 160 | Hy-EDTHFPCIIFGPRSKGWVCM-NH₂ | 873 |
| 139 | 162 | Hy-DTHFPCIIFEPRSKGWVCM-NH₂ | N/A |
| 140 | 163 | Isovaleric acid-DTHFKCIEFGPRSKGWVCK-NH₂ | >1 μM |
| 141 | 164 | Isovaleric acid-DTHFPCIIFGPRSKGWACK-NH₂ | 11 |
| 142 | 165 | Isovaleric acid-DTHFPCIIFEPRSKGWVCK-NH₂ | 9 |
| 143 | 166 | Isovaleric acid-DTHFPCIIFGPRSKGWVCKKKK-NH₂ | 24 |
| 144 | 167 | Isovaleric acid-DTHFPCIIFEPRSKGWVCKKKK-NH₂ | 15 |
| 145 | 168 | Isovaleric acid-DTHFPCIIFGPRSKGWVCKK-NH₂ | 9 |
| 146 | 169 | Isovaleric acid-DTAFPCIIFGPRSKGWVCK-NH₂ | 24 |
| 147 | 170 | Isovaleric acid-DTKFPCIIFGPRSKGWVCK-NH₂ | 20 |
| 148 | 171 | Isovaleric acid-DTHFPC₁IIFVC₂HRPKGC₂YRRVC₁R-NH₂ | 2.2 |
| 149 | 172 | Isovaleric acid-DTHFPCI-K(m-PEG8)-FGPRSKGWVCK- | 9 |
| | | NH₂ | |
| 150 | 173 | Isovaleric acid-DTHFPCIKF-K(m-PEG8)-PRSKGWVCK- | 7 |
| | | NH₂ | |
| 151 | 174 | Isovaleric acid-DTHFPCIKFGP-K(m-PEG8)-SKGWVCK- | 13 |
| | | NH₂ | |
| 152 | 175 | Isovaleric acid-DTHFPCIKFGPRS-K(m-PEG8)-GWVCK- | 16 |
| | | NH₂ | |
| 153 | 176 | Isovaleric acid-DTHFPCIKFGPRSKGWVC-K(m-PEG8)- | 18 |
| | | NH₂ | |
| 154 | 177 | Isovaleric acid-DTHFPCIKFGPRSKGWTCK-NH₂ | 18 |
| 155 | 178 | Isovaleric acid-DTHFPCIEFGPRSKGWTCK-NH₂ | 38 |
| 156 | 179 | Isovaleric acid-DTHFPICIFGPRS-K(Betaine)-GWVC-NH₂ | Not |
| | | | Tested |
| 157 | 180 | Isovaleric acid-DTHFPCIKFGPRS-K(Betaine)-GWVCK- | 18 |
| | | NH₂ | |
| 158 | 181 | Isovaleric acid-DTHFPCI-K(Betaine)-FGPRSKGWVCK- | 16 |
| | | NH₂ | |
| 159 | 182 | Isovaleric acid-DTHFPCIKFGPRSKGWVC-K(Betaine)- | 17 |
| | | NH₂ | |
| 160 | 183 | Ac-DTHFPCIKFGPRSKGWVCK-NH₂ | 464 |
| 161 | 184 | Isovaleric acid-PEG3-DTHFPCIKFGPRSKGWVCK-NH₂ | 666 |
| 162 | 185 | Isobutyric acid-DTHFPCIKFGPRSKGWVCK-NH₂ | 41 |
| 163 | 186 | Valeric acid-DTHFPCIKFGPRSKGWVCK-NH₂ | 64 |
| 164 | 187 | Hy-VDTHFPCIKFGPRSKGWVCK-NH₂ | 146 |
| 165 | 188 | Hy-LDTHFPCIKFGPRSKGWVCK-NH₂ | 107 |
| 166 | 189 | Hexanoic acid-DTHFPCIKFGPRSKGWVCK-NH₂ | 36 |
| 167 | 190 | 5-Methylpentanoic acid-DTHFPCIKFGPRSKGWVCK-NH₂ | 99 |
| 168 | 191 | Cyclohexanoic acid-DTHFPCIKFGPRSKGWVCK-NH₂ | 30 |
| 169 | 192 | Heptanoic acid-DTHFPCIKFGPRSKGWVCK-NH₂ | 91 |
| 170 | 193 | Octanoic acid-DTHFPCIKFGPRSKGWVCK-NH₂ | 183 |
| 171 | 194 | Isovaleric acid-DTHFPCIIFGPRSKGWKCK-NH₂ | 48 |
| 172 | 195 | Isovaleric acid-DTHFPCIIFGPRSKGWECK-NH₂ | 15 |
| 173 | 196 | Isovaleric acid-DTHFPCRRFGPRSKGWVCK-NH₂ | Not |
| | | | Tested |
| 176 | 199 | Isovaleric acid-DTHFPICIFGPRS-K(m-PEG8)- | 6 |
| | | GWVC-NH₂ | |
| 177 | 200 | Isovaleric acid-DTHFPICIFGPRS-K-[(m-PEG4)]- | 6 |
| | | GWVC-NH₂ | |
| 178 | 201 | Isovaleric acid-DTHFPCIIFGPRSRGWVC-K(m-PEG8)- | 3 |
| | | NH₂ | |
| 179 | 202 | Isovaleric acid-DTHFPCIIFGPRSRGWVC-K-[(m-PEG4)]-- | 4 |
| | | NH₂ | |
| 180 | 203 | Isovaleric acid-DTHFPCIIFGPRSRGWVC-K(PEG2)-NH₂ | 9 |
| 181 | 204 | Isovaleric acid-DTHFPCIKFEPRSKGWVCK-NH₂ | 15 |
| 182 | 205 | Isovaleric acid-DTHFPCIKFEPRSKGWTCK-NH₂ | 13 |
| 183 | 206 | Isovaleric acid-DTHFPCIKFEPRSKGWCK-NH₂ | 17 |
| 184 | 207 | Isovaleric acid-DTHFPCIKFEPRSKGCK-NH₂ | 23 |
| 185 | 208 | Isovaleric acid-DTHFPCIFEPRSKGCK-NH₂ | 54 |
| 186 | 209 | Isovaleric acid-DTHFPCIFEPRSKGWCK-NH₂ | 12 |
| 187 | 210 | Isovaleric acid-DTHFPCIKFGPRSKCK-NH₂ | 21 |
| 188 | 211 | Isovaleric acid-DTHFPCIKFGPRSCK-NH₂ | 30 |
| 189 | 212 | Isovaleric acid-DTHFPCIKFGPRCK-NH₂ | 36 |
| 190 | 213 | Isovaleric acid-DTHFPCIKFGPCK-NH₂ | 55 |
| 191 | 214 | Isovaleric acid-DTHFPCIKFGCK-NH₂ | 97 |
| 192 | 215 | Isovaleric acid-DTHFPCIKFCK-NH₂ | 48 |
| 193 | 216 | Isovaleric acid-DTHFPCIKFC-NH₂ | 80 |
| 194 | 217 | Isovaleric acid-DTHFPCI-K(Palm)-FGPRSKGWVCK-NH₂ | 4 |
| 195 | 218 | Isovaleric acid-DTHFPCIKF-K(Palm)-PRSKGWVCK-NH₂ | 9 |
| 196 | 219 | Isovaleric acid-DTHFPCIKFGP-K(Palm)-SKGWVCK-NH₂ | 2 |
| 197 | 220 | Isovaleric acid-DTHFPCIKFGPRS-K(Palm)-GWVCK-NH₂ | 1 |
| 198 | 221 | Isovaleric acid-DTHFPCIKFGPRSKGWVC-K(Palm)-NH₂ | 7 |
| 199 | 222 | Isovaleric acid-DTHFPCI-K(PEG3-Palm)- | 7 |
| | | FGPRSKGWVCK-NH₂ | |
| 200 | 223 | Isovaleric acid-DTHFPCIKF-K(PEG3-Palm)- | 6 |
| | | PRSKGWVCK-NH₂ | |
| 201 | 224 | Isovaleric acid-DTHFPCIKFGP-K(PEG3-Palm)- | 4 |
| | | SKGWVCK-NH₂ | |
| 202 | 225 | Isovaleric acid-DTHFPCIKFGPRS-K(PEG3-Palm)- | 3 |
| | | GWVCK-NH₂ | |
| 203 | 226 | Isovaleric acid-DTHFPCIKFGPRSKGWVC-K(PEG3- | 4 |
| | | Palm)-NH₂ | |
| 204 | 227 | Hy-DTHFPCI-K(IVA)-FGPRSKGWVCK-NH₂ | >300 nM |
| 205 | 228 | Hy-DTHFPCIKF-K(IVA)-PRSKGWVCK-NH₂ | >300 nM |
| 206 | 229 | Hy-DTHFPCIKFGP-K(IVA)-SKGWVCK-NH₂ | 624 |
| 207 | 230 | Hy-DTHFPCIKFGPRS-K(IVA)-GWVCK-NH₂ | 318 |
| 208 | 231 | Hy-DTHFPCIKFGPRSKGWVC-K(IVA)-NH₂ | 109 |
| 209 | 232 | Hy-DTHFPCI-K(PEG3-IVA)-FGPRSKGWVCK-NH₂ | 342 |
| 210 | 233 | Hy-DTHFPCIKF-K(PEG3-IVA)-PRSKGWVCK-NH₂ | 457 |
| 211 | 234 | Hy-DTHFPCIKFGP-K(PEG3-IVA)-SKGWVCK-NH₂ | >300 nM |
| 212 | 235 | Hy-DTHFPCIKFGPRS-K(PEG3-IVA)-GWVCK-NH₂ | >300 nM |
| 213 | 236 | Hy-DTHFPCIKFGPRSKGWVC-K(PEG3-IVA)-NH₂ | 233 |
| 214 | 237 | Isovaleric acid-DTHFPCIKFEPRSKKWVCK-NH₂ | 15 |
| 215 | 238 | Hy-DTHFPCIKFGPRSKGWVCK-NH₂ | >1 μM |
| 216 | 239 | Palm-DTHFPCIKFGPRSKGWVCK-NH₂ | >1 μM |
| 217 | 240 | Palm-PEG3-DTHFPCIKFGPRSKGWVCK-NH₂ | >1 μM |
| 218 | 241 | Isovaleric acid-DTHFPCI-K(isoglu-Palm)-FEPRSKGCK- | 10 |
| | | NH₂ | |
| 219 | 242 | Isovaleric acid-DTHFPCIKF-K(isoglu-Palm)-PRSKGCK- | 9 |
| | | NH₂ | |
| 220 | 243 | Isovaleric acid-DTHFPCIKFEP-K(isoglu-Palm)-SKGCK- | 5 |
| | | NH₂ | |
| 221 | 244 | Isovaleric acid-DTHFPCIKFEPRS-K(isoglu-Palm)-GCK- | 4 |
| | | NH₂ | |
| 222 | 245 | Isovaleric acid-DTHFPCIKFEPRSK-K(isoglu-Palm)-CK- | 4 |
| | | NH₂ | |
| 223 | 246 | Isovaleric acid-DTHFPCIKFEPRSKGC-K(isoglu-Palm)- | 5 |
| | | NH₂ | |
| 224 | 247 | Isovaleric acid-DTHFPCIKFEPRSKGCK-K(isoglu-Palm)- | 4 |
| | | NH₂ | |
| 225 | 248 | Isovaleric acid-DTHFPCI-K(dapa-Palm)-FEPRSKGCK- | 17 |
| | | NH₂ | |
| 226 | 249 | Isovaleric acid-DTHFPCIKF-K(dapa-Palm)-PRSKGCK- | 14 |
| | | NH₂ | |
| 227 | 250 | Isovaleric acid-DTHFPCIKFEP-K(dapa-Palm)-SKGCK- | 10 |
| | | NH₂ | |
| 228 | 251 | Isovaleric acid-DTHFPCIKFEPRS-K(dapa-Palm)-GCK- | 7 |
| | | NH₂ | |
| 229 | 252 | Isovaleric acid-DTHFPCIKFEPRSK-K(dapa-Palm)-CK- | 13 |
| | | NH₂ | |
| 230 | 253 | Isovaleric acid-DTHFPCIKFEPRSKGC-K(dapa-Palm)-K- | 10 |
| | | NH₂ | |
| 231 | 254 | Isovaleric acid-DTHFPCIKFEPRSKGCK-K(dapa-Palm)- | 11 |
| | | NH2 | |
| 232 | 255 | Isovaleric acid-DTHFPCIKFGPRSKGWVCK-NH₂ | Not |
| | | | Tested |
| 233 | 256 | Isovaleric acid-AAHFPCIKFGPRSKGWVCK-NH₂ | 320 |
| 234 | 257 | Isovaleric acid-ATHFPCIKFGPRSKGWVCK-NH₂ | 60 |
| 235 | 258 | Isovaleric acid-DAHFPCIKFGPRSKGWVCK-NH₂ | 203 |
| 236 | 259 | Isovaleric acid-DTHAPCIKFGPRSKGWVCK-NH₂ | >500 nM |
| 237 | 260 | Isovaleric acid-DTHFPCIKAGPRSKGWVCK-NH₂ | 50 |
| 238 | 261 | Isovaleric acid-DTHFPCIKFEPRSKGWVCK-OH | 47 |
| 239 | 262 | Isovaleric acid-DTHFPCIKFEPRSKGWECK-OH | 101 |
| 240 | 263 | Isovaleric acid-DTHFPCIIFEPRSKGWEC-OH | 139 |
| 241 | 264 | Isovaleric acid-DTHFPCIKFK(isoGlu-Palm)- | 6 |
| | | PRSKGWECK-NH₂ | |
| 242 | 265 | Isovaleric acid-DTHFPCIKFEPK(isoGlu-Palm)- | 8 |
| | | SKGWECK-NH₂ | |
| 243 | 266 | Isovaleric acid-DTHAPCIKFEPRSKGWECK-NH₂ | >10 μM |
| 244 | 267 | Ida-THFPCIKFEPRSK-K(isoGlu-Palm)CK-NH₂ | 25 |
| 245 | 268 | Isovaleric acid-DTHFPCI-K(isoGlu-Palm)- | 131 |
| | | FEPRSKGWEC-OH | |
| 246 | 269 | 4,4-5,5-6,6,6-Heptafluorohexanoic acid- | 480 |
| | | DTHFPCIKFGPRSKGWVCK-NH₂ | |
| 247 | 270 | Isovaleric acid-DTHFPCIKF-K(mysteric acid)- | 7 |
| | | PRSKGWVC-NH₂ | |
| 248 | 271 | Isovaleric acid-DTHFPCIKF-K(lauric acid)- | 10 |
| | | PRSKGWVC-NH₂ | |
| 249 | 272 | Isovaleric acid-DTHFPCIKF-K(decanoic acid)- | 22 |
| | | PRSKGWVC-NH₂ | |
| 250 | 273 | Isovaleric acid-DTHFPCIKF-K(octanoic acid)- | 30 |
| | | PRSKGWVC-NH₂ | |
| 251 | 274 | Isovaleric acid-DTHFPCIKF-K(hexanoic acid)- | 21 |
| | | PRSKGWVC-NH₂ | |
| 252 | 275 | Isovaleric acid-DTHFPCIKF-K(butyric acid)- | 37 |
| | | PRSKGWVC-NH₂ | |
| 253 | 276 | Isovaleric acid-DTHFPCIKF-K(Ac)-PRSKGWVC-NH₂ | 29 |
| 254 | 277 | Ida-THFPCIKFEPRSKGWVC-K(mysteric acid)-NH₂ | 20 |
| 255 | 278 | [Ida]-THFPCIKFEPRSKGWVC-K(lauric acid)-NH₂ | 52 |
| 256 | 279 | [Ida]-THFPCIKFEPRSKGWVC-K(decanoic acid)-NH₂ | 116 |
| 257 | 280 | [Ida]-THFPCIKFEPRSKGWVC-K(octanoic acid)-NH₂ | 129 |
| 258 | 281 | [Ida]-THFPCIKFEPRSKGWVC-K(hexanoic acid)-NH₂ | 191 |
| 259 | 282 | [Ida]-THFPCIKFEPRSKGWVC-K(butyric acid)-NH₂ | 355 |
| 260 | 283 | [Ida]-THFPCIKFEPRSKGWVC-K(Ac)-NH₂ | 502 |
| 261 | 284 | Isovaleric acid-HFPCIKFEPRSKGWVC-K(octanoic | >300 nM |
| | | acid)-NH₂ | |
| 262 | 285 | Isovaleric acid-HFPCIKFEPRSKGWVC-K(lauric | 77 |
| | | acid)-NH₂ | |
| 263 | 286 | Isovaleric acid-DTHFPCIKFEPHSKGCK-NH2 | 62 |
| 264 | 287 | Isovaleric acid-DTHFPCIHFEPHSKGC-NH₂ | 118 |
| 265 | 288 | Isovaleric acid-DTHFPCIKFEPHS-K(Albu)-GCK-NH₂ | 6 |
| 266 | 289 | Isovaleric acid-DTHFPCIKFEPREKEC-NH₂ | 183 |
| 267 | 290 | Isovaleric acid-DTAFPCIKFEPRSKEC-NH₂ | >1 μM |
| 268 | 291 | Isovaleric acid-DTHFPCIKFECK-NH₂ | 107 |
| 269 | 292 | Hy-DTHFPIAIFAAGICI-NH₂ | >10 μM |
| 270 | 293 | Hy-DTHFPIAIFAAICI-NH₂ | >10 μM |
| 271 | 294 | Hy-DTHFPIAIFAICI-NH₂ | >10 μM |
| 272 | 295 | Hy-DTHFPIAIFICI-NH₂ | >10 μM |
| 273 | 296 | Hy-DTHFPIAIICI-NH₂ | >10 μM |
| 274 | 297 | Hy-DTHFPIAICI-NH₂ | >10 μM |
| 275 | 298 | Hy-DTHFPIICI-NH₂ | >10 μM |
| 276 | 299 | Hy-DTHICIAIF-NH₂ | >10 μM |
| 277 | 300 | Hy-DTHCPIAIF-NH₂ | >10 μM |
| 278 | 301 | Hy-DTHFPCIIA-NH₂ | >1 μM |
| 279 | 302 | Hy-DTHFPCAIF-NH₂ | >1 μM |
| 280 | 303 | Hy-DTHFACIIF-NH₂ | >1 μM |
| 281 | 304 | Hy-DTHF-(D)-A1a-CIIF-NH₂ | >10 μM |
| 282 | 305 | Hy-DTHAPCIIF-NH₂ | >10 μM |
| 283 | 306 | Hy-DTAFPCIIF-NH₂ | 739 nM |
| 284 | 307 | Hy-ATHFPCIIF-NH2 | >1 μM |
| 285 | 308 | [Ida]-THF-[bhPro]-CIIF-NH₂ | >1 μM |
| 286 | 310 | Hy-DTHFPCIEF-NH₂ | >1 μM |
| 287 | 298 | Hy-DTHFPCIEF-NH₂ | >1 μM |
| 288 | 311 | Isovaleric acid-DTHFPCIIF-NH₂ | 16 nM |
| 289 | 312 | Isovaleric acid-DTHFPAIIF-NH2 | Inactive |
| 290 | 313 | Isovaleric acid-DTHFPSIIF-NH2 | Inactive |
| 291 | 314 | Isovaleric acid-DTHFPCIKF-NH₂ | 7 nM |
| 293 | 316 | Hy-DTHFPCIF-NH₂ | 52% at |
| | | | 1 μM |
| 297 | 320 | Hy-DTHFPCIKFF-NH₂ | 64% at |
| | | | 1 μM |
| 298 | 321 | Hy-YTHFPCIIF-NH₂ | Not |
| | | | Tested |
| 299 | 322 | Hy-LTHFPCIIF-NH₂ | 64% at |
| | | | 1 μM |
| 300 | 323 | Hy-ETHFPCIIF-NH₂ | 77% at |
| | | | 1 μM |
| 301 | 324 | Hy-DRHFPCIIF-NH₂ | Not |
| | | | Tested |
| 302 | 325 | Hy-DTKFPCIIF-NH₂ | 60% at |
| | | | 1 μM |
| 303 | 326 | Hy-DTHFECIIF-NH₂ | Not |
| | | | Tested |
| 304 | 327 | Hy-DTHFPCIIK-NH₂ | 55% at |
| | | | 1 μM |
| 305 | 328 | Hy-DTHFPCIIR-NH₂ | 62% at |
| | | | 1 μM |
| 306 | 329 | Hy-DTHFPCIEF-NH₂ | Not |
| | | | Tested |
| 307 | 330 | Hy-DTHFPCIVF-NH₂ | 75% at |
| | | | 1 μM |
| 308 | 331 | Hy-DTHFPCILF-NH₂ | 89% at |
| | | | 1 μM |
| 309 | 332 | Hy-DTHFPCILK-NH₂ | 55% at |
| | | | 1 μM |
| 310 | 333 | Hy-DTHFPCIEK-NH₂ | 0% at |
| | | | 1 μM |
| 355 | 369 | Isovaleric acid-DTHFPCIKFEPRSKECK-NH₂ | 48 |
| 356 | 370 | Isovaleric acid-DTHFPCIKFEPHSKECK-NH₂ | 181 |
| 357 | 371 | Isovaleric acid-DTHFPCIKKEPHSKECK-NH₂ | >1 μM |
| 358 | 372 | Isovaleric acid-DTHFPCIKF-K(isoglu-Palm)-PHSKECK-NH₂ | 6 |
| 359 | 373 | Isovaleric acid-DTHFPCIKFEPRECK-NH₂ | 64 |
| 360 | 374 | Isovaleric acid-DTHFPCIKFEPHECK-NH₂ | 138 |
| 361 | 375 | Isovaleric acid-DTHFPCIKFEPRCK-NH₂ | 29 |
| | 376 | DTHFPICIFC | |
| | 377 | FPIC | |
| | 378 | HFPIC | |
| | 379 | HFPICI | |
| | 380 | HFPICIF | |
| | 381 | DTHFPIC | |
| | 381 | DTHFPICI | |
| | 382 | DTHFPICIF | |
| | 383 | DTHFPIAIFC | |
| | 384 | DTHAPICIF | |
| | 385 | DTHAPI-[C-StBu]-IF | |
| | 386 | DTHAPI-[C-tBu]-IF | |
| | 387 | DTHFPIAIF | |
| | 388 | DTHFPISIF | |
| | 389 | DTHFPI-([D)-Cys]-IF | |
| | 390 | DTHFPI-[homoCys]-IF | |
| | 391 | DTHFPI-[Pen]-IF | |
| | 392 | DTHFPI-[(D)-Pen]-IF | |
| | 393 | DTHFPI-[Dapa(AcBr)]-IF | |
| | 394 | CDTHFPICIF | |
| | 395 | DTHFPICIF-NHCH2CH2S | |
| | 396 | CHFPICIF | |
| | 397 | HFPICIF-NHCH2CH2S | |
| | 398 | D-[Tle]-H-[Phg]-[Oic]-[Chg]-C-[Chg]-F | |
| | 399 | D-[Tle]-HP-[Oic]-[Chg]-C-[Chg]-F | |
| | 400 | [(D)Phe]-[(D)Ile]-[(D)Cys]-[(D)Ile]-[(D)pro]-[(D)Phe]- | |
| | | [(D)His]-[(D)Thr]-[(D)Asp] | |
| | 401 | [(D)Phe]-[(D)Ile]-[(D)Cys]-[(D)Ile]-[(D)Pro]-[(D)Phe]-[(D)His] | |
| | 402 | Chenodeoxycholate-(Peg11)-[(D)Phe]-[(D)Ile]-[(D)Cys]- | |
| | | [(D)Ile]-[(D)Pro]-[(D)Phe]-[(D)His]-[(D)Thr]-[(D)Asp] | |
| | 403 | Ursodeoxycholate-(Peg11)-[(D)Phe]-[(D)Ile]-[(D)Cys]-[(D)Ile]- | |
| | | [(D)Pro]-[(D)Phe]-[(D)His]-[(D)Thr]-[(D)Asp] | |
| | 404 | F-[(D)Ile]-[(D)Cys]-[(D)Ile]-[(D)Pro]-[(D)Phe]-[(D)His]- | |
| | | [(D)Thr]-[(D)Asp]-(Peg11)- | |
| | | GYIPEAPRDGQAYVRKDGEWVLLSTFL | |
| | 405 | F-[(D)Ile]-[(D)Cys]-[(D)Ile]-[(D)pro]-[(D)Phe]-[(D)His]- | |
| | | [(D)Thr]-[(D)Asp]-([GP-(Hyp])₁₀ | |
| | 406 | Palmitoyl-(Peg11)-[(D)Phe]-[(D)Ile]-[(D)Cys]-[(D)Ile]- | |
| | | [(D)Pro]-[(D)Phe]-[(D)His]-[(D)Thr]-[(D)Asp] | |
| | 407 | 2(Palmitoyl)-[Dapa]-(Peg11)-[(D)Phe]-[(D)Ile]-[(D)Cys]- | |
| | | [(D)Ile]-[(D)Pro]-[(D)Phe]-[(D)His]-[(D)Thr]-[(D)Asp] | |
| | 408 | DTH-[bhPhe]-PIICIF | |
| | 409 | DTH-[Dpa]-PICI | |
| | 410 | DTH-[Bip]-PICIF | |
| | 411 | DTH[1-Nal]-PICIF | |
| | 412 | DTH-[bhDpa]-PICIF | |
| | 413 | DTHFP-ICI-bhPhe | |
| | 414 | DTHFPICI-[Dpa] | |
| | 415 | DTHFPICI-[Bip] | |
| | 416 | DTHFPICI-[1-Nal] | |
| | 417 | DTHFPICI-[bhDpa] | |
| | 418 | DTH-[Dpa]-PICI-[Dpa] | |
| | 419 | D-[Dpa]-PICIF | |
| | 420 | D-[Dpa]-PICI-[Dpa] | |
| | 421 | DTH-[Dpa]-P-[(D)Arg]-CR-[Dpa] | |
| | 422 | DTH-[Dpa]-P-[(D)Arg]-C-[(D)Arg]-[Dpa] | |
| | 423 | DTH-[Dpa]-[Oic]-ICIF | |
| | 424 | DTH-[Dpa]-[Oic]-ICI-[Dpa] | |
| | 425 | DTH-[Dpa]-PCCC-[Dpa] | |
| | 426 | DTHFPICIF-[(D)Pro]-PK | |
| | 427 | DTHFPICIF-[(D)Pro]-PR | |
| | 428 | DTHFPICIF-[bhPro]-PK | |
| | 429 | DTHFPICIF-[bhPro]-PR | |
| | 430 | DTHFPICIF-[(D)Pro]-[bhPro]-K | |
| | 431 | DTHFPICIF-[(D)-Pro]-[bhPro]-R | |
| | 432 | DTHFPICI-[bhPhe]-[(D)Pro]-PK | |
| | 433 | DTHFPICI-[bhPhe]-[(D)Pro]-PR | |
| | 434 | DTHFPICI-[bhPhe]-[(D)Pro]-[bhPro]-K | |
| | 435 | DTHFPICI-[bhPhe]-[(D)Pro]-[bhPro]-R | |
| | 436 | C-[Inp]-[(D)Dpa]-[Amc]-R-[Amc]-[Inp]-[Dpa]-Cysteamide | |
| | 437 | CP-[(D)Dpa]-[Amc]-R-[Amc]-[Inp]-[Dpa]-Cysteamide | |
| | 438 | C-[(D)Pro]-[(D)Dpa]-[Amc]-R-[Amc]-[Inp]-[Dpa]-Cysteamide | |
| | 439 | CG-[(D)Dpa]-[Amc]-R-[Amc]-[Inp]-[Dpa]-Cysteamide | |
| | 440 | Hy-DTHFPCAIF-NH₂ | >1000 |
| | 441 | Hy-DTHFPCRRF-NH₂ | Not active |
| | 442 | [IDA]-TH-[Dpa]-[bhPro]CRR-[bhPhe]-NH₂ | 206 |
| | 443 | Hy-DTHFPCEIF-NH₂ | >1000 |
| | 444 | Hy-DTHFPCFIF-NH₂ | 1191.8 |
| | 445 | Hy-DTHFPCQIF-NH₂ | >1000 |
| | 446 | Hy-DTHFPCRIF-NH₂ | >1000 |
| | 447 | Hy-[pGlu]-THFPCRKF-NH₂ | >1000 |
| | 448 | Hy-DTHFPCLIF-NH₂ | >10 μM |
| | 449 | Hy-DTHFPCVIF-NH₂ | 81% at 10 |
| | | | uM |
| | 450 | Hy-DTHFPCEIF-NH₂ | 19% at 10 |
| | | | uM |
| | 451 | Hy-DTHFPCRIF-NH₂ | 31% at 10 |
| | | | uM |
| | 452 | Hy-DTHFPCKIF-NH₂ | 9% at 10 |
| | | | uM |
| | 453 | Hy-DTHFPCLF-NH₂ | 39% at 1 |
| | | | uM |
| | 454 | Hy-DTHFPCEF-NH₂ | 17% at 10 |
| | | | uM |
| | 455 | Hy-DTHFPCRF-NH₂ | 31% at 10 |
| | | | uM |
| | 456 | Hy-DTHFPRRFGPRSKGWVC-NH₂ | >1000 |
| | 457 | [IDA1-THF-[bhPro]-CRR-[bhPhe]GPRSKGWVC-NH₂ | >1000 |
| | 458 | Hy-DTHFPCIFGPRSKGWVC-NH₂ | >1000 |
| | 459 | Hy-DTHFPCRIFGPRSRGWVCK-NH₂ | >1000 |
| | 460 | Isovaleric acid-DTHFPCLIFGPRSKGWVCK-NH₂ | 19.2 |
| | 461 | Isovaleric acid-DTHFPCVIFGPRSKGWVCK-NH₂ | 41 |
| | 462 | Isovaleric acid-DTHFPCSIFGPRSKGWVCK-NH₂ | 78 |
| | 463 | Isovaleric acid-DTHFPCQIFGPRSKGWVCK-NH₂ | 157 |
| | 464 | Isovaleric acid-DTHFPCKIFGPRSKGWVCK-NH₂ | 86 |
| | 465 | Isovaleric acid-DTHFPC-[Dapa]-IFGPRSKGWDCK-NH₂ | 65 |
| | 466 | Isovaleric acid-DTHFPC-[Dapa]-IFGPRSKGWECK-NH₂ | 151 |
| | 467 | Isovaleric acid-DTHFPCKIFGPRSKGWECK-NH₂ | 163 |
| | 468 | Isovaleric acid-DTHFPCRRFGPRSKGWVCK-NH₂ | >1000 |
| | 469 | Isovaleric acid-DTHFPCTIFGPRSKGWVCK-NH₂ | Not |
| | | | Tested |
| | 470 | Hy-DTHFPIAICI-NH₂ | >10 μM |
| | 471 | Hy-DTHFPIICI-NH₂ | >10 μM |
| | 472 | Hy-DTHICIAIF-NH₂ | >10 μM |
| | 473 | Hy-DTHCPIAIF-NH₂ | >10 μM |
| | 474 | Hy-ATHFPCIIF-NH₂ | >1000 |
| | 475 | Hy-ADHFPCIIF-NH₂ | >1000 |
| | 476 | Hy-DTHFPCIIFKC-NH₂ | 6398.0 |
| | 477 | Hy-DTHFPCIIFAC-NH₂ | >1000 |
| | 478 | Hy-DTHFPCIIFAA-NH₂ | 59% at 1 |
| | | | uM |
| | 479 | Hy-DEHFPCIIF-NH₂ | 34% at 10 |
| | | | uM |
| | 480 | Hy-DPHFPCIIF-NH₂ | 64% at 10 |
| | | | uM |
| | 481 | Hy-DTHKPCIIF-NH₂ | 45% at 10 |
| | | | uM |
| | 482 | Hy-DTHVPCIIF-NH₂ | 34% at 10 |
| | | | uM |
| | 483 | Hy-DTHFVCIIF-NH₂ | 50% at 10 |
| | | | uM |
| | 484 | Hy-DTHFPCIIY-NH₂ | 75% at 10 |
| | | | uM |
| | 485 | Hy-DTHFPCIIT-NH₂ | 23% at 1 |
| | | | uM |
| | 486 | Hy-DTHFPCILY-NH₂ | 85% at 1 |
| | | | uM |
| | 487 | Hy-DTHFPCIEY-NH₂ | 8% at 1 |
| | | | uM |
| | 488 | Isovaleric acid-DTHFPCIIFGPRSKG-[N-MeTrp]-VC-NH₂ | 32 |
| | 489 | Isovaleric acid-DTHFPCIIF-[Sarc]-PRSKG-[N-MeTrp]-VC- | 10 |
| | | NH₂ | |
| | 490 | Isovaleric acid-DTHFPCIIF-[Sarc]-PHSKG-[N-MeTrp]-VC- | 9 |
| | | NH₂ | |
| | 491 | Isovaleric acid-DTHFPCIIFEPRSKHWVCK-NH₂ | 15 |
| | 492 | Isovaleric acid-DTHFPCIIFEPRSKEWVCK-NH₂ | 19 |
| | 493 | Isovaleric acid-DTHFPCIIFEPRSKLWVCK-NH₂ | 7 |
| | 494 | Isovaleric acid-DTHFPCIIFEPRSKFWVCK-NH₂ | 10 |
| | 495 | Isovaleric acid-DTHFPCIKFEPHSK-[Sarc]-CK-NH₂ | 28 |
| | 496 | Isovaleric acid-DTHFPCIKFKPHSKEWVCE-NH₂ | 46 |
| | 497 | Isovaleric acid-DTHFPCIKFEPRSKEWVCK-NH₂ | 20 |
| | 498 | Isovaleric acid-DTHFPCIKFEPRSKLWVCK-NH₂ | 9 |
| | 499 | Isovaleric acid-DTHFPCIKFEPRSKEWVCK-OH | 46 |
| | 500 | Isovaleric acid-DTHFPCIKFEPRS-K(isoGlu-octanoic acid)- | 48 |
| | | ECK-NH₂ | |
| | 501 | Hy-DTHFPCIIFGPRSKGWAVCYW-NH₂ | 197 |
| | 502 | Hy-DTHFPICIFGPHRSKGWVCM-NH₂ | 149 |
| | 503 | Hy-DTHFPCIIFGPRSKGWVAC-NH₂ | 281 |
| | 504 | Hy-DTHFP-[(D)Cys]-IIFGPRSKGWVA-[(D)Cys]-NH₂ | >10 μM |
| | 505 | Hy-DTHFPCIIFGPRSKGWVACY-NH₂ | >10 μM |
| | 506 | Hy-DTHFPCIIFGPRSRGHVCK-NH₂ | >1000 |
| | 507 | Hy-DTHFPCIIFGPRSKGWNCK-NH₂ | >1000 |
| | 508 | Hy-DTHFPCINFGPRSKGWVCK-NH₂ | >1000 |
| | 509 | Hy-DTHFPCIDFGPRSKGWVCK-NH₂ | >1000 |
| | 510 | Isovaleric acid-DTHFECIIFGPRSKGWVCK-NH₂ | >1000 |
| | 511 | Hy-DTHFPCIIFGGPRSRGWVCK-NH₂ | 520 |
| | 512 | Hy-DTHFPCIIFGGPRSKGWNCK-NH₂ | 404 |
| | 513 | Hy-DTHFPCIIFGGPRSKGWDCK-NH₂ | 679 |
| | 514 | Isovaleric acid-DTHFPCIFEPRSKGTCK-NH₂ | 57 |
| | 515 | Isovaleric acid-DTHFPCIIF-[PEG3]-C-NH₂ | 157 |
| | 516 | Isovaleric acid-DTHAPCIKF-[Sarc]-PRSKGWECK-NH₂ | >10 μM |
| | 517 | Isovaleric acid-DTHAPCIKFEPRSK-[Sarc]-WECK-NH₂ | >10 μM |
| | 518 | Isovaleric acid-DTHAPCIKFEPRSKEWECK-NH₂ | >10 μM |
| | 519 | Isovaleric acid-STHAPCIKFEPRSKGWECK-NH₂ | >10 μM |
| | 520 | Isovaleric acid-SKHAPCIKFEPRSKGWECK-NH₂ | >10 μM |
| | 521 | Isovaleric acid-DTHFPCIKFEPHSKEWVCK-NH₂ | 80 |
| | 522 | Isovaleric acid-DTAFPCIKFEPRSKEC-NH₂ | >10 μM |
| | 523 | Isovaleric acid-DTHFGCIKFEPRSKEWVCK-NH₂ | >1000 |
| | 524 | Isovaleric acid-DTEFPCIKFEPRSKEWVCK-NH₂ | >1000 |
| | 525 | Isovaleric acid-DTHFPCIKFEPRS-K(octanoic acid)-EWVCK- | 62 |
| | | NH₂ | |
| | 526 | Isovaleric acid-ETHFPCIKFEPRSKEWVCK-NH₂ | 181 |

To determine whether a given peptide modifies the internalization and degradation of endogenous ferroportin, the protein levels and cellular distribution of ferroportin in hepatocytes and macrophages treated with the peptide may be assayed using Western blotting, immunohistochemistry and ferroportin antibodies known in the art.

### Example 3

### Serum Stability Assay

Serum stability experiments were undertaken to complement the in vivo results and assist in the design of potent, stable Ferroportin agonists. Key peptides (10 μM) were incubated with pre-warmed human serum (Sigma), fresh rat serum or plasma at 37 degrees. Samples were taken at various time points up to 24 hours. The samples were separated from serum proteins and analysed for the presence of the peptide of interest using LC-MS. The amount of intact peptide in each sample was calculated using the analyte peak area in relation to the zero time point. Percent remaining at each timepoint is calculated based on the peak area response ratio of test to compound to internal standard. Time 0 is set to 100%, and all later timepoints are calculated relative to time 0. Half-lives are calculated by fitting to a first-order exponential decay equation using Graphpad. The full list of ex vivo stability human and rat is shown in Table 15.

TABLE 15
Examples of analogues possessing Serum/Plasma Half life

| | | Rat | Rat | Human |
|---|---|---|---|---|
| | | serum | plasma | serum t½ |
| SEQ ID NO | Sequence | t½ (h) | t½ (h) | (h) |
| 547 | Hy-DTHFPICIFCCGCCHRSKCGMCCKT-OH | | | 2.76 |
| | (Hepcidin) | | | (variable) |
| 545 | Hy-DTHFPICIF-NH₂ | — | — | 0.1 |
| 548 | Palm-PEG11-ficipfhtd-NH₂ | — | — | 0.06 |
| 28 | Hy-DTHFPCIIF-NH₂ | — | — | 0.18 |
| 549 | DTHFPICIFGPRSKGWVCM-NH₂ | 0.18 | — | 2.32 |
| 533 | (Hy-DTHFPICIF-NH₂)₂ | — | — | 0.67 |
| 93 | Hy-DTHFPCRRFGPRSKGWVC-NH₂ | — | — | 0.46 |
| 550 | Ida-THF-[bhPro]-CRR-[bhPhe]-GPRSKGWVC-NH₂ | — | — | 1.14 |
| 551 | Hy-[bhAsp]-THFPICIFGPRSKGWVC-NH₂ | — | — | 2.1 |
| 552 | Hy-[bhAsp]-TH-[NMePhe]-PICIFGPRSKGWVC-NH₂ | 0.16 | 4 | 1.93 |
| 29 | Isovaleric acid-DTHFPICIFGPRSKGWVC-NH₂ | 0.15 | 4 | 1.99 |
| 68 | Ac-DTHFPICIFKPRSKGWVCM-NH₂ | 0.31 | 5.9 | — |
| 553 | Ac-DTHFPICIF-K(m-PEG8)-PRSKGWVCM-NH₂ | 1.81 | 19.5 | 40 |
| 554 | Ac-DTHFPICIFGPRS-K(m-PEG8)-GWVCM-NH₂ | 1.82 | 6 | 40 |
| 555 | Ida-Th-Dpa-bhPro-CIIFGPRSRGWVCK-NH₂ | — | — | 0.51 |
| 556 | Hy-IPFIDTCFHGPRSRGWVCK-NH₂ | — | — | 0.18 |
| 30 | Isovaleric acid-DTHFPCIIFGPRSRGWVCK-NH₂ | 0.08 | 0.43 | 0.51 |
| 63 | Ac-DTHFPICIFGPRSKGWVCM-NH₂ | 0.38 | 6 | — |
| 128 | Isosaleric acid-DTHFPCIIFEPRSKGWVCK-NH₂ | 0.68 | — | 2.22 |
| 31 | Isovaleric acid-DTHFPCIIFGPRSKGWVC-NH₂ | 0.13 | 4 | 0.94 |
| 557 | Isovaleric acid-DTHFPCIIFGPRSKGVCK-NH₂ | 0.27 | — | 1.17 |
| 138 | Isovaleric acid-DTHFPCIIFGPRSKGCK-NH₂ | 0.19 | — | 1.33 |
| 558 | Isovaleric acid-DTHFPCIFGPRSKGWCK-NH₂ | 0.21 | — | 0.99 |
| 144 | Isovaleric acid-DTHFPCIKFGPRSKGWECK-NH₂ | 0.38 | — | 1.19 |
| 38 | Isovaleric acid-DTHFPCIIFGPRSKGWVCK-NH₂ | 0.14 | — | — |
| 36 | Isovaleric acid-DTHFPCIKFGPRSKGWVCK-NH₂ | 0.14 | — | 0.57 |
| 37 | Isovaleric acid-DTHFPCIQFGPRSKGWVCK-NH₂ | 0.12 | — | 0.61 |
| 42 | Isovaleric acid-DTHFPCIEFGPRSKGWVCK-NH₂ | 0.15 | — | 0.74 |
| 172 | Isovaleric acid-DTHFPCI-K(m-PEG8)-FGPRSKGWVCK- | 0.32 | — | 1.13 |
| | NH₂ | | | |
| 173 | Isovaleric acid-DTHFPCIKF-K(m-PEG8)-PRSKGWVCK- | 0.42 | — | 1.35 |
| | NH₂ | | | |
| 175 | Isovaleric acid-DTHFPCIKFGPRS-K(m-PEG8)-GWVCK- | 1.16 | — | 11.09 |
| | NH₂ | | | |
| 176 | Isovaleric acid-DTHFPCIKFGPRSKGWVC-K(m-PEG8)- | 0.41 | — | 3.36 |
| | NH₂ | | | |
| 181 | Isovaleric acid-DTHFPCI-K(Betaine)-FGPRSKGWVCK- | 0.14 | — | 1.22 |
| | NH₂ | | | |
| 559 | (Isovaleric acid-DTHFPCIIF-NH₂)₂ | 18 | — | >24 |
| 560 | Isovaleric acid-DTHFPICIFGPRS-K(m-PEG8)-GWVC-NH₂ | 1.62 | — | 15 |
| 561 | Isovaleric acid-DTHFPICIFGPRS-K(m-PEG4)-GWVC-NH₂ | 1.1 | — | 12 |
| 562 | (Isovaleric acid-DTHFPCIIFGPRSRGWVCK)₂-DIG-NH₂ | 0.59 | — | 9 |
| 563 | Isovaleric acid-DTHFPICIFGPRSKG-[NMeTrp]-VC-NH₂ | 0.07 | — | 0.4 |
| 564 | Isovaleric acid-DTHFPICIF-[Sar]-PRSKG-[NMeTrp]-VC- | 0.24 | — | 1.36 |
| | NH₂ | | | |
| 565 | Isovaleric acid-DTHFPICIF-[Sar]-PHSKG-[NMeTrp]-VC- | 11.3 | — | >24 |
| | NH₂ | | | |
| 207 | Isovaleric acid-DTHFPCIKFEPRSKGCK-NH₂ | 2.12 | — | 8.06 |
| 218 | Isovaleric acid-DTHFPCIKF-K(Palm)-PRSKGWVCK-NH₂ | 24 | — | >24 |
| 220 | Isovaleric acid-DTHFPCIKFGPRS-K(Palm)-GWVCK-NH₂ | >24 | — | >>24 |
| 223 | Isovaleric acid-DTHFPCIKF-K(PEG3-Palm)- | 3.95 | — | 22.2 |
| | PRSKGWVCK-NH₂ | | | |
| 228 | DTHFPCIKF-K(IVA)-PRSKGWVCK-NH₂ | 0.19 | — | 0.31 |
| 233 | DTHFPCIKF-K(PEG3-IVA)-PRSKGWVCK-NH₂ | 0.35 | — | 0.58 |
| 491 | Isovaleric acid-DTHFPCIIFEPRSKHWVCK-NH₂ | 1.29 | — | 4.71 |
| 492 | Isovaleric acid-DTHFPCIIFEPRSKEWVCK-NH₂ | 7.7 | — | >24 |
| 493 | Isovaleric acid-DTHFPCIIFEPRSKLWVCK-NH₂ | 3.7 | — | >24 |
| 566 | Isovaleric acid-DTHFPCIIFEPRSKKWVCK-NH₂ | 0.89 | — | 5.06 |
| 494 | Isovaleric acid-DTHFPCIIFEPRSKFWVCK-NH₂ | 2.69 | — | 20 |
| 567 | Isovaleric acid-DTHFPCIIF-PEG3-C-NH₂ | >24 | — | >>24 |
| 568 | DIG-(DTHFPCIIF-NH₂)₂ | >24 | — | >>24 |
| 242 | Isovaleric acid-DTHFPCIKF-K(Isoglu-Palm)-PRSKGCK- | 16 | — | >>24 |
| | NH₂ | | | |
| 569 | Isovaleric acid-DTHFPCIKFK(dapa-Palm)PRSKGCK-NH₂ | 14 | — | 24 |

### Example 4

### Reduction of Free Plasma Iron in Rats

To investigate whether the peptide analogues are effective in decreasing free Fe²⁺ in serum, Retro Inverse mini Hepcidin is used as a reference peptide. Although RI mini-Hep has a very low potency in vitro, it is highly active in vivo as reported by Presza et al. J Clin Invest. 2011.

At Day 1, the animals are monitored for free Fe²⁺ in serum. In order to reach a homogenous serum level, Fe²⁺ is analyzed and a homogenous cohort of 7 or 8 animals is randomized to each treatment group. At Day 2, an acute experiment is performed where the animals are subjected to intraperitoneal (i.p.) dosing of test compound and subsequent tail vein blood samples. Prior to dosing, the animals are put under a heating lamp for 3-5 minutes. Blood samples are drawn from the tail vein from all animals in order to determine serum iron levels prior to vehicle or compound dosing. Animals are dosed i.p. with 1 ml of test substance in vehicle or just vehicle and blood samples of 250 μl are drawn from each animal at t=0, 60, 120, 240, 360 min and 24 hours in the study of the reference compound. The dose response study performed with Retro Inverse (RI) mini-Hepcidin (Reference compound), and the efficacy study performed with test compounds are performed as separate experiments.

Analysis of Fe²⁺ from Day 0 and 1 is done at a later time point not later than 10 days after. The chemicals and equipment used are shown below in Table 13.

TABLE 13
Chemicals and equipment used

| | | SEQ | | Peptide | Peptide | | |
|---|---|---|---|---|---|---|---|
| | Cmpd. | ID | MW | Content | Content | | |
| Drug Name | No. | No. | (g/mol) | Calculated % | Determined % | Purity % | Solvent |
| Isovaleric | 2 | 29 | 2144.52 | 86.2 | 86.2 | 90 | Na- |
| acid- | | | | | | | Acetate |
| DTHFPICIF | | | | | | | buffer |
| GPRSKGW | | | | | | | |
| VC-NH₂ | | | | | | | |
| RI- | | 546 | 1091.3 | 82.7 | 82.7 | 94.2 | Strong |
| Hepcidin1-9 | | | | | | | PBS |

Initially, all compounds, including peptides analogues, are solubilized in acidic H₂O in pH=2.5 and to a concentration of 3 mg/ml API. Compounds are thereafter either dissolved in Na-Acetate buffer (50 mM Acetic Acid, 125 mM NaCl, pH 5.0) or strong PBS, (25 mM sodium phosphate, 125 mM NaCl, pH 7.4).

Male Sprague-Dawley rats weighing 200-250 g are used in the study. They are housed in groups for n=2 in a light-, temperature- and humidity-controlled room (12-hour light: 12-hour dark cycle, lights on/off at 0600/1800 hour; 23 degrees Celsius; 50% relative humidity). Humane endpoints are applied, according to OECD's ‘Guidelines for Endpoints in Animal Study Proposals.” The animals are monitored daily. In case of significantly affected condition (based on signs such as weight loss >30% (obese animals); abnormal posture; rough hair coat; exudate around eyes and/or nose; skin lesions; abnormal breathing; difficulty with ambulation; abnormal food or water intake; or self-mutilation), or other conditions causing significant pain or distress, the animals are euthanized immediately.

Iron content in plasma/serum is measured for iron content using a colorimetric assay on the Cobas c 111 according to instructions from the manufacturer of the assay (assay: IRON2: ACN 661).

The data obtained from the cobas Iron2 analysis is presented as mean values+/−SEM.

Dosing of peptide analogues of the present invention is expected to result in a decrease in serum iron level that is comparable to that observed after injection of the positive control Retro Inverse mini Hepcidin (RI-Mini-Hepcidin).

### Example 5

### In Vivo Validation of Peptide Analogues

Peptide analogues of the present invention were tested for in vivo activity, as described in the previous Example, with the following changes. Instead of rats, mice (C57-BL6) were tested. Peptides or vehicle controls were administered to the mice (n=8/group) with the compounds of the present invention dosed at 3000 nmol/kg, and a hepcidin control administered via subcutaneous injection at 1000 nmol/kg. Peptides tested are shown in Table 14 with internalization/degradation assay potency values.

TABLE 14
Potency of illustrative hepcidin analogues

| | | | Potency |
|---|---|---|---|
| Compound | SEQ ID | | EC50 |
| number | NO: | Sequence | (nM) |
| Hepcidin | 335 | DTHFPICIFCCGCCHRSKCGMCCKT-OH | 34 |
| Cmpd1 | 207 | Isovaleric acid-DTHFPCIKFEPRSKG_ _CK-NH2 | 23 |
| Cmpd2 | 36 | Isovaleric acid-DTHFPCIKFGPRSKGWVCK-NH₂ | 35 |
| Cmpd3 | 76 | Isovaleric acid-DTHFPCIKFGPRSKGWVCK-[(m- | 17 |
| | | PEG8)]--NH₂ | |
| Cmpd4 | 199 | Isovaleric acid-DTHFPICIFGPRSK-[(m-PEG8)]- | 6.4 |
| | | GWVC-NH₂ | |
| Cmpd5 | 492 | Isovaleric acid-DTHFPCIIFEPRSKEWVCK-NH₂ | 19 |
| Cmpd6 | 490 | Isovaleric acid-DTHFPCIIF-[Sarc]-PHSKG-[N- | 9 |
| | | MeTrp]-VC-NH₂ | |

The primary goal of this experiment was to validate, in a mouse model, the activity of peptide analogues of the present invention. Serum iron levels were assessed as in the previous Example two hours after peptide or vehicle administration. A significant reduction in serum iron was observed in compound-treated animals as compared to the vehicle control. Furthermore, the max-dose responses of compounds of the present invention are expected to be similar to the max-dose response achieved with Hepcidin.

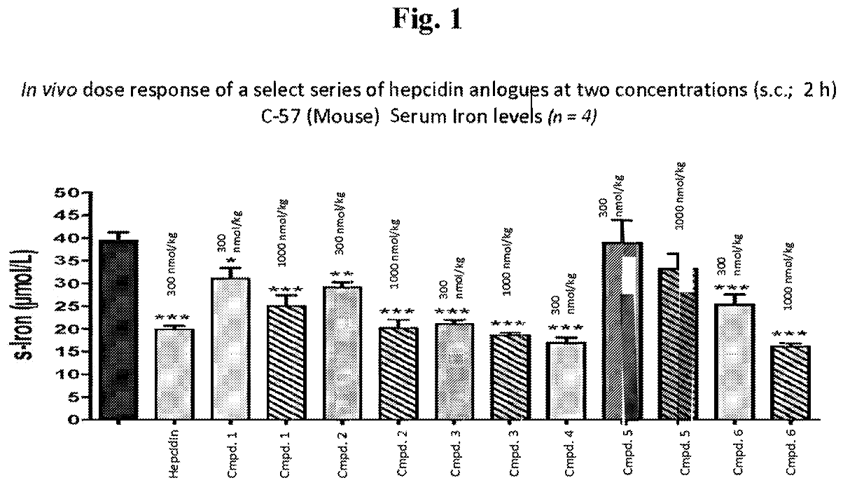

A similar experiment was performed with lower doses to assess the dose response of these compounds for inducing serum iron reduction. Methods were as described above in this Example, except for the following parameters: n=4 mice/group, however n=8 for the vehicle, as two groups are pooled. Mice were administered test compounds at two separate dosages (300 nmol/kg or 1000 nmol/kg), via subcutaneous injection. Serum iron levels were assessed as in the previous Example two hours after peptide or vehicle injection. These peptides induced similar iron reductions as native hepcidin in vivo. The results of this experiment are shown in FIG. 1, which provides an in vivo dose response of illustrative hepcidin analogues at two concentrations, 300 nmol/kg and 1000 nmol/kg (subcutaneous or “s.c.”; 2 h), in C-57 (mouse) presented as serum iron levels (n=4).

Other peptides are tested similarly, either in rats as described in the previous Example, or in mice as described above in the present Example. The route of peptide administration is via subcutaneous injection, unless otherwise indicated as being via intraperitoneal injection

The peptides are also tested for other pharmacokinetic/pharmacodynamic (PK/PD) parameters using methods commonly known by the skilled artisan. These parameters include determinations regarding stability (hours stable in plasma from the indicated human or rat subject), half-life in mice, and in vitro activity (EC₅₀). The PK/PD properties of peptide analogues of the present invention are compared with hepcidin to determine their PK/PD effects in C57BL6 mice. The peptide analogues are expected to produce a decrease in serum iron, which may be transient or sustained.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

1. A hepcidin analogue having the structure of Formula I:
(I)
SEQ ID NO: 1
R¹-X-Y-R²
or a pharmaceutically acceptable salt or solvate thereof,
wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;
R² is OH or NH₂;
X is a peptide sequence having the formula Ia:
(Ia)
SEQ ID NO: 2
X1-X2-X3-X4-X5-X6-X7-X8-X9-X10
wherein
X1 is Asp, Ser, Glu, Ida, pGlu, bhAsp, D-Asp or absent;
X2 is Thr, Ser, Lys, Glu, Pro, Ala or absent;
X3 is His, Ala, or Glu;
X4 is Phe, Ile or Dpa;
X5 is Pro, bhPro, Val, Glu, Sarc or Gly;
X6 is Cys or (D)-Cys;
X7 is absent or any amino acid except Ile, Cys or (D)-Cys;
X8 is absent or any amino acid except Cys or (D)-Cys;
X9 is Phe, Ala, Ile, Thr, Tyr, Lys, Arg, bhPhe, D-Phe or absent; and
X10 is Lys, Phe or absent; and
Y is absent or present;
provided that if Y is present, Y is a peptide having the formula Im:
(Im)
SEQ ID NO: 3
Y1-Y2-Y3-Y4-Y5-Y6-Y7-Y8-Y9-Y10-Y11-Y12
wherein
Y1 is Gly, PEG3, Sarc, Lys, Glu, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Glu, Tyr or absent;
Y5 is Lys, Met, Ser, Arg, Ala or absent;
Y6 is Gly, Sarc, Glu, Lys, Arg, Ser, Lys, Ile, Ala, Pro, Val or absent;
Y7 is Trp, Lys, Gly, Ala Ile, Val or absent;
Y8 is Val, Trp, His, Thr, Gly, Cys, Met, Tyr, Ala, Glu, Lys, Asp, Arg or absent;
Y9 is Val, Asp, Asn, Cys, Tyr or absent;
Y10 is Cys, Met, Lys, Arg, Tyr or absent;
Y11 is Arg, Met, Cys, Lys or absent; and
Y12 is Arg, Lys, Ala or absent.

2. The hepcidin analogue of claim 1, wherein X is a peptide sequence having the formula Ib:
(Ib)
SEQ ID NO: 18
X1-X2-X3-X4-X5-X6-X7-X8-X9-X10
wherein
X1 is Asp, Glu, Ida, pGlu, bhAsp, D-Asp or absent;
X2 is Thr, Ser, Lys, Glu, Pro, Ala or absent;
X3 is His, Ala, or Glu;
X4 is Phe, Ile or Dpa;
X5 is Pro, bhPro, Sarc or Gly;
X6 is Cys;
X7 is absent or any amino acid except Ile, Cys or (D)-Cys;
X8 is absent or any amino acid except Cys or (D)-Cys;
X9 is Phe, Ile, Tyr, bhPhe or D-Phe or absent; and
X10 is Lys, Phe or absent; and
wherein Y is absent or present, provided that if Y is present, Y is a peptide having the formula In:
(In)
SEQ ID NO: 19
Y1-Y2-Y3-Y4-Y5-Y6-Y7-Y8-Y9-Y10-Y11-Y12
wherein
Y1 is Gly, PEG3, Sarc, Lys, Glu, Ala, Phe, Pro, Glu, Lys, D-Pro, Val, Ser or absent;
Y2 is Pro, Ala, Gly or absent;
Y3 is Arg, Lys, Pro, Gly, His, Ala, or absent;
Y4 is Ser, Arg, Glu or absent;
Y5 is Lys, Ser, Met, Arg, Ala or absent;
Y6 is Gly, Sarc, Glu, Leu, Phe, His or absent;
Y7 is Trp, N-Methyl Trp, Lys, Thr, His, Gly, Ala, Ile, Val or absent;
Y8 is Val, Trp, Ala, Asn, Glu or absent;
Y9 is Val, Ala, Asn, Asp, Cys or absent;
Y10 is Cys, (D)Cys, Glu or absent;
Y11 is Tyr, Met or absent; and
Y12 is Trp or absent.

3. The hepcidin analogue of claim 1, wherein the hepcidin analogue comprises an amino acid sequence or a structure shown in Table 2.

4. A hepcidin analogue having the structure of Formula II:
(II)
SEQ ID NO: 4
R¹-X-Y-R²
or a pharmaceutically acceptable salt or solvate thereof,
wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;
R² is OH or NH₂;
X is a peptide sequence having the formula IIa:
(IIa) SEQ ID NO: 5
SEQ ID NO: 5
X1-X2-X3-X4-X5-X6-X7-X8-X9-X10
wherein
X1 is Asp, Glu or Ida;
X2 is Thr, Ser or absent;
X3 is His;
X4 is Phe or Dpa;
X5 is Pro, bhPro, Sarc or Gly;
X6 is Cys or (D)-Cys;
X7 is Arg, Glu, Phe, Gln, Leu, Val, Lys, Ile, Ala, Ser, Dapa or absent;
X8 is Ile, Arg, Lys, Arg, Ala, Gln, Phe, Glu, Asp, Tyr, Ser, Leu, Val, D-Ile, D-Lys, D-Arg, Dapa or absent;
X9 is Phe, Tyr, bhPhe, D-Phe or absent; and
X10 is Lys, Phe or absent; and
wherein Y is absent or present, provided that if Y is present, Y is a peptide having the formula IIm:
(IIm)
SEQ ID NO: 6
Y1-Y2-Y3-Y4-Y5-Y6-Y7-Y8-Y9-Y10-Y11-Y12
wherein
Y1 is Gly, Sarc, Lys, Glu or absent;
Y2 is Pro, Ala, Gly or absent;
Y3 is Arg, Lys, Pro, Gly, His, Ala or absent;
Y4 is Ser, Arg, Glu or absent;
Y5 is Lys, Ser, Met, Arg, Ala or absent;
Y6 is Gly, Sarc, Glu, Leu, Phe, His or absent;
Y7 is Trp, NMe-Trp, Lys, Thr, His, Gly, Ala Ile, Val or absent;
Y8 is Val, Trp, Ala, Asn, Glu or absent;
Y9 is Cys;
Y10 is absent;
Y11 is absent; and
Y12 is absent.

5. The hepcidin analogue of claim 4, wherein the hepcidin analogue comprises an amino acid sequence or a structure shown in Table 3.

6. A dimer comprising two hepcidin analogues, each hepcidin analogue having the structure of Formula I, the structure of Formula II, the structure of Formula III, the structure of Formula IV, the structure of Formula V, the structure of Formula VI, or a sequence or structure shown in any one of Tables 2-4 and 6-8, or 10-12, provided that when the dimer comprises a hepcidin analogue having the structure of Formula III, Formula IV, Formula V, or Formula VI, the two hepcidin analogues are linked via a lysine linker.

7. The dimer of claim 6, wherein one or both hepcidin analogue has the structure of Formula I.

8. The dimer of claim 6, wherein one or both hepcidin analogue has the structure of Formula II.

9. The dimer of claim 6, wherein one or both hepcidin analogue has the Formula III:
(III)
SEQ ID NO: 7
R¹-X-Y-R²
or a pharmaceutically acceptable salt or solvate thereof, wherein
R¹ is hydrogen, a C1-C6 alkyl, a C6-C12 aryl, a C6-C12 aryl C1-C6 alkyl, or a C1-C20 alkanoyl, and including PEGylated versions thereof, alone or as spacers of any of the foregoing;
R² is —NH₂ or —OH;
X is a peptide sequence having the formula (IIIa)
(IIIa)
SEQ ID NO: 8
X1-X2-X3-X4-X5-X6-X7-X8-X9-X10
wherein
X1 is Asp, Glu, Ala, Gly, Thr, Ida, pGlu, bhAsp, D-Asp, Tyr, Leu or absent;
X2 is Thr, Ala, Aib, D-Thr, Arg or absent;
X3 is His, Lys, Ala, or D-His;
X4 is Phe, Ala, Dpa or bhPhe;
X5 is Pro, Glu, Ser, Gly, Arg, Lys, Val, Ala, D-Pro, bhPro, Sarc, Abu or absent;
X6 is Ile, Cys, Arg, Leu, Lys, His, Glu, D-Ile, D-Arg, D-Cys, Val, Ser or Ala;
X7 is Cys, Ile, Ala, Leu, Val, Ser, Phe, Dapa, D-Ile or D-Cys;
X8 is Ile, Lys, Arg, Ala, Gln, Phe, Glu, Asp, Tyr, Ser, Leu, Val, D-Ile, D-Lys, D-Arg, or Dapa;
X9 is Phe, Ala, Ile, Tyr, Lys, Arg, bhPhe or D-Phe; and
X10 is Lys, Phe or absent; and
Y is absent or present, and when present, Y is a peptide having the formula (IIIm)
(IIIm)
SEQ ID NO: 9
Y1-Y2-Y3-Y4-Y5-Y6-Y7-Y8-Y9-Y10-Y11-Y12-Y13-Y14-Y15
wherein
Y1 is Gly, Cys, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Tyr or absent;
Y5 is Lys, Met, Arg, Ala or absent;
Y6 is Gly, Ser, Lys, Ile, Arg, Ala, Pro, Val or absent;
Y7 is Trp, Lys, Gly, Ala, Ile, Val or absent;
Y8 is Val, Thr, Gly, Cys, Met, Tyr, Ala, Glu, Lys, Asp, Arg or absent;
Y9 is Cys, Tyr or absent;
Y10 is Met, Lys, Arg, Tyr or absent;
Y11 is Arg, Met, Cys, Lys or absent;
Y12 is Arg, Lys, Ala or absent;
Y13 is Arg, Cys, Lys, Val or absent;
Y14 is Arg, Lys, Pro, Cys, Thr or absent; and
Y15 is Thr, Arg or absent;
wherein if Y is absent from the peptide of formula (III), X7 is Ile; and
wherein said compound of formula (III) is optionally PEGylated on R¹, X, or Y.

10. The dimer of claim 6, wherein one or both hepcidin analogue has the structure of Formula (IV):
(IV)
SEQ ID NO: 10
R¹-X-Y-R²
or a pharmaceutically acceptable salt or solvate thereof,
wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;
R² is —NH₂ or —OH;
X is a peptide sequence having the formula (IVa)
(IVa)
SEQ ID NO: 11
X1-X2-X3-X4-X5-X6-X7-X8-X9-X10
wherein
X1 is Asp, Glu, Ala, Gly, Thr, Ida, pGlu, bhAsp, D-Asp, Tyr, Leu or absent;
X2 is Thr, Ala, Aib, D-Thr, Arg or absent;
X3 is His, Lys, Ala, or D-His;
X4 is Phe, Ala, Dpa, bhPhe or D-Phe;
X5 is Pro, Glu, Ser, Gly, Arg, Lys, Val, Ala, D-Pro, bhPro, Sarc, Abu or absent;
X6 is Ile, Cys, Arg, Leu, Lys, His, Glu, D-Ile, D-Arg, D-Cys, Val, Ser or Ala;
X7 is Cys, Ile, Ala, Leu, Val, Ser, Phe, Dapa, D-Ile or D-Cys;
X8 is Ile, Lys, Arg, Ala, Gln, Phe, Glu, Asp, Tyr, Ser, Leu, Val, D-Ile, D-Lys, D-Arg or Dapa;
X9 is Phe, Ala, Ile, Tyr, Lys, Arg, bhPhe or D-Phe; and
X10 is Lys, Phe or absent;
wherein Y is present or absent, and provided that if Y is absent, X7 is Ile; and
Y is a peptide having the formula (IVm):
(IVm)
SEQ ID NO: 12
Y1-Y2-Y3-Y4-Y5-Y6-Y7-Y8-Y9-Y10-Y11-Y12-Y13-Y14-Y15
wherein
Y1 is Gly, Cys, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Tyr or absent;
Y5 is Lys, Met, Arg, Ala or absent;
Y6 is Gly, Ser, Lys, Ile, Arg, Ala, Pro, Val or absent;
Y7 is Trp, Lys, Gly, Ala, Ile, Val or absent;
Y8 is Val, Thr, Gly, Cys, Met, Tyr, Ala, Glu, Lys, Asp, Arg or absent;
Y9 is Cys, Tyr or absent;
Y10 is Met, Lys, Arg, Tyr or absent;
Y11 is Arg, Met, Cys, Lys or absent;
Y12 is Arg, Lys, Ala or absent;
Y13 is Arg, Cys, Lys, Val or absent;
Y14 is Arg, Lys, Pro, Cys, Thr or absent; and
Y15 is Thr, Arg or absent;
wherein said compound of formula (IV) is optionally PEGylated on R¹, X, or Y; and
wherein when said compound of formula (IV) comprises two or more cysteine residues, at least two of said cysteine residues being linked via a disulfide bond.

11. The dimer of claim 6, wherein one or both hepcidin analogue has the structure of Formula V:
(V)
SEQ ID NO: 13
R¹-X-Y-R²
or a pharmaceutically acceptable salt or solvate thereof, wherein
wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;
R² is —NH₂ or —OH;
X is a peptide sequence having the formula (Va):
(Va)
SEQ ID NO: 14
X1-X2-X3-X4-X5-X6-X7-X8-X9-X10
wherein
X1 is Asp, Glu, Ala, Gly, Thr, Ida, pGlu, bhAsp, D-Asp, Tyr, Leu or absent;
X2 is Thr, Ala, Aib, D-Thr, Arg or absent;
X3 is His, Lys, Ala, D-His or Lys;
X4 is Phe, Ala, Dpa, bhPhe or D-Phe;
X5 is Pro, Glu, Ser, Gly, Arg, Lys, Val, Ala, D-Pro, bhPro, Sarc, Abu or absent;
X6 is Ile, Cys, Arg, Leu, Lys, His, Glu, D-Ile, D-Arg, D-Cys, Val, Ser or Ala;
X7 is Cys, Ile, Ala, Leu, Val, Ser, Phe, Dapa, D-Ile or D-Cys;
X8 is Ile, Lys, Arg, Ala, Gln, Phe, Glu, Asp, Tyr, Ser, Leu, Val, D-Ile, D-Lys, D-Arg, or Dapa;
X9 is Phe, Ala, Ile, Tyr, Lys, Arg, bhPhe or D-Phe; and
X10 is Lys, Phe or absent;
wherein Y is present or absent, and provided that if Y is absent, X7 is Ile;
wherein said compound of formula V is optionally PEGylated on R¹, X, or Y; and
wherein when said compound of formula V comprises two or more cysteine residues, at least two of said cysteine residues being linked via a disulfide bond.

12. The dimer of claim 6, wherein one or both hepcidin analogue has the structure of formula VI:
(VI)
SEQ ID NO: 15
R¹-X-Y-R²
or a pharmaceutically acceptable salt or solvate thereof, wherein
wherein R¹ is hydrogen, a C1-C6 alkyl, a C6-C12 aryl, a C6-C12 aryl C1-C6 alkyl, or a C1-C20 alkanoyl, and including PEGylated versions alone or as spacers of any of the foregoing;
R² is —NH₂ or —OH;
X is a peptide sequence having the formula (VIa):
(VIa)
SEQ ID NO: 16
X1-X2-X3-X4-X5-X6-X7-X8-X9-X10
wherein
X1 is Asp, Glu, Ida or absent;
X2 is Thr, Ser, Pro, Ala or absent;
X3 is His, Ala, or Glu;
X4 is Phe or Dpa;
X5 is Pro, bhPro, Sarc or Gly;
X6 is Cys, (D)-Cys, Arg, Glu, Phe, Gln, Leu, Val, Lys, Ala, Ser, Dapa or absent;
X7 is Cys, (D)-Cys, Arg, Glu, Phe, Gln, Leu, Val, Lys, Ala, Ser, Dapa or absent;
X8 is Ile, Arg, Lys, Ala, Gln, Phe, Glu, Asp, Tyr, Ser, Leu, Val, D-Ile, D-Lys, D-Arg, Dapa or absent;
X9 is Phe, Ala, Ile, Thr, Tyr, Lys, Arg, bhPhe, D-Phe or absent; and
X10 is Lys, Phe or absent;
Y is absent or present, provided that if Y is present, Y is a peptide having the formula (VIm)
(VIm)
SEQ ID NO: 17
Y1-Y2-Y3
wherein
Y1 is Ile, Arg, Lys, Ala, Gln, Phe, Glu, Asp, Tyr, Ser, Leu, Val, D-Ile, D-Lys, D-Arg, Dapa or absent;
Y2 is Phe, Ala, Ile, Thr, Tyr, Lys, Arg, bhPhe or D-Phe or absent; and
Y3 is Lys, Phe or absent.

13. The dimer of claim 6, wherein one or both hepcidin analogue has a sequence or structure shown in Table 4.

14. The dimer of claim 6, having a sequence or structure shown in any one of Tables 6, 7, 8, and any one of compounds 1-361 in Table 12.

15.-18. (canceled)

19. The hepcidin analogue of claim 1, wherein two cysteine residues of one or more hepcidin analogue are linked by an intramolecular disulfide bridge.

20.-22. (canceled)

23. A pharmaceutical composition comprising the hepcidin analogue of claim 1, and a pharmaceutically acceptable carrier, excipient or vehicle.

24. A method of binding a ferroportin or inducing ferroportin internalization and degradation, comprising contacting the ferroportin with at least one hepcidin analogue of claim 1.

25.-27. (canceled)

28. A device comprising the hepcidin analogue of claim 1, for delivery of the hepcidin analogue to a subject.

29. A kit comprising at least one hepcidin analogue of claim 1, packaged with a reagent, a device, or an instructional material, or a combination thereof.

30.-31. (canceled)

32. The dimer of claim 6, comprising a monomer peptide having a sequence or structure shown in Table 14 or 15.


SEQUENCE LISTING (606 sequences)

SEQ ID NO: 1 - protein, 22 aa, Artificial Sequence
  Description of Artificial Sequence: Synthetic peptide
  N-term hydrogen, a C1-C6 alkyl, a C6-C12 aryl, a C6-C12 aryl, C1-C6 alkyl or a C1-C20 alkanoyl, and including PEGylated versions alone or as spacers of any of the foregoing
  MOD_RES (1)..(1): Asp, Ser, Glu, Ida, pGlu, bhAsp, D-Asp or absent
  MOD_RES (2)..(2): Thr, Ser, Lys, Glu, Pro, Ala or absent
  MOD_RES (3)..(3): His, Ala or Glu
  MOD_RES (4)..(4): Phe, Ile or Dpa
  MOD_RES (5)..(5): Pro, bhPro, Val, Glu, Sarc or Gly
  MOD_RES (6)..(6): Cys or (D)-Cys
  MOD_RES (7)..(7): absent or any amino acid except Ile, Cys or (D)-Cys
  MOD_RES (8)..(8): absent or any amino acid except Cys or (D)-Cys
  MOD_RES (9)..(9): Phe, Ala, Ile, Thr, Tyr, Lys, Arg, bhPhe, D-Phe or absent
  MOD_RES (10)..(10): Lys, Phe or absent
  MOD_RES (11)..(11): Gly, Sarc, Lys, Glu, Ala, Phe, Pro, Glu, Lys, D-Pro, Val, Ser or absent
  MOD_RES (12)..(12): Pro, Ala, Cys, Gly or absent
  MOD_RES (13)..(13): Arg, Lys, Pro, Gly, His, Ala, Trp or absent
  MOD_RES (14)..(14): Ser, Arg, Gly, Trp, Ala, His, Glu, Tyr or absent
  MOD_RES (15)..(15): Lys, Met, Ser, Arg, Ala or absent
  MOD_RES (16)..(16): Gly, Sarc, Glu, Lys, Arg, Ser, Lys, Ile, Ala, Pro, Val or absent
  MOD_RES (17)..(17): Trp, Lys, Gly, Ala, Ile, Val or absent
  MOD_RES (18)..(18): Val, Trp, His, Thr, Gly, Cys, Met, Tyr, Ala, Glu, Lys, Asp, Arg or absent
  MOD_RES (19)..(19): Val, Asp, Asn, Cys, Tyr or absent
  MOD_RES (20)..(20): Cys, Met, Lys, Arg, Tyr or absent
  MOD_RES (21)..(21): Arg, Met, Cys, Lys or absent
  MOD_RES (22)..(22): Arg, Lys, Ala or absent
  misc_feature (11)..(22): This region may or may not be present
  C-term -OH or -NH2
XXXXXCXXXXXXXXXXXXXXXX

SEQ ID NO: 2 - protein, 10 aa, Artificial Sequence
  Description of Artificial Sequence: Synthetic peptide
  MOD_RES (1)..(1): Asp, Ser, Glu, Ida, pGlu, bhAsp, D-Asp or absent
  MOD_RES (2)..(2): Thr, Ser, Lys, Glu, Pro, Ala or absent
  MOD_RES (3)..(3): His, Ala or Glu
  MOD_RES (4)..(4): Phe, Ile or Dpa
  MOD_RES (5)..(5): Pro, bhPro, Val, Glu, Sarc or Gly
  MOD_RES (6)..(6): Cys or (D)-Cys
  MOD_RES (7)..(7): absent or any amino acid except Ile, Cys or (D)-Cys
  MOD_RES (8)..(8): absent or any amino acid except Cys or (D)-Cys
  MOD_RES (9)..(9): Phe, Ala, Ile, Thr, Tyr, Lys, Arg, bhPhe, D-Phe or absent
  MOD_RES (10)..(10): Lys, Phe or absent
XXXXXCXXXX

SEQ ID NO: 3 - protein, 12 aa, Artificial Sequence
  Description of Artificial Sequence: Synthetic peptide
  N-term may or may not include PEG3
  MOD_RES (1)..(1): Gly, Sarc, Lys, Glu, Ala, Phe, Pro, Glu, Lys, D-Pro, Val, Ser or absent
  MOD_RES (2)..(2): Pro, Ala, Cys, Gly or absent
  MOD_RES (3)..(3): Arg, Lys, Pro, Gly, His, Ala, Trp or absent
  MOD_RES (4)..(4): Ser, Arg, Gly, Trp, Ala, His, Glu, Tyr or absent
  MOD_RES (5)..(5): Lys, Met, Ser, Arg, Ala or absent
  MOD_RES (6)..(6): Gly, Sarc, Glu, Lys, Arg, Ser, Lys, Ile, Ala, Pro, Val or absent
  MOD_RES (7)..(7): Trp, Lys, Gly, Ala, Ile, Val or absent
  MOD_RES (8)..(8): Val, Trp, His, Thr, Gly, Cys, Met, Tyr, Ala, Glu, Lys, Asp, Arg or absent
  MOD_RES (9)..(9): Val, Asp, Asn, Cys, Tyr or absent
  MOD_RES (10)..(10): Cys, Met, Lys, Arg, Tyr or absent
  MOD_RES (11)..(11): Arg, Met, Cys, Lys or absent
  MOD_RES (12)..(12): Arg, Lys, Ala or absent
XXXXXXXXXXXX

SEQ ID NO: 4 - protein, 22 aa, Artificial Sequence
  Description of Artificial Sequence: Synthetic peptide
  N-term hydrogen, a C1-C6 alkyl, a C6-C12 aryl, a C6-C12 aryl, C1-C6 alkyl or a C1-C20 alkanoyl, and including PEGylated versions alone or as spacers of any of the foregoing
  MOD_RES (1)..(1): Asp, Glu or Ida
  MOD_RES (2)..(2): Thr, Ser or absent
  MOD_RES (4)..(4): Phe or Dpa
  MOD_RES (5)..(5): Pro, bhPro, Sarc or Gly
  MOD_RES (6)..(6): Cys or D-Cys
  MOD_RES (7)..(7): Arg, Glu, Phe, Gln, Leu, Val, Lys, Ile, Ala, Ser, Dapa or absent
  MOD_RES (8)..(8): Ile, Arg, Lys, Arg, Ala, Gln, Phe, Glu, Asp, Tyr, Ser, Leu, Val, D-Ile, D-Lys, D-Arg, Dapa or absent
  MOD_RES (9)..(9): Phe, Tyr, bhPhe, D-Phe or absent
  MOD_RES (10)..(10): Lys, Phe or absent
  MOD_RES (11)..(11): Gly, Sarc, Lys, Glu or absent
  MOD_RES (12)..(12): Pro, Ala, Gly or absent
  MOD_RES (13)..(13): Arg, Lys, Pro, Gly, His, Ala or absent
  MOD_RES (14)..(14): Ser, Arg, Glu or absent
  MOD_RES (15)..(15): Lys, Ser, Met, Arg, Ala or absent
  MOD_RES (16)..(16): Gly, Sarc, Glu, Leu, Phe, His or absent
  MOD_RES (17)..(17): Trp, N-Methyl-Trp, Lys, Thr, His, Gly, Ala, Ile, Val or absent
  MOD_RES (18)..(18): Val, Trp, Ala, Asn, Glu or absent
  MOD_RES (20)..(20): Met or absent
  MOD_RES (21)..(21): Tyr, Met or absent
  MOD_RES (22)..(22): Trp or absent
  misc_feature (11)..(22): This region may or may not be present
  C-term -OH or -NH2
XXHXXCXXXXXXXXXXXXCXXX

SEQ ID NO: 5 - protein, 10 aa, Artificial Sequence
  Description of Artificial Sequence: Synthetic peptide
  MOD_RES (1)..(1): Asp, Glu or Ida
  MOD_RES (2)..(2): Thr, Ser or absent
  MOD_RES (4)..(4): Phe or Dpa
  MOD_RES (5)..(5): Pro, bhPro, Sarc or Gly
  MOD_RES (6)..(6): Cys or D-Cys
  MOD_RES (7)..(7): Arg, Glu, Phe, Gln, Leu, Val, Lys, Ile, Ala, Ser, Dapa or absent
  MOD_RES (8)..(8): Ile, Arg, Lys, Arg, Ala, Gln, Phe, Glu, Asp, Tyr, Ser, Leu, Val, D-Ile, D-Lys, D-Arg, Dapa or absent
  MOD_RES (9)..(9): Phe, Tyr, bhPhe, D-Phe or absent
  MOD_RES (10)..(10): Lys, Phe or absent
XXHXXCXXXX

SEQ ID NO: 6 - protein, 12 aa, Artificial Sequence
  Description of Artificial Sequence: Synthetic peptide
  MOD_RES (1)..(1): Gly, Sarc, Lys, Glu or absent
  MOD_RES (2)..(2): Pro, Ala, Gly or absent
  MOD_RES (3)..(3): Arg, Lys, Pro, Gly, His, Ala or absent
  MOD_RES (4)..(4): Ser, Arg, Glu or absent
  MOD_RES (5)..(5): Lys, Ser, Met, Arg, Ala or absent
  MOD_RES (6)..(6): Gly, Sarc, Glu, Leu, Phe, His or absent
  MOD_RES (7)..(7): Trp, N-Methyl-Trp, Lys, Thr, His, Gly, Ala, Ile, Val or absent
  MOD_RES (8)..(8): Val, Trp, Ala, Asn, Glu or absent
  MOD_RES (10)..(10): Met or absent
  MOD_RES (11)..(11): Tyr, Met or absent
  MOD_RES (12)..(12): Trp or absent
XXXXXXXXCXXX

SEQ ID NO: 7 - protein, 25 aa, Artificial Sequence
  Description of Artificial Sequence: Synthetic peptide
  N-term hydrogen, a C1-C6 alkyl, a C6-C12 aryl, a C6-C12 aryl, C1-C6 alkyl or a C1-C20 alkanoyl, and including PEGylated versions thereof, alone or as spacers of any of the foregoing
  MOD_RES (1)..(1): Asp, Glu, Ala, Gly, Thr, Ida, pGlu, bhAsp, D-Asp, Tyr, Leu or absent
  MOD_RES (2)..(2): Thr, Ala, Aib, D-Thr, Arg or absent
  MOD_RES (3)..(3): His, Lys, Ala or D-His
  MOD_RES (4)..(4): Phe, Ala, Dpa or bhPhe
  MOD_RES (5)..(5): Pro, Glu, Ser, Gly, Arg, Lys, Val, Ala, D-Pro, bhPro, Sarc, Abu or absent
  MOD_RES (6)..(6): Ile, Cys, Arg, Leu, Lys, His, Glu, D-Ile, D-Arg, D-Cys, Val, Ser or Ala
  MOD_RES (7)..(7): Cys, Ile, Ala, Leu, Val, Ser, Phe, Dapa, D-Ile or D-Cys
  MOD_RES (8)..(8): Ile, Lys, Arg, Ala, Gln, Phe, Glu, Asp, Tyr, Ser, Leu, Val, D-Ile, D-Lys, D-Arg or Dapa
  MOD_RES (9)..(9): Phe, Ala, Ile, Tyr, Lys, Arg, bhPhe or D-Phe
  MOD_RES (10)..(10): Lys, Phe or absent
  MOD_RES (11)..(11): Gly, Cys, Ala, Phe, Pro, Glu, Lys, D-Pro, Val, Ser or absent
  MOD_RES (12)..(12): Pro, Ala, Cys, Gly or absent
  MOD_RES (13)..(13): Arg, Lys, Pro, Gly, His, Ala, Trp or absent
  MOD_RES (14)..(14): Ser, Arg, Gly, Trp, Ala, His, Tyr or absent
  MOD_RES (15)..(15): Lys, Met, Arg, Ala or absent
  MOD_RES (16)..(16): Gly, Ser, Lys, Ile, Arg, Ala, Pro, Val or absent
  MOD_RES (17)..(17): Trp, Lys, Gly, Ala, Ile, Val or absent
  MOD_RES (18)..(18): Val, Thr, Gly, Cys, Met, Tyr, Ala, Glu, Lys, Asp, Arg or absent
  MOD_RES (19)..(19): Cys, Tyr or absent
  MOD_RES (20)..(20): Met, Lys, Arg, Tyr or absent
  MOD_RES (21)..(21): Arg, Met, Cys, Lys or absent
  MOD_RES (22)..(22): Arg, Lys, Ala or absent
  MOD_RES (23)..(23): Arg, Cys, Lys, Val or absent
  MOD_RES (24)..(24): Arg, Lys, Pro, Cys, Thr or absent
  MOD_RES (25)..(25): Thr, Arg or absent
  misc_feature (11)..(25): This region may or may not be present
  C-term -NH2 or -OH
XXXXXXXXXXXXXXXXXXXXXXXXX

SEQ ID NO: 8 - protein, 10 aa, Artificial Sequence
  Description of Artificial Sequence: Synthetic peptide
  MOD_RES (1)..(1): Asp, Glu, Ala, Gly, Thr, Ida, pGlu, bhAsp, D-Asp, Tyr, Leu or absent
  MOD_RES (2)..(2): Thr, Ala, Aib, D-Thr, Arg or absent
  MOD_RES (3)..(3): His, Lys, Ala or D-His
  MOD_RES (4)..(4): Phe, Ala, Dpa or bhPhe
  MOD_RES (5)..(5): Pro, Glu, Ser, Gly, Arg, Lys, Val, Ala, D-Pro, bhPro, Sarc, Abu or absent
  MOD_RES (6)..(6): Ile, Cys, Arg, Leu, Lys, His, Glu, D-Ile, D-Arg, D-Cys, Val, Ser or Ala
  MOD_RES (7)..(7): Cys, Ile, Ala, Leu, Val, Ser, Phe, Dapa, D-Ile or D-Cys
  MOD_RES (8)..(8): Ile, Lys, Arg, Ala, Gln, Phe, Glu, Asp, Tyr, Ser, Leu, Val, D-Ile, D-Lys, D-Arg or Dapa
  MOD_RES (9)..(9): Phe, Ala, Ile, Tyr, Lys, Arg, bhPhe or D-Phe
  MOD_RES (10)..(10): Lys, Phe or absent
XXXXXXXXXX

SEQ ID NO: 9 - protein, 15 aa, Artificial Sequence
  Description of Artificial Sequence: Synthetic peptide
  MOD_RES (1)..(1): Gly, Cys, Ala, Phe, Pro, Glu, Lys, D-Pro, Val, Ser or absent
  MOD_RES (2)..(2): Pro, Ala, Cys, Gly or absent
  MOD_RES (3)..(3): Arg, Lys, Pro, Gly, His, Ala, Trp or absent
  MOD_RES (4)..(4): Ser, Arg, Gly, Trp, Ala, His, Tyr or absent
  MOD_RES (5)..(5): Lys, Met, Arg, Ala or absent
  MOD_RES (6)..(6): Gly, Ser, Lys, Ile, Arg, Ala, Pro, Val or absent
  MOD_RES (7)..(7): Trp, Lys, Gly, Ala, Ile, Val or absent
  MOD_RES (8)..(8): Val, Thr, Gly, Cys, Met, Tyr, Ala, Glu, Lys, Asp, Arg or absent
  MOD_RES (9)..(9): Cys, Tyr or absent
  MOD_RES (10)..(10): Met, Lys, Arg, Tyr or absent
  MOD_RES (11)..(11): Arg, Met, Cys, Lys or absent
  MOD_RES (12)..(12): Arg, Lys, Ala or absent
  MOD_RES (13)..(13): Arg, Cys, Lys, Val or absent
  MOD_RES (14)..(14): Arg, Lys, Pro, Cys, Thr or absent
  MOD_RES (15)..(15): Thr, Arg or absent
XXXXXXXXXXXXXXX

SEQ ID NO: 10 - protein, 25 aa, Artificial Sequence
  Description of Artificial Sequence: Synthetic peptide
  N-term hydrogen, a C1-C6 alkyl, a C6-C12 aryl, a C6-C12 aryl, C1-C6 alkyl or a C1-C20 alkanoyl, and including PEGylated versions alone or as spacers of any of the foregoing
  MOD_RES (1)..(1): Asp, Glu, Ala, Gly, Thr, Ida, pGlu, bhAsp, D-Asp, Tyr, Leu or absent
  MOD_RES (2)..(2): Thr, Ala, Aib, D-Thr, Arg or absent
  MOD_RES (3)..(3): His, Lys, Ala or D-His
  MOD_RES (4)..(4): Phe, Ala, Dpa, bhPhe or D-Phe
  MOD_RES (5)..(5): Pro, Glu, Ser, Gly, Arg, Lys, Val, Ala, D-Pro, bhPro, Sarc, Abu or absent
  MOD_RES (6)..(6): Ile, Cys, Arg, Leu, Lys, His, Glu, D-Ile, D-Arg, D-Cys, Val, Ser or Ala
  MOD_RES (7)..(7): Cys, Ile, Ala, Leu, Val, Ser, Phe, Dapa, D-Ile or D-Cys
  MOD_RES (8)..(8): Ile, Lys, Arg, Ala, Gln, Phe, Glu, Asp, Tyr, Ser, Leu, Val, D-Ile, D-Lys, D-Arg or Dapa
  MOD_RES (9)..(9): Phe, Ala, Ile, Tyr, Lys, Arg, bhPhe or D-Phe
  MOD_RES (10)..(10): Lys, Phe or absent
  MOD_RES (11)..(11): Gly, Cys, Ala, Phe, Pro, Glu, Lys, D-Pro, Val, Ser or absent
  MOD_RES (12)..(12): Pro, Ala, Cys, Gly or absent
  MOD_RES (13)..(13): Arg, Lys, Pro, Gly, His, Ala, Trp or absent
  MOD_RES (14)..(14): Ser, Arg, Gly, Trp, Ala, His, Tyr or absent
  MOD_RES (15)..(15): Lys, Met, Arg, Ala or absent
  MOD_RES (16)..(16): Gly, Ser, Lys, Ile, Arg, Ala, Pro, Val or absent
  MOD_RES (17)..(17): Trp, Lys, Gly, Ala, Ile, Val or absent
  MOD_RES (18)..(18): Val, Thr, Gly, Cys, Met, Tyr, Ala, Glu, Lys, Asp, Arg or absent
  MOD_RES (19)..(19): Cys, Tyr or absent
  MOD_RES (20)..(20): Met, Lys, Arg, Tyr or absent
  MOD_RES (21)..(21): Arg, Met, Cys, Lys or absent
  MOD_RES (22)..(22): Arg, Lys, Ala or absent
  MOD_RES (23)..(23): Arg, Cys, Lys, Val or absent
  MOD_RES (24)..(24): Arg, Lys, Pro, Cys, Thr or absent
  MOD_RES (25)..(25): Thr, Arg or absent
  misc_feature (11)..(25): This region may or may not be present
  C-term -NH2 or -OH
XXXXXXXXXXXXXXXXXXXXXXXXX

SEQ ID NO: 11 - protein, 10 aa, Artificial Sequence
  Description of Artificial Sequence: Synthetic peptide
  MOD_RES (1)..(1): Asp, Glu, Ala, Gly, Thr, Ida, pGlu, bhAsp, D-Asp, Tyr, Leu or absent
  MOD_RES (2)..(2): Thr, Ala, Aib, D-Thr, Arg or absent
  MOD_RES (3)..(3): His, Lys, Ala or D-His
  MOD_RES (4)..(4): Phe, Ala, Dpa, bhPhe or D-Phe
  MOD_RES (5)..(5): Pro, Glu, Ser, Gly, Arg, Lys, Val, Ala, D-Pro, bhPro, Sarc, Abu or absent
  MOD_RES (6)..(6): Ile, Cys, Arg, Leu, Lys, His, Glu, D-Ile, D-Arg, D-Cys, Val, Ser or Ala
  MOD_RES (7)..(7): Cys, Ile, Ala, Leu, Val, Ser, Phe, Dapa, D-Ile or D-Cys
  MOD_RES (8)..(8): Ile, Lys, Arg, Ala, Gln, Phe, Glu, Asp, Tyr, Ser, Leu, Val, D-Ile, D-Lys, D-Arg or Dapa
  MOD_RES (9)..(9): Phe, Ala, Ile, Tyr, Lys, Arg, bhPhe or D-Phe
  MOD_RES (10)..(10): Lys, Phe or absent
XXXXXXXXXX

SEQ ID NO: 12 - protein, 15 aa, Artificial Sequence
  Description of Artificial Sequence: Synthetic peptide
  MOD_RES (1)..(1): Gly, Cys, Ala, Phe, Pro, Glu, Lys, D-Pro, Val, Ser or absent
  MOD_RES (2)..(2): Pro, Ala, Cys, Gly or absent
  MOD_RES (3)..(3): Arg, Lys, Pro, Gly, His, Ala, Trp or absent
  MOD_RES (4)..(4): Ser, Arg, Gly, Trp, Ala, His, Tyr or absent
  MOD_RES (5)..(5): Lys, Met, Arg, Ala or absent
  MOD_RES (6)..(6): Gly, Ser, Lys, Ile, Arg, Ala, Pro, Val or absent
  MOD_RES (7)..(7): Trp, Lys, Gly, Ala, Ile, Val or absent
  MOD_RES (8)..(8): Val, Thr, Gly, Cys, Met, Tyr, Ala, Glu, Lys, Asp, Arg or absent
  MOD_RES (9)..(9): Cys, Tyr or absent
  MOD_RES (10)..(10): Met, Lys, Arg, Tyr or absent
  MOD_RES (11)..(11): Arg, Met, Cys, Lys or absent
  MOD_RES (12)..(12): Arg, Lys, Ala or absent
  MOD_RES (13)..(13): Arg, Cys, Lys, Val or absent
  MOD_RES (14)..(14): Arg, Lys, Pro, Cys, Thr or absent
  MOD_RES (15)..(15): Thr, Arg or absent
XXXXXXXXXXXXXXX

SEQ ID NO: 13 - protein, 10 aa, Artificial Sequence
  Description of Artificial Sequence: Synthetic peptide
  N-term hydrogen, a C1-C6 alkyl, a C6-C12 aryl, a C6-C12 aryl, C1-C6 alkyl or a C1-C20 alkanoyl, and including PEGylated versions alone or as spacers of any of the foregoing
  MOD_RES (1)..(1): Asp, Glu, Ala, Gly, Thr, Ida, pGlu, bhAsp, D-Asp, Tyr, Leu or absent
  MOD_RES (2)..(2): Thr, Ala, Aib, D-Thr, Arg or absent
  MOD_RES (3)..(3): His, Lys, Ala, D-His or Lys
  MOD_RES (4)..(4): Phe, Ala, Dpa, bhPhe or D-Phe
  MOD_RES (5)..(5): Pro, Glu, Ser, Gly, Arg, Lys, Val, Ala, D-Pro, bhPro, Sarc, Abu or absent
  MOD_RES (6)..(6): Ile, Cys, Arg, Leu, Lys, His, Glu, D-Ile, D-Arg, D-Cys, Val, Ser or Ala
  MOD_RES (7)..(7): Cys, Ile, Ala, Leu, Val, Ser, Phe, Dapa, D-Ile or D-Cys
  MOD_RES (8)..(8): Ile, Lys, Arg, Ala, Gln, Phe, Glu, Asp, Tyr, Ser, Leu, Val, D-Ile, D-Lys, D-Arg or Dapa
  MOD_RES (9)..(9): Phe, Ala, Ile, Tyr, Lys, Arg, bhPhe or D-Phe
  MOD_RES (10)..(10): Lys, Phe or absent
XXXXXXXXXX

SEQ ID NO: 14 - protein, 10 aa, Artificial Sequence
  Description of Artificial Sequence: Synthetic peptide
  MOD_RES (1)..(1): Asp, Glu, Ala, Gly, Thr, Ida, pGlu, bhAsp, D-Asp, Tyr, Leu or absent
  MOD_RES (2)..(2): Thr, Ala, Aib, D-Thr, Arg or absent
  MOD_RES (3)..(3): His, Lys, Ala, D-His or Lys
  MOD_RES (4)..(4): Phe, Ala, Dpa, bhPhe or D-Phe
  MOD_RES (5)..(5): Pro, Glu, Ser, Gly, Arg, Lys, Val, Ala, D-Pro, bhPro, Sarc, Abu or absent
  MOD_RES (6)..(6): Ile, Cys, Arg, Leu, Lys, His, Glu, D-Ile, D-Arg, D-Cys, Val, Ser or Ala
  MOD_RES (7)..(7): Cys, Ile, Ala, Leu, Val, Ser, Phe, Dapa, D-Ile or D-Cys
  MOD_RES (8)..(8): Ile, Lys, Arg, Ala, Gln, Phe, Glu, Asp, Tyr, Ser, Leu, Val, D-Ile, D-Lys, D-Arg or Dapa
  MOD_RES (9)..(9): Phe, Ala, Ile, Tyr, Lys, Arg, bhPhe or D-Phe
  MOD_RES (10)..(10): Lys, Phe or absent
XXXXXXXXXX

SEQ ID NO: 15 - protein, 13 aa, Artificial Sequence
  Description of Artificial Sequence: Synthetic peptide
  N-term hydrogen, a C1-C6 alkyl, a C6-C12 aryl, a C6-C12 aryl, C1-C6 alkyl or a C1-C20 alkanoyl, and including PEGylated, versions alone or as spacers of any of the foregoing
  MOD_RES (1)..(1): Asp, Glu, Ida or absent
  MOD_RES (2)..(2): Thr, Ser, Pro, Ala or absent
  MOD_RES (3)..(3): His, Ala or Glu
  MOD_RES (4)..(4): Phe or Dpa
  MOD_RES (5)..(5): Pro, bhPro, Sarc or Gly
  MOD_RES (6)..(6): Cys, (D)-Cys, Arg, Glu, Phe, Gln, Leu, Val, Lys, Ala, Ser, Dapa or absent
  MOD_RES (7)..(7): Cys, (D)-Cys, Arg, Glu, Phe, Gln, Leu, Val, Lys, Ala, Ser, Dapa or absent
  MOD_RES (8)..(8): Ile, Arg, Lys, Ala, Gln, Phe, Glu, Asp, Tyr, Ser, Leu, Val, D-Ile, D-Lys, D-Arg, Dapa or absent
  MOD_RES (9)..(9): Phe, Ala, Ile, Thr, Tyr, Lys, Arg, bhPhe, D-Phe or absent
  MOD_RES (10)..(10): Lys, Phe or absent
  MOD_RES (11)..(11): Ile, Arg, Lys, Ala, Gln, Phe, Glu, Asp, Tyr, Ser, Leu, Val, D-Ile, D-Lys, D-Arg, Dapa or absent
  MOD_RES (12)..(12): Phe, Ala, Ile, Thr, Tyr, Lys, Arg, bhPhe or D-Phe or absent
  MOD_RES (13)..(13): Lys, Phe or absent
  misc_feature (11)..(13): This region may or may not be present
  C-term -NH2 or -OH
XXXXXXXXXXXXX

SEQ ID NO: 16 - protein, 10 aa, Artificial Sequence
  Description of Artificial Sequence: Synthetic peptide
  MOD_RES (1)..(1): Asp, Glu, Ida or absent
  MOD_RES (2)..(2): Thr, Ser, Pro, Ala or absent
  MOD_RES (3)..(3): His, Ala or Glu
  MOD_RES (4)..(4): Phe or Dpa
  MOD_RES (5)..(5): Pro, bhPro, Sarc or Gly
  MOD_RES (6)..(6): Cys, (D)-Cys, Arg, Glu, Phe, Gln, Leu, Val, Lys, Ala, Ser, Dapa or absent
  MOD_RES (7)..(7): Cys, (D)-Cys, Arg, Glu, Phe, Gln, Leu, Val, Lys, Ala, Ser, Dapa or absent
  MOD_RES (8)..(8): Ile, Arg, Lys, Ala, Gln, Phe, Glu, Asp, Tyr, Ser, Leu, Val, D-Ile, D-Lys, D-Arg, Dapa or absent
  MOD_RES (9)..(9): Phe, Ala, Ile, Thr, Tyr, Lys, Arg, bhPhe, D-Phe or absent
  MOD_RES (10)..(10): Lys, Phe or absent
XXXXXXXXXX

SEQ ID NO: 17 - protein, 3 aa, Artificial Sequence
  Description of Artificial Sequence: Synthetic peptide
  MOD_RES (1)..(1): Ile, Arg, Lys, Ala, Gln, Phe, Glu, Asp, Tyr, Ser, Leu, Val, D-Ile, D-Lys, D-Arg, Dapa or absent
  MOD_RES (2)..(2): Phe, Ala, Ile, Thr, Tyr, Lys, Arg, bhPhe or D-Phe or absent
  MOD_RES (3)..(3): Lys, Phe or absent
XXX

SEQ ID NO: 18 - protein, 10 aa, Artificial Sequence
  Description of Artificial Sequence: Synthetic peptide
  MOD_RES (1)..(1): Asp, Glu, Ida, pGlu, bhAsp, D-Asp or absent
  MOD_RES (2)..(2): Thr, Ser, Lys, Glu, Pro, Ala or absent
  MOD_RES (3)..(3): His, Ala or Glu
  MOD_RES (4)..(4): Phe, Ile or Dpa
  MOD_RES (5)..(5): Pro, bhPro, Sarc or Gly
  MOD_RES (7)..(7): absent or any amino acid except Ile, Cys or (D)-Cys
  MOD_RES (8)..(8): absent or any amino acid except Cys or (D)-Cys
  MOD_RES (9)..(9): Phe, Ile, Tyr, bhPhe or D-Phe or absent
  MOD_RES (10)..(10): Lys, Phe or absent
XXXXXCXXXX

SEQ ID NO: 19 - protein, 12 aa, Artificial Sequence
  Description of Artificial Sequence: Synthetic peptide
  N-term may or may not include PEG3
  MOD_RES (1)..(1): Gly, Sarc, Lys, Glu, Ala, Phe, Pro, Glu, Lys, D-Pro, Val, Ser or absent
  MOD_RES (2)..(2): Pro, Ala, Gly or absent
  MOD_RES (3)..(3): Arg, Lys, Pro, Gly, His, Ala or absent
  MOD_RES (4)..(4): Ser, Arg, Glu or absent
  MOD_RES (5)..(5): Lys, Ser, Met, Arg, Ala or absent
  MOD_RES (6)..(6): Gly, Sarc, Glu, Leu, Phe, His or absent
  MOD_RES (7)..(7): Trp, N-Methyl Trp, Lys, Thr, His, Gly, Ala, Ile, Val or absent
  MOD_RES (8)..(8): Val, Trp, Ala, Asn, Glu or absent
  MOD_RES (9)..(9): Val, Ala, Asn, Asp, Cys or absent
  MOD_RES (10)..(10): Cys, (D)-Cys, Glu or absent
  MOD_RES (11)..(11): Tyr, Met or absent
  MOD_RES (12)..(12): Trp or absent
XXXXXXXXXXXX

SEQ ID NO: 20 - protein, 25 aa, Artificial Sequence
  Description of Artificial Sequence: Synthetic peptide
  MOD_RES (1)..(25): Any amino acid
  misc_feature (1)..(10): This region may encompass 1-10 residues
  misc_feature (11)..(25): This region may encompass 1-15 residues
  See specification as filed for detailed description of substitutions and preferred embodiments
XXXXXXXXXXXXXXXXXXXXXXXXX

SEQ ID NO: 21 - protein, 10 aa, Artificial Sequence
  Description of Artificial Sequence: Synthetic peptide
  MOD_RES (1)..(1): Asp, Glu, Ida, Lys or absent
  MOD_RES (2)..(2): Thr, Ser, Lys or absent
  MOD_RES (3)..(3): His, Ala or Lys
  MOD_RES (4)..(4): Phe, Dpa or Lys
  MOD_RES (5)..(5): Pro, bhPro, Gly or Lys
  MOD_RES (7)..(7): Arg, Glu, Phe, Gln, Leu, Val, Lys, Ala, Ser, Dapa, Thr or absent
  MOD_RES (8)..(8): Ile, Arg, Lys, Glu, Asn, Asp, Ala, Gln, Phe, Glu, Tyr, Ser, Leu, Val, D-Ile, D-Lys, D-Arg, Dapa or absent
  MOD_RES (9)..(9): Phe, Tyr, bhPhe, Lys or absent
  MOD_RES (10)..(10): Lys, Phe or absent
XXXXXCXXXX

SEQ ID NO: 22 - protein, 10 aa, Artificial Sequence
  Description of Artificial Sequence: Synthetic peptide
  MOD_RES (1)..(1): Asp, Glu, Ida, Lys or absent
  MOD_RES (2)..(2): Thr, Ser, Lys or absent
  MOD_RES (3)..(3): His, Ala, Lys
  MOD_RES (4)..(4): Phe, Dpa or Lys
  MOD_RES (5)..(5): Pro, bhPro, Gly or Lys
  MOD_RES (7)..(7): Arg, Glu, Phe, Gln, Leu, Val, Lys, Ala, Ser, Dapa, Thr or absent
  MOD_RES (8)..(8): Ile, Arg, Lys, Glu, Asn, Asp, Ala, Gln, Phe, Glu, Tyr, Ser, Leu, Val, D-Ile, D-Lys, D-Arg, Dapa or absent
  MOD_RES (9)..(9): Phe, Tyr, bhPhe, Lys or absent
  MOD_RES (10)..(10): Lys, Phe or absent
XXXXXCXXXX

SEQ ID NO: 23 - protein, 35 aa, Artificial Sequence
  Description of Artificial Sequence: Synthetic polypeptide
  N-term bond (e.g., a covalent bond) or selected from hydrogen, a C1-C6 alkyl, a C6-C12 aryl, a C6-C12 aryl C1-C6 alkyl, a C1-C20 alkanoyl, and including PEGylated versions alone or as spacers of any of the foregoing
  MOD_RES (1)..(35): Any amino acid
  misc_feature (1)..(35): This sequence may encompass 7 to 35, 8 to 35, 9 to 35, 10 to 35, 7 to 25, 8 to 25, 9 to 25, 10 to 25, 7 to 18, 8 to 18, 9 to 18, or 10 to 18 amino acid residues
  C-term may not may not include a bond (e.g., a covalent bond), -OH or NH2
XXXXXXXXXXXXXXXXXXXXXXXXXXXXXXXXXXX

SEQ ID NO: 24 - protein, 14 aa, Artificial Sequence
  Description of Artificial Sequence: Synthetic peptide
  N-term may include a bond (e.g., a covalent bond), hydrogen, a C1-C6 alkyl, a C6-C12 aryl, a C6-C12 aryl C1-C6 alkyl, a C1-C20 alkanoyl, and including PEGylated versions alone or as spacers of any of the foregoing
  MOD_RES (1)..(1): Asp, Glu, Ida or absent
  MOD_RES (2)..(2): Thr, Ser, Pro, Ala or absent
  MOD_RES (3)..(3): His, Ala, Glu or Ala
  MOD_RES (4)..(4): Phe or Dpa
  MOD_RES (5)..(5): Pro, bhPro, Sarc or Gly
  MOD_RES (6)..(6): Cys, (D)-Cys, Arg, Glu, Phe, Gln, Leu, Val, Lys, Ala, Ser, Dapa or absent
  MOD_RES (7)..(7): Cys, (D)-Cys, Arg, Glu, Phe, Gln, Leu, Val, Lys, Ala, Ser, Dapa or absent
  MOD_RES (8)..(8): Ile, Arg, Lys, Ala, Gln, Phe, Glu, Asp, Tyr, Ser, Leu, Val, D-Ile, D-Lys, D-Arg, Dapa or absent
  MOD_RES (9)..(9): Phe, Ala, Ile, Thr, Tyr, Lys, Arg, bhPhe, D-Phe or absent
  MOD_RES (10)..(10): Lys, Phe or absent
  MOD_RES (12)..(12): Ile, Arg, Lys, Ala, Gln, Phe, Glu, Asp, Tyr, Ser, Leu, Val, D-Ile, D-Lys, D-Arg, Dapa or absent
  MOD_RES (13)..(13): Phe, Ala, Ile, Thr, Tyr, Lys, Arg, bhPhe, D-Phe or absent
  MOD_RES (14)..(14): Lys, Phe or absent
  misc_feature (12)..(14): This region may be present or absent
  C-term may include a bond (e.g., a covalent bond), -OH or -NH2
XXXXXXXXXXKXXX

SEQ ID NO: 25 - protein, 10 aa, Artificial Sequence
  Description of Artificial Sequence: Synthetic peptide
  MOD_RES (1)..(1): Asp, Glu, Ida or absent
  MOD_RES (2)..(2): Thr, Ser, Pro, Ala or absent
  MOD_RES (3)..(3): His, Ala, Glu or Ala
  MOD_RES (4)..(4): Phe or Dpa
  MOD_RES (5)..(5): Pro, bhPro, Sarc or Gly
  MOD_RES (6)..(6): Cys, (D)-Cys, Arg, Glu, Phe, Gln, Leu, Val, Lys, Ala, Ser, Dapa or absent
  MOD_RES (7)..(7): Cys, (D)-Cys, Arg, Glu, Phe, Gln, Leu, Val, Lys, Ala, Ser, Dapa or absent
  MOD_RES (8)..(8): Ile, Arg, Lys, Ala, Gln, Phe, Glu, Asp, Tyr, Ser, Leu, Val, D-Ile, D-Lys, D-Arg, Dapa or absent
  MOD_RES (9)..(9): Phe, Ala, Ile, Thr, Tyr, Lys, Arg, bhPhe, D-Phe or absent
  MOD_RES (10)..(10): Lys, Phe or absent
XXXXXXXXXX

SEQ ID NO: 26 - protein, 3 aa, Artificial Sequence
  Description of Artificial Sequence: Synthetic peptide
  MOD_RES (1)..(1): Ile, Arg, Lys, Ala, Gln, Phe, Glu, Asp, Tyr, Ser, Leu, Val, D-Ile, D-Lys, D-Arg, Dapa or absent
  MOD_RES (2)..(2): Phe, Ala, Ile, Thr, Tyr, Lys, Arg, bhPhe, D-Phe or absent
  MOD_RES (3)..(3): Lys, Phe or absent
XXX

SEQ ID NO: 27 - protein, 10 aa, Artificial Sequence
  Description of Artificial Sequence: Synthetic peptide
  MOD_RES (1)..(1): Asp, Ida, pGlu, bhAsp or absent
  MOD_RES (4)..(4): Phe or Dpa
  MOD_RES (5)..(5): Pro or bhPro
  MOD_RES (6)..(6): Ile, Cys or Arg
  MOD_RES (7)..(7): Cys, Ile, Leu or Val
  MOD_RES (8)..(8): Ile, Lys, Glu, Phe, Gln or Arg
  MOD_RES (10)..(10): Lys, Phe or absent
XTHXPXXXFX

SEQ ID NO: 28 - protein, 9 aa, Artificial Sequence
  Description of Artificial Sequence: Synthetic peptide
  N-term Hy
  C-term NH2
DTHFPCIIF

SEQ ID NO: 29 - protein, 18 aa, Artificial Sequence
  Description of Artificial Sequence: Synthetic peptide
  N-term Isovaleric acid
  C-term NH2
DTHFPICIFGPRSKGWVC

SEQ ID NO: 30 - protein, 19 aa, Artificial Sequence
  Description of Artificial Sequence: Synthetic peptide
  N-term Isovaleric acid
  C-term NH2
DTHFPCIIFGPRSRGWVCK

SEQ ID NO: 31 - protein, 18 aa, Artificial Sequence
  Description of Artificial Sequence: Synthetic peptide
  N-term Isovaleric acid
  C-term NH2
DTHFPCIIFGPRSKGWVC

SEQ ID NO: 32 - protein, 19 aa, Artificial Sequence
  Description of Artificial Sequence: Synthetic peptide
  MOD_RES (1)..(1): Ida
  MOD_RES (4)..(4): Dpa
  MOD_RES (5)..(5): bhPro
  C-term NH2
XTHXPICIFGPRSKGWVCM

SEQ ID NO: 33 - protein, 19 aa, Artificial Sequence
  Description of Artificial Sequence: Synthetic peptide
  N-term Isovaleric acid
  C-term NH2
DTHFPCIFFGPRSKGWVCK

SEQ ID NO: 34 - protein, 19 aa, Artificial Sequence
  Description of Artificial Sequence: Synthetic peptide
  N-term Isovaleric acid
  C-term NH2
DTHFPCIIFGPRSKGWTCK

SEQ ID NO: 35 - protein, 19 aa, Artificial Sequence
  Description of Artificial Sequence: Synthetic peptide
  MOD_RES (1)..(1): Ida
  MOD_RES (4)..(4): Dpa
  MOD_RES (5)..(5): bhPro
  C-term NH2
XTHXPCIIFGPRSRGWVCK

SEQ ID NO: 36 - protein, 19 aa, Artificial Sequence
  Description of Artificial Sequence: Synthetic peptide
  N-term Isovaleric acid
  C-term NH2
DTHFPCIKFGPRSKGWVCK

SEQ ID NO: 37 - protein, 19 aa, Artificial Sequence
  Description of Artificial Sequence: Synthetic peptide
  N-term Isovaleric acid
  C-term NH2
DTHFPCIQFGPRSKGWVCK

SEQ ID NO: 38 - protein, 19 aa, Artificial Sequence
  Description of Artificial Sequence: Synthetic peptide
  N-term Isovaleric acid
  C-term NH2
DTHFPCIIFGPRSKGWVCK

SEQ ID NO: 39 - protein, 22 aa, Artificial Sequence
  Description of Artificial Sequence: Synthetic peptide
  N-term Hy
  C-term NH2
DTHFPICIFVCGHRSICYRRCR

SEQ ID NO: 40 - protein, 22 aa, Artificial Sequence
  Description of Artificial Sequence: Synthetic peptide
  N-term Isobutyric acid
  C-term NH2
DTHFPICIFVCHRSKGCYRRCR

SEQ ID NO: 41 - protein, 21 aa, Artificial Sequence
  Description of Artificial Sequence: Synthetic peptide
  N-term Hy
  C-term NH2
DTHFPICIFVCHRSKGCYRAC

SEQ ID NO: 42 - protein, 19 aa, Artificial Sequence
  Description of Artificial Sequence: Synthetic peptide
  N-term Isovaleric acid
  C-term NH2
DTHFPCIEFGPRSKGWVCK

SEQ ID NO: 43 - protein, 19 aa, Artificial Sequence
  Description of Artificial Sequence: Synthetic peptide
  N-term Hy
  C-term NH2
DTHFPICIFGPRAKGWVCM

SEQ ID NO: 44 - protein, 22 aa, Artificial Sequence
  Description of Artificial Sequence: Synthetic peptide
  N-term Isobutyric acid
  C-term NH2
DTHFPICIFVCHRSKGCYRRCR

SEQ ID NO: 45 - protein, 19 aa, Artificial Sequence
  Description of Artificial Sequence: Synthetic peptide
  N-term Hy
  C-term NH2
DTHFPICIFGPRSKGWVCM

SEQ ID NO: 46 - protein, 22 aa, Artificial Sequence
  Description of Artificial Sequence: Synthetic peptide
  N-term Hy
  C-term NH2
DTHFPICIFVCHRSKGCYRRCR

SEQ ID NO: 47 - protein, 19 aa, Artificial Sequence
  Description of Artificial Sequence: Synthetic peptide
  N-term Hy
  C-term NH2
DTHFPICIFGPRSRGWVCK

SEQ ID NO: 48 - protein, 19 aa, Artificial Sequence
  Description of Artificial Sequence: Synthetic peptide
  N-term Hy
  C-term NH2
DTHFPCIIFGPRSKGWVCM

SEQ ID NO: 49 - protein, 19 aa, Artificial Sequence
  Description of Artificial Sequence: Synthetic peptide
  N-term Hy
  C-term NH2
DTHFPICIFAPRSKGWVCM

SEQ ID NO: 50 - protein, 19 aa, Artificial Sequence
  Description of Artificial Sequence: Synthetic peptide
  N-term Hy
  C-term OH
DTHFPICIFGPRSKGWVCM

SEQ ID NO: 51 - protein, 9 aa, Artificial Sequence
  Description of Artificial Sequence: Synthetic peptide
  N-term Hy
  C-term NH2
DTHFPCIQF

SEQ ID NO: 52 - protein, 23 aa, Artificial Sequence
  Description of Artificial Sequence: Synthetic peptide
  N-term Hy
  C-term NH2
DTHFPICIFVCGHRSKGCYRRCR

SEQ ID NO: 53 - protein, 19 aa, Artificial Sequence
  Description of Artificial Sequence: Synthetic peptide
  N-term Hy
  C-term NH2
DTHFAICIFGPRSKGWVCM

SEQ ID NO: 54 - protein, 20 aa, Artificial Sequence
  Description of Artificial Sequence: Synthetic peptide
  N-term Hy
  C-term NH2
DTHFPICIFGPHRSKGWVCM

SEQ ID NO: 55 - protein, 19 aa, Artificial Sequence
  Description of Artificial Sequence: Synthetic peptide
  N-term Hy
  C-term NH2
DTHFPICIFGPRAKGWVCM

SEQ ID NO: 56 - protein, 19 aa, Artificial Sequence
  Description of Artificial Sequence: Synthetic peptide
  N-term Hy
  C-term NH2
DTHFPACIFGPRSKGWVCM

SEQ ID NO: 57 - protein, 23 aa, Artificial Sequence
  Description of Artificial Sequence: Synthetic peptide
  N-term Hy
  C-term NH2
DTHFPCIIFVCHRPKGCYRRVCR

SEQ ID NO: 58 - protein, 19 aa, Artificial Sequence
  Description of Artificial Sequence: Synthetic peptide
  N-term Hy
  C-term NH2
DTHFPICIFGPRSKAWVCM

SEQ ID NO: 59 - protein, 23 aa, Artificial Sequence
  Description of Artificial Sequence: Synthetic peptide
  N-term Hy
  C-term NH2
DTHFPICIFVCGHRGKGCYRRCR

SEQ ID NO: 60 - protein, 19 aa, Artificial Sequence
  Description of Artificial Sequence: Synthetic peptide
  N-term Hy
  C-term NH2
ATHFPICIFGPRSKGWVCM

SEQ ID NO: 61 - protein, 19 aa, Artificial Sequence
  Description of Artificial Sequence: Synthetic peptide
  N-term Hy
  C-term NH2
DTHFPICIFGPASKGWVCM

SEQ ID NO: 62 - protein, 21 aa, Artificial Sequence
  Description of Artificial Sequence: Synthetic peptide
  N-term Hy
  C-term NH2
DTHFPICIFVCHRSKGCYARC

SEQ ID NO: 63 - protein, 19 aa, Artificial Sequence
  Description of Artificial Sequence: Synthetic peptide
  N-term Ac
  C-term NH2
DTHFPICIFGPRSKGWVCM

SEQ ID NO: 64 - protein, 19 aa, Artificial Sequence
  Description of Artificial Sequence: Synthetic peptide
  N-term Hy
  C-term NH2
DTHFPICIFGPRSAGWVCM

SEQ ID NO: 65 - protein, 19 aa, Artificial Sequence
  Description of Artificial Sequence: Synthetic peptide
  N-term Hy
  C-term NH2
DTHAPICIFGPRSKGWVCM

SEQ ID NO: 66 - protein, 21 aa, Artificial Sequence
  Description of Artificial Sequence: Synthetic peptide
  N-term Hy
  C-term NH2
DTHFPICIFVCHRSKGCYRRC

SEQ ID NO: 67 - protein, 22 aa, Artificial Sequence
  Description of Artificial Sequence: Synthetic peptide
  MOD_RES (1)..(1): pGlu
  C-term NH2
ETHFPICIFVCHRSKGCYRRCR

SEQ ID NO: 68 - protein, 19 aa, Artificial Sequence
  Description of Artificial Sequence: Synthetic peptide
  N-term Ac
  C-term NH2
DTHFPICIFKPRSKGWVCM

SEQ ID NO: 69 - protein, 24 aa, Artificial Sequence
  Description of Artificial Sequence: Synthetic peptide
  N-term Hy
  C-term NH2
DTHFPICIFVCGHRSKGCYMRCKT

SEQ ID NO: 70 - protein, 19 aa, Artificial Sequence
  Description of Artificial Sequence: Synthetic peptide
  N-term Hy
  C-term NH2
DAHFPICIFGPRSKGWVCM

SEQ ID NO: 71 - protein, 21 aa, Artificial Sequence
  Description of Artificial Sequence: Synthetic peptide
  N-term Hy
  C-term NH2
DTHFPICIFVCYRGICYRRCR

SEQ ID NO: 72 - protein, 19 aa, Artificial Sequence
  Description of Artificial Sequence: Synthetic peptide
  N-term Ac
  C-term NH2
DTHFPICIFGPRSKGWVCM

SEQ ID NO: 73 - protein, 18 aa, Artificial Sequence
  Description of Artificial Sequence: Synthetic peptide
  N-term Hy
  MOD_RES (1)..(1): bhAsp
  C-term NH2
DTHFPICIFGPRSKGWVC

SEQ ID NO: 74 - protein, 19 aa, Artificial Sequence
  Description of Artificial Sequence: Synthetic peptide
  N-term Hy
  C-term NH2
DTHFPICIFGPRSKGWACM

SEQ ID NO: 75 - protein, 19 aa, Artificial Sequence
  Description of Artificial Sequence: Synthetic peptide
  MOD_RES (1)..(1): Ida
  MOD_RES (4)..(4): Dpa
  MOD_RES (5)..(5): bhPro
  MOD_RES (9)..(9): bhPhe
  C-term NH2
XTHXPRCRFGPRSKGWVCM

SEQ ID NO: 76 - protein, 9 aa, Artificial Sequence
  Description of Artificial Sequence: Synthetic peptide
  N-term Hy
  C-term NH2
DTHFPCIRF

SEQ ID NO: 77 - protein, 18 aa, Artificial Sequence
  Description of Artificial Sequence: Synthetic peptide
  N-term Isovaleric acid
  C-term NH2
THFPCIIFGPRSKGWVCM

SEQ ID NO: 78 - protein, 9 aa, Artificial Sequence
  Description of Artificial Sequence: Synthetic peptide
  N-term Hy
  C-term NH2
DTHFPCIAF

SEQ ID NO: 79 - protein, 9 aa, Artificial Sequence
  Description of Artificial Sequence: Synthetic peptide
  N-term Hy
  C-term NH2
DAHFPCIIF

SEQ ID NO: 80 - protein, 22 aa, Artificial Sequence
  Description of Artificial Sequence: Synthetic peptide
  N-term Hy
  C-term NH2
DTHFPICIFVCHRPKGCYRRCP

SEQ ID NO: 81 - protein, 19 aa, Artificial Sequence
  Description of Artificial Sequence: Synthetic peptide
  N-term Ac
  MOD_RES (14)..(14): Lys(m-PEG8)
  C-term NH2
DTHFPICIFKPRSKGWVCM

SEQ ID NO: 82 - protein, 10 aa, Artificial Sequence
  Description of Artificial Sequence: Synthetic peptide
  N-term Hy
  C-term NH2
DTHFPCIIFK

SEQ ID NO: 83 - protein, 9 aa, Artificial Sequence
  Description of Artificial Sequence: Synthetic peptide
  N-term Hy
  C-term NH2
DTHFPCIFF

SEQ ID NO: 84 - protein, 19 aa, Artificial Sequence
  Description of Artificial Sequence: Synthetic peptide
  N-term Hy
  MOD_RES (15)..(15): Lys(m-PEG8)
  C-term NH2
DTHFPICIFGPRSKKWVCM

SEQ ID NO: 85 - protein, 19 aa, Artificial Sequence
  Description of Artificial Sequence: Synthetic peptide
  N-term Ac
  C-term NH2
DTHFPICIFGPRSKKWVCM

SEQ ID NO: 86 - protein, 17 aa, Artificial Sequence
  Description of Artificial Sequence: Synthetic peptide
  N-term Hy
  C-term NH2
DTHFPICIFCPWGMCCK

SEQ ID NO: 87 - protein, 19 aa, Artificial Sequence
  Description of Artificial Sequence: Synthetic peptide
  N-term Hy
  C-term NH2
DTAFPICIFGPRSKGWVCM

SEQ ID NO: 88 - protein, 22 aa, Artificial Sequence
  Description of Artificial Sequence: Synthetic peptide
  N-term Hy
  C-term NH2
DTHFPICIFVCYRGICYMRCKT

SEQ ID NO: 89 - protein, 19 aa, Artificial Sequence
  Description of Artificial Sequence: Synthetic peptide
  N-term Hy
  C-term NH2
DTHFPICIFGPRSKGAVCM

SEQ ID NO: 90 - protein, 19 aa, Artificial Sequence
  Description of Artificial Sequence: Synthetic peptide
  N-term Hy
  C-term NH2
DTHFPICIAGPRSKGWVCM

SEQ ID NO: 91 - protein, 19 aa, Artificial Sequence
  Description of Artificial Sequence: Synthetic peptide
  N-term Hy
  C-term NH2
DTHFPICAFGPRSKGWVCM

SEQ ID NO: 92 - protein, 19 aa, Artificial Sequence
  Description of Artificial Sequence: Synthetic peptide
  N-term Hy
  C-term NH2
DTHFPIAIFGPRSKGWVAM

SEQ ID NO: 93 - protein, 18 aa, Artificial Sequence
  Description of Artificial Sequence: Synthetic peptide
  N-term Hy
  C-term NH2
DTHFPCRRFGPRSKGWVC

SEQ ID NO: 94 - protein, 18 aa, Artificial Sequence
  Description of Artificial Sequence: Synthetic peptide
  MOD_RES (1)..(1): Ida
  MOD_RES (5)..(5): bhPro
  MOD_RES (9)..(9): bhPhe
  C-term NH2
XTHFPCRRFGPRSKGWVC

SEQ ID NO: 95 - skipped

SEQ ID NO: 96 - protein, 22 aa, Artificial Sequence
  Description of Artificial Sequence: Synthetic peptide
  N-term Hy
  C-term NH2
DTHFPCIIFVCHRSKGCYWAVC

SEQ ID NO: 97 - protein, 23 aa, Artificial Sequence
  Description of Artificial Sequence: Synthetic peptide
  N-term Hy
  MOD_RES (6)..(6): D-Cys
  MOD_RES (22)..(22): D-Cys
  C-term NH2
DTHFPCIIFVCHRSKGCYWAVCF

SEQ ID NO: 98 - protein, 24 aa, Artificial Sequence
  Description of Artificial Sequence: Synthetic peptide
  N-term Hy
  C-term NH2
DTHFPCIIFVCHRSKGCYWAVCFW

SEQ ID NO: 99 - protein, 19 aa, Artificial Sequence
  Description of Artificial Sequence: Synthetic peptide
  N-term Ac
  MOD_RES (10)..(10): Lys(m-PEG8)
  C-term NH2
DTHFPICIFKPRSKGWVCM

SEQ ID NO: 100 - skipped

SEQ ID NO: 101 - protein, 19 aa, Artificial Sequence
  Description of Artificial Sequence: Synthetic peptide
  N-term Hy
  MOD_RES (4)..(4): Dpa
  C-term NH2
DTHXPCIIFGPRSRGWVCK

SEQ ID NO: 102 - protein, 19 aa, Artificial Sequence
  Description of Artificial Sequence: Synthetic peptide
  N-term Hy
  MOD_RES (5)..(5): bhPro
  C-term NH2
DTHFPCIIFGPRSRGWVCK

SEQ ID NO: 103 - protein, 19 aa, Artificial Sequence
  Description of Artificial Sequence: Synthetic peptide
  N-term Hy
  C-term NH2
DTHFPCIIFGPRSRGWRCK

SEQ ID NO: 104 - protein, 19 aa, Artificial Sequence
  Description of Artificial Sequence: Synthetic peptide
  N-term Hy
  C-term NH2
DTHFPCIRFGPRSRGWVCK

SEQ ID NO: 105 - protein, 19 aa, Artificial Sequence
  Description of Artificial Sequence: Synthetic peptide
  N-term Hy
  C-term NH2
DTHFPCIRFGPRSRGWRCK

SEQ ID NO: 106 - protein, 19 aa, Artificial Sequence
  Description of Artificial Sequence: Synthetic peptide
  N-term Hy
  C-term NH2
DTHFPCIIFGPRSRGWVCK

SEQ ID NO: 107 - protein, 18 aa, Artificial Sequence
  Description of Artificial Sequence: Synthetic peptide
  N-term Hy
  C-term NH2
DTHFPCIIFGPRSRGVCK

SEQ ID NO: 108 - protein, 19 aa, Artificial Sequence
  Description of Artificial Sequence: Synthetic peptide
  N-term Hy
  C-term NH2
DTHFPCIYFGPRSKGWVCK

SEQ ID NO: 109 - protein, 19 aa, Artificial Sequence
  Description of Artificial Sequence: Synthetic peptide
  N-term Hy
  C-term NH2
DTHFPCIIFGPRSKGWVCK

SEQ ID NO: 110 - protein, 19 aa, Artificial Sequence
  Description of Artificial Sequence: Synthetic peptide
  N-term Hy
  C-term NH2
DTHFPCIIFGPRARGWVCK

SEQ ID NO: 111 - protein, 19 aa, Artificial Sequence
  Description of Artificial Sequence: Synthetic peptide
  N-term Octanoic acid
  C-term NH2
DTHFPCIIFGPRSRGWVCK

SEQ ID NO: 112 - protein, 19 aa, Artificial Sequence
  Description of Artificial Sequence: Synthetic peptide
  N-term Palm-PEG11
  C-term NH2
DTHFPCIIFGPRSRGWVCK

SEQ ID NO: 113 - protein, 19 aa, Artificial Sequence
  Description of Artificial Sequence: Synthetic peptide
  N-term Ac
  MOD_RES (10)..(10): Lys(2K-PEG)
  C-term NH2
DTHFPICIFKPRSKGWVCK

SEQ ID NO: 114 - protein, 19 aa, Artificial Sequence
  Description of Artificial Sequence: Synthetic peptide
  N-term Hy
  C-term NH2
DTHFPCIIFGPRSKGWKCK

SEQ ID NO: 115 - protein, 19 aa, Artificial Sequence
  Description of Artificial Sequence: Synthetic peptide
  N-term Hy
  C-term NH2
DTHFPCIKFGPRSKGWKCK

SEQ ID NO: 116 - protein, 19 aa, Artificial Sequence
  Description of Artificial Sequence: Synthetic peptide
  N-term Isovaleric acid
  C-term NH2
DTHFPCLIFGPRSKGWVCK

SEQ ID NO: 117 - protein, 19 aa, Artificial Sequence
  Description of Artificial Sequence: Synthetic peptide
  N-term Isovaleric acid
  C-term NH2
DTHFPCVIFGPRSKGWVCK

SEQ ID NO: 118 - protein, 19 aa, Artificial Sequence
  Description of Artificial Sequence: Synthetic peptide
  N-term Isovaleric acid
  C-term NH2
DTHFPCSIFGPRSKGWVCK

SEQ ID NO: 119 - protein, 19 aa, Artificial Sequence
  Description of Artificial Sequence: Synthetic peptide
  N-term Isovaleric acid
  C-term NH2
DTHFPCQIFGPRSKGWVCK

SEQ ID NO: 120 - protein, 18 aa, Artificial Sequence
  Description of Artificial Sequence: Synthetic peptide
  N-term Hy
  C-term NH2
THFPCIIFGPRSKGWVCK

SEQ ID NO: 121 - protein, 18 aa, Artificial Sequence
  Description of Artificial Sequence: Synthetic peptide
  N-term Isovaleric acid
  C-term NH2
THFPCIIFGPRSKGWVCK

SEQ ID NO: 122 - protein, 17 aa, Artificial Sequence
  Description of Artificial Sequence: Synthetic peptide
  N-term Hy
  C-term NH2
HFPCIIFGPRSKGWVCK

SEQ ID NO: 123 - protein, 17 aa, Artificial Sequence
  Description of Artificial Sequence: Synthetic peptide
  N-term Isovaleric acid
  C-term NH2
HFPCIIFGPRSKGWVCK

SEQ ID NO: 124 - protein, 19 aa, Artificial Sequence
  Description of Artificial Sequence: Synthetic peptide
  N-term Hy
  C-term NH2
DTHFPCISFGPRSKGWVCK

SEQ ID NO: 125 - protein, 19 aa, Artificial Sequence
  Description of Artificial Sequence: Synthetic peptide
  N-term Hy
  C-term NH2
DTHFPCIKFGPRSKGWVCK

SEQ ID NO: 126 - protein, 20 aa, Artificial Sequence
  Description of Artificial Sequence: Synthetic peptide
  N-term Hy
  C-term NH2
EDTHFPCIIFGPRSKGWVCK

SEQ ID NO: 127 - skipped

SEQ ID NO: 128 - protein, 19 aa, Artificial Sequence
  Description of Artificial Sequence: Synthetic peptide
  N-term Isovaleric acid
  C-term NH2
DTHFPCIIFEPRSKGWVCK

SEQ ID NO: 129 - protein, 19 aa, Artificial Sequence
  Description of Artificial Sequence: Synthetic peptide
  N-term Isovaleric acid
  C-term NH2
DTHFPCIIFSPRSKGWVCK

SEQ ID NO: 130 - protein, 19 aa, Artificial Sequence
  Description of Artificial Sequence: Synthetic peptide
  N-term Isovaleric acid
  C-term NH2
DTHFSCIIFGPRSKGWVCK

SEQ ID NO: 131 - protein, 19 aa, Artificial Sequence
  Description of Artificial Sequence: Synthetic peptide
  N-term Octanoic acid-PEG11
  C-term NH2
DTHFPCIIFGPRSRGWVCK

SEQ ID NO: 132 - protein, 19 aa, Artificial Sequence
  Description of Artificial Sequence: Synthetic peptide
  N-term Isobutyric acid-PEG11
  C-term NH2
DTHFPCIIFGPRSRGWVCK

SEQ ID NO: 133 - protein, 19 aa, Artificial Sequence
  Description of Artificial Sequence: Synthetic peptide
  MOD_RES (1)..(1): Ida
  C-term NH2
XTHFPCIIFGPRSRGWVCK

SEQ ID NO: 134 - protein, 19 aa, Artificial Sequence
  Description of Artificial Sequence: Synthetic peptide
  N-term Isovaleric acid
  C-term NH2
DTHFPCIIFGPKSKGWVCK

SEQ ID NO: 135 - protein, 19 aa, Artificial Sequence
  Description of Artificial Sequence: Synthetic peptide
  N-term Isovaleric acid
  C-term NH2
DTHFPCIKFGPKSKGWVCK

SEQ ID NO: 136 - protein, 18 aa, Artificial Sequence
  Description of Artificial Sequence: Synthetic peptide
  N-term Isovaleric acid
  C-term NH2
DTHFPCIIFGPRSKGWCK

SEQ ID NO: 137 - protein, 17 aa, Artificial Sequence
  Description of Artificial Sequence: Synthetic peptide
  N-term Isovaleric acid
  C-term NH2
DTHFPCIIFGPRSKGVC

SEQ ID NO: 138 - protein, 17 aa, Artificial Sequence
  Description of Artificial Sequence: Synthetic peptide
  N-term Isovaleric acid
  C-term NH2
DTHFPCIIFGPRSKGCK

SEQ ID NO: 139 - skipped

SEQ ID NO: 140 - protein, 19 aa, Artificial Sequence
  Description of Artificial Sequence: Synthetic peptide
  N-term Isovaleric acid
  MOD_RES (7)..(7): Dapa
  C-term NH2
DTHFPCXIFGPRSKGWDCK

SEQ ID NO: 141 - protein, 19 aa, Artificial Sequence
  Description of Artificial Sequence: Synthetic peptide
  N-term Isovaleric acid
  MOD_RES (8)..(8): Dapa
  C-term NH2
DTHFPCIXFGPRSKGWDCK

SEQ ID NO: 142 - protein, 19 aa, Artificial Sequence
  Description of Artificial Sequence: Synthetic peptide
  N-term Isovaleric acid
  MOD_RES (7)..(7): Dapa
  C-term NH2
DTHFPCXIFGPRSKGWECK

SEQ ID NO: 143 - protein, 19 aa, Artificial Sequence
  Description of Artificial Sequence: Synthetic peptide
  N-term Isovaleric acid
  MOD_RES (8)..(8): Dapa
  C-term NH2
DTHFPCIXFGPRSKGWECK

SEQ ID NO: 144 - protein, 19 aa, Artificial Sequence
  Description of Artificial Sequence: Synthetic peptide
  N-term Isovaleric acid
  C-term NH2
DTHFPCIKFGPRSKGWECK

SEQ ID NO: 145 - protein, 19 aa, Artificial Sequence
  Description of Artificial Sequence: Synthetic peptide
  N-term Isovaleric acid
  C-term NH2
DTHFGCIIFGPRSKGWVCK

SEQ ID NO: 146 - protein, 19 aa, Artificial Sequence
  Description of Artificial Sequence: Synthetic peptide
  N-term Hy
  C-term NH2
DTHFGCIIFGPRSKGWVCK

SEQ ID NO: 147 - protein, 19 aa, Artificial Sequence
  Description of Artificial Sequence: Synthetic peptide
  N-term Isovaleric acid
  C-term NH2
DTHFRCIIFGPRSKGWVCK

SEQ ID NO: 148 - protein, 19 aa, Artificial Sequence
  Description of Artificial Sequence: Synthetic peptide
  N-term Hy
  C-term NH2
DTHFRCIIFGPRSKGWVCK

SEQ ID NO: 149 - protein, 19 aa, Artificial Sequence
  Description of Artificial Sequence: Synthetic peptide
  N-term Isovaleric acid
  MOD_RES (5)..(5): Sarc
  C-term NH2
DTHFXCIIFGPRSKGWVCK

SEQ ID NO: 150 - protein, 19 aa, Artificial Sequence
  Description of Artificial Sequence: Synthetic peptide
  N-term Hy
  MOD_RES (5)..(5): Sarc
  C-term NH2
DTHFXCIIFGPRSKGWVCK

SEQ ID NO: 151 - protein, 19 aa, Artificial Sequence
  Description of Artificial Sequence: Synthetic peptide
  N-term Isovaleric acid
  MOD_RES (5)..(5): Beta-Ala
  C-term NH2
DTHFACIIFGPRSKGWVCK

SEQ ID NO: 152 - protein, 19 aa, Artificial Sequence
  Description of Artificial Sequence: Synthetic peptide
  N-term Hy
  MOD_RES (5)..(5): Beta-Ala
  C-term NH2
DTHFACIIFGPRSKGWVCK

SEQ ID NO: 153 - protein, 19 aa, Artificial Sequence
  Description of Artificial Sequence: Synthetic peptide
  N-term Isovaleric acid
  C-term NH2
DTHFKCIIFGPRSKGWVCK

SEQ ID NO: 154 - protein, 19 aa, Artificial Sequence
  Description of Artificial Sequence: Synthetic peptide
  N-term Hy
  C-term NH2
DTHFKCIIFGPRSKGWVCK

SEQ ID NO: 155 - protein, 18 aa, Artificial Sequence
  Description of Artificial Sequence: Synthetic peptide
  N-term Hy
  C-term NH2
THFPCIIFGPRSKGWVCM

SEQ ID NO: 156 - protein, 17 aa, Artificial Sequence
  Description of Artificial Sequence: Synthetic peptide
  N-term Hy
  C-term NH2
HFPCIIFGPRSKGWVCM

SEQ ID NO: 157 - protein, 17 aa, Artificial Sequence
  Description of Artificial Sequence: Synthetic peptide
  N-term Isovaleric acid
  C-term NH2
HFPCIIFGPRSKGWVCM

SEQ ID NO: 158 - protein, 19 aa, Artificial Sequence
  Description of Artificial Sequence: Synthetic peptide
  N-term Hy
  C-term NH2
DTHFPCISFGPRSKGWVCM

SEQ ID NO: 159 - protein, 19 aa, Artificial Sequence
  Description of Artificial Sequence: Synthetic peptide
  N-term Hy
  C-term NH2
DTHFPCIKFGPRSKGWVCM

SEQ ID NO: 160 - protein, 20 aa, Artificial Sequence
  Description of Artificial Sequence: Synthetic peptide
  N-term Hy
  C-term NH2
EDTHFPCIIFGPRSKGWVCM

SEQ ID NO: 161 - skipped

SEQ ID NO: 162 - protein, 19 aa, Artificial Sequence
  Description of Artificial Sequence: Synthetic peptide
  N-term Hy
  C-term NH2
DTHFPCIIFEPRSKGWVCM

SEQ ID NO: 163 - protein, 19 aa, Artificial Sequence
  Description of Artificial Sequence: Synthetic peptide
  N-term Isovaleric acid
  C-term NH2
DTHFKCIEFGPRSKGWVCK

SEQ ID NO: 164 - protein, 19 aa, Artificial Sequence
  Description of Artificial Sequence: Synthetic peptide
  N-term Isovaleric acid
  C-term NH2
DTHFPCIIFGPRSKGWACK

SEQ ID NO: 165 - protein, 19 aa, Artificial Sequence
  Description of Artificial Sequence: Synthetic peptide
  N-term Isovaleric acid
  C-term NH2
DTHFPCIIFEPRSKGWVCK

SEQ ID NO: 166 - protein, 22 aa, Artificial Sequence
  Description of Artificial Sequence: Synthetic peptide
  N-term Isovaleric acid
  C-term NH2
DTHFPCIIFGPRSKGWVCKKKK

SEQ ID NO: 167 - protein, 22 aa, Artificial Sequence
  Description of Artificial Sequence: Synthetic peptide
  N-term Isovaleric acid
  C-term NH2
DTHFPCIIFEPRSKGWVCKKKK

SEQ ID NO: 168 - protein, 20 aa, Artificial Sequence
  Description of Artificial Sequence: Synthetic peptide
  N-term Isovaleric acid
  C-term NH2
DTHFPCIIFGPRSKGWVCKK

SEQ ID NO: 169 - protein, 19 aa, Artificial Sequence
  Description of Artificial Sequence: Synthetic peptide
  N-term Isovaleric acid
  C-term NH2
DTAFPCIIFGPRSKGWVCK

SEQ ID NO: 170 - protein, 19 aa, Artificial Sequence
  Description of Artificial Sequence: Synthetic peptide
  N-term Isovaleric acid
  C-term NH2
DTKFPCIIFGPRSKGWVCK

SEQ ID NO: 171 - protein, 23 aa, Artificial Sequence
  Description of Artificial Sequence: Synthetic peptide
  N-term Isovaleric acid
  C-term NH2
DTHFPCIIFVCHRPKGCYRRVCR

SEQ ID NO: 172 - protein, 19 aa, Artificial Sequence
  Description of Artificial Sequence: Synthetic peptide
  N-term Isovaleric acid
  MOD_RES (8)..(8): Lys(m-PEG8)
  C-term NH2
DTHFPCIKFGPRSKGWVCK

SEQ ID NO: 173 - protein, 19 aa, Artificial Sequence
  Description of Artificial Sequence: Synthetic peptide
  N-term Isovaleric acid
  MOD_RES (10)..(10): Lys(m-PEG8)
  C-term NH2
DTHFPCIKFKPRSKGWVCK

SEQ ID NO: 174 - protein, 19 aa, Artificial Sequence
  Description of Artificial Sequence: Synthetic peptide
  N-term Isovaleric acid
  MOD_RES (12)..(12): Lys(m-PEG8)
  C-term NH2
DTHFPCIKFGPKSKGWVCK

SEQ ID NO: 175 - protein, 19 aa, Artificial Sequence
  Description of Artificial Sequence: Synthetic peptide
  N-term Isovaleric acid
  MOD_RES (14)..(14): Lys(m-PEG8)
  C-term NH2
DTHFPCIKFGPRSKGWVCK

SEQ ID NO: 176 - protein, 19 aa, Artificial Sequence
  Description of Artificial Sequence: Synthetic peptide
  N-term Isovaleric acid
  MOD_RES (19)..(19): Lys(m-PEG8)
  C-term NH2
DTHFPCIKFGPRSKGWVCK

SEQ ID NO: 177 - protein, 19 aa, Artificial Sequence
  Description of Artificial Sequence: Synthetic peptide
  N-term Isovaleric acid
  C-term NH2
DTHFPCIKFGPRSKGWTCK

SEQ ID NO: 178 - protein, 19 aa, Artificial Sequence
  Description of Artificial Sequence: Synthetic peptide
  N-term Isovaleric acid
  C-term NH2
DTHFPCIEFGPRSKGWTCK

SEQ ID NO: 179 - protein, 18 aa, Artificial Sequence
  Description of Artificial Sequence: Synthetic peptide
  N-term Isovaleric acid
  MOD_RES (14)..(14): Lys(Betaine)
  C-term NH2
DTHFPICIFGPRSKGWVC

SEQ ID NO: 180 - protein, 19 aa, Artificial Sequence
  Description of Artificial Sequence: Synthetic peptide
  N-term Isovaleric acid
  MOD_RES (14)..(14): Lys(Betaine)
  C-term NH2
DTHFPCIKFGPRSKGWVCK

SEQ ID NO: 181 - protein, 19 aa, Artificial Sequence
  Description of Artificial Sequence: Synthetic peptide
  N-term Isovaleric acid
  MOD_RES (8)..(8): Lys(Betaine)
  C-term NH2
DTHFPCIKFGPRSKGWVCK

SEQ ID NO: 182 - protein, 19 aa, Artificial Sequence
  Description of Artificial Sequence: Synthetic peptide
  N-term Isovaleric acid
  MOD_RES (19)..(19): Lys(Betaine)
  C-term NH2
DTHFPCIKFGPRSKGWVCK

SEQ ID NO: 183 - protein, 19 aa, Artificial Sequence
  Description of Artificial Sequence: Synthetic peptide
  N-term Ac
  C-term NH2
DTHFPCIKFGPRSKGWVCK

SEQ ID NO: 184 - protein, 19 aa, Artificial Sequence
  Description of Artificial Sequence: Synthetic peptide
  N-term Isovaleric acid-PEG3
  C-term NH2
DTHFPCIKFGPRSKGWVCK

SEQ ID NO: 185 - protein, 19 aa, Artificial Sequence
  Description of Artificial Sequence: Synthetic peptide
  N-term Isobutyric acid
  C-term NH2
DTHFPCIKFGPRSKGWVCK

SEQ ID NO: 186 - protein, 19 aa, Artificial Sequence
  Description of Artificial Sequence: Synthetic peptide
  N-term Valeric acid
  C-term NH2
DTHFPCIKFGPRSKGWVCK

SEQ ID NO: 187 - protein, 20 aa, Artificial Sequence
  Description of Artificial Sequence: Synthetic peptide
  N-term Hy
  C-term NH2
VDTHFPCIKFGPRSKGWVCK

SEQ ID NO: 188 - protein, 20 aa, Artificial Sequence
  Description of Artificial Sequence: Synthetic peptide
  N-term Hy
  C-term NH2
LDTHFPCIKFGPRSKGWVCK

SEQ ID NO: 189 - protein, 19 aa, Artificial Sequence
  Description of Artificial Sequence: Synthetic peptide
  N-term Hexanoic acid
  C-term NH2
DTHFPCIKFGPRSKGWVCK

SEQ ID NO: 190 - protein, 19 aa, Artificial Sequence
  Description of Artificial Sequence: Synthetic peptide
  N-term 5-Methylpentanoic
  C-term NH2
DTHFPCIKFGPRSKGWVCK

SEQ ID NO: 191 - protein, 19 aa, Artificial Sequence
  Description of Artificial Sequence: Synthetic peptide
  N-term Cyclohexanoic acid
  C-term NH2
DTHFPCIKFGPRSKGWVCK

SEQ ID NO: 192 - protein, 19 aa, Artificial Sequence
  Description of Artificial Sequence: Synthetic peptide
  N-term Heptanoic acid
  C-term NH2
DTHFPCIKFGPRSKGWVCK

SEQ ID NO: 193 - protein, 19 aa, Artificial Sequence
  Description of Artificial Sequence: Synthetic peptide
  N-term Octanoic acid
  C-term NH2
DTHFPCIKFGPRSKGWVCK

SEQ ID NO: 194 - protein, 19 aa, Artificial Sequence
  Description of Artificial Sequence: Synthetic peptide
  N-term Isovaleric acid
  C-term NH2
DTHFPCIIFGPRSKGWKCK

SEQ ID NO: 195 - protein, 19 aa, Artificial Sequence
  Description of Artificial Sequence: Synthetic peptide
  N-term Isovaleric acid
  C-term NH2
DTHFPCIIFGPRSKGWECK

SEQ ID NO: 196 - protein, 19 aa, Artificial Sequence
  Description of Artificial Sequence: Synthetic peptide
  N-term Isovaleric acid
  C-term NH2
DTHFPCRRFGPRSKGWVCK

SEQ ID NO: 197-198 - skipped (2 entries)

SEQ ID NO: 199 - protein, 18 aa, Artificial Sequence
  Description of Artificial Sequence: Synthetic peptide
  N-term Isovaleric acid
  MOD_RES (14)..(14): Lys(m-PEG8)
  C-term NH2
DTHFPICIFGPRSKGWVC

SEQ ID NO: 200 - protein, 18 aa, Artificial Sequence
  Description of Artificial Sequence: Synthetic peptide
  N-term Isovaleric acid
  MOD_RES (14)..(14): Lys(m-PEG4)
  C-term NH2
DTHFPICIFGPRSKGWVC

SEQ ID NO: 201 - protein, 19 aa, Artificial Sequence
  Description of Artificial Sequence: Synthetic peptide
  N-term Isovaleric acid
  MOD_RES (19)..(19): Lys(m-PEG8)
  C-term NH2
DTHFPCIIFGPRSRGWVCK

SEQ ID NO: 202 - protein, 19 aa, Artificial Sequence
  Description of Artificial Sequence: Synthetic peptide
  N-term Isovaleric acid
  MOD_RES (19)..(19): Lys(m-PEG4)
  C-term NH2
DTHFPCIIFGPRSRGWVCK

SEQ ID NO: 203 - protein, 19 aa, Artificial Sequence
  Description of Artificial Sequence: Synthetic peptide
  N-term Isovaleric acid
  MOD_RES (19)..(19): Lys(PEG2)
  C-term NH2
DTHFPCIIFGPRSRGWVCK

SEQ ID NO: 204 - protein, 19 aa, Artificial Sequence
  Description of Artificial Sequence: Synthetic peptide
  N-term Isovaleric acid
  C-term NH2
DTHFPCIKFEPRSKGWVCK

SEQ ID NO: 205 - protein, 19 aa, Artificial Sequence
  Description of Artificial Sequence: Synthetic peptide
  N-term Isovaleric acid
  C-term NH2
DTHFPCIKFEPRSKGWTCK

SEQ ID NO: 206 - protein, 18 aa, Artificial Sequence
  Description of Artificial Sequence: Synthetic peptide
  N-term Isovaleric acid
  C-term NH2
DTHFPCIKFEPRSKGWCK

SEQ ID NO: 207 - protein, 17 aa, Artificial Sequence
  Description of Artificial Sequence: Synthetic peptide
  N-term Isovaleric acid
  C-term NH2
DTHFPCIKFEPRSKGCK

SEQ ID NO: 208 - protein, 16 aa, Artificial Sequence
  Description of Artificial Sequence: Synthetic peptide
  N-term Isovaleric acid
  C-term NH2
DTHFPCIFEPRSKGCK

SEQ ID NO: 209 - protein, 17 aa, Artificial Sequence
  Description of Artificial Sequence: Synthetic peptide
  N-term Isovaleric acid
  C-term NH2
DTHFPCIFEPRSKGWCK

SEQ ID NO: 210 - protein, 16 aa, Artificial Sequence
  Description of Artificial Sequence: Synthetic peptide
  N-term Isovaleric acid
  C-term NH2
DTHFPCIKFGPRSKCK

SEQ ID NO: 211 - protein, 15 aa, Artificial Sequence
  Description of Artificial Sequence: Synthetic peptide
  N-term Isovaleric acid
  C-term NH2
DTHFPCIKFGPRSCK

SEQ ID NO: 212 - protein, 14 aa, Artificial Sequence
  Description of Artificial Sequence: Synthetic peptide
  N-term Isovaleric acid
  C-term NH2
DTHFPCIKFGPRCK

SEQ ID NO: 213 - protein, 13 aa, Artificial Sequence
  Description of Artificial Sequence: Synthetic peptide
  N-term Isovaleric acid
  C-term NH2
DTHFPCIKFGPCK

SEQ ID NO: 214 - protein, 12 aa, Artificial Sequence
  Description of Artificial Sequence: Synthetic peptide
  N-term Isovaleric acid
  C-term NH2
DTHFPCIKFGCK

SEQ ID NO: 215 - protein, 11 aa, Artificial Sequence
  Description of Artificial Sequence: Synthetic peptide
  N-term Isovaleric acid
  C-term NH2
DTHFPCIKFCK

SEQ ID NO: 216 - protein, 10 aa, Artificial Sequence
  Description of Artificial Sequence: Synthetic peptide
  N-term Isovaleric acid
  C-term NH2
DTHFPCIKFC

SEQ ID NO: 217 - protein, 19 aa, Artificial Sequence
  Description of Artificial Sequence: Synthetic peptide
  N-term Isovaleric acid
  MOD_RES (8)..(8): Lys(Palm)
  C-term NH2
DTHFPCIKFGPRSKGWVCK

SEQ ID NO: 218 - protein, 19 aa, Artificial Sequence
  Description of Artificial Sequence: Synthetic peptide
  N-term Isovaleric acid
  MOD_RES (10)..(10): Lys(Palm)
  C-term NH2
DTHFPCIKFKPRSKGWVCK

SEQ ID NO: 219 - protein, 19 aa, Artificial Sequence
  Description of Artificial Sequence: Synthetic peptide
  N-term Isovaleric acid
  MOD_RES (12)..(12): Lys(m-PEG8)
  C-term NH2
DTHFPCIKFGPKSKGWVCK

SEQ ID NO: 220 - protein, 19 aa, Artificial Sequence
  Description of Artificial Sequence: Synthetic peptide
  N-term Isovaleric acid
  MOD_RES (14)..(14): Lys(Palm)
  C-term NH2
DTHFPCIKFGPRSKGWVCK

SEQ ID NO: 221 - protein, 19 aa, Artificial Sequence
  Description of Artificial Sequence: Synthetic peptide
  N-term Isovaleric acid
  MOD_RES (19)..(19): Lys(Palm)
  C-term NH2
DTHFPCIKFGPRSKGWVCK

SEQ ID NO: 222 - protein, 19 aa, Artificial Sequence
  Description of Artificial Sequence: Synthetic peptide
  N-term Isovaleric acid
  MOD_RES (8)..(8): Lys(PEG3-Palm)
  C-term NH2
DTHFPCIKFGPRSKGWVCK

SEQ ID NO: 223 - protein, 19 aa, Artificial Sequence
  Description of Artificial Sequence: Synthetic peptide
  N-term Isovaleric acid
  MOD_RES (10)..(10): Lys(PEG3-Palm)
  C-term NH2
DTHFPCIKFKPRSKGWVCK

SEQ ID NO: 224 - protein, 19 aa, Artificial Sequence
  Description of Artificial Sequence: Synthetic peptide
  N-term Isovaleric acid
  MOD_RES (12)..(12): Lys(PEG3-Palm)
  C-term NH2
DTHFPCIKFGPKSKGWVCK

SEQ ID NO: 225 - protein, 19 aa, Artificial Sequence
  Description of Artificial Sequence: Synthetic peptide
  N-term Isovaleric acid
  MOD_RES (14)..(14): Lys(PEG3-Palm)
  C-term NH2
DTHFPCIKFGPRSKGWVCK

SEQ ID NO: 226 - protein, 19 aa, Artificial Sequence
  Description of Artificial Sequence: Synthetic peptide
  N-term Isovaleric acid
  MOD_RES (19)..(19): Lys(PEG3-Palm)
  C-term NH2
DTHFPCIKFGPRSKGWVCK

SEQ ID NO: 227 - protein, 19 aa, Artificial Sequence
  Description of Artificial Sequence: Synthetic peptide
  N-term Hy
  MOD_RES (8)..(8): Lys(IVA)
  C-term NH2
DTHFPCIKFGPRSKGWVCK

SEQ ID NO: 228 - protein, 19 aa, Artificial Sequence
  Description of Artificial Sequence: Synthetic peptide
  N-term Hy
  MOD_RES (10)..(10): Lys(IVA)
  C-term NH2
DTHFPCIKFKPRSKGWVCK

SEQ ID NO: 229 - protein, 19 aa, Artificial Sequence
  Description of Artificial Sequence: Synthetic peptide
  N-term Hy
  MOD_RES (12)..(12): Lys(IVA)
  C-term NH2
DTHFPCIKFGPKSKGWVCK

SEQ ID NO: 230 - protein, 19 aa, Artificial Sequence
  Description of Artificial Sequence: Synthetic peptide
  N-term Hy
  MOD_RES (14)..(14): Lys(IVA)
  C-term NH2
DTHFPCIKFGPRSKGWVCK

SEQ ID NO: 231 - protein, 19 aa, Artificial Sequence
  Description of Artificial Sequence: Synthetic peptide
  N-term Hy
  MOD_RES (19)..(19): Lys(IVA)
  C-term NH2
DTHFPCIKFGPRSKGWVCK

SEQ ID NO: 232 - protein, 19 aa, Artificial Sequence
  Description of Artificial Sequence: Synthetic peptide
  N-term Hy
  MOD_RES (8)..(8): Lys(PEG3-IVA)
  C-term NH2
DTHFPCIKFGPRSKGWVCK

SEQ ID NO: 233 - protein, 19 aa, Artificial Sequence
  Description of Artificial Sequence: Synthetic peptide
  N-term Hy
  MOD_RES (10)..(10): Lys(PEG3-IVA)
  C-term NH2
DTHFPCIKFKPRSKGWVCK

SEQ ID NO: 234 - protein, 19 aa, Artificial Sequence
  Description of Artificial Sequence: Synthetic peptide
  N-term Hy
  MOD_RES (12)..(12): Lys(PEG3-IVA)
  C-term NH2
DTHFPCIKFGPKSKGWVCK

SEQ ID NO: 235 - protein, 19 aa, Artificial Sequence
  Description of Artificial Sequence: Synthetic peptide
  N-term Hy
  MOD_RES (14)..(14): Lys(PEG3-IVA)
  C-term NH2
DTHFPCIKFGPRSKGWVCK

SEQ ID NO: 236 - protein, 19 aa, Artificial Sequence
  Description of Artificial Sequence: Synthetic peptide
  N-term Hy
  MOD_RES (19)..(19): Lys(PEG3-IVA)
  C-term NH2
DTHFPCIKFGPRSKGWVCK

SEQ ID NO: 237 - protein, 19 aa, Artificial Sequence
  Description of Artificial Sequence: Synthetic peptide
  N-term Isovaleric acid
  C-term NH2
DTHFPCIKFEPRSKKWVCK

SEQ ID NO: 238 - protein, 19 aa, Artificial Sequence
  Description of Artificial Sequence: Synthetic peptide
  N-term Hy
  C-term NH2
DTHFPCIKFGPRSKGWVCK

SEQ ID NO: 239 - protein, 19 aa, Artificial Sequence
  Description of Artificial Sequence: Synthetic peptide
  N-term Palm
  C-term NH2
DTHFPCIKFGPRSKGWVCK

SEQ ID NO: 240 - protein, 19 aa, Artificial Sequence
  Description of Artificial Sequence: Synthetic peptide
  N-term Palm-PEG3
  C-term NH2
DTHFPCIKFGPRSKGWVCK

SEQ ID NO: 241 - protein, 17 aa, Artificial Sequence
  Description of Artificial Sequence: Synthetic peptide
  N-term Isovaleric acid
  MOD_RES (8)..(8): Lys(isoGlu-Palm)
  C-term NH2
DTHFPCIKFEPRSKGCK

SEQ ID NO: 242 - protein, 17 aa, Artificial Sequence
  Description of Artificial Sequence: Synthetic peptide
  N-term Isovaleric acid
  MOD_RES (10)..(10): Lys(isoGlu-Palm)
  C-term NH2
DTHFPCIKFKPRSKGCK

SEQ ID NO: 243 - protein, 17 aa, Artificial Sequence
  Description of Artificial Sequence: Synthetic peptide
  N-term Isovaleric acid
  MOD_RES (12)..(12): Lys(isoGlu-Palm)
  C-term NH2
DTHFPCIKFEPKSKGCK

SEQ ID NO: 244 - protein, 17 aa, Artificial Sequence
  Description of Artificial Sequence: Synthetic peptide
  N-term Isovaleric acid
  MOD_RES (14)..(14): Lys(isoGlu-Palm)
  C-term NH2
DTHFPCIKFEPRSKGCK

SEQ ID NO: 245 - protein, 17 aa, Artificial Sequence
  Description of Artificial Sequence: Synthetic peptide
  N-term Isovaleric acid
  MOD_RES (15)..(15): Lys(isoGlu-Palm)
  C-term NH2
DTHFPCIKFEPRSKKCK

SEQ ID NO: 246 - protein, 17 aa, Artificial Sequence
  Description of Artificial Sequence: Synthetic peptide
  N-term Isovaleric acid
  MOD_RES (17)..(17): Lys(isoGlu-Palm)
  C-term NH2
DTHFPCIKFEPRSKGCK

SEQ ID NO: 247 - protein, 18 aa, Artificial Sequence
  Description of Artificial Sequence: Synthetic peptide
  N-term Isovaleric acid
  MOD_RES (18)..(18): Lys(isoGlu-Palm)
  C-term NH2
DTHFPCIKFEPRSKGCKK

SEQ ID NO: 248 - protein, 17 aa, Artificial Sequence
  Description of Artificial Sequence: Synthetic peptide
  N-term Isovaleric acid
  MOD_RES (8)..(8): Lys(Dapa-Palm)
  C-term NH2
DTHFPCIKFEPRSKGCK

SEQ ID NO: 249 - protein, 17 aa, Artificial Sequence
  Description of Artificial Sequence: Synthetic peptide
  N-term Isovaleric acid
  MOD_RES (10)..(10): Lys(Dapa-Palm)
  C-term NH2
DTHFPCIKFKPRSKGCK

SEQ ID NO: 250 - protein, 17 aa, Artificial Sequence
  Description of Artificial Sequence: Synthetic peptide
  N-term Isovaleric acid
  MOD_RES (12)..(12): Lys(Dapa-Palm)
  C-term NH2
DTHFPCIKFEPKSKGCK

SEQ ID NO: 251 - protein, 17 aa, Artificial Sequence
  Description of Artificial Sequence: Synthetic peptide
  N-term Isovaleric acid
  MOD_RES (14)..(14): Lys(Dapa-Palm)
  C-term NH2
DTHFPCIKFEPRSKGCK

SEQ ID NO: 252 - protein, 17 aa, Artificial Sequence
  Description of Artificial Sequence: Synthetic peptide
  N-term Isovaleric acid
  MOD_RES (15)..(15): Lys(Dapa-Palm)
  C-term NH2
DTHFPCIKFEPRSKKCK

SEQ ID NO: 253 - protein, 18 aa, Artificial Sequence
  Description of Artificial Sequence: Synthetic peptide
  N-term Isovaleric acid
  MOD_RES (17)..(17): Lys(Dapa-Palm)
  C-term NH2
DTHFPCIKFEPRSKGCKK

SEQ ID NO: 254 - protein, 18 aa, Artificial Sequence
  Description of Artificial Sequence: Synthetic peptide
  N-term Isovaleric acid
  MOD_RES (18)..(18): Lys(Dapa-Palm)
  C-term NH2
DTHFPCIKFEPRSKGCKK

SEQ ID NO: 255 - protein, 19 aa, Artificial Sequence
  Description of Artificial Sequence: Synthetic peptide
  N-term Isovaleric acid
  C-term NH2
DTHFPCIKFGPRSKGWVCK

SEQ ID NO: 256 - protein, 19 aa, Artificial Sequence
  Description of Artificial Sequence: Synthetic peptide
  N-term Isovaleric acid
  C-term NH2
AAHFPCIKFGPRSKGWVCK

SEQ ID NO: 257 - protein, 19 aa, Artificial Sequence
  Description of Artificial Sequence: Synthetic peptide
  N-term Isovaleric acid
  C-term NH2
ATHFPCIKFGPRSKGWVCK

SEQ ID NO: 258 - protein, 19 aa, Artificial Sequence
  Description of Artificial Sequence: Synthetic peptide
  N-term Isovaleric acid
  C-term NH2
DAHFPCIKFGPRSKGWVCK

SEQ ID NO: 259 - protein, 19 aa, Artificial Sequence
  Description of Artificial Sequence: Synthetic peptide
  N-term Isovaleric acid
  C-term NH2
DTHAPCIKFGPRSKGWVCK

SEQ ID NO: 260 - protein, 19 aa, Artificial Sequence
  Description of Artificial Sequence: Synthetic peptide
  N-term Isovaleric acid
  C-term OH
DTHFPCIKAGPRSKGWVCK

SEQ ID NO: 261 - protein, 19 aa, Artificial Sequence
  Description of Artificial Sequence: Synthetic peptide
  N-term Isovaleric acid
  C-term OH
DTHFPCIKFEPRSKGWVCK

SEQ ID NO: 262 - protein, 19 aa, Artificial Sequence
  Description of Artificial Sequence: Synthetic peptide
  N-term Isovaleric acid
  C-term OH
DTHFPCIKFEPRSKGWECK

SEQ ID NO: 263 - protein, 18 aa, Artificial Sequence
  Description of Artificial Sequence: Synthetic peptide
  N-term Isovaleric acid
  C-term OH
DTHFPCIIFEPRSKGWEC

SEQ ID NO: 264 - protein, 19 aa, Artificial Sequence
  Description of Artificial Sequence: Synthetic peptide
  N-term Isovaleric acid
  MOD_RES (10)..(10): Lys(isoGlu-Palm)
  C-term NH2
DTHFPCIKFKPRSKGWECK

SEQ ID NO: 265 - protein, 19 aa, Artificial Sequence
  Description of Artificial Sequence: Synthetic peptide
  N-term Isovaleric acid
  MOD_RES (12)..(12): Lys(isoGlu-Palm)
  C-term NH2
DTHFPCIKFEPKSKGWECK

SEQ ID NO: 266 - protein, 19 aa, Artificial Sequence
  Description of Artificial Sequence: Synthetic peptide
  N-term Isovaleric acid
  C-term NH2
DTHAPCIKFEPRSKGWECK

SEQ ID NO: 267 - protein, 17 aa, Artificial Sequence
  Description of Artificial Sequence: Synthetic peptide
  MOD_RES (1)..(1): Ida
  MOD_RES (15)..(15): Lys(isoGlu-Palm)
  C-term NH2
XTHFPCIKFEPRSKKCK

SEQ ID NO: 268 - protein, 18 aa, Artificial Sequence
  Description of Artificial Sequence: Synthetic peptide
  N-term Isovaleric acid
  MOD_RES (8)..(8): Lys(isoGlu-Palm)
  C-term OH
DTHFPCIKFEPRSKGWEC

SEQ ID NO: 269 - protein, 19 aa, Artificial Sequence
  Description of Artificial Sequence: Synthetic peptide
  N-term 4,4-5,5-6,6,6-Heptafluorohexanoic acid
  C-term NH2
DTHFPCIKFGPRSKGWVCK

SEQ ID NO: 270 - protein, 18 aa, Artificial Sequence
  Description of Artificial Sequence: Synthetic peptide
  N-term Isovaleric acid
  MOD_RES (10)..(10): Lys(mysteric acid)
  C-term NH2
DTHFPCIKFKPRSKGWVC

SEQ ID NO: 271 - protein, 18 aa, Artificial Sequence
  Description of Artificial Sequence: Synthetic peptide
  N-term Isovaleric acid
  MOD_RES (10)..(10): Lys(lauric acid)
  C-term NH2
DTHFPCIKFKPRSKGWVC

SEQ ID NO: 272 - protein, 18 aa, Artificial Sequence
  Description of Artificial Sequence: Synthetic peptide
  N-term Isovaleric acid
  MOD_RES (10)..(10): Lys(decanoic acid)
  C-term NH2
DTHFPCIKFKPRSKGWVC

SEQ ID NO: 273 - protein, 18 aa, Artificial Sequence
  Description of Artificial Sequence: Synthetic peptide
  N-term Isovaleric acid
  MOD_RES (10)..(10): Lys(Octanoic acid)
  C-term NH2
DTHFPCIKFKPRSKGWVC

SEQ ID NO: 274 - protein, 18 aa, Artificial Sequence
  Description of Artificial Sequence: Synthetic peptide
  N-term Isovaleric acid
  MOD_RES (10)..(10): Lys(Hexanoic acid)
  C-term NH2
DTHFPCIKFKPRSKGWVC

SEQ ID NO: 275 - protein, 18 aa, Artificial Sequence
  Description of Artificial Sequence: Synthetic peptide
  N-term Isovaleric acid
  MOD_RES (10)..(10): Lys(Butyric acid)
  C-term NH2
DTHFPCIKFKPRSKGWVC

SEQ ID NO: 276 - protein, 18 aa, Artificial Sequence
  Description of Artificial Sequence: Synthetic peptide
  N-term Isovaleric acid
  MOD_RES (10)..(10): Lys(Ac)
  C-term NH2
DTHFPCIKFKPRSKGWVC

SEQ ID NO: 277 - protein, 19 aa, Artificial Sequence
  Description of Artificial Sequence: Synthetic peptide
  MOD_RES (1)..(1): Ida
  MOD_RES (19)..(19): Lys(mysteric acid)
  C-term NH2
XTHFPCIKFEPRSKGWVCK

SEQ ID NO: 278 - protein, 19 aa, Artificial Sequence
  Description of Artificial Sequence: Synthetic peptide
  MOD_RES (1)..(1): Ida
  MOD_RES (19)..(19): Lys(lauric acid)
  C-term NH2
XTHFPCIKFEPRSKGWVCK

SEQ ID NO: 279 - protein, 19 aa, Artificial Sequence
  Description of Artificial Sequence: Synthetic peptide
  MOD_RES (1)..(1): Ida
  MOD_RES (19)..(19): Lys(decanoic acid)
  C-term NH2
XTHFPCIKFEPRSKGWVCK

SEQ ID NO: 280 - protein, 19 aa, Artificial Sequence
  Description of Artificial Sequence: Synthetic peptide
  MOD_RES (1)..(1): Ida
  MOD_RES (19)..(19): Lys(octanoic acid)
  C-term NH2
XTHFPCIKFEPRSKGWVCK

SEQ ID NO: 281 - protein, 19 aa, Artificial Sequence
  Description of Artificial Sequence: Synthetic peptide
  MOD_RES (1)..(1): Ida
  MOD_RES (19)..(19): Lys(hexanoic acid)
  C-term NH2
XTHFPCIKFEPRSKGWVCK

SEQ ID NO: 282 - protein, 19 aa, Artificial Sequence
  Description of Artificial Sequence: Synthetic peptide
  MOD_RES (1)..(1): Ida
  MOD_RES (19)..(19): Lys(butyric acid)
  C-term NH2
XTHFPCIKFEPRSKGWVCK

SEQ ID NO: 283 - protein, 19 aa, Artificial Sequence
  Description of Artificial Sequence: Synthetic peptide
  N-term Isovaleric acid
  MOD_RES (1)..(1): Ida
  MOD_RES (19)..(19): Lys(Ac)
  C-term NH2
XTHFPCIKFEPRSKGWVCK

SEQ ID NO: 284 - protein, 17 aa, Artificial Sequence
  Description of Artificial Sequence: Synthetic peptide
  N-term Isovaleric acid
  MOD_RES (17)..(17): Lys(Octanoic acid)
  C-term NH2
HFPCIKFEPRSKGWVCK

SEQ ID NO: 285 - protein, 17 aa, Artificial Sequence
  Description of Artificial Sequence: Synthetic peptide
  N-term Isovaleric acid
  MOD_RES (17)..(17): Lys(lauric acid)
  C-term NH2
HFPCIKFEPRSKGWVCK

SEQ ID NO: 286 - protein, 17 aa, Artificial Sequence
  Description of Artificial Sequence: Synthetic peptide
  N-term Isovaleric acid
  C-term NH2
DTHFPCIKFEPHSKGCK

SEQ ID NO: 287 - protein, 16 aa, Artificial Sequence
  Description of Artificial Sequence: Synthetic peptide
  N-term Isovaleric acid
  C-term NH2
DTHFPCIHFEPHSKGC

SEQ ID NO: 288 - protein, 17 aa, Artificial Sequence
  Description of Artificial Sequence: Synthetic peptide
  N-term Isovaleric acid
  MOD_RES (14)..(14): Lys(Albu)
  C-term NH2
DTHFPCIKFEPHSKGCK

SEQ ID NO: 289 - protein, 16 aa, Artificial Sequence
  Description of Artificial Sequence: Synthetic peptide
  N-term Isovaleric acid
  C-term NH2
DTHFPCIKFEPREKEC

SEQ ID NO: 290 - protein, 16 aa, Artificial Sequence
  Description of Artificial Sequence: Synthetic peptide
  N-term Isovaleric acid
  C-term NH2
DTAFPCIKFEPRSKEC

SEQ ID NO: 291 - protein, 12 aa, Artificial Sequence
  Description of Artificial Sequence: Synthetic peptide
  N-term Isovaleric acid
  C-term NH2
DTHFPCIKFECK

SEQ ID NO: 292 - protein, 15 aa, Artificial Sequence
  Description of Artificial Sequence: Synthetic peptide
  N-term Hy
  C-term NH2
DTHFPIAIFAAGICI

SEQ ID NO: 293 - protein, 14 aa, Artificial Sequence
  Description of Artificial Sequence: Synthetic peptide
  N-term Hy
  C-term NH2
DTHFPIAIFAAICI

SEQ ID NO: 294 - protein, 13 aa, Artificial Sequence
  Description of Artificial Sequence: Synthetic peptide
  N-term Hy
  C-term NH2
DTHFPIAIFAICI

SEQ ID NO: 295 - protein, 12 aa, Artificial Sequence
  Description of Artificial Sequence: Synthetic peptide
  N-term Hy
  C-term NH2
DTHFPIAIFICI

SEQ ID NO: 296 - protein, 11 aa, Artificial Sequence
  Description of Artificial Sequence: Synthetic peptide
  N-term Hy
  C-term NH2
DTHFPIAIICI

SEQ ID NO: 297 - protein, 10 aa, Artificial Sequence
  Description of Artificial Sequence: Synthetic peptide
  N-term Hy
  C-term NH2
DTHFPIAICI

SEQ ID NO: 298 - protein, 9 aa, Artificial Sequence
  Description of Artificial Sequence: Synthetic peptide
  N-term Hy
  C-term NH2
DTHFPIICI

SEQ ID NO: 299 - protein, 9 aa, Artificial Sequence
  Description of Artificial Sequence: Synthetic peptide
  N-term Hy
  C-term NH2
DTHICIAIF

SEQ ID NO: 300 - protein, 9 aa, Artificial Sequence
  Description of Artificial Sequence: Synthetic peptide
  N-term Hy
  C-term NH2
DTHCPIAIF

SEQ ID NO: 301 - protein, 9 aa, Artificial Sequence
  Description of Artificial Sequence: Synthetic peptide
  N-term Hy
  C-term NH2
DTHFPCIIA

SEQ ID NO: 302 - protein, 9 aa, Artificial Sequence
  Description of Artificial Sequence: Synthetic peptide
  N-term Hy
  C-term NH2
DTHFPCAIF

SEQ ID NO: 303 - protein, 9 aa, Artificial Sequence
  Description of Artificial Sequence: Synthetic peptide
  N-term Hy
  C-term NH2
DTHFACIIF

SEQ ID NO: 304 - protein, 9 aa, Artificial Sequence
  Description of Artificial Sequence: Synthetic peptide
  N-term Hy
  MOD_RES (5)..(5): D-Ala
  C-term NH2
DTHFACIIF

SEQ ID NO: 305 - protein, 9 aa, Artificial Sequence
  Description of Artificial Sequence: Synthetic peptide
  N-term Hy
  C-term NH2
DTHAPCIIF

SEQ ID NO: 306 - protein, 9 aa, Artificial Sequence
  Description of Artificial Sequence: Synthetic peptide
  N-term Hy
  C-term NH2
DTAFPCIIF

SEQ ID NO: 307 - protein, 9 aa, Artificial Sequence
  Description of Artificial Sequence: Synthetic peptide
  N-term Hy
  C-term NH2
ATHFPCIIF

SEQ ID NO: 308 - protein, 9 aa, Artificial Sequence
  Description of Artificial Sequence: Synthetic peptide
  MOD_RES (1)..(1): Ida
  MOD_RES (5)..(5): bhPro
  C-term NH2
XTHFPCIIF

SEQ ID NO: 309 - protein, 9 aa, Artificial Sequence
  Description of Artificial Sequence: Synthetic peptide
  N-term Hy
  C-term NH2
DTHFPCIEF

SEQ ID NO: 310 - protein, 9 aa, Artificial Sequence
  Description of Artificial Sequence: Synthetic peptide
  N-term Hy
  C-term NH2
DTHFPCIEF

SEQ ID NO: 311 - protein, 9 aa, Artificial Sequence
  Description of Artificial Sequence: Synthetic peptide
  N-term Isovaleric acid
  C-term NH2
DTHFPCIIF

SEQ ID NO: 312 - protein, 9 aa, Artificial Sequence
  Description of Artificial Sequence: Synthetic peptide
  N-term Isovaleric acid
  C-term NH2
DTHFPAIIF

SEQ ID NO: 313 - protein, 9 aa, Artificial Sequence
  Description of Artificial Sequence: Synthetic peptide
  N-term Isovaleric acid
  C-term NH2
DTHFPSIIF

SEQ ID NO: 314 - protein, 9 aa, Artificial Sequence
  Description of Artificial Sequence: Synthetic peptide
  N-term Isovaleric acid
  C-term NH2
DTHFPCIKF

SEQ ID NO: 315 - skipped

SEQ ID NO: 316 - protein, 8 aa, Artificial Sequence
  Description of Artificial Sequence: Synthetic peptide
  N-term Hy
  C-term NH2
DTHFPCIF

SEQ ID NO: 317-319 - skipped (3 entries)

SEQ ID NO: 320 - protein, 10 aa, Artificial Sequence
  Description of Artificial Sequence: Synthetic peptide
  N-term Hy
  C-term NH2
DTHFPCIKFF

SEQ ID NO: 321 - protein, 9 aa, Artificial Sequence
  Description of Artificial Sequence: Synthetic peptide
  N-term Hy
  C-term NH2
YTHFPCIIF

SEQ ID NO: 322 - protein, 9 aa, Artificial Sequence
  Description of Artificial Sequence: Synthetic peptide
  N-term Hy
  C-term NH2
LTHFPCIIF

SEQ ID NO: 323 - protein, 9 aa, Artificial Sequence
  Description of Artificial Sequence: Synthetic peptide
  N-term Hy
  C-term NH2
ETHFPCIIF

SEQ ID NO: 324 - protein, 9 aa, Artificial Sequence
  Description of Artificial Sequence: Synthetic peptide
  N-term Hy
  C-term NH2
DRHFPCIIF

SEQ ID NO: 325 - protein, 9 aa, Artificial Sequence
  Description of Artificial Sequence: Synthetic peptide
  N-term Hy
  C-term NH2
DTKFPCIIF

SEQ ID NO: 326 - protein, 9 aa, Artificial Sequence
  Description of Artificial Sequence: Synthetic peptide
  N-term Hy
  C-term NH2
DTHFECIIF

SEQ ID NO: 327 - protein, 9 aa, Artificial Sequence
  Description of Artificial Sequence: Synthetic peptide
  N-term Hy
  C-term NH2
DTHFPCIIK

SEQ ID NO: 328 - protein, 9 aa, Artificial Sequence
  Description of Artificial Sequence: Synthetic peptide
  N-term Hy
  C-term NH2
DTHFPCIIR

SEQ ID NO: 329 - protein, 9 aa, Artificial Sequence
  Description of Artificial Sequence: Synthetic peptide
  N-term Hy
  C-term NH2
DTHFPCIEF

SEQ ID NO: 330 - protein, 9 aa, Artificial Sequence
  Description of Artificial Sequence: Synthetic peptide
  N-term Hy
  C-term NH2
DTHFPCIVF

SEQ ID NO: 331 - protein, 9 aa, Artificial Sequence
  Description of Artificial Sequence: Synthetic peptide
  N-term Hy
  C-term NH2
DTHFPCILF

SEQ ID NO: 332 - protein, 9 aa, Artificial Sequence
  Description of Artificial Sequence: Synthetic peptide
  N-term Hy
  C-term NH2
DTHFPCILK

SEQ ID NO: 333 - protein, 9 aa, Artificial Sequence
  Description of Artificial Sequence: Synthetic peptide
  N-term Hy
  C-term NH2
DTHFPCIEK

SEQ ID NO: 334 - skipped

SEQ ID NO: 335 - protein, 25 aa, Artificial Sequence
  Description of Artificial Sequence: Synthetic peptide
  N-term Hy
  C-term OH
DTHFPICIFCCGCCHRSKCGMCCKT

SEQ ID NO: 336-368 - skipped (33 entries)

SEQ ID NO: 369 - protein, 17 aa, Artificial Sequence
  Description of Artificial Sequence: Synthetic peptide
  N-term Isovaleric acid
  C-term NH2
DTHFPCIKFEPRSKECK

SEQ ID NO: 370 - protein, 17 aa, Artificial Sequence
  Description of Artificial Sequence: Synthetic peptide
  N-term Isovaleric acid
  C-term NH2
DTHFPCIKFEPHSKECK

SEQ ID NO: 371 - protein, 17 aa, Artificial Sequence
  Description of Artificial Sequence: Synthetic peptide
  N-term Isovaleric acid
  C-term NH2
DTHFPCIKKEPHSKECK

SEQ ID NO: 372 - protein, 17 aa, Artificial Sequence
  Description of Artificial Sequence: Synthetic peptide
  N-term Isovaleric acid
  MOD_RES (10)..(10): Lys(isoGlu-Palm)
  C-term NH2
DTHFPCIKFKPHSKECK

SEQ ID NO: 373 - protein, 15 aa, Artificial Sequence
  Description of Artificial Sequence: Synthetic peptide
  N-term Isovaleric acid
  C-term NH2
DTHFPCIKFEPRECK

SEQ ID NO: 374 - protein, 15 aa, Artificial Sequence
  Description of Artificial Sequence: Synthetic peptide
  N-term Isovaleric acid
  C-term NH2
DTHFPCIKFEPHECK

SEQ ID NO: 375 - protein, 14 aa, Artificial Sequence
  Description of Artificial Sequence: Synthetic peptide
  N-term Isovaleric acid
  C-term NH2
DTHFPCIKFEPRCK

SEQ ID NO: 376 - protein, 10 aa, Artificial Sequence
  Description of Artificial Sequence: Synthetic peptide
DTHFPICIFC

SEQ ID NO: 377 - protein, 4 aa, Artificial Sequence
  Description of Artificial Sequence: Synthetic peptide
FPIC

SEQ ID NO: 378 - protein, 5 aa, Artificial Sequence
  Description of Artificial Sequence: Synthetic peptide
HFPIC

SEQ ID NO: 379 - protein, 6 aa, Artificial Sequence
  Description of Artificial Sequence: Synthetic peptide
HFPICI

SEQ ID NO: 380 - protein, 7 aa, Artificial Sequence
  Description of Artificial Sequence: Synthetic peptide
HFPICIF

SEQ ID NO: 381 - protein, 7 aa, Artificial Sequence
  Description of Artificial Sequence: Synthetic peptide
DTHFPIC

SEQ ID NO: 382 - protein, 9 aa, Artificial Sequence
  Description of Artificial Sequence: Synthetic peptide
DTHFPICIF

SEQ ID NO: 383 - protein, 10 aa, Artificial Sequence
  Description of Artificial Sequence: Synthetic peptide
DTHFPIAIFC

SEQ ID NO: 384 - protein, 9 aa, Artificial Sequence
  Description of Artificial Sequence: Synthetic peptide
DTHAPICIF

SEQ ID NO: 385 - protein, 9 aa, Artificial Sequence
  Description of Artificial Sequence: Synthetic peptide
  MOD_RES (7)..(7): Cys(S-tBu)
DTHAPICIF

SEQ ID NO: 386 - protein, 9 aa, Artificial Sequence
  Description of Artificial Sequence: Synthetic peptide
  MOD_RES (7)..(7): Cys(tBu)
DTHAPICIF

SEQ ID NO: 387 - protein, 9 aa, Artificial Sequence
  Description of Artificial Sequence: Synthetic peptide
DTHFPIAIF

SEQ ID NO: 388 - protein, 9 aa, Artificial Sequence
  Description of Artificial Sequence: Synthetic peptide
DTHFPISIF

SEQ ID NO: 389 - protein, 9 aa, Artificial Sequence
  Description of Artificial Sequence: Synthetic peptide
  MOD_RES (7)..(7): D-Cys
DTHFPICIF

SEQ ID NO: 390 - protein, 9 aa, Artificial Sequence
  Description of Artificial Sequence: Synthetic peptide
  MOD_RES (7)..(7): homoCys
DTHFPICIF

SEQ ID NO: 391 - protein, 9 aa, Artificial Sequence
  Description of Artificial Sequence: Synthetic peptide
  MOD_RES (7)..(7): Pen
DTHFPIXIF

SEQ ID NO: 392 - protein, 9 aa, Artificial Sequence
  Description of Artificial Sequence: Synthetic peptide
  N-term Isovaleric acid
  MOD_RES (7)..(7): D-Pen
DTHFPIXIF

SEQ ID NO: 393 - protein, 9 aa, Artificial Sequence
  Description of Artificial Sequence: Synthetic peptide
  MOD_RES (7)..(7): Dapa(AcBr)
DTHFPIXIF

SEQ ID NO: 394 - protein, 10 aa, Artificial Sequence
  Description of Artificial Sequence: Synthetic peptide
CDTHFPICIF

SEQ ID NO: 395 - protein, 9 aa, Artificial Sequence
  Description of Artificial Sequence: Synthetic peptide
  C-term NHCH2CH2S
DTHFPICIF

SEQ ID NO: 396 - protein, 8 aa, Artificial Sequence
  Description of Artificial Sequence: Synthetic peptide
CHFPICIF

SEQ ID NO: 397 - protein, 7 aa, Artificial Sequence
  Description of Artificial Sequence: Synthetic peptide
  C-term NHCH2CH2S
HFPICIF

SEQ ID NO: 398 - protein, 9 aa, Artificial Sequence
  Description of Artificial Sequence: Synthetic peptide
  MOD_RES (2)..(2): Tle
  MOD_RES (4)..(4): Phg
  MOD_RES (5)..(5): Oic
  MOD_RES (6)..(6): Chg
  MOD_RES (8)..(8): Chg
DXHXXXCXF

SEQ ID NO: 399 - protein, 9 aa, Artificial Sequence
  Description of Artificial Sequence: Synthetic peptide
  MOD_RES (2)..(2): Tle
  MOD_RES (5)..(5): Oic
  MOD_RES (6)..(6): Chg
  MOD_RES (8)..(8): Chg
DXHPXXCXF

SEQ ID NO: 400 - protein, 9 aa, Artificial Sequence
  Description of Artificial Sequence: Synthetic peptide
  MOD_RES (1)..(1): D-Phe
  MOD_RES (2)..(2): D-Ile
  MOD_RES (3)..(3): D-Cys
  MOD_RES (4)..(4): D-Ile
  MOD_RES (5)..(5): D-Pro
  MOD_RES (6)..(6): D-Phe
  MOD_RES (7)..(7): D-His
  MOD_RES (8)..(8): D-Thr
  MOD_RES (9)..(9): D-Asp
FICIPFHTD

SEQ ID NO: 401 - protein, 7 aa, Artificial Sequence
  Description of Artificial Sequence: Synthetic peptide
  MOD_RES (1)..(1): D-Phe
  MOD_RES (2)..(2): D-Ile
  MOD_RES (3)..(3): D-Cys
  MOD_RES (4)..(4): D-Ile
  MOD_RES (5)..(5): D-Pro
  MOD_RES (6)..(6): D-Phe
  MOD_RES (7)..(7): D-His
FICIPFH

SEQ ID NO: 402 - protein, 9 aa, Artificial Sequence
  Description of Artificial Sequence: Synthetic peptide
  N-term Chenodeoxycholate-PEG11
  MOD_RES (1)..(1): D-Phe
  MOD_RES (2)..(2): D-Ile
  MOD_RES (3)..(3): D-Cys
  MOD_RES (4)..(4): D-Ile
  MOD_RES (5)..(5): D-Pro
  MOD_RES (6)..(6): D-Phe
  MOD_RES (7)..(7): D-His
  MOD_RES (8)..(8): D-Thr
  MOD_RES (9)..(9): D-Asp
FICIPFHTD

SEQ ID NO: 403 - protein, 9 aa, Artificial Sequence
  Description of Artificial Sequence: Synthetic peptide
  N-term Ursodeoxycholate-PEG11
  MOD_RES (1)..(1): D-Phe
  MOD_RES (2)..(2): D-Ile
  MOD_RES (3)..(3): D-Cys
  MOD_RES (4)..(4): D-Ile
  MOD_RES (5)..(5): D-Pro
  MOD_RES (6)..(6): D-Phe
  MOD_RES (7)..(7): D-His
  MOD_RES (8)..(8): D-Thr
  MOD_RES (9)..(9): D-Asp
FICIPFHTD

SEQ ID NO: 404 - protein, 9 aa, Artificial Sequence
  Description of Artificial Sequence: Synthetic peptide
  MOD_RES (2)..(2): D-Ile
  MOD_RES (3)..(3): D-Cys
  MOD_RES (4)..(4): D-Ile
  MOD_RES (5)..(5): D-Pro
  MOD_RES (6)..(6): D-Phe
  MOD_RES (7)..(7): D-His
  MOD_RES (8)..(8): D-Thr
  MOD_RES (9)..(9): D-Asp
  C-term PEG11 conjugated to the peptide “GYIPEAPRDGQAYVRKDGEWVLLSTFL”
FICIPFHTD

SEQ ID NO: 405 - protein, 39 aa, Artificial Sequence
  Description of Artificial Sequence: Synthetic polypeptide
  MOD_RES (2)..(2): D-Ile
  MOD_RES (3)..(3): D-Cys
  MOD_RES (4)..(4): D-Ile
  MOD_RES (5)..(5): D-Pro
  MOD_RES (6)..(6): D-Phe
  MOD_RES (7)..(7): D-His
  MOD_RES (8)..(8): D-Thr
  MOD_RES (9)..(9): D-Asp
  MOD_RES (12)..(12): Hyp
  MOD_RES (15)..(15): Hyp
  MOD_RES (18)..(18): Hyp
  MOD_RES (21)..(21): Hyp
  MOD_RES (24)..(24): Hyp
  MOD_RES (27)..(27): Hyp
  MOD_RES (30)..(30): Hyp
  MOD_RES (33)..(33): Hyp
  MOD_RES (36)..(36): Hyp
  MOD_RES (39)..(39): Hyp
FICIPFHTDGPXGPXGPXGPXGPXGPXGPXGPXGPXGPX

SEQ ID NO: 406 - protein, 9 aa, Artificial Sequence
  Description of Artificial Sequence: Synthetic peptide
  N-term Palmitoyl-PEG11
  MOD_RES (1)..(1): D-Phe
  MOD_RES (2)..(2): D-Ile
  MOD_RES (3)..(3): D-Cys
  MOD_RES (4)..(4): D-Ile
  MOD_RES (5)..(5): D-Pro
  MOD_RES (6)..(6): D-Phe
  MOD_RES (7)..(7): D-His
  MOD_RES (8)..(8): D-Thr
  MOD_RES (9)..(9): D-Asp
FICIPFHTD

SEQ ID NO: 407 - protein, 9 aa, Artificial Sequence
  Description of Artificial Sequence: Synthetic peptide
  N-term 2(Palmitoyl)-Dapa-PEG11
  MOD_RES (1)..(1): D-Phe
  MOD_RES (2)..(2): D-Ile
  MOD_RES (3)..(3): D-Cys
  MOD_RES (4)..(4): D-Ile
  MOD_RES (5)..(5): D-Pro
  MOD_RES (6)..(6): D-Phe
  MOD_RES (7)..(7): D-His
  MOD_RES (8)..(8): D-Thr
  MOD_RES (9)..(9): D-Asp
FICIPFHTD

SEQ ID NO: 408 - protein, 10 aa, Artificial Sequence
  Description of Artificial Sequence: Synthetic peptide
  MOD_RES (4)..(4): bhPhe
DTHFPIICIF

SEQ ID NO: 409 - protein, 8 aa, Artificial Sequence
  Description of Artificial Sequence: Synthetic peptide
  MOD_RES (4)..(4): Dpa
DTHXPICI

SEQ ID NO: 410 - protein, 9 aa, Artificial Sequence
  Description of Artificial Sequence: Synthetic peptide
  MOD_RES (4)..(4): Bip
DTHXPICIF

SEQ ID NO: 411 - protein, 9 aa, Artificial Sequence
  Description of Artificial Sequence: Synthetic peptide
  MOD_RES (4)..(4): 1-Nal
DTHXPICIF

SEQ ID NO: 412 - protein, 9 aa, Artificial Sequence
  Description of Artificial Sequence: Synthetic peptide
  MOD_RES (4)..(4): bhDpa
DTHXPICIF

SEQ ID NO: 413 - protein, 9 aa, Artificial Sequence
  Description of Artificial Sequence: Synthetic peptide
  MOD_RES (9)..(9): bhPhe
DTHFPICIF

SEQ ID NO: 414 - protein, 9 aa, Artificial Sequence
  Description of Artificial Sequence: Synthetic peptide
  MOD_RES (9)..(9): Dpa
DTHFPICIX

SEQ ID NO: 415 - protein, 9 aa, Artificial Sequence
  Description of Artificial Sequence: Synthetic peptide
  MOD_RES (9)..(9): Bip
DTHFPICIX

SEQ ID NO: 416 - protein, 9 aa, Artificial Sequence
  Description of Artificial Sequence: Synthetic peptide
  MOD_RES (9)..(9): 1-Nal
DTHFPICIX

SEQ ID NO: 417 - protein, 9 aa, Artificial Sequence
  Description of Artificial Sequence: Synthetic peptide
  MOD_RES (9)..(9): bhDpa
DTHFPICIX

SEQ ID NO: 418 - protein, 9 aa, Artificial Sequence
  Description of Artificial Sequence: Synthetic peptide
  MOD_RES (4)..(4): Dpa
  MOD_RES (9)..(9): Dpa
DTHXPICIX

SEQ ID NO: 419 - protein, 7 aa, Artificial Sequence
  Description of Artificial Sequence: Synthetic peptide
  MOD_RES (2)..(2): Dpa
DXPICIF

SEQ ID NO: 420 - protein, 7 aa, Artificial Sequence
  Description of Artificial Sequence: Synthetic peptide
  MOD_RES (2)..(2): Dpa
  MOD_RES (7)..(7): Dpa
DXPICIX

SEQ ID NO: 421 - protein, 9 aa, Artificial Sequence
  Description of Artificial Sequence: Synthetic peptide
  MOD_RES (4)..(4): Dpa
  MOD_RES (6)..(6): D-Arg
  MOD_RES (9)..(9): Dpa
DTHXPRCRX

SEQ ID NO: 422 - protein, 9 aa, Artificial Sequence
  Description of Artificial Sequence: Synthetic peptide
  MOD_RES (4)..(4): Dpa
  MOD_RES (6)..(6): D-Arg
  MOD_RES (8)..(8): D-Arg
  MOD_RES (9)..(9): Dpa
DTHXPRCRX

SEQ ID NO: 423 - protein, 9 aa, Artificial Sequence
  Description of Artificial Sequence: Synthetic peptide
  MOD_RES (4)..(4): Dpa
  MOD_RES (5)..(5): Oic
DTHXXICIF

SEQ ID NO: 424 - protein, 9 aa, Artificial Sequence
  Description of Artificial Sequence: Synthetic peptide
  MOD_RES (4)..(4): Dpa
  MOD_RES (5)..(5): Oic
  MOD_RES (9)..(9): Dpa
DTHXXICIX

SEQ ID NO: 425 - protein, 9 aa, Artificial Sequence
  Description of Artificial Sequence: Synthetic peptide
  MOD_RES (4)..(4): Dpa
  MOD_RES (9)..(9): Dpa
DTHXPCCCX

SEQ ID NO: 426 - protein, 12 aa, Artificial Sequence
  Description of Artificial Sequence: Synthetic peptide
  MOD_RES (10)..(10): D-Pro
DTHFPICIFPPK

SEQ ID NO: 427 - protein, 12 aa, Artificial Sequence
  Description of Artificial Sequence: Synthetic peptide
  MOD_RES (10)..(10): D-Pro
DTHFPICIFPPR

SEQ ID NO: 428 - protein, 12 aa, Artificial Sequence
  Description of Artificial Sequence: Synthetic peptide
  MOD_RES (10)..(10): bhPro
DTHFPICIFPPK

SEQ ID NO: 429 - protein, 12 aa, Artificial Sequence
  Description of Artificial Sequence: Synthetic peptide
  MOD_RES (10)..(10): bhPro
DTHFPICIFPPR

SEQ ID NO: 430 - protein, 12 aa, Artificial Sequence
  Description of Artificial Sequence: Synthetic peptide
  MOD_RES (10)..(10): D-Pro
  MOD_RES (11)..(11): bhPro
DTHFPICIFPPK

SEQ ID NO: 431 - protein, 12 aa, Artificial Sequence
  Description of Artificial Sequence: Synthetic peptide
  MOD_RES (10)..(10): D-Pro
  MOD_RES (11)..(11): bhPro
DTHFPICIFPPR

SEQ ID NO: 432 - protein, 12 aa, Artificial Sequence
  Description of Artificial Sequence: Synthetic peptide
  MOD_RES (9)..(9): bhPhe
  MOD_RES (10)..(10): D-Pro
DTHFPICIFPPK

SEQ ID NO: 433 - protein, 12 aa, Artificial Sequence
  Description of Artificial Sequence: Synthetic peptide
  MOD_RES (9)..(9): bhPhe
  MOD_RES (10)..(10): D-Pro
DTHFPICIFPPR

SEQ ID NO: 434 - protein, 12 aa, Artificial Sequence
  Description of Artificial Sequence: Synthetic peptide
  MOD_RES (9)..(9): bhPhe
  MOD_RES (10)..(10): D-Pro
  MOD_RES (11)..(11): bhPro
DTHFPICIFPPK

SEQ ID NO: 435 - protein, 12 aa, Artificial Sequence
  Description of Artificial Sequence: Synthetic peptide
  MOD_RES (9)..(9): bhPhe
  MOD_RES (10)..(10): D-Pro
  MOD_RES (11)..(11): bhPro
DTHFPICIFPPR

SEQ ID NO: 436 - protein, 8 aa, Artificial Sequence
  Description of Artificial Sequence: Synthetic peptide
  MOD_RES (2)..(2): Inp
  MOD_RES (3)..(3): D-Dpa
  MOD_RES (4)..(4): Amc
  MOD_RES (6)..(6): Amc
  MOD_RES (7)..(7): Inp
  MOD_RES (8)..(8): Dpa
  C-term Cysteamide
CXXXRXXX

SEQ ID NO: 437 - protein, 8 aa, Artificial Sequence
  Description of Artificial Sequence: Synthetic peptide
  MOD_RES (3)..(3): D-Dpa
  MOD_RES (4)..(4): Amc
  MOD_RES (6)..(6): Amc
  MOD_RES (7)..(7): Inp
  MOD_RES (8)..(8): Dpa
  C-term Cysteamide
CPXXRXXX

SEQ ID NO: 438 - protein, 8 aa, Artificial Sequence
  Description of Artificial Sequence: Synthetic peptide
  MOD_RES (2)..(2): bhPro
  MOD_RES (3)..(3): D-Dpa
  MOD_RES (4)..(4): Amc
  MOD_RES (6)..(6): Amc
  MOD_RES (7)..(7): Inp
  MOD_RES (8)..(8): Dpa
  C-term Cysteamide
CPXXRXXX

SEQ ID NO: 439 - protein, 8 aa, Artificial Sequence
  Description of Artificial Sequence: Synthetic peptide
  MOD_RES (3)..(3): D-Dpa
  MOD_RES (4)..(4): Amc
  MOD_RES (6)..(6): Amc
  MOD_RES (7)..(7): Inp
  MOD_RES (8)..(8): Dpa
  C-term Cysteamide
CGXXRXXX

SEQ ID NO: 440 - protein, 9 aa, Artificial Sequence
  Description of Artificial Sequence: Synthetic peptide
  N-term Hy
  C-term NH2
DTHFPCAIF

SEQ ID NO: 441 - protein, 9 aa, Artificial Sequence
  Description of Artificial Sequence: Synthetic peptide
  N-term Hy
  C-term NH2
DTHFPCRRF

SEQ ID NO: 442 - protein, 9 aa, Artificial Sequence
  Description of Artificial Sequence: Synthetic peptide
  MOD_RES (1)..(1): Ida
  MOD_RES (4)..(4): Dpa
  MOD_RES (5)..(5): bhPro
  MOD_RES (9)..(9): bhPhe
  C-term NH2
XTHXPCRRF

SEQ ID NO: 443 - protein, 9 aa, Artificial Sequence
  Description of Artificial Sequence: Synthetic peptide
  N-term Hy
  C-term NH2
DTHFPCEIF

SEQ ID NO: 444 - protein, 9 aa, Artificial Sequence
  Description of Artificial Sequence: Synthetic peptide
  N-term Hy
  C-term NH2
DTHFPCFIF

SEQ ID NO: 445 - protein, 9 aa, Artificial Sequence
  Description of Artificial Sequence: Synthetic peptide
  N-term Hy
  C-term NH2
DTHFPCQIF

SEQ ID NO: 446 - protein, 9 aa, Artificial Sequence
  Description of Artificial Sequence: Synthetic peptide
  N-term Hy
  C-term NH2
DTHFPCRIF

SEQ ID NO: 447 - protein, 9 aa, Artificial Sequence
  Description of Artificial Sequence: Synthetic peptide
  N-term Hy
  MOD_RES (1)..(1): pGlu
  C-term NH2
ETHFPCRKF

SEQ ID NO: 448 - protein, 9 aa, Artificial Sequence
  Description of Artificial Sequence: Synthetic peptide
  N-term Hy
  C-term NH2
DTHFPCLIF

SEQ ID NO: 449 - protein, 9 aa, Artificial Sequence
  Description of Artificial Sequence: Synthetic peptide
  N-term Hy
  C-term NH2
DTHFPCVIF

SEQ ID NO: 450 - protein, 9 aa, Artificial Sequence
  Description of Artificial Sequence: Synthetic peptide
  N-term Hy
  C-term NH2
DTHFPCEIF

SEQ ID NO: 451 - protein, 9 aa, Artificial Sequence
  Description of Artificial Sequence: Synthetic peptide
  N-term Hy
  C-term NH2
DTHFPCRIF

SEQ ID NO: 452 - protein, 9 aa, Artificial Sequence
  Description of Artificial Sequence: Synthetic peptide
  N-term Hy
  C-term NH2
DTHFPCKIF

SEQ ID NO: 453 - protein, 8 aa, Artificial Sequence
  Description of Artificial Sequence: Synthetic peptide
  N-term Hy
  C-term NH2
DTHFPCLF

SEQ ID NO: 454 - protein, 8 aa, Artificial Sequence
  Description of Artificial Sequence: Synthetic peptide
  N-term Hy
  C-term NH2
DTHFPCEF

SEQ ID NO: 455 - protein, 8 aa, Artificial Sequence
  Description of Artificial Sequence: Synthetic peptide
  N-term Hy
  C-term NH2
DTHFPCRF

SEQ ID NO: 456 - protein, 17 aa, Artificial Sequence
  Description of Artificial Sequence: Synthetic peptide
  N-term Hy
  C-term NH2
DTHFPRRFGPRSKGWVC

SEQ ID NO: 457 - protein, 18 aa, Artificial Sequence
  Description of Artificial Sequence: Synthetic peptide
  MOD_RES (1)..(1): Ida
  MOD_RES (5)..(5): bhPro
  MOD_RES (9)..(9): bhPhe
  C-term NH2
XTHFPCRRFGPRSKGWVC

SEQ ID NO: 458 - protein, 17 aa, Artificial Sequence
  Description of Artificial Sequence: Synthetic peptide
  N-term Hy
  C-term NH2
DTHFPCIFGPRSKGWVC

SEQ ID NO: 459 - protein, 19 aa, Artificial Sequence
  Description of Artificial Sequence: Synthetic peptide
  N-term Hy
  C-term NH2
DTHFPCRIFGPRSRGWVCK

SEQ ID NO: 460 - protein, 19 aa, Artificial Sequence
  Description of Artificial Sequence: Synthetic peptide
  N-term Isovaleric acid
  C-term NH2
DTHFPCLIFGPRSKGWVCK

SEQ ID NO: 461 - protein, 19 aa, Artificial Sequence
  Description of Artificial Sequence: Synthetic peptide
  N-term Isovaleric acid
  C-term NH2
DTHFPCVIFGPRSKGWVCK

SEQ ID NO: 462 - protein, 19 aa, Artificial Sequence
  Description of Artificial Sequence: Synthetic peptide
  N-term Isovaleric acid
  C-term NH2
DTHFPCSIFGPRSKGWVCK

SEQ ID NO: 463 - protein, 19 aa, Artificial Sequence
  Description of Artificial Sequence: Synthetic peptide
  N-term Isovaleric acid
  C-term NH2
DTHFPCQIFGPRSKGWVCK

SEQ ID NO: 464 - protein, 19 aa, Artificial Sequence
  Description of Artificial Sequence: Synthetic peptide
  N-term Isovaleric acid
  C-term NH2
DTHFPCKIFGPRSKGWVCK

SEQ ID NO: 465 - protein, 19 aa, Artificial Sequence
  Description of Artificial Sequence: Synthetic peptide
  N-term Isovaleric acid
  MOD_RES (7)..(7): Dapa
  C-term NH2
DTHFPCXIFGPRSKGWDCK

SEQ ID NO: 466 - protein, 19 aa, Artificial Sequence
  Description of Artificial Sequence: Synthetic peptide
  N-term Isovaleric acid
  MOD_RES (7)..(7): Dapa
  C-term NH2
DTHFPCXIFGPRSKGWECK

SEQ ID NO: 467 - protein, 19 aa, Artificial Sequence
  Description of Artificial Sequence: Synthetic peptide
  N-term Isovaleric acid
  C-term NH2
DTHFPCKIFGPRSKGWECK

SEQ ID NO: 468 - protein, 19 aa, Artificial Sequence
  Description of Artificial Sequence: Synthetic peptide
  N-term Isovaleric acid
  C-term NH2
DTHFPCRRFGPRSKGWVCK

SEQ ID NO: 469 - protein, 19 aa, Artificial Sequence
  Description of Artificial Sequence: Synthetic peptide
  N-term Isovaleric acid
  C-term NH2
DTHFPCTIFGPRSKGWVCK

SEQ ID NO: 470 - protein, 10 aa, Artificial Sequence
  Description of Artificial Sequence: Synthetic peptide
  N-term Hy
  C-term NH2
DTHFPIAICI

SEQ ID NO: 471 - protein, 9 aa, Artificial Sequence
  Description of Artificial Sequence: Synthetic peptide
  N-term Hy
  C-term NH2
DTHFPIICI

SEQ ID NO: 472 - protein, 9 aa, Artificial Sequence
  Description of Artificial Sequence: Synthetic peptide
  N-term Hy
  C-term NH2
DTHICIAIF

SEQ ID NO: 473 - protein, 9 aa, Artificial Sequence
  Description of Artificial Sequence: Synthetic peptide
  N-term Hy
  C-term NH2
DTHCPIAIF

SEQ ID NO: 474 - protein, 9 aa, Artificial Sequence
  Description of Artificial Sequence: Synthetic peptide
  N-term Hy
  C-term NH2
ATHFPCIIF

SEQ ID NO: 475 - protein, 9 aa, Artificial Sequence
  Description of Artificial Sequence: Synthetic peptide
  N-term Hy
  C-term NH2
ADHFPCIIF

SEQ ID NO: 476 - protein, 11 aa, Artificial Sequence
  Description of Artificial Sequence: Synthetic peptide
  N-term Hy
  C-term NH2
DTHFPCIIFKC

SEQ ID NO: 477 - protein, 11 aa, Artificial Sequence
  Description of Artificial Sequence: Synthetic peptide
  N-term Hy
  C-term NH2
DTHFPCIIFAC

SEQ ID NO: 478 - protein, 11 aa, Artificial Sequence
  Description of Artificial Sequence: Synthetic peptide
  N-term Hy
  C-term NH2
DTHFPCIIFAA

SEQ ID NO: 479 - protein, 9 aa, Artificial Sequence
  Description of Artificial Sequence: Synthetic peptide
  N-term Hy
  C-term NH2
DEHFPCIIF

SEQ ID NO: 480 - protein, 9 aa, Artificial Sequence
  Description of Artificial Sequence: Synthetic peptide
  N-term Hy
  C-term NH2
DPHFPCIIF

SEQ ID NO: 481 - protein, 9 aa, Artificial Sequence
  Description of Artificial Sequence: Synthetic peptide
  N-term Hy
  C-term NH2
DTHKPCIIF

SEQ ID NO: 482 - protein, 9 aa, Artificial Sequence
  Description of Artificial Sequence: Synthetic peptide
  N-term Hy
  C-term NH2
DTHVPCIIF

SEQ ID NO: 483 - protein, 9 aa, Artificial Sequence
  Description of Artificial Sequence: Synthetic peptide
  N-term Hy
  C-term NH2
DTHFVCIIF

SEQ ID NO: 484 - protein, 9 aa, Artificial Sequence
  Description of Artificial Sequence: Synthetic peptide
  N-term Hy
  C-term NH2
DTHFPCIIY

SEQ ID NO: 485 - protein, 9 aa, Artificial Sequence
  Description of Artificial Sequence: Synthetic peptide
  N-term Hy
  C-term NH2
DTHFPCIIT

SEQ ID NO: 486 - protein, 9 aa, Artificial Sequence
  Description of Artificial Sequence: Synthetic peptide
  N-term Hy
  C-term NH2
DTHFPCILY

SEQ ID NO: 487 - protein, 9 aa, Artificial Sequence
  Description of Artificial Sequence: Synthetic peptide
  N-term Hy
  C-term NH2
DTHFPCIEY

SEQ ID NO: 488 - protein, 18 aa, Artificial Sequence
  Description of Artificial Sequence: Synthetic peptide
  N-term Isovaleric acid
  MOD_RES (16)..(16): N-Me-Trp
  C-term NH2
DTHFPCIIFGPRSKGWVC

SEQ ID NO: 489 - protein, 18 aa, Artificial Sequence
  Description of Artificial Sequence: Synthetic peptide
  N-term Isovaleric acid
  MOD_RES (10)..(10): Sarc
  MOD_RES (16)..(16): N-Me-Trp
  C-term NH2
DTHFPCIIFXPRSKGWVC

SEQ ID NO: 490 - protein, 18 aa, Artificial Sequence
  Description of Artificial Sequence: Synthetic peptide
  N-term Isovaleric acid
  MOD_RES (10)..(10): Sarc
  MOD_RES (16)..(16): N-Me-Trp
  C-term NH2
DTHFPCIIFXPHSKGWVC

SEQ ID NO: 491 - protein, 19 aa, Artificial Sequence
  Description of Artificial Sequence: Synthetic peptide
  N-term Isovaleric acid
  C-term NH2
DTHFPCIIFEPRSKHWVCK

SEQ ID NO: 492 - protein, 19 aa, Artificial Sequence
  Description of Artificial Sequence: Synthetic peptide
  N-term Isovaleric acid
  C-term NH2
DTHFPCIIFEPRSKEWVCK

SEQ ID NO: 493 - protein, 19 aa, Artificial Sequence
  Description of Artificial Sequence: Synthetic peptide
  N-term Isovaleric acid
  C-term NH2
DTHFPCIIFEPRSKLWVCK

SEQ ID NO: 494 - protein, 19 aa, Artificial Sequence
  Description of Artificial Sequence: Synthetic peptide
  N-term Isovaleric acid
  C-term NH2
DTHFPCIIFEPRSKFWVCK

SEQ ID NO: 495 - protein, 17 aa, Artificial Sequence
  Description of Artificial Sequence: Synthetic peptide
  N-term Isovaleric acid
  MOD_RES (15)..(15): Sarc
  C-term NH2
DTHFPCIKFEPHSKXCK

SEQ ID NO: 496 - protein, 19 aa, Artificial Sequence
  Description of Artificial Sequence: Synthetic peptide
  N-term Isovaleric acid
  C-term NH2
DTHFPCIKFKPHSKEWVCE

SEQ ID NO: 497 - protein, 19 aa, Artificial Sequence
  Description of Artificial Sequence: Synthetic peptide
  N-term Isovaleric acid
  C-term NH2
DTHFPCIKFEPRSKEWVCK

SEQ ID NO: 498 - protein, 19 aa, Artificial Sequence
  Description of Artificial Sequence: Synthetic peptide
  N-term Isovaleric acid
  C-term NH2
DTHFPCIKFEPRSKLWVCK

SEQ ID NO: 499 - protein, 19 aa, Artificial Sequence
  Description of Artificial Sequence: Synthetic peptide
  N-term Isovaleric acid
  C-term NH2
DTHFPCIKFEPRSKEWVCK

SEQ ID NO: 500 - protein, 17 aa, Artificial Sequence
  Description of Artificial Sequence: Synthetic peptide
  N-term Isovaleric acid
  MOD_RES (14)..(14): Lys(isoGlu-octanoic acid)
  C-term NH2
DTHFPCIKFEPRSKECK

SEQ ID NO: 501 - protein, 21 aa, Artificial Sequence
  Description of Artificial Sequence: Synthetic peptide
  N-term Hy
  C-term NH2
DTHFPCIIFGPRSKGWAVCYW

SEQ ID NO: 502 - protein, 20 aa, Artificial Sequence
  Description of Artificial Sequence: Synthetic peptide
  N-term Hy
  C-term NH2
DTHFPICIFGPHRSKGWVCM

SEQ ID NO: 503 - protein, 19 aa, Artificial Sequence
  Description of Artificial Sequence: Synthetic peptide
  N-term Hy
  C-term NH2
DTHFPCIIFGPRSKGWVAC

SEQ ID NO: 504 - protein, 19 aa, Artificial Sequence
  Description of Artificial Sequence: Synthetic peptide
  N-term Hy
  MOD_RES (6)..(6): D-Cys
  MOD_RES (19)..(19): D-Cys
  C-term NH2
DTHFPCIIFGPRSKGWVAC

SEQ ID NO: 505 - protein, 20 aa, Artificial Sequence
  Description of Artificial Sequence: Synthetic peptide
  N-term Hy
  C-term NH2
DTHFPCIIFGPRSKGWVACY

SEQ ID NO: 506 - protein, 19 aa, Artificial Sequence
  Description of Artificial Sequence: Synthetic peptide
  N-term Hy
  C-term NH2
DTHFPCIIFGPRSRGHVCK

SEQ ID NO: 507 - protein, 19 aa, Artificial Sequence
  Description of Artificial Sequence: Synthetic peptide
  N-term Hy
  C-term NH2
DTHFPCIIFGPRSKGWNCK

SEQ ID NO: 508 - protein, 19 aa, Artificial Sequence
  Description of Artificial Sequence: Synthetic peptide
  N-term Hy
  C-term NH2
DTHFPCINFGPRSKGWVCK

SEQ ID NO: 509 - protein, 19 aa, Artificial Sequence
  Description of Artificial Sequence: Synthetic peptide
  N-term Hy
  C-term NH2
DTHFPCIDFGPRSKGWVCK

SEQ ID NO: 510 - protein, 19 aa, Artificial Sequence
  Description of Artificial Sequence: Synthetic peptide
  N-term Isovaleric acid
  C-term NH2
DTHFECIIFGPRSKGWVCK

SEQ ID NO: 511 - protein, 20 aa, Artificial Sequence
  Description of Artificial Sequence: Synthetic peptide
  N-term Hy
  C-term NH2
DTHFPCIIFGGPRSRGWVCK

SEQ ID NO: 512 - protein, 20 aa, Artificial Sequence
  Description of Artificial Sequence: Synthetic peptide
  N-term Hy
  C-term NH2
DTHFPCIIFGGPRSKGWNCK

SEQ ID NO: 513 - protein, 20 aa, Artificial Sequence
  Description of Artificial Sequence: Synthetic peptide
  N-term Hy
  C-term NH2
DTHFPCIIFGGPRSKGWDCK

SEQ ID NO: 514 - protein, 17 aa, Artificial Sequence
  Description of Artificial Sequence: Synthetic peptide
  N-term Isovaleric acid
  C-term NH2
DTHFPCIFEPRSKGTCK

SEQ ID NO: 515 - protein, 9 aa, Artificial Sequence
  Description of Artificial Sequence: Synthetic peptide
  N-term Isovaleric acid
  C-term PEG3-Cys-NH2
DTHFPCIIF

SEQ ID NO: 516 - protein, 19 aa, Artificial Sequence
  Description of Artificial Sequence: Synthetic peptide
  N-term Isovaleric acid
  MOD_RES (10)..(10): Sarc
  C-term NH2
DTHAPCIKFXPRSKGWECK

SEQ ID NO: 517 - protein, 19 aa, Artificial Sequence
  Description of Artificial Sequence: Synthetic peptide
  N-term Isovaleric acid
  MOD_RES (15)..(15): Sarc
  C-term NH2
DTHAPCIKFEPRSKXWECK

SEQ ID NO: 518 - protein, 19 aa, Artificial Sequence
  Description of Artificial Sequence: Synthetic peptide
  N-term Isovaleric acid
  C-term NH2
DTHAPCIKFEPRSKEWECK

SEQ ID NO: 519 - protein, 19 aa, Artificial Sequence
  Description of Artificial Sequence: Synthetic peptide
  N-term Isovaleric acid
  C-term NH2
STHAPCIKFEPRSKGWECK

SEQ ID NO: 520 - protein, 19 aa, Artificial Sequence
  Description of Artificial Sequence: Synthetic peptide
  N-term Isovaleric acid
  C-term NH2
SKHAPCIKFEPRSKGWECK

SEQ ID NO: 521 - protein, 19 aa, Artificial Sequence
  Description of Artificial Sequence: Synthetic peptide
  N-term Isovaleric acid
  C-term NH2
DTHFPCIKFEPHSKEWVCK

SEQ ID NO: 522 - protein, 16 aa, Artificial Sequence
  Description of Artificial Sequence: Synthetic peptide
  N-term Isovaleric acid
  C-term NH2
DTAFPCIKFEPRSKEC

SEQ ID NO: 523 - protein, 19 aa, Artificial Sequence
  Description of Artificial Sequence: Synthetic peptide
  N-term Isovaleric acid
  C-term NH2
DTHFGCIKFEPRSKEWVCK

SEQ ID NO: 524 - protein, 19 aa, Artificial Sequence
  Description of Artificial Sequence: Synthetic peptide
  N-term Isovaleric acid
  C-term NH2
DTEFPCIKFEPRSKEWVCK

SEQ ID NO: 525 - protein, 19 aa, Artificial Sequence
  Description of Artificial Sequence: Synthetic peptide
  N-term Isovaleric acid
  MOD_RES (14)..(14): Lys(octanoic acid)
  C-term NH2
DTHFPCIKFEPRSKEWVCK

SEQ ID NO: 526 - protein, 19 aa, Artificial Sequence
  Description of Artificial Sequence: Synthetic peptide
  N-term Isovaleric acid
  C-term NH2
ETHFPCIKFEPRSKEWVCK

SEQ ID NO: 527 - protein, 9 aa, Artificial Sequence
  Description of Artificial Sequence: Synthetic peptide
  MOD_RES (1)..(1): pGlu
  C-term NH2
ETHFPCRKF

SEQ ID NO: 528 - protein, 8 aa, Artificial Sequence
  Description of Artificial Sequence: Synthetic peptide
  N-term Hy
  C-term NH2
DTHFPCLF

SEQ ID NO: 529 - protein, 10 aa, Artificial Sequence
  Description of Artificial Sequence: Synthetic peptide
  N-term Isovaleric acid
  MOD_RES (10)..(10): Lys(Palm)
  C-term NH2
DTHFPICIFK

SEQ ID NO: 530 - protein, 9 aa, Artificial Sequence
  Description of Artificial Sequence: Synthetic peptide
  N-term Isovaleric acid
  MOD_RES (8)..(8): Lys(Palm)
  C-term NH2
DTHFPCIKF

SEQ ID NO: 531 - protein, 11 aa, Artificial Sequence
  Description of Artificial Sequence: Synthetic peptide
  N-term Isovaleric acid
  C-term NH2
DTHFPCIKFAA

SEQ ID NO: 532 - protein, 9 aa, Artificial Sequence
  Description of Artificial Sequence: Synthetic peptide
  MOD_RES (1)..(1): D-Phe
  MOD_RES (2)..(2): D-Ile
  MOD_RES (3)..(3): D-Cys
  MOD_RES (4)..(4): D-Ile
  MOD_RES (5)..(5): D-Pro
  MOD_RES (6)..(6): D-Phe
  MOD_RES (7)..(7): D-His
  MOD_RES (8)..(8): D-Thr
  MOD_RES (9)..(9): D-Asp
FICIPFHTD

SEQ ID NO: 533 - protein, 9 aa, Artificial Sequence
  Description of Artificial Sequence: Synthetic peptide
  N-term Hy
  C-term NH2
DTHFPICIF

SEQ ID NO: 534 - protein, 9 aa, Artificial Sequence
  Description of Artificial Sequence: Synthetic peptide
  MOD_RES (1)..(1): Ida
  MOD_RES (4)..(4): Dpa
  MOD_RES (5)..(5): bhPro
  MOD_RES (9)..(9): bhPhe
  C-term PEG3-Palm
XTHXPRCRF

SEQ ID NO: 535 - protein, 19 aa, Artificial Sequence
  Description of Artificial Sequence: Synthetic peptide
  MOD_RES (19)..(19): Lys(PEG8)
  C-term NH2
DTHFPCIKFGPRSKGWVCK

SEQ ID NO: 536 - protein, 17 aa, Artificial Sequence
  Description of Artificial Sequence: Synthetic peptide
  MOD_RES (10)..(10): Lys(HO-isoGlu-Palm)
  C-term NH2
DTHFPCIKFKPRSKGCK

SEQ ID NO: 537 - protein, 9 aa, Artificial Sequence
  Description of Artificial Sequence: Synthetic peptide
  MOD_RES (8)..(8): Lys(beta-Ala-Palm)
  C-term NH2
DTHFPCIKF

SEQ ID NO: 538 - protein, 9 aa, Artificial Sequence
  Description of Artificial Sequence: Synthetic peptide
  C-term NH2
DTHFPCIIF

SEQ ID NO: 539 - protein, 9 aa, Artificial Sequence
  Description of Artificial Sequence: Synthetic peptide
  N-term Isobutyric acid
DTHFPCIKF

SEQ ID NO: 540 - protein, 9 aa, Artificial Sequence
  Description of Artificial Sequence: Synthetic peptide
  N-term Isovaleric acid
DTHFPCIKF

SEQ ID NO: 541 - protein, 9 aa, Artificial Sequence
  Description of Artificial Sequence: Synthetic peptide
  N-term Cyclohexanoic acid
DTHFPCIKF

SEQ ID NO: 542 - protein, 9 aa, Artificial Sequence
  Description of Artificial Sequence: Synthetic peptide
  N-term Isovaleric acid
DTHFPCIRF

SEQ ID NO: 543 - protein, 9 aa, Artificial Sequence
  Description of Artificial Sequence: Synthetic peptide
  N-term Isovaleric acid
DTHFPCIKF

SEQ ID NO: 544 - protein, 9 aa, Artificial Sequence
  Description of Artificial Sequence: Synthetic peptide
  N-term Isovaleric acid
DTHFPCIKF

SEQ ID NO: 545 - protein, 9 aa, Artificial Sequence
  Description of Artificial Sequence: Synthetic peptide
  N-term Hy
  C-term NH2
DTHFPICIF

SEQ ID NO: 546 - protein, 9 aa, Artificial Sequence
  Description of Artificial Sequence: Synthetic peptide
  N-term Hy
  MOD_RES (1)..(1): D-Phe
  MOD_RES (2)..(2): D-Ile
  MOD_RES (3)..(3): D-Cys
  MOD_RES (4)..(4): D-Ile
  MOD_RES (5)..(5): D-Pro
  MOD_RES (6)..(6): D-Phe
  MOD_RES (7)..(7): D-His
  MOD_RES (8)..(8): D-Thr
  MOD_RES (9)..(9): D-Asp
  C-term NH2
FICIPFHTD

SEQ ID NO: 547 - protein, 25 aa, Artificial Sequence
  Description of Artificial Sequence: Synthetic peptide
  N-term Hy
  C-term OH
DTHFPICIFCCGCCHRSKCGMCCKT

SEQ ID NO: 548 - protein, 9 aa, Artificial Sequence
  Description of Artificial Sequence: Synthetic peptide
  N-term Palm-PEG11
  MOD_RES (1)..(1): D-Phe
  MOD_RES (2)..(2): D-Ile
  MOD_RES (3)..(3): D-Cys
  MOD_RES (4)..(4): D-Ile
  MOD_RES (5)..(5): D-Pro
  MOD_RES (6)..(6): D-Phe
  MOD_RES (7)..(7): D-His
  MOD_RES (8)..(8): D-Thr
  MOD_RES (9)..(9): D-Asp
  C-term NH2
FICIPFHTD

SEQ ID NO: 549 - protein, 19 aa, Artificial Sequence
  Description of Artificial Sequence: Synthetic peptide
  C-term NH2
DTHFPICIFGPRSKGWVCM

SEQ ID NO: 550 - protein, 18 aa, Artificial Sequence
  Description of Artificial Sequence: Synthetic peptide
  MOD_RES (1)..(1): Ida
  MOD_RES (5)..(5): bhPro
  MOD_RES (9)..(9): bhPhe
  C-term NH2
XTHFPCRRFGPRSKGWVC

SEQ ID NO: 551 - protein, 18 aa, Artificial Sequence
  Description of Artificial Sequence: Synthetic peptide
  N-term Hy
  MOD_RES (1)..(1): bhAsp
  C-term NH2
DTHFPICIFGPRSKGWVC

SEQ ID NO: 552 - protein, 18 aa, Artificial Sequence
  Description of Artificial Sequence: Synthetic peptide
  N-term Hy
  MOD_RES (1)..(1): bhAsp
  MOD_RES (4)..(4): N-Me-Phe
  C-term NH2
DTHFPICIFGPRSKGWVC

SEQ ID NO: 553 - protein, 19 aa, Artificial Sequence
  Description of Artificial Sequence: Synthetic peptide
  N-term Ac
  MOD_RES (10)..(10): Lys(m-PEG8)
  C-term NH2
DTHFPICIFKPRSKGWVCM

SEQ ID NO: 554 - protein, 19 aa, Artificial Sequence
  Description of Artificial Sequence: Synthetic peptide
  N-term Ac
  MOD_RES (14)..(14): Lys(m-PEG8)
  C-term NH2
DTHFPICIFGPRSKGWVCM

SEQ ID NO: 555 - protein, 19 aa, Artificial Sequence
  Description of Artificial Sequence: Synthetic peptide
  MOD_RES (1)..(1): Ida
  MOD_RES (4)..(4): Dpa
  MOD_RES (5)..(5): bhPro
  C-term NH2
XTHXPCIIFGPRSRGWVCK

SEQ ID NO: 556 - protein, 19 aa, Artificial Sequence
  Description of Artificial Sequence: Synthetic peptide
  N-term Hy
  C-term NH2
IPFIDTCFHGPRSRGWVCK

SEQ ID NO: 557 - protein, 18 aa, Artificial Sequence
  Description of Artificial Sequence: Synthetic peptide
  N-term Isovaleric acid
  C-term NH2
DTHFPCIIFGPRSKGVCK

SEQ ID NO: 558 - protein, 17 aa, Artificial Sequence
  Description of Artificial Sequence: Synthetic peptide
  N-term Isovaleric acid
  C-term NH2
DTHFPCIFGPRSKGWCK

SEQ ID NO: 559 - protein, 9 aa, Artificial Sequence
  Description of Artificial Sequence: Synthetic peptide
  N-term Isovaleric acid
  C-term NH2
DTHFPCIIF

SEQ ID NO: 560 - protein, 18 aa, Artificial Sequence
  Description of Artificial Sequence: Synthetic peptide
  N-term Isovaleric acid
  MOD_RES (14)..(14): Lys(m-PEG8)
  C-term NH2
DTHFPICIFGPRSKGWVC

SEQ ID NO: 561 - protein, 18 aa, Artificial Sequence
  Description of Artificial Sequence: Synthetic peptide
  N-term Isovaleric acid
  MOD_RES (14)..(14): Lys(m-PEG4)
  C-term NH2
DTHFPICIFGPRSKGWVC

SEQ ID NO: 562 - protein, 19 aa, Artificial Sequence
  Description of Artificial Sequence: Synthetic peptide
  N-term Isovaleric acid
  C-term NH2
DTHFPCIIFGPRSRGWVCK

SEQ ID NO: 563 - protein, 18 aa, Artificial Sequence
  Description of Artificial Sequence: Synthetic peptide
  N-term Isovaleric acid
  MOD_RES (16)..(16): N-Me-Trp
  C-term NH2
DTHFPICIFGPRSKGWVC

SEQ ID NO: 564 - protein, 18 aa, Artificial Sequence
  Description of Artificial Sequence: Synthetic peptide
  N-term Isovaleric acid
  MOD_RES (10)..(10): Sarc
  MOD_RES (16)..(16): N-Me-Trp
  C-term NH2
DTHFPICIFXPRSKGWVC

SEQ ID NO: 565 - protein, 18 aa, Artificial Sequence
  Description of Artificial Sequence: Synthetic peptide
  N-term Isovaleric acid
  MOD_RES (10)..(10): Sarc
  MOD_RES (16)..(16): N-Me-Trp
  C-term NH2
DTHFPICIFXPHSKGWVC

SEQ ID NO: 566 - protein, 19 aa, Artificial Sequence
  Description of Artificial Sequence: Synthetic peptide
  N-term Isovaleric acid
  C-term NH2
DTHFPCIIFEPRSKKWVCK

SEQ ID NO: 567 - protein, 9 aa, Artificial Sequence
  Description of Artificial Sequence: Synthetic peptide
  N-term Isovaleric acid
  C-term PEG3-Cys-NH2
DTHFPCIIF

SEQ ID NO: 568 - protein, 9 aa, Artificial Sequence
  Description of Artificial Sequence: Synthetic peptide
  C-term NH2
DTHFPCIIF

SEQ ID NO: 569 - protein, 17 aa, Artificial Sequence
  Description of Artificial Sequence: Synthetic peptide
  N-term Isovaleric acid
  MOD_RES (10)..(10): Lys(Dapa-Palm)
  C-term NH2
DTHFPCIKFKPRSKGCK

SEQ ID NO: 570 - protein, 9 aa, Artificial Sequence
  Description of Artificial Sequence: Synthetic peptide
  N-term Isovaleric acid
DTHFPCIKF

SEQ ID NO: 571 - protein, 10 aa, Artificial Sequence
  Description of Artificial Sequence: Synthetic peptide
  MOD_RES (1)..(1): Asp, Glu, Ida, pGlu, bhAsp or absent
  MOD_RES (4)..(4): Phe or Dpa
  MOD_RES (5)..(5): Pro or bhPro
  MOD_RES (8)..(8): Ile, Lys, Glu, Phe, Gln or Arg
  MOD_RES (10)..(10): Lys or absent
XTHXPCIXFX

SEQ ID NO: 572 - protein, 9 aa, Artificial Sequence
  Description of Artificial Sequence: Synthetic peptide
  MOD_RES (1)..(1): Asp, Glu or Ida
  MOD_RES (5)..(5): Pro or bhPro
  MOD_RES (8)..(8): Ile, Lys or Phe
XTHFPCIXF

SEQ ID NO: 573 - protein, 10 aa, Artificial Sequence
  Description of Artificial Sequence: Synthetic peptide
  MOD_RES (1)..(1): Gly, Ala, Lys, Pro or D-Pro
  MOD_RES (2)..(2): Pro, Ala or Gly
  MOD_RES (3)..(3): Arg, Ala, Lys or Trp
  MOD_RES (4)..(4): Ser, Gly or Ala
  MOD_RES (5)..(5): Lys, Met, Arg or Ala
  MOD_RES (6)..(6): Gly, Arg or Ala
  MOD_RES (7)..(7): Trp, Ala or absent
  MOD_RES (8)..(8): Val, Thr, Lys, Ala, Glu or absent
  MOD_RES (10)..(10): Met, Lys or absent
XXXXXXXXCX

SEQ ID NO: 574 - protein, 10 aa, Artificial Sequence
  Description of Artificial Sequence: Synthetic peptide
  MOD_RES (1)..(1): Gly, Pro or D-Pro
  MOD_RES (2)..(2): Pro or Gly
  MOD_RES (3)..(3): Arg or Lys
  MOD_RES (8)..(8): Val or Thr
  MOD_RES (10)..(10): Met, Lys or absent
XXXSKGWXCX

SEQ ID NO: 575 - protein, 15 aa, Artificial Sequence
  Description of Artificial Sequence: Synthetic peptide
  MOD_RES (1)..(1): Val, Ala or absent
  MOD_RES (3)..(3): Gly, Pro or absent
  MOD_RES (4)..(4): His, Trp or Tyr
  MOD_RES (6)..(6): Ser, Gly or Pro
  MOD_RES (7)..(7): Ile, Gly or Lys
  MOD_RES (8)..(8): Gly, Met or absent
  MOD_RES (10)..(10): Tyr or Cys
  MOD_RES (11)..(11): Arg, Lys, Met or Ala
  MOD_RES (12)..(12): Arg or Ala
  MOD_RES (13)..(13): Cys, Val or absent
  MOD_RES (14)..(14): Cys, Lys, Pro, Arg, Thr or absent
  MOD_RES (15)..(15): Arg, Thr or absent
XCXXRXXXCXXXXXX

SEQ ID NO: 576 - protein, 10 aa, Artificial Sequence
  Description of Artificial Sequence: Synthetic peptide
  MOD_RES (1)..(1): Gly, Glu, Val or Lys
  MOD_RES (3)..(3): Arg or Lys
  MOD_RES (5)..(5): Arg or Lys
  MOD_RES (6)..(6): Gly, Ser, Lys, Ile or Arg
  MOD_RES (7)..(7): Trp or absent
  MOD_RES (8)..(8): Val, Thr, Asp, Glu or absent
  MOD_RES (10)..(10): Lys or absent
XPXSXXXXCX

SEQ ID NO: 577 - protein, 10 aa, Artificial Sequence
  Description of Artificial Sequence: Synthetic peptide
  MOD_RES (1)..(1): Glu or Lys
  MOD_RES (3)..(3): Arg or Lys
  MOD_RES (5)..(5): Arg or Lys
  MOD_RES (6)..(6): Gly, Ser, Lys, Ile or Arg
  MOD_RES (7)..(7): Trp or absent
  MOD_RES (8)..(8): Val or absent
  MOD_RES (10)..(10): Lys or absent
XPXSXXXXCX

SEQ ID NO: 578 - protein, 10 aa, Artificial Sequence
  Description of Artificial Sequence: Synthetic peptide
  MOD_RES (1)..(1): Asp, Ida, pGlu, bhAsp or absent
  MOD_RES (4)..(4): Phe or Dpa
  MOD_RES (5)..(5): Pro or bhPro
  MOD_RES (6)..(6): Ile, Cys or Arg
  MOD_RES (7)..(7): Cys, Ile, Leu or Val
  MOD_RES (8)..(8): Ile, Lys, Glu, Phe, Gln or Arg
  MOD_RES (10)..(10): Lys or absent
XTHXPXXXFX

SEQ ID NO: 579 - protein, 10 aa, Artificial Sequence
  Description of Artificial Sequence: Synthetic polypeptide
  MOD_RES (1)..(1): Asp, Ida, pGlu, bhAsp or absent
  MOD_RES (4)..(4): Phe or Dpa
  MOD_RES (5)..(5): Pro or bhPro
  MOD_RES (8)..(8): Ile, Lys, Glu, Phe, Gln or Arg
  MOD_RES (10)..(10): Lys or absent
XTHXPCIXFX

SEQ ID NO: 580 - protein, 9 aa, Artificial Sequence
  Description of Artificial Sequence: Synthetic polypeptide
  MOD_RES (1)..(1): Asp, Glu or Ida
  MOD_RES (5)..(5): Pro or bhPro
  MOD_RES (8)..(8): Ile, Lys or Phe
XTHFPCIXF

SEQ ID NO: 581 - protein, 10 aa, Artificial Sequence
  Description of Artificial Sequence: Synthetic peptide
  MOD_RES (1)..(1): Gly, Ala, Lys, Pro or D-Pro
  MOD_RES (2)..(2): Pro, Ala or Gly
  MOD_RES (3)..(3): Arg, Ala, Lys or Trp
  MOD_RES (4)..(4): Ser, Gly or Ala
  MOD_RES (5)..(5): Lys, Met, Arg or Ala
  MOD_RES (6)..(6): Gly, Arg or Ala
  MOD_RES (7)..(7): Trp or Ala
  MOD_RES (8)..(8): Val, Thr, Ala or Glu
  MOD_RES (10)..(10): Met, Lys or absent
XXXXXXXXCX

SEQ ID NO: 582 - protein, 10 aa, Artificial Sequence
  Description of Artificial Sequence: Synthetic peptide
  MOD_RES (1)..(1): Gly, Pro or D-Pro
  MOD_RES (2)..(2): Pro or Gly
  MOD_RES (3)..(3): Arg or Lys
  MOD_RES (8)..(8): Val or Thr
  MOD_RES (10)..(10): Met, Lys or absent
XXXSKGWXCX

SEQ ID NO: 583 - protein, 15 aa, Artificial Sequence
  Description of Artificial Sequence: Synthetic peptide
  MOD_RES (1)..(1): Val, Ala or absent
  MOD_RES (3)..(3): Gly, Pro or absent
  MOD_RES (4)..(4): His, Trp or Tyr
  MOD_RES (6)..(6): Ser, Gly or Pro
  MOD_RES (7)..(7): Ile, Gly or Lys
  MOD_RES (8)..(8): Gly, Met or absent
  MOD_RES (10)..(10): Tyr or Cys
  MOD_RES (11)..(11): Arg, Lys, Met or Ala
  MOD_RES (12)..(12): Arg or Ala
  MOD_RES (13)..(13): Cys, Val or absent
  MOD_RES (14)..(14): Cys, Lys, Pro, Arg, Thr or absent
  MOD_RES (15)..(15): Arg, Thr or absent
XCXXRXXXCXXXXXX

SEQ ID NO: 584 - protein, 10 aa, Artificial Sequence
  Description of Artificial Sequence: Synthetic peptide
  MOD_RES (1)..(1): Asp, Ida, pGlu, bhAsp or absent
  MOD_RES (4)..(4): Phe or Dpa
  MOD_RES (5)..(5): Pro or bhPro
  MOD_RES (6)..(6): Ile, Cys or Arg
  MOD_RES (7)..(7): Cys, Ile, Leu or Val
  MOD_RES (8)..(8): Ile, Lys, Glu, Phe, Gln or Arg
  MOD_RES (10)..(10): Lys, Phe or absent
XTHXPXXXFX

SEQ ID NO: 585 - protein, 10 aa, Artificial Sequence
  Description of Artificial Sequence: Synthetic peptide
  MOD_RES (1)..(1): Asp, Ida, pGlu, bhAsp or absent
  MOD_RES (4)..(4): Phe or Dpa
  MOD_RES (5)..(5): Pro or bhPro
  MOD_RES (8)..(8): Ile, Lys, Glu, Phe, Gln or Arg
  MOD_RES (10)..(10): Lys or absent
XTHXPCIXFX

SEQ ID NO: 586 - protein, 9 aa, Artificial Sequence
  Description of Artificial Sequence: Synthetic peptide
  MOD_RES (1)..(1): Asp, Glu or Ida
  MOD_RES (5)..(5): Pro or bhPro
  MOD_RES (8)..(8): Ile, Lys or Phe
XTHFPCIXF

SEQ ID NO: 587 - protein, 10 aa, Artificial Sequence
  Description of Artificial Sequence: Synthetic peptide
  MOD_RES (1)..(1): Gly, Ala, Lys, Pro or D-Pro
  MOD_RES (2)..(2): Pro, Ala or Gly
  MOD_RES (3)..(3): Arg, Ala, Lys or Trp
  MOD_RES (4)..(4): Ser, Gly or Ala
  MOD_RES (5)..(5): Lys, Met, Arg or Ala
  MOD_RES (6)..(6): Gly, Arg or Ala
  MOD_RES (7)..(7): Trp, Ala or absent
  MOD_RES (8)..(8): Val, Thr, Lys, Ala, Glu or absent
  MOD_RES (10)..(10): Met, Lys or absent
XXXXXXXXCX

SEQ ID NO: 588 - protein, 10 aa, Artificial Sequence
  Description of Artificial Sequence: Synthetic peptide
  MOD_RES (1)..(1): Gly, Pro or D-Pro
  MOD_RES (2)..(2): Pro or Gly
  MOD_RES (3)..(3): Arg or Lys
  MOD_RES (8)..(8): Val or Thr
  MOD_RES (10)..(10): Met, Lys or absent
XXXSKGWXCX

SEQ ID NO: 589 - protein, 8 aa, Artificial Sequence
  Description of Artificial Sequence: Synthetic peptide
DTHFPICI

SEQ ID NO: 590 - protein, 6 aa, Artificial Sequence
  Description of Artificial Sequence: Synthetic peptide
  MOD_RES (3)..(3): Any amino acid
DTXFPC

SEQ ID NO: 591 - protein, 9 aa, Artificial Sequence
  Description of Artificial Sequence: Synthetic peptide
  MOD_RES (3)..(3): Any amino acid
  MOD_RES (7)..(7): Any amino acid
  MOD_RES (8)..(8): Any amino acid or absent
DTXFPCXXF

SEQ ID NO: 592 - protein, 9 aa, Artificial Sequence
  Description of Artificial Sequence: Synthetic peptide
  MOD_RES (1)..(3): Any amino acid or absent
  MOD_RES (5)..(5): Any amino acid
  MOD_RES (7)..(7): Any amino acid except for D-Cys, D-Ser, D-Ala, Cys(S-tBut), homoC, Pen, (D)Pen, Dap(AcBr), Inp or D-His
  MOD_RES (8)..(8): Any amino acid
XXXFXCXXF

SEQ ID NO: 593 - protein, 9 aa, Artificial Sequence
  Description of Artificial Sequence: Synthetic peptide
  MOD_RES (3)..(3): Any amino acid
  MOD_RES (5)..(5): Any amino acid or absent
  MOD_RES (7)..(7): Any amino acid except for D-Cys, D-Ser, D-Ala, Cys(S-tBut), homoC, Pen, (D)Pen, Dap(AcBr), Inp or D-His
  MOD_RES (8)..(8): Any amino acid
DTXFXCXXF

SEQ ID NO: 594 - protein, 7 aa, Artificial Sequence
  Description of Artificial Sequence: Synthetic peptide
  MOD_RES (3)..(3): Any amino acid
  MOD_RES (6)..(6): Any amino acid
DTXFPXC

SEQ ID NO: 595 - protein, 9 aa, Artificial Sequence
  Description of Artificial Sequence: Synthetic peptide
  MOD_RES (3)..(3): Any amino acid
  MOD_RES (6)..(6): Any amino acid
  MOD_RES (8)..(8): Any amino acid or absent
DTXFPXCXF

SEQ ID NO: 596 - protein, 9 aa, Artificial Sequence
  Description of Artificial Sequence: Synthetic peptide
  MOD_RES (1)..(3): Any amino acid or absent
  MOD_RES (5)..(6): Any amino acid
  MOD_RES (8)..(8): Any amino acid
XXXFXXCXF

SEQ ID NO: 597 - protein, 9 aa, Artificial Sequence
  Description of Artificial Sequence: Synthetic peptide
  MOD_RES (3)..(3): Any amino acid
  MOD_RES (5)..(5): Any amino acid or absent
  MOD_RES (6)..(6): Any amino acid
  MOD_RES (8)..(8): Any amino acid
DTXFXXCXF

SEQ ID NO: 598 - protein, 9 aa, Artificial Sequence
  Description of Artificial Sequence: Synthetic peptide
  MOD_RES (1)..(3): Any amino acid or absent
  MOD_RES (5)..(6): Any amino acid
  MOD_RES (8)..(8): Any amino acid
XXXFXXCXF

SEQ ID NO: 599 - protein, 9 aa, Artificial Sequence
  Description of Artificial Sequence: Synthetic peptide
  MOD_RES (3)..(3): Any amino acid
  MOD_RES (5)..(6): Any amino acid
  MOD_RES (8)..(8): Any amino acid
DTXFXXCXF

SEQ ID NO: 600 - protein, 27 aa, Artificial Sequence
  Description of Artificial Sequence: Synthetic peptide
GYIPEAPRDGQAYVRKDGEWVLLSTFL

SEQ ID NO: 601 - protein, 19 aa, Artificial Sequence
  Description of Artificial Sequence: Synthetic peptide
  N-term Isovaleric acid
  MOD_RES (19)..(19): Lys(m-PEG8)
  C-term NH2
DTHFPCIKFGPRSKGWVCK

SEQ ID NO: 602 - protein, 9 aa, Artificial Sequence
  Description of Artificial Sequence: Synthetic peptide
  N-term Isovaleric acid
  MOD_RES (1)..(1): Gly, Sarc, Lys, Glu or absent
  MOD_RES (2)..(2): Pro, Ala, Gly or absent
  MOD_RES (3)..(3): Arg, Lys, Pro, Gly, His, Ala or absent
  MOD_RES (4)..(4): Ser, Arg, Glu or absent
  MOD_RES (5)..(5): Lys, Ser, Met, Arg, Ala or absent
  MOD_RES (6)..(6): Gly, Sarc, Glu, Leu, Phe, His or absent
  MOD_RES (7)..(7): Trp, N-Me-Trp, Lys, Thr, His, Gly, Ala, Ile, Val or absent
  MOD_RES (8)..(8): Val, Trp, Ala, Asn, Glu or absent
XXXXXXXXC

SEQ ID NO: 603 - protein, 15 aa, Artificial Sequence
  Description of Artificial Sequence: Synthetic polypeptide
  MOD_RES (3)..(3): Gly or absent
  MOD_RES (6)..(6): Ser or Pro
  MOD_RES (7)..(7): Ile or Lys
  MOD_RES (8)..(8): Gly or absent
  MOD_RES (12)..(12): Arg or Ala
  MOD_RES (13)..(13): Cys, Val or absent
  MOD_RES (14)..(14): Cys, Arg, Thr or absent
  MOD_RES (15)..(15): Arg or absent
VCXHRXXXCYRXXXX

SEQ ID NO: 604 - protein, 15 aa, Artificial Sequence
  Description of Artificial Sequence: Synthetic polypeptide
  MOD_RES (3)..(3): Gly or absent
  MOD_RES (6)..(6): Ser or Pro
  MOD_RES (7)..(7): Ile or Lys
  MOD_RES (8)..(8): Gly or absent
  MOD_RES (12)..(12): Arg or Ala
  MOD_RES (13)..(13): Cys, Val or absent
  MOD_RES (14)..(14): Cys, Arg, Thr or absent
  MOD_RES (15)..(15): Arg or absent
VCXHRXXXCYRXXXX

SEQ ID NO: 605 - protein, 10 aa, Artificial Sequence
  Description of Artificial Sequence: Synthetic peptide
  MOD_RES (1)..(1): Gly, Glu, Val or Lys
  MOD_RES (3)..(3): Arg or Lys
  MOD_RES (5)..(5): Arg or Lys
  MOD_RES (6)..(6): Gly, Ser, Lys, Ile or Arg
  MOD_RES (7)..(7): Trp or absent
  MOD_RES (8)..(8): Val, Thr, Asp, Glu or absent
  MOD_RES (10)..(10): Lys or absent
XPXSXXXXCX

SEQ ID NO: 606 - protein, 10 aa, Artificial Sequence
  Description of Artificial Sequence: Synthetic peptide
  MOD_RES (1)..(1): Glu or Lys
  MOD_RES (3)..(3): Arg or Lys
  MOD_RES (5)..(5): Arg or Lys
  MOD_RES (6)..(6): Gly, Ser, Lys, Ile or Arg
  MOD_RES (7)..(7): Trp or absent
  MOD_RES (8)..(8): Val or absent
  MOD_RES (10)..(10): Lys or absent
XPXSXXXXCX